(19) United States
(12) Patent Application Publication
(10) Pub. No.: US 2025/0327055 A1
(43) Pub. Date: Oct. 23, 2025

(54) ADENINE DEAMINASES AND COMPOSITIONS, SYSTEMS, AND METHODS THEREOF

(71) Applicant: Profluent Bio Inc., Emeryville, CA (US)

(72) Inventors: Ali Madani, Emeryville, CA (US); Jeffrey A. Ruffolo, Emeryville, CA (US); Stephen Nayfach, Emeryville, CA (US); Joel Beazer, Emeryville, CA (US)

(21) Appl. No.: 19/255,359

(22) Filed: Jun. 30, 2025

Related U.S. Application Data
(63) Continuation of application No. PCT/US2024/056236, filed on Nov. 15, 2024.
(60) Provisional application No. 63/599,141, filed on Nov. 15, 2023.

(51) Int. Cl. C12N 9/78; C12N 9/22; C12N 15/11; C12N 15/90

(52) U.S. Cl. CPC C12N 9/78; C12N 9/226; C12N 15/11; C12N 15/907; C12Y 305/04004; C07K 2319/00; C12N 2310/20

(57) ABSTRACT

The present disclosure provides components, compositions, methods, and systems thereof for nucleic acid editing. Particularly, the invention relates to adenine deaminases, fusion proteins of the adenine deaminases, systems including the adenine deaminases, and methods of using thereof.

### CROSS-REFERENCE TO RELATED APPLICATIONS

This application is a continuation of PCT International Application No. PCT/US2024/056236, filed Nov. 15, 2024, which claims the benefit of U.S. Provisional Application No. 63/599,141, filed Nov. 15, 2023, the contents of each are herein incorporated by reference in their entirety.

### FIELD

The present disclosure relates to components, compositions, methods, and systems thereof for nucleic acid editing. Particularly, the disclosure relates to adenine deaminases, fusion proteins of the adenine deaminases, systems including the adenine deaminases, and methods of using thereof.

### SEQUENCE LISTING STATEMENT

The content of the electronic sequence listing titled PROF_42422_601_SequenceListing.xml (Size: 817,420 bytes; and Date of Creation: Nov. 14, 2024) is herein incorporated by reference in its entirety.

### BACKGROUND

Methods for precisely and efficiently editing nucleic acid sequences, particularly in vivo, are challenging to develop but when successful enable studies of gene function and open doors to new therapies for human genetic diseases. Theoretically, genetic diseases can be treated by altering nucleic acid sequences as specific locations in the genome, even a single nucleotide alteration from T to C or A to G can affect gene product expression and function resulting in a change in disease state. Deaminases, enzymes utilized in metabolic and salvage pathways, can be harnessed to facilitate single nucleotide modifications of a nucleic acid. For example, cytidine deaminases can ultimately result in conversion of C-G base pairs to T-A base pairs whereas adenosine deaminases can support conversion of A-T base pairs to G-C base pairs. However, there is a continuing need to expand the available deaminases which are efficient, precise, and suitable for use in genetic engineering methods and therapies, particularly in eukaryotic cells and organisms.

### SUMMARY

Provided herein are polypeptides comprising an adenosine deaminase having an amino acid sequence with at least 75% identity to any of SEQ ID NOs: 1-23. In some embodiments, the adenosine deaminase has an amino acid sequence of any of SEQ ID NO: 1-23. In some embodiments, the adenosine deaminase has an amino acid sequence with at least 75% identity to any of SEQ ID NOs: 24-776. In some embodiments, the adenosine deaminase has an amino acid sequence of any of SEQ ID NO: 24-776.

Also provided herein are fusion proteins comprising a polypeptide or deaminase disclosed herein and a nucleic acid binding domain.

In some embodiments, the nucleic acid binding domain comprises a programmable nucleic acid binding domain.

In some embodiments, the nucleic acid binding domain is a Clustered Regularly Interspaced Short Palindromic Repeats associated (Cas) protein or a fragment or variant thereof capable of nucleic acid binding. In some embodiments, the Cas protein is at least partially catalytically inactivated. In some embodiments, the Cas protein is catalytically inactivated Cas9.

In some embodiments, the fusion proteins further comprise a linker separating the polypeptide and the nucleic acid binding domain.

In some embodiments, the fusion proteins further comprise at least one nuclear localization sequence.

Further provided are nucleic acids encoding a polypeptide, deaminase, or fusion protein disclosed herein and vectors comprising the nucleic acid.

Additionally provided are systems comprising a polypeptide or deaminase disclosed herein and a nucleic acid binding polypeptide. In some embodiments, the polypeptide or deaminase and the nucleic acid binding polypeptide are fused as a single protein. In some embodiments, the polypeptide or deaminase is linked to a first half of a binding pair and the nucleic acid binding polypeptide is linked to a second half of the binding pair.

In some embodiments, the nucleic acid binding domain comprises a programmable nucleic acid binding domain.

In some embodiments, the nucleic acid binding polypeptide is a Cas protein. In some embodiments, the Cas protein is at least partially catalytically inactivated. In some embodiments, the Cas protein is catalytically inactivated Cas9.

In some embodiments, the systems further comprise at least one guide RNA. In some embodiments, at least one gRNA is complexed with the Cas protein.

Compositions and cells comprising a nucleic acid binding polypeptide or deaminase, a fusion protein, a nucleic acid, a vector, or a system as disclosed herein are also provided.

In some embodiments, the cell is a prokaryotic cell or a eukaryotic cell. In some embodiments, the cell is a mammalian cell. In some embodiments, the cell is a human cell.

Methods of modifying a target nucleic acid are likewise provided. In some embodiments, the methods comprise contacting the target nucleic acid with a polypeptide or deaminase, a fusion protein, a nucleic acid, a vector, or a system as disclosed herein. In some embodiments, the target nucleic acid is DNA. In some embodiments, the target nucleic acid is RNA.

In some embodiments, the target nucleic acid is associated with a disease or disorder. In some embodiments, the disease or disorder is associated with a point mutation in the target nucleic acid.

In some embodiments, the target nucleic acid encodes a gene product.

In some embodiments, the target nucleic acid is in a cell. In some embodiments, the contacting comprises introducing into the cell. In some embodiments, the cell is in vitro or ex vivo. In some embodiments, the cell is in vivo. In some embodiments, the introducing comprises administering to a subject.

In some embodiments, the cell is in a plant. In some embodiments, the method comprises administering to a plant, plant cell, seed, fruit, plant part, or propagation material of a plant the polypeptide, fusion protein, nucleic acid, vector, or system.

In some embodiments, the methods treat a disease or disorder in a subject. In some embodiments, the methods comprise administering to the subject in need thereof an effective amount of a polypeptide or deaminase, a fusion protein, a nucleic acid, a vector, or a system as disclosed herein. In some embodiments, the subject is a human.

In some embodiments, the target nucleic acid encodes a gene product. In some embodiments, the target nucleic acid is a disease-associated gene. In some embodiments, the disease-associated gene is associated with a point mutation, single nucleotide variant (SNV), or single nucleotide polymorphism (SNP).

Other aspects and embodiments of the disclosure will be apparent in light of the following detailed description.

### BRIEF DESCRIPTION OF THE DRAWINGS

FIG. 1 is a graph of the A to G editing efficiency of deaminase enzymes linked to Cas9n as disclosed herein (SEQ ID NOs: 1-23, as indicated).

FIG. 2 is a graph showing the specificity of the deaminase enzymes linked to Cas9n on the target strand as disclosed herein (SEQ ID NOs: 1-23, as indicated). Efficiency of nucleotide changes at each position on the target strand, including at the target site, are shown.

FIG. 3 is graphs showing the A to G editing efficiency of deaminase enzymes as disclosed herein (SEQ ID NOs: 1-2, as indicated) at three different target sites (HEK2, T39, and CD3G1) using when linked to two different nucleases, OpenCRISPR-1 (Ruffolo, et al., bioRxiv 2024.04.22.590591) and spCas9, both partially catalytically inactivated (nickases).

FIG. 4 is graphs of the A to G editing efficiency of deaminase enzymes as disclosed herein (SEQ ID NOs: 1-23 and 574-776, as indicated) when tethered to spCas9n.

FIG. 5 is graphs of the A to G editing efficiency of deaminase enzymes, as indicated, when tether to spCas9n. The editing efficiency was determined by NGS analysis with CRISPResso.

### DETAILED DESCRIPTION

The disclosed polypeptides, compositions, systems, kits, and methods include deaminases useful for nucleic acid modification.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, 793-800 (Worth Pub. 1982)). The present technology contemplates any deoxyribonucleotide, ribonucleotide, or nucleoprotein component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (sec Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, “peptide,” “polypeptide,” or “protein” refer to a sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide,” “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The term “amino acid” or “any amino acid” as used here refers to any and all amino acids, including naturally occurring amino acids (e.g., a-amino acids), unnatural amino acids, modified amino acids, and non-natural amino acids. It includes both D-and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is retained by the peptide.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-60 (2005), Altschul et al., Nucleic Acids Res., 25(17): 3389-3402 (1997), and Gusfield, Algorithms on Strings, Trees and Sequences, Cambridge University Press, Cambridge UK (1997)).

The term “gene” refers to a nucleic acid sequence that comprises control and coding sequences necessary for the production of a gene product (e.g., an RNA having a non-coding function (e.g., a ribosomal or transfer RNA), a polypeptide, or a precursor of any of the foregoing). The RNA or polypeptide can be encoded by a full-length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which it is naturally associated in nature and as found in nature, and/or the nucleic acid molecule or the polypeptide is associated with at least one other component with which it is not naturally associated in nature and/or that there is one or more changes in nucleic acid or amino acid sequence as compared with such sequence as it is found in nature and/or that the nucleic acid or polypeptide sequence was generated de novo, e.g., not based on or derived from any naturally occurring sequence.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “contacting” as used herein refers to bring or put in contact, to be in or come into contact. The term “contact” as used herein refers to a state or condition of touching or of immediate or local proximity.

As used herein, the terms “providing,” “administering,” and “introducing,” are used interchangeably herein and refer to the placement of the composition or systems of the disclosure into a cell, organism, or subject by a method or route which results in at least partial localization to a desired site. The composition or systems can be administered by any appropriate route which results in delivery to a desired location in the cell, organism, or subject.

A “subject” or “patient” may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents such as rats, mice, and guinea pigs, and the like. Examples of non-mammals include, but are not limited to, birds, fish, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Deaminases

Disclosed herein are synthetic deaminases. A deaminase catalyzes removal of an amino group from a compound or molecule (e.g., a nucleic acid/nucleotide or protein/amino acid). In some embodiments, the deaminase is an adenosine deaminase, also sometimes referred to as an adenine deaminase. Adenosine deaminases catalyze the deamination of adenosine and deoxyadenosine to inosine and deoxyinosine, respectively. Accordingly, with repair and replication mechanisms adenosine deaminase can ultimately lead to the conversion of an A:T base pair to a G:C base pair.

In some embodiments, the deaminases comprise an amino acid sequence having at least 75% identity (e.g., at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to any one of SEQ ID NOs: 1-23. In some embodiments, the deaminases comprise an amino acid sequence having at least 70% identity to any one of SEQ ID NOs: 24-776. In some embodiments, the deaminases comprise an amino acid sequence having at least 70% identity to any one of SEQ ID NOs: 1-776. In some embodiments, the deaminases comprise an amino acid sequence having any one of SEQ ID NOs: 1-776.

Any of the deaminases described herein may comprise one or more (e.g., 1, 2, 3, 4, 5, 6, 7, 8, 9, 10, 15, 20, 25, or more, etc.) amino acid substitutions as compared to SEQ ID NOs: 1-776. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of aromatic amino acids include histidine (H or His), phenylalanine (F or Phe), tyrosine (Y or Tyr), and tryptophan (W or Trp). Non-aromatic amino acids are broadly grouped as aliphatic. Examples of aliphatic amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other and therefore resemble each other most in their impact on the overall protein structure (Schulz and Schirmer). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

### Fusion Proteins

The present disclosure also provides fusion proteins comprising one or more of the deaminases fused to a nucleic acid binding domain. The fusion proteins are not limited by orientation or directionality of the deaminase and the nucleic acid binding domain. For example, the nucleic acid binding domain may be fused to the N-terminus or C-terminus of the deaminase, in any orientation, e.g., N-terminus to N-terminus, C-terminus to C-terminus, N-terminus to C-terminus, or C-terminus to N-terminus.

Nucleic acid binding domains include polypeptides, proteins, or moieties which are capable of binding double-or single-stranded DNA, RNA, or combinations thereof, generally or with sequence specificity either alone or in coordination with another molecule. In some embodiments, the nucleic acid binding domain is capable of binding directly to the target nucleic acid sequences. In some embodiments, the nucleic acid binding domain is capable of binding indirectly to the target nucleic acid sequences, through an additional molecule. Exemplary nucleic acid binding domains include polypeptides having helix-turn-helix motifs, zinc fingers, leucine zippers, HMG-box (high mobility group box) domains, winged helix regions, winged helix-turn-helix regions, helix-loop-helix regions, immunoglobulin folds, B3 domains, Wor3 domains, TAL effector DNA-binding domains, and the like. The nucleic acid binding domain may be a natural binding domain. In some embodiments, the nucleic acid binding domain comprises a programmable nucleic acid binding domain, e.g., a nucleic acid binding domain engineered, for example by altering one or more amino acid of a natural nucleic acid binding domain, to bind to a predetermined nucleotide sequence.

The nucleic acid binding domain may be derived from domains found in naturally occurring transcription activator-like effectors (TALEs) such as AvrBs3, Hax2, Hax3 or Hax4 (Bonas et al. Mol Gen Genet 218(1):127-36, 1989; Kay et al. Mol Plant Microbe Interact 18(8): 838-48, 2005). TALEs have a modular binding domain consisting of repetitive sequences of residues; each repeat region consists of 34 amino acids. A pair of residues at the 12th and 13th position of each repeat region determines the nucleotide specificity and combining of the regions allows synthesis of sequence-specific TALE binding domains. In some embodiments, the TALE binding domains may be engineered using known methods to provide a binding domain with chosen specificity for any target sequence. The binding domain may comprise multiple (e.g., 2, 3, 4, 5, 6, 10, 20, or more) TALE effector binding motifs. In particular, any number of nucleotide-specific TALE effector motifs can be combined to form a sequence-specific binding domain to be employed in the fusion protein.

In some embodiments, the nucleic acid binding domain is derived from an RNA-guided protein (e.g., an RNA-guided nuclease). These proteins associate with an RNA molecule which guides the protein to the target DNA based on sequence complementarity of the RNA molecule to the target DNA. Exemplary RNA-guided proteins include for example, Cas proteins, transposon proteins (e.g., ISC transposon proteins or TnpB proteins, and other homologous proteins), and the Fanzor protein.

In some embodiments, the nucleic acid binding domain is derived from a Clustered Regularly Interspaced Short Palindromic Repeats associated (Cas) protein and associates with the target nucleic acid through a guide RNA (gRNA), a full description of which is provided elsewhere herein. The gRNA itself comprises a sequence complementary to one strand of the target sequence and a scaffold sequence binds and recruits the Cas protein to the target sequence. Thus, the disclosure provides base editors, which are fusion proteins of a Cas protein, generally a fully or partially catalytically inactivated Cas nuclease, as described below, and a deaminase or “base editing” enzyme.

The Cas protein can be from any Type or Class of CRISPR-Cas systems (e.g., Class 1, Class 2, Class 3, Types I-VI, or any subtypes thereof) from any species. Exemplary Cas proteins include: Cas1, Cas2, Cas3, Cas4, Cas5, Cas6, Cas7, Cas8, Cas9, Cas10, Cas12, Cas13, and the like. In some embodiments, the Cas protein is Cas9 or Cas 12. Fragments or variants of any Cas protein which retain their nucleic acid binding capability may also be suitable for use as a nucleic acid binding domain in a fusion protein disclosed herein.

In some embodiments, the Cas protein is Cas9, or a fragment thereof. The Cas9 protein may be obtained from any suitable organism. For example, a number of bacteria express Cas9 protein orthologs or variants. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank, UniProt, and JGI Integrated Microbial Genomes and Microbiomes (IMG/M) databases. The Cas9 protein may be from Streptococcus pyogenes, Staphylococcus aureus (S. aureus), Campylobacter jejuni, Corynebacterium diphtheria, Eubacterium ventriosum, Streptococcus pasteurianus, Lactobacillus farciminis, Sphaerochaeta globus, Azospirillum (strain B510), Gluconacetobacter diazotrophicus, Neisseria cinerea, Roseburia intestinalis, Parvibaculum lavamentivorans, Nitratifractor salsuginis (strain DSM 16511), Campylobacter lari (strain CF89-12), or Streptococcus thermophilus (strain LMD-9). In some embodiments, the Cas9 is from Streptococcus pyogenes or Staphylococcus aureus.

Engineered Cas protein variants having one or more inactivated nuclease domains; alterations in the PAM requirements of target nucleic acids; decreased off-target binding or increased on-target binding; decreased editing windows on target nucleic acid; decreased ‘bystander’ effects, editing of nucleotides outside but near editing window; and the like are suitable for use in the disclosed fusion proteins. For example, Streptococcus pyogenes Cas 9 (SpCas9) variants SpCas9-VQR, -VRQR, -EQR, -VRER, xCas9, SpCas9-NG, SpG, and SaKKHn allow targeting of genomic regions containing non-NGG PAMs and SpRY is a near-PAMless variant of SpCas9 (See, Kleinstiver B P et al., Nature. 523, 481-5 (2015); Kleinstiver B P et al., Nature. 529, 490-5 (2016); Kim et al., Nat. Biotechnol. 35, 371-376 (2017); Nishimasu, H. et al., Science 361, 1259-1262 (2018); Hu J H, et al., Nature. 556, 57-63 (2018); Miller, et al. Nat. Biotechnol. 38, 471-481(2020); Yang, L. et al., Protein Cell 9, 814-819 (2018); Walton, et al., Science 268, 290-296 (2020), incorporated herein by reference).

In some embodiments, the Cas9 protein is a Cas9 nickase (Cas9n). Wild-type Cas9 has two catalytic nuclease domains (HNH and RuvC) facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas9 protein is a catalytically dead Cas9. Catalytically dead Cas 9 (dCas9) can be obtained, for example, by introducing point mutations (e.g., substitutions, deletions, or additions) in the Cas9 molecule at the DNA-cleavage domain, e.g., the nuclease domain, the RuvC and/or HNH domain. Sec, e.g., Jinek et al., Science 337:816-21 (2012), incorporated by reference herein in its entirety. For example, introducing two point mutations in the RuvC and HNH domains reduces the Cas9 nuclease activity while retaining the Cas9 nucleic acid binding activity. For example, Streptococcus pyogenes Cas9 may be rendered catalytically dead by mutations of D10 and at least one of E762, H840, N854, N863, or D986, typically H840 and/or N863A (see, e.g., U.S. Patent Application Publication 2017/0051312, incorporated herein by reference). Mutations in corresponding orthologs are known, such as N580 in Staphylococcus aureus Cas9. Similar mutations can also apply to any other naturally occurring Cas9 (e.g., Cas9 from other species) or engineered Cas9 molecules. Oftentimes, such mutations result in the catalytically dead Cas9 possessing no more than 3% of the normal nuclease activity.

In some embodiments, the deaminase and the nucleic acid binding domain are covalently linked in a single amino acid chain through a linker. The linker may have any of a variety of amino acid sequences. Proteins can be joined by a linker polypeptide, generally of a flexible nature, although other chemical linkages are not excluded. Suitable linkers include polypeptides of between 1 amino acid and 100 amino acids in length, 4 amino acids and 40 amino acids in length, or between 4 amino acids and 25 amino acids in length. The linking peptides may have virtually any amino acid sequence, bearing in mind that the preferred linkers will have a sequence that results in a generally flexible peptide. Small amino acids, such as glycine and alanine, are useful in creating a flexible peptide linker. A variety of different linkers are considered suitable for use, including but not limited to, glycine-serine polymers, glycine-alanine polymers, and alanine-serine polymers. Such fusion proteins can be expressed recombinantly from a single nucleic acid encoding the amino acid chain.

Alternatively, the nucleic acid binding domain and the deaminase may be individually fused to one half of a binding pair (e.g., from a recruitment system) and, when introduced into the same system or location, the deaminase and nucleic acid binding domain form a protein conjugate through the recruitment system. The recruitment system can comprise any binding pair. For example, the recruitment system may comprise an aptamer and an aptamer binding protein. The recruitment system may be a so-called split system. Split systems include two or more polypeptide chains that reassemble into an operable fusion protein or protein conjugate upon association of the two binding partners. Split systems include, but are not limited to, intein, MS2, or SunTag based systems.

In some embodiments, the aptamer sequence is a nucleic acid (e.g., RNA aptamer) sequence. In some embodiments, the guide RNA also comprises a sequence of one or more RNA aptamers, or distinct RNA secondary structures or sequences that can recruit and bind another molecular species, an adaptor molecule, such as a nucleic acid or protein. Any RNA aptamer/aptamer binding protein pair known may be selected and used in connection with the present disclosure (see, e.g., Jayasena, S. D., Clinical Chemistry. 45(9): p. 1628-1650, (1999); Gelinas, et al., Current Opinion in Structural Biology 36: p. 122-132, (2016); and Hasegawa, H., Molecules, 21(4): p. 421 (2016), incorporated herein by reference).

In some embodiments, the aptamer sequence is a peptide aptamer sequence. In some embodiments, the nucleic acid binding domain comprises the peptide aptamer sequence and the deaminase comprises the peptide aptamer binding protein. In some embodiments, the deaminase comprises the peptide aptamer sequence and the nucleic acid binding domain comprises the peptide aptamer binding protein. The peptide aptamer sequence or peptide aptamer binding protein may be fused in any orientation (e.g., N-terminus to C-terminus, C-terminus to N-terminus, N-terminus to N-terminus). The peptide aptamer sequence or peptide aptamer binding protein may be fused by a linker region. Suitable linker regions are known in the art. The linker may be flexible or configured to allow the functionality and association with the DNA or other proteins with decreased steric hindrance. The linker sequences may provide an unstructured or linear region of the polypeptide, for example, with the inclusion of one or more glycine and/or serine residues. The linker sequences can be at least about 2, 3, 4, 5, 6, 7, 8, 9, 10 or more amino acids in length.

The peptide aptamers can be naturally occurring or synthetic peptides that are specifically recognized by an affinity agent. Such aptamers include, but are not limited to, a c-Myc affinity tag, an HA affinity tag, a His affinity tag, an S affinity tag, a methionine-His affinity tag, an RGD-His affinity tag, a 7× His tag, a FLAG octapeptide, a strep tag or strep tag II, a V5 tag, or a VSV-G epitope. Corresponding aptamer binding proteins are well-known in the art and include, for example, primary antibodies, biotin, affimers, single domain antibodies, and antibody mimetics.

Any of the deaminases and/or fusion proteins disclosed herein may further comprise one or more proteins, polypeptides (e.g., protein domain sequences), or peptides. For example, the deaminases and/or fusion proteins disclosed herein may be fused to another protein or protein domain that provides for tagging or visualization (e.g., GFP). The one or more proteins, polypeptides (e.g., protein domain sequences), or peptides may be appended at an N-terminus, a C-terminus, internally, or a combination thereof. The one or more proteins, polypeptides (e.g., protein domain sequences), or peptides may be fused in any orientation in relationship to the disclosed protein. The one or more proteins, polypeptides (e.g., protein domain sequences), or peptides may be fused via a linker, as described above.

In some embodiments, the deaminases and/or fusion proteins comprise one or more nuclear localization sequences (NLSs). The nuclear localization sequence may be appended, for example, to the N-terminus, a C-terminus, internally, or a combination thereof. In such cases when the deaminase and/or fusion protein comprises two or more NLSs, the NLSs may be in tandem, separated by a linker, at either end of the protein, or one or more may be embedded in the protein.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine. The NLS may be appended by a linker.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins. In some embodiments, the NLS is a bipartite sequence. Bipartite NLSs comprise two clusters of basic amino acids, separated by a spacer of about 9-12 amino acids. Exemplary bipartite NLSs include the nuclear localization sequences of nucleoplasmin, EGL-12, or bipartite SV40. In some embodiments, the NLS comprises a sequence of: KR(K/R)R (SEQ ID NOs: 792-793); K(K/R)RK (SEQ ID NOs: 794-795); (R/P)XXKR(K/R)(\hat{ }DE) (SEQ ID NOS: 796-799) or (R/P)XXKR(\hat{ }DE) (K/R) (SEQ ID NOs: 800-803) wherein (\hat{ }DE) represents any amino acid except for Asp or Glu; KRX(W/F/Y)XXAF (SEQ ID NOs: 804-806); LGKR(K/R)(W/F/Y) (SEQ ID NO: 807-808); or a bipartite sequence thereof.

The deaminases and/or fusion proteins may also comprise an epitope tag (e.g., 3xFLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein or polypeptide.

In some embodiments, the deaminases and/or fusion proteins may be fused with one or more (e.g., two, three, four, or more) protein transduction moieties. A protein transduction moiety is a polypeptide, polynucleotide, carbohydrate, or organic or inorganic compound that facilitates traversing a lipid bilayer, micelle, cell membrane, organelle membrane, or vesicle membrane. A protein transduction moiety attached to another molecule facilitates the molecule traversing a membrane, for example going from extracellular space to intracellular space, or cytosol to within an organelle. The protein transduction moiety may be linked to the terminus of the deaminase or fusion protein, or alternatively be inserted internally. Examples of protein transduction moieties include but are not limited to a minimal undecapeptide protein transduction domain (corresponding to residues 47-57 of HIV-1 TAT comprising); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); Transportan, and the like.

### Systems and Compositions

Disclosed herein are systems and compositions that comprise a deaminase or a nucleic acid encoding thereof. In some embodiments, the systems and compositions comprise a deaminase and a nucleic acid binding polypeptide, or one or more nucleic acids encoding thereof. In some embodiments, the deaminase and the nucleic acid binding polypeptide are fused as a single protein, for example as described above for the fusion protein. In some embodiments, the deaminase and the nucleic acid binding polypeptide are each linked to a half of a binding pair. Descriptions of the deaminases, nucleic acid binding polypeptides, and fusion protein provided above are equally applicable to the systems and compositions.

In some embodiments, the nucleic acid sequences that encode the deaminase and the nucleic acid binding polypeptide are on the same nucleic acid. In some embodiments, the nucleic acid sequences that encode the deaminase and the nucleic acid binding polypeptide are on different nucleic acids. In some embodiments, the nucleic acids comprise one or more messenger RNAs, one or more vectors, or any combination thereof.

In some embodiments, for example when the nucleic acid binding polypeptide is an RNA-guided protein (e.g., a Cas protein) or fragment thereof, the compositions or systems further comprise at least one guide RNA (gRNA) or one or more nucleic acids comprising a sequence encoding the least one gRNA. In instances when the composition or system comprises more than one gRNA, each may be encoded on the same or different nucleic acid as the other gRNA, together or separate from either or both of the deaminase and the nucleic acid binding polypeptide. For example, the system and compositions may comprise a first nucleic acid encoding the deaminase and the nucleic acid binding polypeptide and a second nucleic acid encoding the gRNA; a first nucleic acid encoding the deaminase and the gRNA and a second nucleic acid encoding the nucleic acid binding polypeptide; a first nucleic acid encoding the deaminase and a second nucleic acid encoding the gRNA and the nucleic acid binding polypeptide; or a single nucleic acid encoding the deaminase, the nucleic acid binding polypeptide, and the gRNA. In some embodiments, the at least one gRNA is provided in a ribonucleoprotein (RNP) complex with the RNA-guided protein.

The gRNA may contain separate crRNA and tracrRNA sequences (or dual guide RNA) or having the crRNA and tracrRNA fused by a flexible linker (or single guide RNA, sgRNA). The terms “gRNA,” “guide RNA,” and “guide sequence” may be used interchangeably throughout and refer to a nucleic acid comprising a sequence that determines the sequence specificity of the CRISPR-associated protein. A gRNA hybridizes to (complementary to, partially or completely) a target nucleic acid sequence (e.g., the genome in a host cell).

In some embodiments, at least one gRNA is encoded in a CRISPR array. CRISPR arrays contain a series of direct repeats separated by short sequences called spacers. The CRISPR-associated protein described herein may have a preference for direct repeat sequences. These can be determined by methods known in the art. For example, the CRISPR RNA (crRNA) may contain multiple gRNAs or may contain more than one different sequence each configured to hybridize a distinct target nucleic acid sequence.

The gRNA or portion thereof that hybridizes to the target nucleic acid (a target site) may be between 15-40 nucleotides in length. gRNAs or sgRNA(s) used in the present disclosure can be between about 5 and 100 nucleotides long, or longer. The gRNA may be a non-naturally occurring or engineered gRNA.

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp. 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In some embodiments, the gRNA sequence that binds to the target nucleic acid may be fused to a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the targeting gRNA sequence and tracrRNA scaffold sequence are expressed as separate transcripts. In some embodiments, this may be referred to as a dual guide RNA In such embodiments, the gRNA sequence further comprises an additional sequence that is complementary to a portion of the scaffold sequence and functions to bind (hybridize) the scaffold sequence.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

In some embodiments, the compositions and systems may further comprise one or more additional genome engineering tools. For example, the compositions may further comprise nucleases, such as zinc finger nucleases (ZFNs) and/or transcription activator like effector nucleases (TALENs); transcriptional activators, transcriptional repressors, histone-modifying proteins, integrases, recombinases, and the like.

The compositions or systems may further comprise an excipient or carrier. Excipients and carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

In some embodiments, the excipient or carrier is pharmaceutically acceptable. Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. Sec, e.g., Remington: The Science and Practice of Pharmacy 20th Ed. (2000) Lippincott Williams and Wilkins, Ed. K. E. Hoover.

The carrier may include a delivery vehicle. Delivery vehicles such as nanoparticle-and lipid-based delivery systems can be used. Exemplary delivery vehicles include, but are not limited to, microparticle compositions comprising a variety of polymers, liposomes or lipid nanoparticles, viral vectors, ribonucleoprotein (RNP) complexes, and the like.

Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin, lipids, and phospholipids, in any combination thereof.

In some embodiments, a liposome or lipid nanoparticle encapsulates the disclosed systems, nucleic acids, or proteins (e.g., deaminases and nucleic acid binding polypeptides). Methods of making lipid compositions include, for example, lipid film hydration, optionally coupled with sonication or extrusion, solvent evaporation (e.g., ethanol injection, ether injection, or reverse phase evaporation), solvent-diffusion method, hot homogenization process, detergent removal methods, or combinations thereof. Any naturally occurring or synthetic vesicle forming lipid or combinations thereof can be used, including for example, di-aliphatic chain lipids, such as phospholipids; diglycerides; di-aliphatic glycolipids; single lipids such as sphingomyelin or glycosphingolipid; steroidal lipids; hydrophilic polymer derivatized lipids; or mixtures thereof. Liposome and lipid nanoparticle compositions of the disclosure may include one or more cationic and/or ionizable lipids, phospholipids, neutral or non-cationic lipids, polyethyleneglycol (PEG)-lipid conjugates, and/or sterols. In some embodiments, the lipid nanoparticle comprises a cationic lipid and/or ionizable lipid, a neutral or non-cationic lipid, and cholesterol.

The liposomes and lipid nanoparticles described herein may also include other components typically used in the formation of vesicles (e.g., for stabilization). Examples of such other components includes, without being limited thereto, fatty alcohols, fatty acids, and/or any other pharmaceutically acceptable excipients which may affect the surface charge, the membrane fluidity and assist in the incorporation of the lipid into the lipid assembly.

The liposome and lipid nanoparticle compositions of the disclosure can also be targeting compositions, e.g., contain one or more targeting moieties or biodistribution modifiers on the surface. A targeting moiety can be any agent that is capable of specifically binding or interacting with a desired target and are generally known in the art, for example ligands such as folic acid, proteins, antibody or antibody fragments, and the like).

The phrase “pharmaceutically acceptable,” as used in connection with the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the composition (e.g., the nucleic acids, vectors, cells, proteins, or polypeptides) and does not negatively affect the subject to which the composition(s) are administered. Any of the compositions to be used in the present methods can comprise pharmaceutically acceptable carriers, excipients, or stabilizers in the form of lyophilized formations or aqueous solutions.

### Nucleic Acids

Also disclosed herein are nucleic acids encoding the deaminases or fusion proteins as described herein. The nucleic acids may be DNA, RNA, or combinations thereof. In some embodiments, the nucleic acids comprise one or more messenger RNAs, one or more vectors, or any combination thereof.

In certain embodiments, the nucleic acids are engineered for codon-optimization. It will be appreciated altering codons to those most frequently used in the cells or subject of interest allows for maximum expression. Such modified nucleic acid sequences are commonly described in the art as “codon-optimized.” In some embodiments, the nucleic acid sequence is considered codon-optimized if at least about 60% (e.g., about 65%, about 70%, about 75%, about 80%, about 85%, about 90%, about 95%, or about 98%) of the codons encoded therein are preferred codons to the subject of interest.

The present disclosure also provides for DNA segments encoding the deaminases or fusion proteins disclosed herein, vectors containing these segments, and cells containing the vectors. The vectors may be used to propagate the DNA segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode the deaminases, fusion proteins, or one or more or all of the components of the systems or compositions, as disclosed herein. The vector(s) can be introduced into a cell that is capable of expressing the polypeptide encoded thereby, including any suitable prokaryotic or eukaryotic cell.

The vectors of the present disclosure may be delivered to a eukaryotic cell. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), nucleic acids, and nucleic acids complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration.

Drug selection strategies may be adopted by positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver deaminases, fusion proteins, or one or more or all of the components of the system or compositions (such as a deaminase, fusion protein, and/or a guide RNA) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding deaminases, fusion proteins, or one or more or all of the components of the system or compositions is contained in a plasmid vector that allows expression of the protein(s) and subsequent isolation and purification produced by the recombinant vector. Accordingly, the proteins disclosed herein can be purified following expression, obtained by chemical synthesis, or obtained by recombinant methods.

To construct cells that express the deaminases, fusion proteins, or one or more or all of the components of the system or compositions, expression vectors for stable or transient expression may be constructed via conventional methods as described herein and introduced into cells. For example, nucleic acids encoding the deaminases, fusion proteins, or one or more or all of the components of the system or compositions may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), Hl (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, mycoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRPI genes of S. cerevisiae.

When introduced into the cell, the vectors may be maintained as an autonomously replicating sequence or extrachromosomal element or may be integrated into host DNA.

The proteins, polynucleotides encoding these proteins, and systems and compositions comprising the proteins and/or polynucleotides described herein may be delivered by any suitable means. In certain embodiments, the delivery is in vivo. In other embodiments, the delivery is to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the deaminases, fusion proteins, or one or more or all of the components of the system or compositions is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA 110(6): 2082-2087 (2013) incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle-and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459(1-2):70-83), incorporated herein by reference.

In some embodiments, the deaminases, fusion proteins, or one or more or all of the components of the system or compositions may be mixed, individually or in any combination, with a carrier which are also within the scope of the present disclosure. Exemplary carriers include buffers, antioxidants, preservatives, carbohydrates, surfactants, and the like, and are described in detail elsewhere herein.

Also disclosed is a cell comprising the deaminases, fusion proteins, nucleic acids, or one or more or all of the components of the system or compositions described herein. In some embodiments, the cell is a prokaryotic cell. In some embodiments, the cell is a eukaryotic cell. In some embodiments, the cell is a mammalian cell. In some embodiments, the cell is a human cell.

### Methods

The disclosure also provides methods of modifying a target nucleic acid sequence (e.g., DNA or RNA). The phrase “modifying a nucleic acid sequence,” as used herein, refers to modifying at least one physical feature of a nucleic acid sequence of interest. In some embodiments, the modifications comprise base editing. In some embodiments, the base editing edits an adenine in the target nucleic acid.

The methods comprise contacting a target nucleic acid sequence with a deaminase, fusion protein, composition, or system as described herein. In some embodiments, contacting a target nucleic acid sequence comprises introducing the deaminase, fusion protein, composition, or system into the cell. The deaminase, fusion protein, composition, or system may be introduced into eukaryotic or prokaryotic cells by methods known in the art, as described elsewhere herein.

The cell may be a prokaryotic cell, a plant cell, an insect cell, a vertebrate cell, an invertebrate cell, an animal cell, a mammalian cell, or a human cell. In some embodiments, the cell is a stem cell.

In some embodiments, the cell is ex vivo (e.g., fresh isolate—early passage). In some cases, the cell is in vivo. In some cases, the cell is in culture or in vitro (e.g., immortalized cell line). Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

In some embodiments, introducing the deaminase, fusion protein, composition, or system into a cell comprises administering the deaminase, fusion protein, composition, or system to a subject. In some embodiments, the subject is human. The administering may comprise in vivo administration of the deaminase, fusion protein, composition, system, or a nucleic acid encoding the deaminase, fusion protein, or system. In alternative embodiments, an in vitro or ex vivo treated cell is transplanted into a subject.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, microRNA (miRNA), and small interfering RNA (siRNA), and coding RNA, such as messenger RNA (mRNA). In some embodiments, the target nucleic acid sequence encodes a protein or polypeptide.

In some embodiments, the methods described herein result in an adenine (A) to guanine (G) point mutation within a gene (e.g., a coding region or non-coding region (e.g., a promoter or regulatory sequences) of a gene).

In some embodiments, the methods described herein alter the regulatory sequence of a gene (e.g., a gene promoter or gene repressor). Accordingly, in some embodiments, the methods described herein lead to modulation (increase, decrease, or cessation) of transcription of a gene. In some embodiments, the methods described herein alter the splicing of a gene (e.g., introduce or remove a splice site). Accordingly, in some embodiments, the method results in the introduction of a splice site in a gene. In alternative embodiments, the method results in the removal of a splice site.

In some embodiments, the methods described herein alter the coding sequence of a gene. In some embodiments, the alteration is silent or synonymous. In some embodiments, the alteration is non-synonymous and results in an amino-acid change. In some embodiments, the methods described herein generate a stop codon, for example, a premature stop codon within the coding region of a gene. In some embodiments, the methods described herein eliminate a stop codon, e.g., a stop codon in a target nucleic acid comprising the nucleic acid sequence TAG, TAA, or TGA.

In some embodiments, the methods described herein may be used to correct one or more defects or mutations in one or more genes (referred to as “gene correction”). In some cases, the target sequence encodes a defective version of a gene, and the disclosed systems and compositions are configured to correct, or ablate e.g., by mutating the start codon (ATG) and abolishing gene expression, the defective version of the gene. Alternatively, the target sequence may encode the wild-type version of the gene, and the disclosed systems and compositions are configured to confer the disease-causing mutation to the gene, for example, for use in cell and organismal models of the disease or disorder.

In some embodiments, the defective version of the gene includes a point mutation. Point mutations are the largest class of known pathogenic genetic variants and approximately half of which result from a change of a G-C to an A-T base pair. The deaminases and fusion proteins (e.g., base editors) disclosed herein may install or reverse point mutations, thereby providing methods for the study and treatment of disease-associated point mutations.

The present methods may be used in various bacterial hosts, including human pathogens that are medically important, and bacterial pests that are key targets within the agricultural industry, as well as antibiotic resistant versions thereof. The method may be designed to target any gene or any set of genes, such as virulence or metabolic genes, for clinical and industrial applications in other embodiments. The present methods may be used to inactivate microbial genes. In some embodiments, the gene is an antibiotic resistance gene. The present methods may be used to treat a multi-drug resistance bacterial infection in a subject. The present methods may also be used for genomic engineering within complex bacterial consortia.

The methods described here also provide for treating a disease or disorder in a subject. The method may comprise administering to the subject, in vivo, an effective amount of the deaminase, fusion protein, composition, or system, or by transplantation of ex vivo treated cells. A “subject” or “patient” may be human or non-human and may include, for example, animal strains or species used as “model systems” for research purposes, such a mouse model as described herein. Within the context of the present disclosure, the term “effective amount” refers to that quantity such that modification of the target nucleic acid is achieved.

In some embodiments, the systems and methods target one or more “disease-associated” genes. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may refer to a gene, the mutation or genetic variation of which is directly responsible or is in linkage disequilibrium with a gene(s) that is responsible for the etiology of a disease. In another embodiment, the target genomic DNA sequence can comprise a gene, the mutation of which contributes to a particular disease in combination with mutations in other genes.

In some embodiments, the one or more disease-associated gene comprises one or more point mutations, single nucleotide variants (SNVs), or single-nucleotide polymorphisms (SNPs). In some embodiments, the point mutation, SNV, or SNP comprises a mutation of a wild-type base to an adenine. In some embodiments, the point mutation, SNV, or SNP comprises a G to A point mutation. Accordingly, correction of the G to A point mutation, SNV, or SNP with the deaminases or fusion proteins disclosed herein results in a reversion to wild-type or non-disease-associated sequence. In some embodiments, the point mutation, SNV, or SNP comprises a C to T point mutation. Accordingly, correction of the A which base pairs with the T mutation results in a wild-type or non-disease-associated sequence following cellular replication/repair processes. Exemplary diseases associated with a point mutation, SNV, or SNP that may be treated with the disclosed methods include, but are not limited to, proliferative diseases, metabolic diseases, and lysosomal storage diseases.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

A wide range of additional therapies may be used in conjunction with the methods of the present disclosure. The additional therapy may be administration of a therapeutic agent or may be an additional therapy not connected to administration of a therapeutic agent. Such additional therapies include, but are not limited to, surgery, immunotherapy, radiotherapy. The additional therapy may be administered at the same time as the above methods. In some embodiments, the additional therapy may precede or follow the treatment of the disclosed methods by time intervals ranging from hours to months.

In some embodiments, effective combination therapy is achieved with a single composition or pharmacological formulation or with two distinct compositions or formulations, administered at the same time or separated by a time interval. The therapeutic agent may comprise any manner of therapeutic, including protein, small molecule, nucleic acids, and the like. For example, therapeutic agents include, but are not limited to, immune modulators, chemotherapeutic agents, a nucleic acid (e.g., mRNA, aptamers, antisense oligonucleotides, ribozyme nucleic acids, interfering RNAs, antigene nucleic acids), decongestants, steroids, analgesics, antimicrobial agents, immunotherapies, or any combination thereof.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean elimination or reduction of a patient's tumor burden, or prevention, delay, or inhibition of metastasis, etc.

The methods disclosed herein are also applicable to plants. For example, the methods can be used to generate novel engineered plants to improve agronomic traits, for example, herbicidal resistance, resistance to environmental stress, resistance to pests, etc.

The disclosed deaminases, fusion proteins, compositions, and systems can be introduced into a plant, or a plant cell, seed, fruit, plant part, or propagation material of the plant. The term “plant propagation material” refers to generative parts of a plant, which can be used for the multiplication of the plant, and vegetative plant material such as cuttings and tubers (e.g., potatoes). In some embodiments, the propagation material is a root, a corm, a tuber, a bulb, a slip, a cutting of the plant, and a rhizome. Parts of a plant are any sections of a plant (e.g., roots, cotyledons, tendrils, leaves, flowers, seeds, stems, callus tissue, nuts, and fruit) that develop from a plant propagation material or grow at a later time. The methods described herein can be used on any plant part. Examples of plant parts include but are not limited to the root, corm, tuber, bulb, slip and rhizome.

Methods of introducing exogenous nucleic acids into plant cells are well known in the art. Such plant cells are considered transformed. DNA constructs can be introduced into plant cells by various methods, including, but not limited to PEG-or electroporation-mediated protoplast transformation, tissue culture or plant tissue transformation by biolistic bombardment, or the Agrobacterium-mediated transient and stable transformation.

The transformation can be a transient or a stable transformation. As used herein, the term “stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant. In select embodiments, the nucleic acid encoding the RNA hairpin may be stably integrated into the plant genome, for example via Agrobacterium-mediated transformation.

Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (e.g., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of co-culturing Agrobacterium with cultured plant cells or wounded tissue such as leaf tissue, root explants, hypocotyledons, stem pieces or tubers, for example, also are well known in the art. Sec., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

As such, the disclosure also provides plants and plant propagation materials (e.g., plant cell, seed, fruit, or plant parts) produced using the methods disclosed herein. Genetically modified, transformed or transgenic plants include a plant into which an exogenous polynucleotide, e.g., a polynucleotide encoding the deaminase or fusion protein disclosed herein, has been introduced.

The methods disclosed herein are suitable for use with any plant, for example, grain crops, fruit crops, forage crops, root vegetable crops, leafy vegetable crops, flowering plants, conifers, trees, oil crops, plants used in phytoremediation, industrial crops, medicinal crops, laboratory model plants, and the like. As such, non-limiting examples of plants that may be used with the present methods include: grains, forage crops, fruits, vegetables, oil seed crops, palms, forestry, vines, maize (corn, Zea mays), banana, peanut, field peas, sunflower, tomato, canola, tobacco, wheat, barley, oats, potato, soybeans, cotton, carnations, sorghum, lupin, rice, rutabaga, celery, switchgrass, apple, petunias, Arabidopsis thaliana, Medicago truncatula, Medicago sativa, Brachypodium distachyon, Nicotiana benthamiana, or Setaria viridis.

Additionally, the disclosed methods can also be used as a synthetic biology tool to record cellular signaling and exposure to stimuli. The disclosed deaminases and fusion proteins can generate predictable single point mutations and by coupling the stimulus of interest to the activity or expression of the deaminase or fusion protein, the resulting stimulus-dependent single point mutations can be used to record exposure to signals into the genome. Accordingly, the methods may further comprise exposing the cell, subject, or plant, to stimulus of interest, wherein the deaminase or fusion protein is configured for activation to the stimulus of interest (e.g., transcriptionally or translationally controlled activation).

### Kits

Also within the scope of the present disclosure are kits that include the deaminases, fusion proteins, nucleic acids, cells, compositions, systems, or components thereof as disclosed herein.

The kits may contain one or more reagents or other components useful, necessary, or sufficient for practicing any of the methods described herein, such as, transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The kit may include instructions for use in any of the methods described herein. Kits optionally may provide additional components such as buffers and interpretive information. Normally, the kit comprises a container and a label or package insert(s) on or associated with the container. Instructions supplied in the kits of the disclosure are typically written instructions on a label or package insert. In some embodiment, the disclosure provides articles of manufacture comprising contents of the kits described above.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses.

The kit will typically be provided with its various components in one or more packages, e.g., a fiber-based, a cardboard, a polymeric, or a Styrofoam box. The enclosure(s) can be configured so as to maintain a temperature differential between the interior and the exterior, for example, to provide insulating properties to keep the reagents at a preselected temperature for a preselected time. The packaging can be air-tight, waterproof (e.g., impermeable to changes in moisture or evaporation), and/or light-tight.

### EXAMPLES

The following are examples of the present invention and are not to be construed as limiting.

### Materials and Methods

### Cell Culture, Plasmid Construction, and Transient Transfection

HEK293T cells (ATCC) were cultured at 37° C. and 5% (v/v) CO₂ in high glucose DMEM with 4 mM L-glutamine, 1 mM sodium pyruvate and phenol red pH indicator (Gibco), supplemented with 10% FBS and 1× penicillin-streptomycin. 24 hours prior to transfection, cells were seeded at a density of 10³ cells/well in 96 well tissue culture-treated plates.

guide RNAs (gRNA) were cloned into pGuide plasmid with a U6 promoter driving their expression and a CMV-driven GFP transfection reporter using HiFi DNA Assembly protocols (New England Biolabs). Deaminase enzyme constructs, e.g., deaminases linked to Cas9n (nickase (D10A)), in pTwist CMV were purchased from Twist Bioscience, or a nickase version of OpenCRISPR-1 (Ruffolo, et al., bioRxiv 2024.04.22.590591).

For each transfection well, 50 ng of gRNA plasmid and 50 ng of deaminase enzyme plasmid were added to 5 μL of Opti-MEM (Gibco). One non-targeting guide RNA negative control was included for each experiment. 0.2 μL of TransIT®-2020 transfection reagent (Mirus Bio) was diluted into 4 μL of Opti-MEM. Plasmid and TransIT®-2020 mixtures were combined, incubated for 15-30 min at room temperature, and added to HEK293T cells in a dropwise manner. Plates were gently rocked to mix and incubated for 72 hours. Guide RNA sequences are listed in Table 1.

### Microscopy, Sample Preparation, and Sequencing

72 hours post-transfection, qualitative assessments of transfection efficiency and cell health were made using fluorescence and brightfield microscopy (Revolve Echo). Quantitative transfection efficiency measurements were made using 96 well plate-based fluorescence measurements (Tecan Spark). Culture media was aspirated from cells prior to washing with PBS (Gibco). 35 μL lysis buffer (100 mM Tris-HCl, pH 7.5; 0.05% SDS; 25 μg/mL Proteinase K) was added to each well. The samples were then incubated at 37° C. for 1 hour before being transferred to 96-well PCR plates and boiled at 98° C. for 15 min. Guide-specific primers were used to PCR amplify the genomic target region of interest from the cell lysates (Q5 DNA Polymerase NEB). Five samples per plate were spot-checked via gel electrophoresis to confirm the presence of the expected amplicon size. The Mag-Bind® RxnPure Plus (Omega Bio-tek) PCR clean-up kit was used to purify the PCR products prior to sequencing submission. Once cluted, DNA yields were quantified via the QuantiFluor® kit (Promega). DNA concentrations were normalized to 2 ng/100 bp of amplicon length and submitted to the UC Berkeley Sequencing facility for Sanger sequencing along with the appropriate forward PCR primer. PCR and Sanger sequencing primers are listed in Table 2.

### Sequencing Analysis

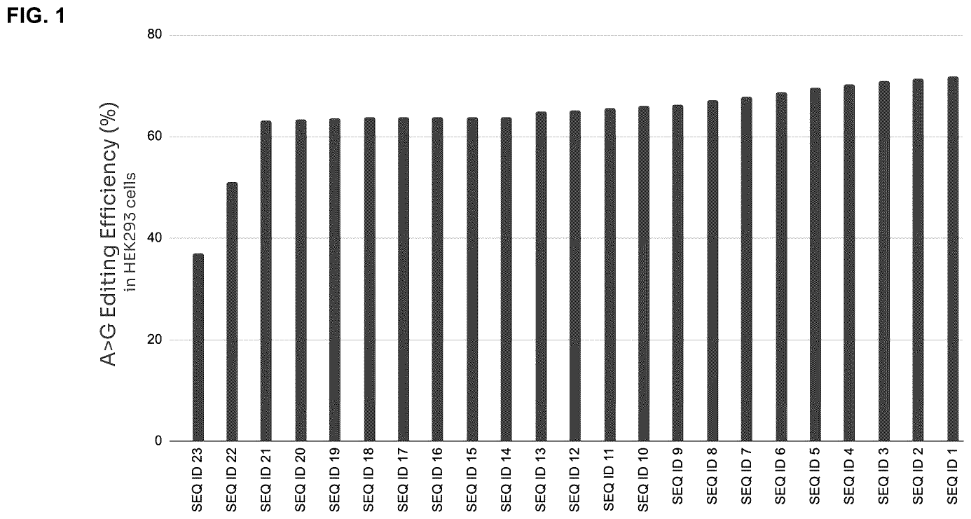

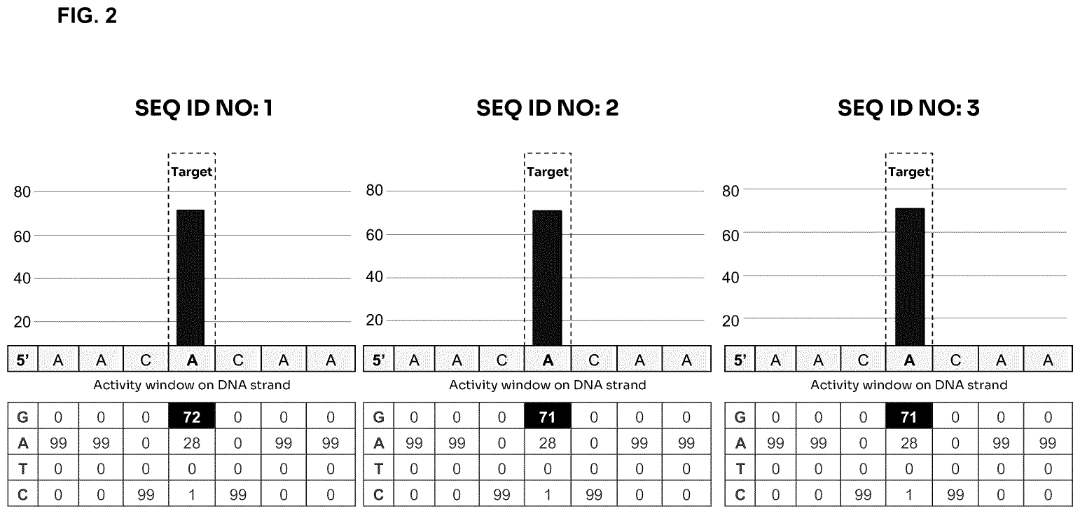

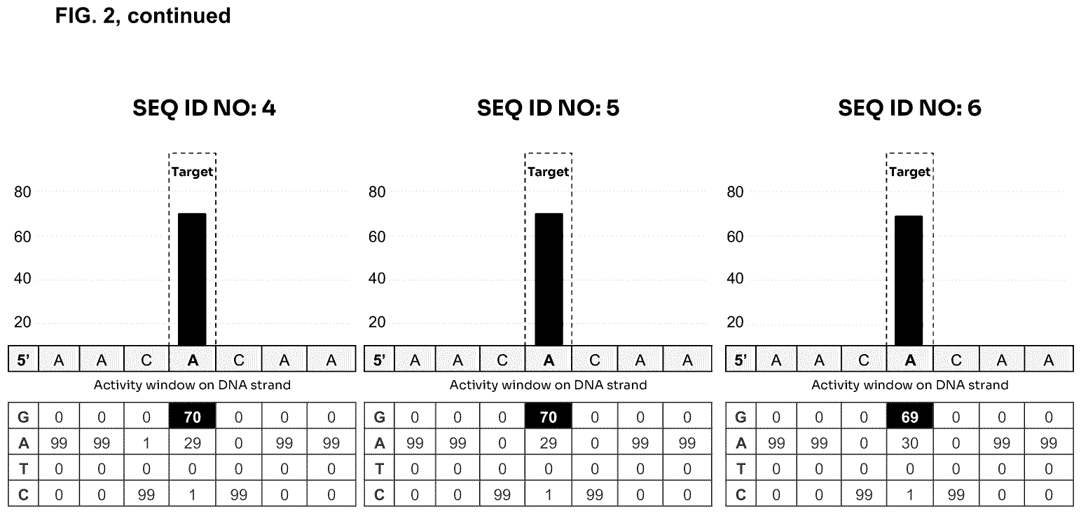

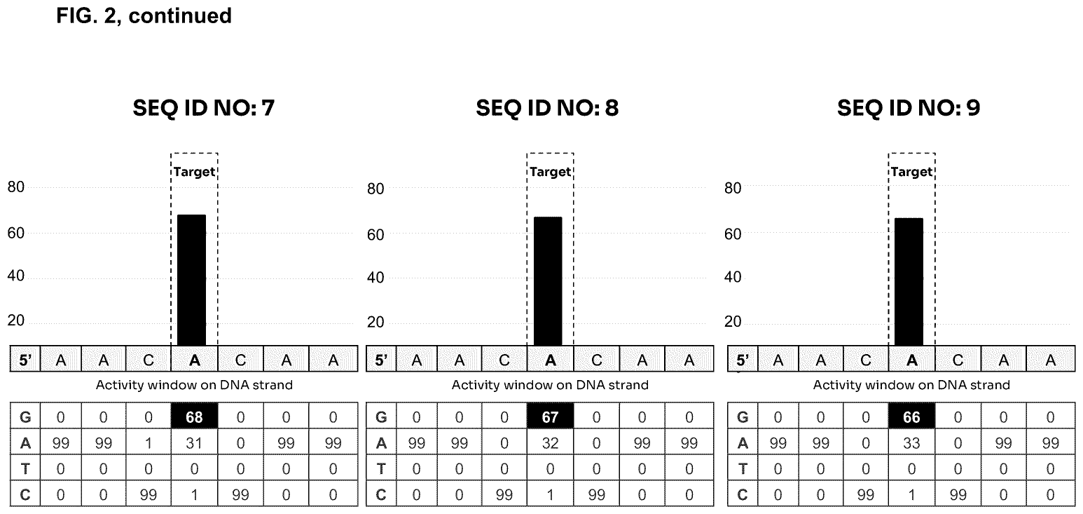

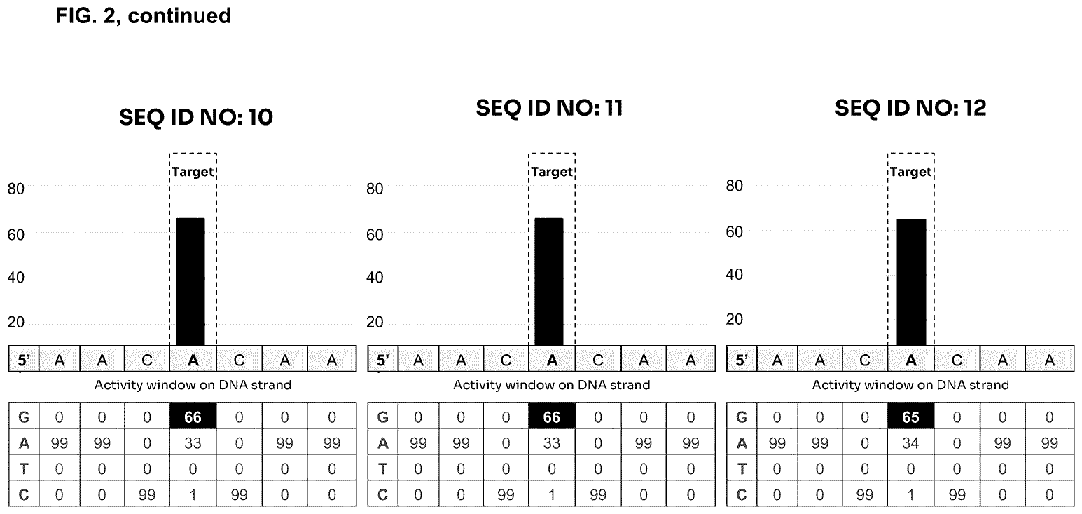

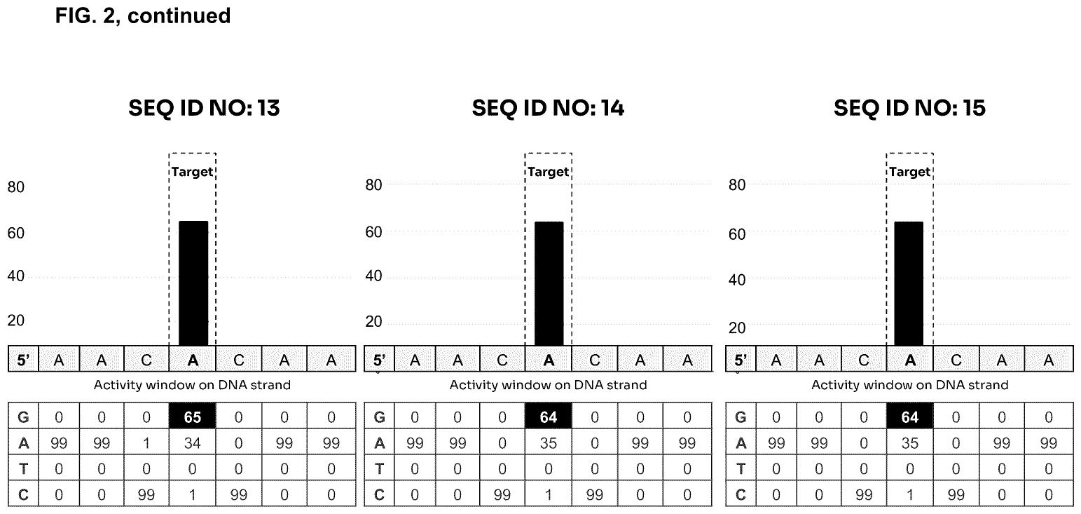

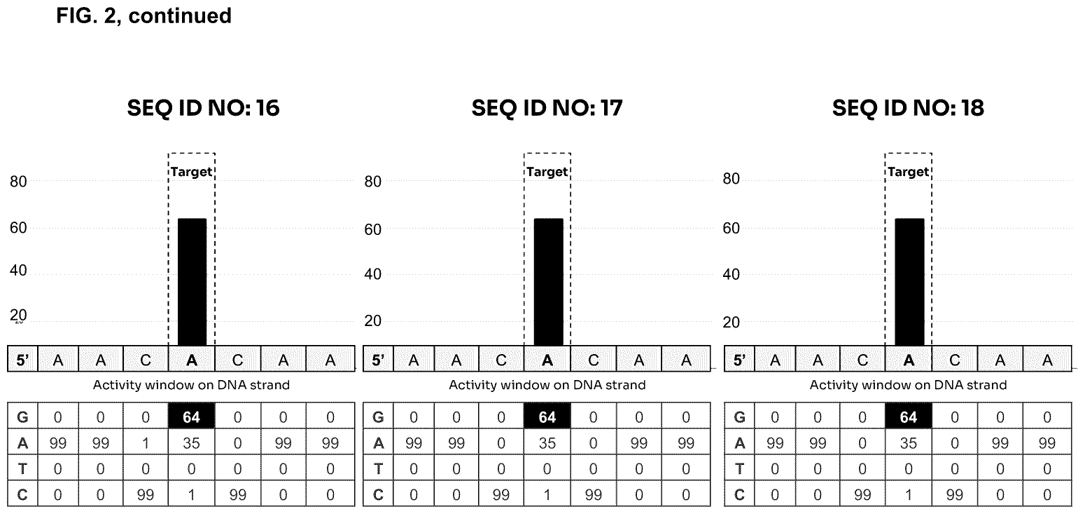

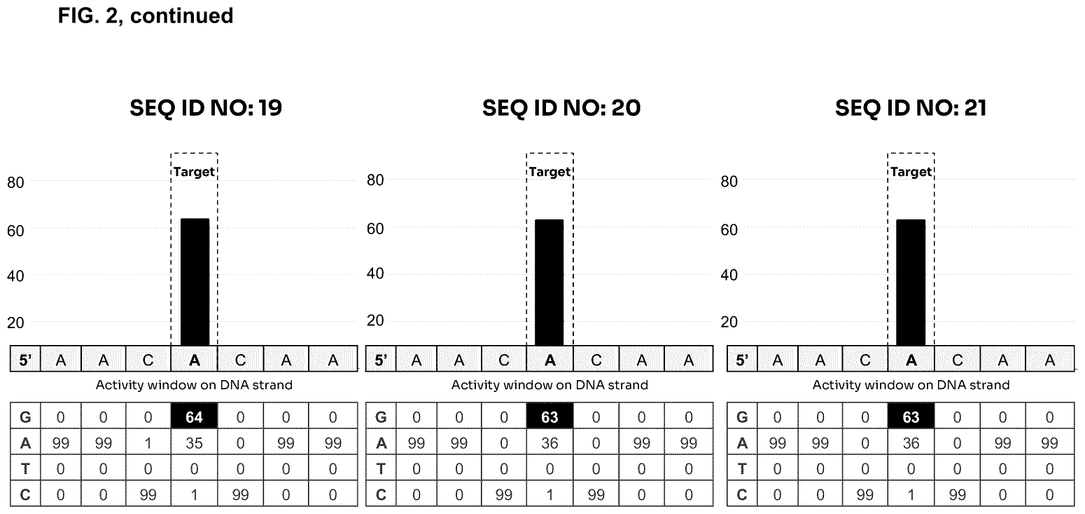

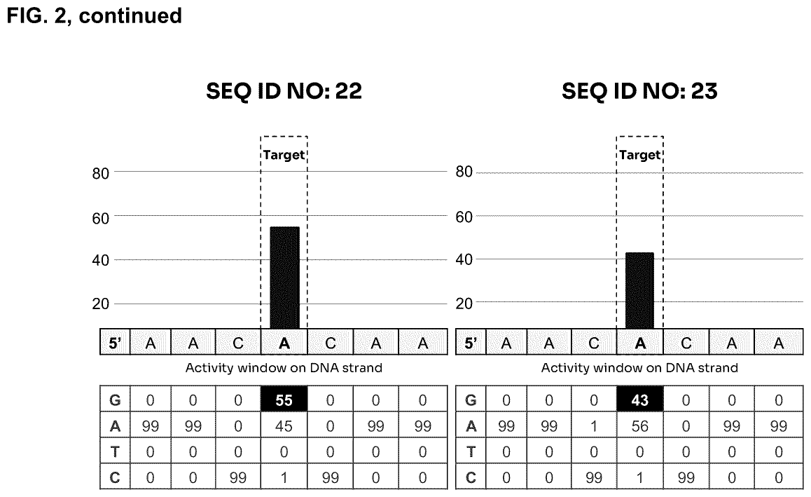

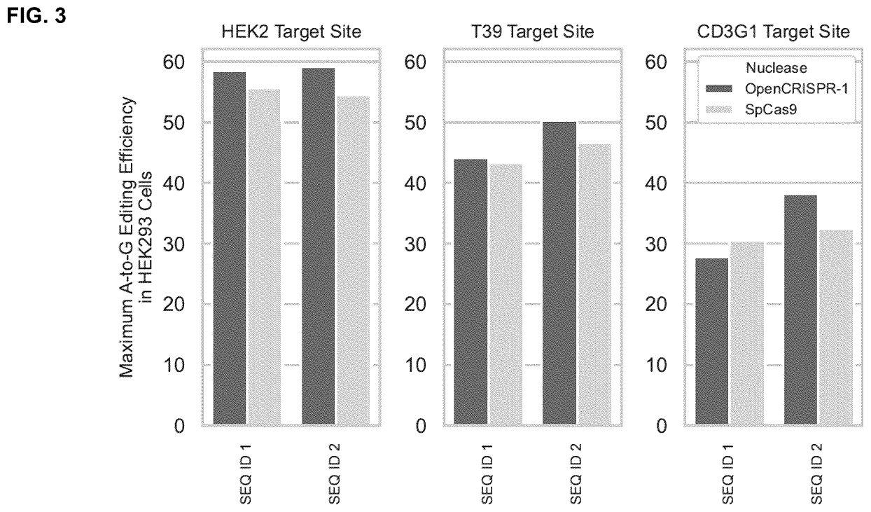

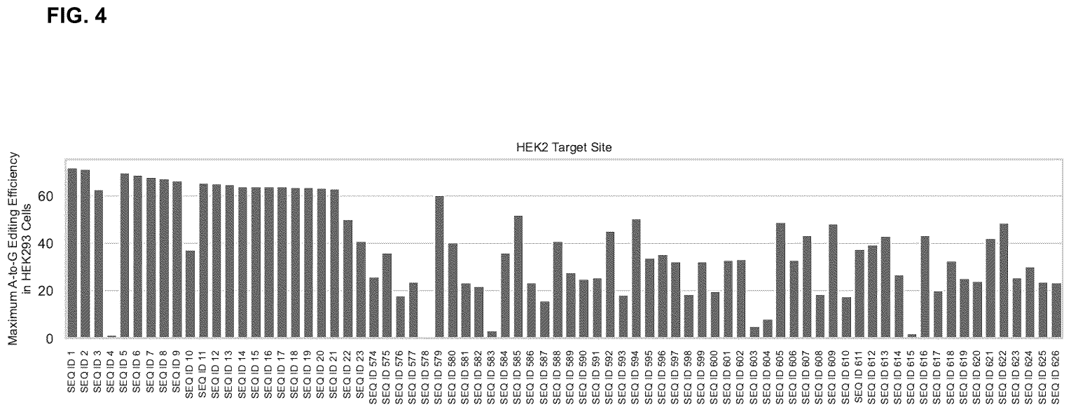

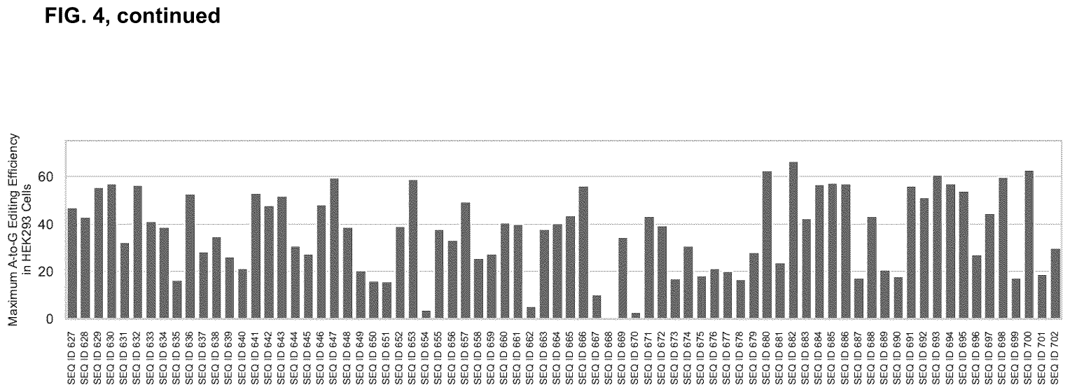

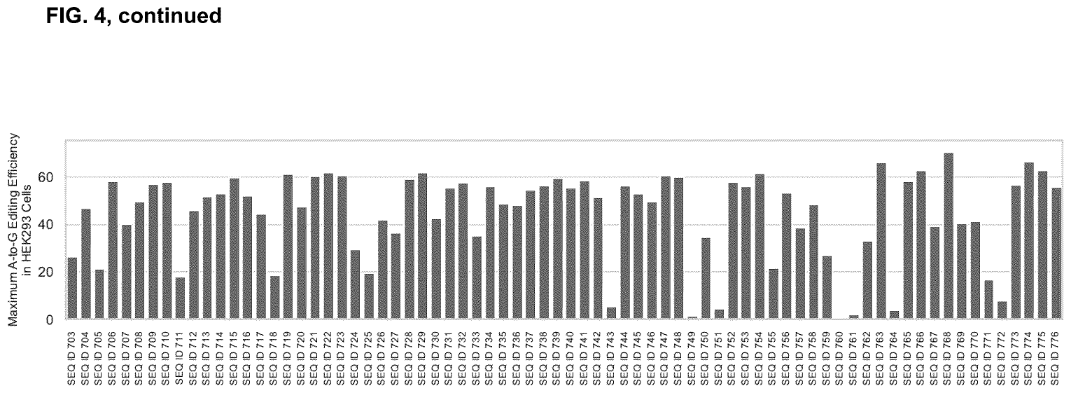

To quantify base editing efficiency from Sanger sequencing data (FIGS. 1-4), an open-source program called Beat using default parameters (Xu et al. CRISPR J 2019). Beat is a Python-based program that determines editing efficiency after subtracting the background noise and without the need to normalize to control samples. Beat produces results that are concordant with EditR (another commonly used software for base editing) and agree with base editing quantification based on NGS.

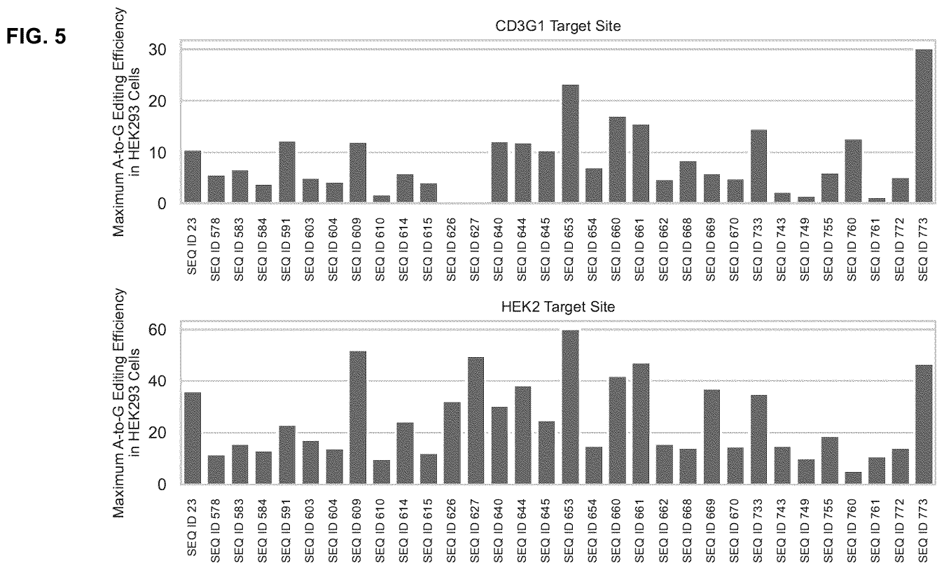

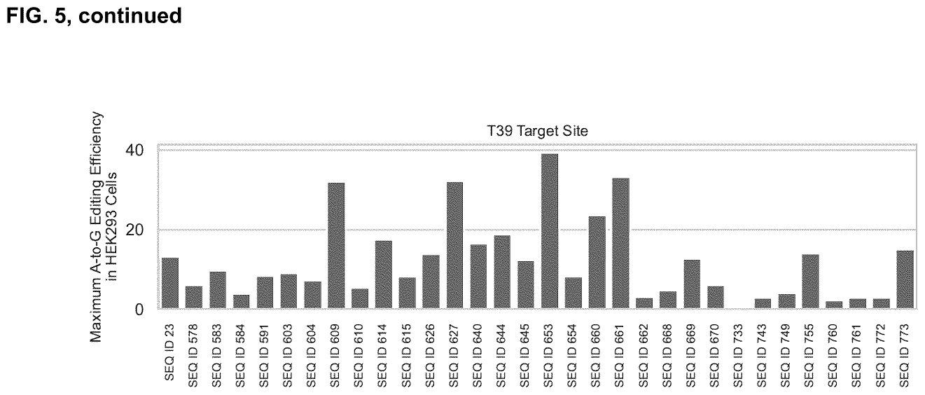

For short read Illumina NGS analysis, target sites were amplified for base editing quantification using a two-step PCR reaction. First, 5 μL of lysate (corresponding to 1×10\hat{ }3 cells) was used as a template for PCR (Invitrogen™ Platinum™ SuperFi II PCR Master Mix) with unique primer pairs containing an internal locus-specific region and an outer Illumina-compatible adapter sequence (Table 2). The resulting product was then diluted 1:100 in nuclease-free water and used as a template in a second PCR reaction targeting the outer-adapter sequence, appending unique indices (xGen UDI 10nt Primer Plates 1-16, IDT) to each amplicon for pooled sequencing. Amplicons were pooled 1:1 and sequenced on a NovaseqX with 2×151 paired end reads (Seqmatic). All amplicons across experiments included reference samples that were not treated with active base editors to control for any variant reads relative to reference genome that are not due to editing. Base editing efficiencies were calculated using CRISPResso analysis (FIG. 5). Reads were trimmed and filtered using fastp. CRISPResso2 was run in base editing mode with default parameters using the processed reads, amplicon sequences, and spacer sequences as input.

TABLE 1

| Guide RNA | Sequence | SEQ ID NO: |
|---|---|---|
| HEK2-gRNA | GAACACAAAGCAUAGACUGC | 777 |
| HEK3-gRNA | GGCCCAGACUGAGCACGUGA | 778 |
| CD3G1 | ACAUACUUCUGUAAUACACU | 779 |
| T39 | GGACAGCUUUUCCUAGACAG | 780 |
| Non-targeting- | GAGCAGAUCGUUGAUUGUAG | 781 |
| gRNA control | | |

TABLE 2

| Sequencing | | SEQ |
|---|---|---|
| Primer | Sequence | ID NO: |
| HEK2 forward(A) | CTGGTGGTACTTGAATCAAGCAC | 782 |
| HEK2 reverse(A) | GAAGGAGACTTGTGCACATTCTATAG | 783 |
| HEK3 forward | ATGTGGGCTGCCTAGAAAGG | 784 |
| HEK3 reverse | CCCAGCCAAACTTGTCAACC | 785 |
| HEK2 forward(B) | ACACTCTTTCCCTACACGACGCTCTT | 786 |
| | CCGATCTCAAGACCTGGCTGAGCTAA | |
| | C | |
| HEK2 reverse(B) | GACTGGAGTTCAGACGTGTGCTCTTC | 787 |
| | CGATCTAAATTGTCCAGCCCCATCTG | |
| CD3G1 forward | ACACTCTTTCCCTACACGACGCTCTT | 788 |
| | CCGATCTTGATCGGCTTCCTAACTGA | |
| | AG | |
| CD3G1 reverse | GACTGGAGTTCAGACGTGTGCTCTTC | 789 |
| | CGATCTAAGCTCACCAGAACAGCAAA | |
| T39 forward | ACACTCTTTCCCTACACGACGCTCTT | 790 |
| | CCGATCTCTGGCCTGGGTCAATCCTT | |
| | G | |
| T39 reverse | GACTGGAGTTCAGACGTGTGCTCTTC | 791 |
| | CGATCTTCCCTAGGTGCTGGCTTCCA | |

Sequences

| SEQ ID | |
|---|---|
| NO: | Deaminase Sequence |
| 1 | MDDQGWMKLALEEARASRAAGEVPVGAVVVRDGQIVASAGNRTRELCDPTAHAEI |
| | VALRQAARALGNYRLPGCTLYVTLEPCAMCAGAMVHARLDRLVYGVPNPKAGAAG |
| | SVLDVLHHPRLNHRLEVTGGVLEDECGALLRDFFRAR |
| 2 | MDPEDVAFMRKALDEARKAREAGEVPVGAVVVKDGEIVARAHNRTIQKSDPTAHA |
| | EILALRKAARALGNYRLTGCTLYATLEPCAMCAGAILHARIERLVYGVPNPKAGATG |
| | SVLNVLNHPRLNHRVEVTGGVLAEECGALLRGFFRAR |
| 3 | MDPTDLAFMREALAEARAAAEAGEVPVGAVVVCDGRIVARAHNRPIALSDPTAHAE |
| | ILALREAARALGNYRLTGCTLYATLEPCAMCAGAILHARIARLVYGVANPKAGACGS |
| | VLDVMNHPRLNHRVEVTGGVLAEECGALLSGFFRAR |
| 4 | MDDAGFMRLALEEARAAAAAGEVPVGAVVVRDGEVLARAGNRTVRDNDPTAHAEI |
| | VALREAARRLGSYRLAGCTLYVTLEPCPMCAGAMIHARLDRLVYGVANPKAGAAG |
| | TVLDVLGHPRLNHRMEVEGGVLAAECGALLRDFFRAR |
| 5 | MDDKGFMQLALEEARAAQAAGEVPVGAVVVRDGEVLARAGNRTRELCDPTAHAEI |
| | VALREAARKRGNYRLPGCTLYVTLEPCAMCAGAMLHARLDRLVYGVANPKAGAAG |
| | SVLDVLGHPRLNHRLEVTGGVLEAECGALLRDFFRAR |
| 6 | MDDKGWMRLALEEARAAKAAGEVPVGAVVVRDGQVLARAGNQVRELCDPTAHAE |
| | IVALREAARKLGNYRLPGCTLYVTLEPCAMCAGAMLHARLDRLVYGVANPKAGAA |
| | GSVLDVLGHPRLNHRLEVEGGVLADECGQLLRDFFRAR |
| 7 | MDPEDLRFMRQALAEARRAAEAGEVPVGAVVVLDGEVVAQAFNRTRAQSDPTAHA |
| | EILALREAARATGNYRLTGCTLYVTLEPCAMCAGAILHARIARLVYGVANPKAGAAG |
| | SVLDVLNHPRLNHRVEVTGGVLAEECGALLRDFFRAR |
| 8 | MNDEGWMQLALEEARKAKAAGEVPVGAVVVRDGEVIAMAGNRTRERCDPTAHAEI |
| | VALREAARRLGNYRLPGCTLYVTLEPCAMCAGAMLHARLDRLVYGVPNPKAGAAG |
| | SVLDVLGHPRLNHRMEVTGGVLEEECGRLLRDFFRAR |
| 9 | MDDEGWMKLALEEARAARAAGEVPVGAVVVKDGEVIARAGNRTRELCDPTAHAEII |
| | ALREAARRLGNYRLPGCTLYVTLEPCAMCAGAMLHARLDRLVYGVANPKAGAAGS |
| | VLDVLGHPRLNHRMEVTGGVLEAECGQLLRDFFRAR |
| 10 | MDPTDLAFMREALAEARAAAEAGEVPVGAVVVCDGRIVARAHNRPIALNDPTAHAE |
| | ILALREAAKALGNYRLTGCTLYATLEPCPMCAGAILHARIARLVYGVANPKAGACGT |
| | VLDVMNHPRLNHRVEVTGGVLAEECGALLSGFFRAR |
| 11 | MDPTDIAFMRQALAEARKAKDAGEVPVGAVVVHDGEIVARAHNRTILESDPTAHAEI |
| | LALRAAARRLGNYRLTGCTLYVTLEPCAMCAGAILHARIERLVYGVANPKAGAAGS |
| | VLNVLNHPRLNHRVEVTGGVLADECGALLSGFFRAR |
| 12 | MDDEDWMRLALEEARKARAAGEVPVGAVVVRDGQVLARAGNRTRELCDPTAHAEI |
| | IALRQAARRRGNYRLAGCTLYVTLEPCAMCAGAMIHARLDRLVYGVPNPKAGAAGS |
| | VLDVLHHPRLNHRLEVTGGVLEAECGALLRDFFRAR |
| 13 | MDPDDVEFMRQALDEARRARDAGEVPVGAVVVHDGRVVARAHNRVIAESDPTAHA |
| | EIRALREAARALGNYRLTGCTLYATLEPCAMCAGAILHARIARLVYGVPNPKAGATG |
| | SVLDVLNHPRLNHRVEVTGGVLEEECGRLLSGFFRAR |
| 14 | MDDQGWMQLALEEARAARAAGEVPVGAVVVRDGEVLARAGNRTRELCDPTAHAEI |
| | RALREAARALGNYRLAGCTLYVTLEPCAMCAGAMIHARLDRLVYGVRNPKAGAAG |
| | SVLDVLGHPRLNHRLEVTGGVLEAECGALLRDFFRAR |
| 15 | MDPEDIEFMKKALAEARAAKEAGEVPVGAVVVSDGEVVARAHNQTIERSDPTAHAE |
| | ILALRAAARALGNYRLTGCTLYVTLEPCAMCAGAILHARIERLVYGVANPKAGAAGS |
| | VLDVLNHPRLNHRVEVTGGVLAEECGALLSDFFRAR |
| 16 | MDDEGWMRLALEEARAARAAGEVPVGAVVVRDGQVLAQAGNRTRELCDPTAHAEI |
| | VALREAARALGNYRLAGCTLYVTLEPCAMCAGAMLHARLDRLVYGVANPKAGAA |
| | GSVLDVLGHPRLNHRAEVEGGVLADECGALLRDFFRAR |
| 17 | MDPEDREFMRKALEEARKAREAGEVPVGAVVVLDGRIVARAHNQTRAESDPTAHAE |
| | ILALREAARALGNYRLTGCTLYVTLEPCAMCAGAILHARIERLVYGVPNPKAGATGS |
| | VLNVLNHPRLNHRVEVTGGVLAEECGALLSGFFRAR |
| 18 | MDDEGWMQLALEEARASKAAGEVPVGAVVVRDGRVLARAGNRTRERCDPTAHAEI |
| | VALREAARRLGNYRLTGCTLYVTLEPCAMCAGAMLHARLDRLVYGVPNPKAGAAG |
| | SVLDVLHHPRLNHRLEVEGGVLEAECGQLLRDFFRAR |
| 19 | MDDRGWMKLALEEARAAKAAGEVPVGAVVVRDGEVIARAGNRTRLKCDPTAHAEI |
| | VALREAARRLGNYRLPGCTLYVTLEPCAMCAGAMLHARLDRLVYGVPNPKAGAAG |
| | SVLDVLGHPRLNHRLEVTGGVLAAECAALLRDFFRAR |
| 20 | MDDEGWMRLALEEARKAKAAGEVPVGAVVVRNGEVIARAGNRTRELCDPTAHAEI |
| | VALREAARRLGNYRLAGCTLYVTLEPCAMCAGAMIHARLDRLVYGVANPKAGAAG |
| | SVLDVLGHPRLNHRLEVEGGVLEAECGALLRDFFRAR |
| 21 | MDPMDIAFMQQALDEARKAKEAGEVPVGAVVVHDGQIVARAHNRTIALSDPTAHA |
| | EILALREAARALGNYRLQGCTLYATLEPCAMCAGAILHARIARLVYGVPNPKAGACG |
| | SVLNVLNHPRLNHRVEVTGGVLAEECGALLRGFFRAR |
| 22 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPKAGAAG |
| | SVLDVLGHPRLNHRMEVEGGVLAAECGALLRDFFRAR |
| 23 | MTDEYFMRQALREARRAYDEGEVPVGAVVVRDGRVIARGRNQVERLKDPTAHAEM |
| | LALTAAAGHLGSKYLTGCTLYVTVEPCAMCAGALVWARVERVVFGVRNPKAGACG |
| | SVFDIPGDRRLNHRPEVVGGVLEAECAALMRDFFRER |
| 24 | MDAALEEARRAAAAGEVPVGAVLVRDGAVLARGGNRTIRDCDPTAHAEIVALRAA |
| | ARAAGNYRLPGATLYVTLEPCAMCAGAMIHARLDRLVYGAADPRAGAAGSVFDVL |
| | RHPALNHQMAVEGGVRAAECGALLRDFFRARR |
| 25 | MTDADFMALALEEARAAAALGEVPVGAVVVRDGAVIARAGNRTIRDCDPTAHAEIV |
| | ALREAARALGNYRLAGCTLYVTLEPCAMCAGAMIHARLDRLVYGAADPKTGAAGS |
| | VLDVLNHPKLNHQMQVEAGVLAEECGAMLRDFFQQRR |
| 26 | MRDALAEARKAADAGEVPVGAVVVRGGEILARAHNRTVADHDPTAHAEILALREA |
| | ARVLGNHRLTGCTLYVTLEPCAMCAGAIVHARLDRLVYGAADPKAGAAGSVLDVL |
| | NHPRLNHRMEVEGGVLAEESGELLRGFFRARR |
| 27 | MDDAGFMREALAEARAAAAAGEVPVGAVVVKDGEIIARAGNRTLRDNDPTAHAEIV |
| | ALRAAARALGNHRLTGCTLYVTLEPCAMCAGAISHARIARLVYGADDPKGGAVAHG |
| | PRFFAQPTCHHRPEVTGGVGAEEAGALLRDFFRARR |
| 28 | MREALAEARAAAEAGEVPVGAVVVRDGEIIARARNRMVADCDPTAHAEIVALREAA |
| | RVLGNHRLTGCTLYATLEPCAMCAGAIAHARIARLVYAADDPKGGAVWHGPRFFEQ |
| | PTCHHRPEVTSGVLADEAAALLRDFFRARR |
| 29 | MREALAEARAAAAAGEVPVGAVVVRDGAVLARAGNASIRARDPTAHAEVLALRAA |
| | ARALGNHRLTGCTLYVTLEPCAMCAGAIAHARVARLVYGAADPKGGAVAHGPRFFE |
| | QPTCHHRPEVTGGVLAEEAGALLRDFFRARR |
| 30 | MREALAEARAAAAAGEVPVGAVVVRDGAVLARAGNRTVRDADPTAHAEIVALREA |
| | ARALGNHRLGGCTLYVTLEPCAMCAGAIAQARVARLVYGADDPKGGAVAHGPRLF |
| | AQPTCHHRPEVVGGVGAAEAGALLRDFFAARR |
| 31 | MTEALAEARKAAAAGEVPVGAVLVRDGAVLARGGNRTIRDCDPTAHAEIVALRAA |
| | ARALGNYRLPGTTLYVTLEPCAMCAGAMIHARVARLVYGAADPKTGAAGSVEDVL |
| | RHPALNHRMEVEGGVLAEECGALLREFFRARR |
| 32 | MREALAEARAAAAAGEVPVGAVVVRDGAVLARAGNRTVRDADPTAHAEIVALREA |
| | ARALGNHRLTGCTLYVTLEPCAMCAGAISHARVARLVYGADDPKGGAVAHGPRFFA |
| | QPTCHHRPEVTGGVLAAEAGELLRGFFRARR |
| 33 | MRDALAEARAAAAAGEVPVGAVVVRDGAIVARARNRMVADCDPTAHAEIVALRAA |
| | ARALGNHRVDGCTLYVTLEPCAMCAGAMIHARLARLVYGAADPRAGAAGSVLDVL |
| | GHPALNHRMEVTAGVLAEECGALLRDFFAARR |
| 34 | MTEALAEARKAAAAGEVPVGAVLVRDGAVLARGGNRTIRDCDPTAHAEIVALRAA |
| | ARALGNYRLPGTTLYVTLEPCAMCAGAMIHARVARLVYGAADPKTGAAGSVFDILR |
| | HPALNHRMEVEGGVLAEECGALLRDFFRARR |
| 35 | MREALAEARAAAAAGEVPVGAVVVRDGAVLARAGNRTVRDADPTAHAEIVALRAA |
| | ARALGNHRLDGCTLYVTLEPCAMCAGAIAQARVARLVYGAADPKGGAVAHGPRLF |
| | EQPTTHHRPEVVGGVLAEEAAALLRGFFAARR |
| 36 | MRAALAEARAAAAAGEVPVGAVVVRDGAVLARAGNASIRARDPTAHAEILALRAA |
| | ARALGNHRLTGCTLYVTLEPCAMCAGAIAHARIARLVYGAADPKGGAVAHGPRFFE |
| | QPTCHHRPEVTGGVLAEEAGALLRAFFRARR |
| 37 | MRDALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPEVVGGVGAAEAGALLRDFFRARR |
| 38 | MREALAEARAAAAAGEVPVGAVVVRDGAVLARAGNRTVRDADPTAHAEIVALRAA |
| | ARALGNHRLDGCTLYVTLEPCAMCAGAMVQARLARLVYGAADPKAGAVDSVLDV |
| | LDHPRLNHRMEVTGGVLAEECGALLREFFAARR |
| 39 | MRDALAEARAAAAAGEVPVGAVVVRDGAILARAGNASIRARDPTAHAEILALRAAA |
| | RALGNHRLDGCTLYVTLEPCAMCAGAIAHARIARLVYGAADPKGGAVAHGPRFFEQ |
| | PTCHHRPDVTGGVGAAEAAALLRDFFRARR |
| 40 | MDDAGFMRLALEEARAAAAAGEVPVGAVVVRDGEVLARARNAPVAAHDPTAHAEI |
| | LALRAAAAALGNYRLDGCTLYVTLEPCAMCSGAMLHARLARVVYGAADPKTGAAG |
| | SVLDLFAQPRLNHHTAVEGGVLAAECGALLRDFFRARRG |
| 41 | MDDEFFMREALRLAEEAAAAGEVPVGAVVVRDGEIVGRGRNRVLEDRDPTAHAEIV |
| | ALREAARRLGNYRLEGCELYVTLEPCAMCAGAMVHARLARLVYGAADPKAGAAGS |
| | VLDVLGHPRLNHRMEVTGGVLAEECGALLREFFRARR |
| 42 | MRDALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPDVTGGVGAAEAAALLRDFFRARR |
| 43 | MREALAEARAAAAAGEVPVGAVVVRDGAVLARAGNRTVRDADPTAHAEVVALRE |
| | AARALGNHRLGGCTLYVTLEPCAMCAGAIAQARVARLVYGADDPKGGAVAHGPRL |
| | FAQPTCHHRPEVVGGVGAAEAAALLRGFFAARR |
| 44 | MDDAGFMRLALAEARAAAAAGEVPVGAVVVRDGAVLAAAGNRTLRDCDPTAHAE |
| | VVALRAAARALGNHRLGGCDLYVTLEPCAMCAGAISFARIRRLYFGADDPKGGAVE |
| | HGPRFFAQPTCHHAPEVYGGLAESEAAALLRDFFRARR |
| 45 | MDDAGFMRLALAEARAAAAAGEVPVGAVVVRDGAVLAAAGNRTLRDCDPTAHAE |
| | VVALRAAARALGNHRLEGCTLYVTLEPCAMCAGAMLHARLARVVYGAADPKTGA |
| | AGSVLDLFANPRLNHHTRVEGGVLAEECGALLQDFFRARRG |
| 46 | MSDEDYMRLALEEARAAAAAGEVPIGAVVVRDGEVLARAGNRTVRDCDPTAHAEI |
| | VALREAARKLGNYRLAGCTLYVTVEPCAMCAGALVWARVDRLVYGADDPKAGAV |
| | RSALAVVDHPRLNHRMEVVSGVLAGECAALLQEFFAARR |
| 47 | MREALAEARAAAAAGEVPVGAVVVRDGAVLARAGNASIRARDPTAHAEILALRAA |
| | ARALGNHRLTGCTLYVTLEPCAMCAGAIAHARVARLVYGAADPKGGAVAHGPRFFE |
| | QPTCHHRPEVTGGVLAEEAGALLRDFFRARR |
| 48 | MREALAEARAAAAAGEVPVGAVVVRDGAVLARAGNASIRARDPTAHAEILALRAA |
| | ARALGNHRLDGCTLYVTLEPCAMCAGAIAQARVARLVYGAADPKGGAVAHGPRLF |
| | AQPTCHHRPEVTGGVGEAEAAALLRDFFAARR |
| 49 | MTDEYFMRQALREARKAYDEDEVPVGAVVVRDGKVIARGRNQVERLKDPTAHAEM |
| | IALTSAANYLGSKRLEGCTVYVTLEPCPMCAGALVLARVERLVFGAFDPKAGACGTL |
| | YDIPGDRRLNHRIEVVGGVLEEESAALLREFFEKRR |
| 50 | MREALAEARAAAAAGEVPVGAVVVRDGAVLARAGNASIRARDPTAHAEMLALRAA |
| | ARALGNHRLDGCTLYVTLEPCAMCAGAIAHARIARLVYGAADPKGGAVAHGPRFFA |
| | QPTCHHRPEVTGGVLAEEAGALLRDFFRARR |
| 51 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPEVVGGVGAGEAAALLRDFFAARR |
| 52 | MDDAGFMRLALAEARAAAAAGEVPVGAVVVRDGAVLAAAGNRTLRDCDPTAHAE |
| | VVALRAAARALGNHRLEGCDLYVTLEPCAMCAGAISFARIRRLYFGAADPKGGAVE |
| | HGPRFFAQPTCHHAPEVYGGLAESEAAALLRGFFAARR |
| 53 | MREALAEARAAAAAGEVPVGAVVVRDGAVLARAGNRTVRDADPTAHAEIVALRAA |
| | ARALGNHRLDGCTLYVTLEPCAMCAGAIAQARVARLVYGAADPKGGAVAHGPRLF |
| | EQPTTHHRPEVVGGVLAAEAGALLRAFFAARR |
| 54 | MTEALAEARKAAALGEVPVGAVVVRDGAVIARGHNRTVTDADPTAHAEIVALREA |
| | ARALGNHRLTGCTLYVTLEPCAMCAGAIVHARVDRLVYGAADPKAGAAGSVLDVIG |
| | HPRLNHRPEVEGGVLGEECGALLRDFFRARR |
| 55 | MRSALDLAAAAAAAGEVPVGAVVVRDGAIVGRGENRVLRDSDPTAHAEIVAMREA |
| | ARALGNYRLTGCTLYVTLEPCAMCAGAMVHARLDRLVYGAADPKAGAAGSVLDVL |
| | GHPRLNHQMEVEGGVLAAESAALLRDFFRARR |
| 56 | MREALAEARAAAAAGEVPVGAVVVRDGAVLARAGNASIRARDPTAHAEILALRAA |
| | ARALGNHRLTGCTLYVTLEPCAMCAGAIAHARIARLVYGAADPKGGAVAHGPRFFE |
| | QPTCHHRPEVTGGVLAEEAGALLRDFFRARR |
| 57 | MDDAGFMRLALAEAEAAAAAGEVPVGAVVVRDGEVIARAGNRTVRDCDPTAHAEI |
| | VALRAAARALGNHRLTGCTLYVTLEPCAMCAGAISHARVARLVYGADDPKGGAVA |
| | HGPRFFAQPTCHHRPEVAGGVLAEEAGALLRGFFRARR |
| 58 | MDDAGFMRLALAEARAAAAAGEVPVGAVVVRDGAVLAAAGNRTLRDCDPTAHAEI |
| | VALRAAARALGNYRLDGCDLYVTLEPCAMCAGAMLHARLRRVVFGAADPKTGAA |
| | GSVLDLFAERRLNHRTAVAGGVLADECGALLRDFFRARR |
| 59 | MDDAGFMRLALAEARAAAAAGEVPVGAVVVRDGAVLAAAGNRTLRDCDPTAHAE |
| | VVALRAAARALGNHRLDGCDLYVTLEPCAMCAGAISHARIRRLYYGADDPKGGAV |
| | DNGVRFFASPTCHHAPEVYGGLAEGEAAALLRDFFRER |
| 60 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGADDPKGGAVAHGPRFF |
| | AQPTCHHRPEVVGGVGAEEAGALLRGFFAARR |
| 61 | MTEALREARRAAAAGEVPVGAVVVRDGAVLARAGNRTVRDADPTAHAEIVALREA |
| | ARALGNHRLTGCTLYVTLEPCAMCAGAISHARIARLVYGAADPKGGAVAHGPRFFE |
| | QPTCHHRPEVTGGVGEAEAAALLRDFFRARR |
| 62 | MREALAEARAAAAAGEVPVGAVVVRDGAVLARAGNRTVRDADPTAHAEVVALRA |
| | AARALGNHRLGGCTLYVTLEPCAMCAGAIAQARVARLVYGAADPKGGAVAHGPRL |
| | FAQPTCHHRPEVVGGVGEAEAAALLRDFFAARR |
| 63 | MREALAEARAAAAAGEVPVGAVVVRDGAVLARAGNASIRARDPTAHAEVLALRAA |
| | ARALGNHRLTGCTLYVTLEPCAMCAGAIAHARIARLVYGAADPKGGAVAHGPRFFE |
| | QPTCHHRPEVTGGVLAEEAGALLRDFFRARR |
| 64 | MTDEYFMRQALREARRAYEEDEVPVGAVVVRDGRVIARGRNQVERLKDPTAHAEM |
| | IALTAAANHLGSKRLEGCTVYVTLEPCPMCAGALVLARVPRLVFGAFDPKAGACGTL |
| | YDIVRDPRLNHRVEVVGGVLEEECGELLKRFFRERR |
| 65 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVAHGPRLF |
| | AQPTCHHRPEVVGGVGAAEAGALLRDFFAARR |
| 66 | MTEALAEARKAAAEGEVPVGAVVVRDGVVLARAHNRTVADHDPTAHAELLAIREA |
| | ARVLGNHRLTGCTLYVTLEPCAMCAGAIVHARLDRLVYGAADPKAGAAGSVLDVL |
| | GHPRLNHRPEVEGGVLAAESAALLRDFFRARR |
| 67 | MDDAGFMRLALAEAERAAALGEVPVGAVLVRDGEVLAAAGNRTVADCDPTAHAE |
| | MLALREGARRLGNYRLTGCTLYVTLEPCAMCAGAMVHARLDRLVYAAADPKAGA |
| | AGSVLDVLNHPALNHRMQVEGGVLAEESAALLRGFFRARR |
| 68 | MREALAEARAAAAAGEVPVGAVVVRDGAVLARAGNRTVRDADPTAHAEIVALREA |
| | ARALGNHRVGGATLYVTLEPCAMCAGAISQARVARLVYGADDPKGGAVAHGPRFF |
| | EQPTCHHRPEVTGGVLAEESAALLRGFFAARR |
| 69 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPEVVGGVGAAEAGALLRDFFRARR |
| 70 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVAHGPRFF |
| | EQPTCHHRPDVTGGVLAEEAGALLRDFFRARR |
| 71 | MTDEYFMRQALREARRAYDEGEVPVGAVVVRDGRVIARGRNQVERLKDPTAHAEM |
| | IALTAAADYLGSKRLEGCTLYVTLEPCAMCAGAIVLARIPRLVFGAFDPKAGACGTL |
| | YDIVRDRRLNHRAEVVSGVLEEECGALLKEFFARLR |
| 72 | MDDEFFMREALRLAEEAAAAGEVPVGAVVVRDGEIVGRGRNRVLEDRDPTAHAEIV |
| | AMREAARRLGNYRLEGCTLYVTLEPCAMCAGAMVHARVARLVYGAADPKAGAAG |
| | SVLDVLGHPRLNHRMEVTGGVLAEECGALLREFFRRRR |
| 73 | MREALAEARAAAAAGEVPVGAVVVRDGAVLARAGNRTVRDADPTAHAELVAIRAA |
| | AAALGQERLTGCTLYVTLEPCAMCAGAISHARIARLVYGAADPKGGAVAHGPRFFE |
| | QPTCHHRPEVTGGVLAEEAGALLRDFFRARR |
| 74 | MREALAEARAAAAAGEVPVGAVVVRDGAIVARARNRMVADCDPTAHAEIVALREA |
| | ARALGNHRLDGCTLYVTLEPCAMCAGAMVHARVARLVYGAADPRAGAAGSVLDV |
| | LGHPALNHRMEVAGGVLAEECGALLREFFAARR |
| 75 | MRAALAEARAAAAAGEVPVGAVVVRDGAVLARAGNASIRARDPTAHAEILALRAA |
| | ARALGNHRLDGCTLYVTLEPCAMCAGAIAHARVARLVYGAADPKGGAVAHGPRFF |
| | EQPTCHHRPEVTGGVLAEEAGALLRDFFRARR |
| 76 | MRAALDEARAAAAAGEVPVGAVVVRDGAILARAGNRTVRDCDPTAHAEIVALREA |
| | ARALGNHRLTGCTLYVTLEPCAMCAGAISHARVARLVYGADDPKGGAVRHGPRFFE |
| | QPTCHHRPEVVGGVGAEEAAALLRGFFAARR |
| 77 | MDDEAWMRRAIALAHQAEAEGEVPVGAVLVKDGEVIAEGWNRSIGHHDATAHAEI |
| | ETLRQAGQALQNYRLLDTTLYVTLEPCPMCAGALLHSRVKRVVFGAPDLKAGAAGT |
| | VLNLFESQASYHYAEVESGLLEQECREQLQAFFKRRRKEIKALRQAQRDAE |
| 78 | MDDAGFMRLALAEARAAAAAGEVPVGAVVVRDGAVLAAAGNRTLRDCDPTAHAE |
| | VVALRAAARALGNHRLEGCDLYVTLEPCAMCAGAIAHARIARLYYGAADPKGGAV |
| | EHGARVFDQPQCLHRPEVYGGIGEAEAAALLRGFFAARR |
| 79 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGADDPKGGAVAHGPRFF |
| | AQPTCHHRPEVAGGLGEAEAGALLRDFFAARR |
| 80 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGAADPKGGAVAHGPRLF |
| | AQPTCHHRPEVVGGVGEAEAAALLRAFFAARR |
| 81 | MRDALAEARAAAAAGEVPVGAVVVRDGAVLARAGNASIRARDPTAHAEILALRAA |
| | ARALGNHRLDGCTLYVTLEPCAMCAGAIAHARIARLVYGAADPKGGAVAHGPRFFA |
| | QPTCHHRPEVTGGVGEAEAAALLRAFFAARR |
| 82 | MTDEYFMRQALREARRAYEEDEVPVGAVVVRDGRVIARGRNQVERLKDPTAHAEM |
| | IALTAAANHLGSKRLEGCTVYVTLEPCPMCAGALVLARVERLVFGAFDPKAGACGT |
| | LYDIPGDRRLNHRLEVVGGVLEEESAALLREFFRKRR |
| 83 | MTDEDYMRLALEEARAAAAAGEVPIGAVVVRDGEVLARAGNRTVRDCDPTAHAEI |
| | VALREAARVLGNYRLAGCTLYVTVEPCAMCAGAIVHARVARLVYGADDPKGGAVR |
| | SCLEVLDHPRLNHRVEVTAGVLAGECAALLQDFFAARR |
| 84 | MREALAEARAAAAAGEVPVGAVVVRDGAVLARAGNRTVRDADPTAHAEVLALRA |
| | AARALGSERLTGCTLYVTLEPCAMCAGAISHARVARLVYGAADPKGGAVEHGARVF |
| | EQPTCHHRPEVVGGVGAAEAGALLRDFFAARR |
| 85 | MREALAEARAAAAAGEVPVGAVVVRDGAILARAGNASIRARDPTAHAEILALRAAA |
| | RALGNHRLTGCTLYVTLEPCAMCAGAIAHARIARLVYGAADPKGGAVAHGPRFFEQ |
| | PTCHHRPEVTGGVLAEEAGALLRAFFRARR |
| 86 | MDDAGFMRLALEEARAAAAAGEVPVGAVVVRDGAVLARAGNRTLRDRDPTAHAE |
| | MVALRAAARALGSERLTGCDLYVTLEPCAMCAGAISFARIRRLYFGAADPKGGAVE |
| | NGVRFFASPTCHHAPEVYGGLAESEAAALLRDFFRARR |
| 87 | MTDEYFMRQALREARRAYDEGEVPVGAVVVRDGRVIARGRNQVERLKDPTAHAEM |
| | LALTAAAGHLGSKYLTGCTLYVTVEPCAMCAGALVWARVERVVFGARDPKAGACG |
| | SVFDIPGDRRLNHRPEVVGGVLEAECAALMRDFFRERR |
| 88 | MREALAEARAAAAAGEVPVGAVVVRDGAVLARAGNASIRARDPTAHAEMLALRAA |
| | ARALGNHRLDGCTLYVTLEPCAMCAGAIAHARVARLVYGAADPKGGAVAHGPRFF |
| | EQPTCHHRPEVTGGVLAEEAGALLREFFRARR |
| 89 | MREALAEARAAAAAGEVPVGAVVVRDGAILARAGNASIRARDPTAHAEILALRAAA |
| | RALGNHRLTGCTLYVTLEPCAMCAGAIAHARIARLVYGAADPKGGAVAHGPRFFAQ |
| | PTCHHRPEVTGGVLAEEAGALLRAFFRARR |
| 90 | MSDEQYMRRALELARQAEQAGEVPVGAVLVKDGEIIAEGWNQSISAHDATAHAEIM |
| | ALRAAGQALENYRLLDTTLYVTLEPCPMCAGALLHSRVKRVVFGAPDLKAGAAGTV |
| | LNLFESQASYHYADVESGLLEDECREQLQAFFKRRRKEIKALRQAQREAEE |
| 91 | MSEEQYMRRALELARQAEQEGEVPVGAVLVKDGEVIAEGWNRSIGSHDATGHAEIM |
| | ALRAAGEKLENYRLLDTTLYVTLEPCPMCAGALLHSRVKRVVFGAPDLKAGAAGTV |
| | LNLFESQASYHYADVESGLLEEECREQLQAFFKRRRKEIKALRQAQKEAE |
| 92 | MDDAGFMRLALAEARRAAEAGEVPVGAVVVRDGAVLAAAGNRTLRDCDPTAHAE |
| | VVALREAARALGNHRLGGCDLYVTLEPCAMCAGAISHARIARLYYGADDPKGGAV |
| | AHGARVFDQPTCHHRPEVYGGIGAAEAAALLRDFFRARR |
| 93 | MREALAEARAAAAAGEVPVGAVVVRDGAVLARAGNASIRARDPTAHAEVLALRAA |
| | ARALGNHRLTGCTLYVTLEPCAMCAGAIAHARVARLVYGAADPKGGAVAHGPRFFE |
| | QPTCHHRPEVTGGVLAAEAGALLRDFFRARR |
| 94 | MREALAEARAAAAAGEVPVGAVVVRDGAVLARAGNASIRARDPTAHAEMLALRAA |
| | ARALGNHRLDGCTLYVTLEPCAMCAGAIAHARIARLVYGAADPKGGAVAHGPRFFA |
| | QPTCHHRPDVTGGVLAEEAGALLRDFFRARR |
| 95 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGADDPKGGAVAHGPRFF |
| | AQPTCHHRPEVVGGVGEAEAGALLRDFFAARR |
| 96 | MTDEYFMRQALREARRAYDEGEVPVGAVVVRDGRVIARGRNQVERLKDPTAHAEM |
| | LALTAAAAHLGSKRLEGCTVYVTLEPCAMCAGALVLARVDRLVFGAFDPKAGACGT |
| | VYDIPRDRRLNHRVEVVGGVLEEESAALLRAFFEERR |
| 97 | MREALAEARAAAAAGEVPVGAVVVRDGAVLARAGNASIRARDPTAHAEILALRAA |
| | ARALGNHRLDGCTLYVTLEPCAMCAGAIAQARVARLVYGAADPKGGAVAHGPRLF |
| | AQPTCHHRPEVVGGVGAEEAAALLRGFFAARR |
| 98 | MTDEYFMRQALREARRAYDEGEVPVGAVVVRDGRVIARGRNQVERLKDPTAHAEM |
| | IALTAAADHLGSKRLEGCTLYVTLEPCAMCAGAIVLARIPRLVFGAFDPKAGACGTL |
| | YDIVRDRRLNHRVEVVSGVLEEESAALLREFFAERR |
| 99 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPDVVGGVGAAEAGALLRDFFRARR |
| 100 | MTDEYFMRQALREARRAYDEDEVPVGAVVVRDGRVIARGRNQVERLKDPTAHAEM |
| | IALTSAANHLGSKRLEGCTVYVTLEPCPMCAGALVLARVPRLVFGAFDPKAGACGTL |
| | YDIPRDRRLNHRVEVVGGVLEEESAALLREFFARRR |
| 101 | MTEALAEARKAAAAGEVPVGAVLVRDGEILARGGNRTIRDCDPTAHAEIVALREAA |
| | RKLGNYRLAGCTLYVTLEPCAMCAGAMIHARLDRLVYGADDPKAGAAGSVLDVLN |
| | HPRLNHQMEVTRGVLADECGALLRDFFQARR |
| 102 | MTDEYFMRQALREARRAFDEDEVPVGAVVVRDGRVIARGRNQVERLKDPTAHAEM |
| | IAITSAANYLGSKRLEGCTVYVTLEPCPMCAGALVLARVPRLVFGAFDPKAGACGTL |
| | YDIPRDRRLNHRLEVVGGVLEEESAALLREFFARRR |
| 103 | MTDEDYMRLALEEARAAAAAGEVPVGAVVVRDGAVLARAGNRTVRDCDPTAHAEI |
| | VALRAAARALGNYRLPGCTLYVTVEPCAMCAGAMIHARLDRLVYGADDPKAGAVR |
| | STLRVLDHPALNHRMAVTAGVLADECAALLQDFFRARR |
| 104 | MREALAEARAAAAAGEVPIGAVVVRDGAVLARAGNRTVRDCDPTAHAEIVALREA |
| | ARALGNHRLTGCTLYVTVEPCAMCAGAISWARVARLVYGAADPKGGAVRHGPRLF |
| | EQPTCHHAPEVVDGVCAEEAAALLRDFFRARR |
| 105 | MDDEALMGLALDEARAAAAAGEAPIGAVVARGGEVLAAAGNRTLRDCDPTAHAEI |
| | LALREAARKLGNYRLTGCTLYATLEPCAMCAGAISHARIARLVYGADDPKGGAVRH |
| | GPRFFEQPTCHHRPEVAGGVGAAEAGALLRDFFRARR |
| 106 | MDDEALMGLALDEARAAAAAGEAPIGAVVARGGEVLAAAGNRTLRDCDPTAHAEV |
| | LALREAARRLGNYRLTGCTLYVTLEPCAMCAGAISHARVARLVYGAADPKGGAVA |
| | HGPRFFEQPTCHHRPEVTGGVGAAEAGALLRDFFRARR |
| 107 | MSDEQYMRRALELARQAEAEGEVPVGAVLVRDGEVIAEGWNRSIGSHDATGHAEIM |
| | ALRAAGQALQNYRLLDTTLYVTLEPCPMCAGALLHSRVKRVVFGAPDLKAGAAGT |
| | VLNLFESQASYHYADVESGLLEEECREQLQAFFKRRRKEIKALRQAQREAEEK |
| 108 | MTEALAEARKAAAAGEVPVGAVLVRDGAVLARGGNRTIRDCDPTAHAEIVALRAA |
| | ARALGNYRLPGTTLYVTLEPCAMCAGAMIHARVDRLVYGAADPRAGAAGSVLDVIG |
| | HPALNHRMAVEGGVLAEECGALLRDFFRARR |
| 109 | MTDEYFMRQALREARRAYDEGEVPVGAVVVRDGRVIARGRNQVERLKDPTAHAEM |
| | LALTAAAAHLGSKRLEGCTLYVTLEPCAMCAGAIVLARVPRLVFGAFDPKAGACGT |
| | LYDIVRDRRLNHRVEVVSGVLEEESAALLREFFARLR |
| 110 | MREALAEARAAAAAGEVPVGAVVVRDGAVLARAGNRTVRDADPTAHAEIVALRAA |
| | ARALGNHRLDGCTLYVTLEPCAMCAGAIAQARVARLVYGAADPKGGAVAHGPRLF |
| | EQPTTHHRPEVVGGVLAEEAGALLRGFFAARR |
| 111 | MREALAEARAAAAAGEVPVGAVVVRDGAVLARAGNASIRARDPTAHAEILALRAA |
| | ARALGNHRLDGCTLYVTLEPCAMCAGAIAHARVARLVYGAADPKGGAVAHGPRFF |
| | EQPTCHHRPEVTGGVLAAEAGALLRDFFRARR |
| 112 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPEVVGGVGAAEAGALLREFFAARR |
| 113 | MRDALAEARAAAAAGEVPVGAVVVRDGAILARAGNASIRARDPTAHAEILALRAAA |
| | RALGNHRLTGCTLYVTLEPCAMCAGAIAHARIARLVYGAADPKGGAVAHGPRFFEQ |
| | PTCHHRPEVTGGVLAEEAGALLRDFFRARR |
| 114 | MDDAGFMRLALAEAEAAAAAGEVPVGAVVVRDGEVIARAGNRTVRDCDPTAHAEI |
| | VALREAARKLGNYRLPGCTLYVTLEPCAMCAGAMIHARLDRVVYGAADPKTGAAG |
| | SVLDLFADRRLNHHTAVVGGVLAEEAGALLRAFFAARR |
| 115 | MREALAEARAAAAAGEVPVGAVVVRDGAVLARAGNASIRARDPTAHAEILALRAA |
| | ARALGNHRLTGCTLYVTLEPCAMCAGAIAHARVARLVYGAADPKGGAVAHGPRFFE |
| | QPTCHHRPEVTGGVLAEEAGALLRAFFRARR |
| 116 | MREALAEARAAAAAGEVPVGAVVVRDGAVLARAGNASIRARDPTAHAEMLALRAA |
| | ARALGNHRLTGCTLYVTLEPCAMCAGAIAHARVARLVYGAADPKGGAVAHGPRFFE |
| | QPTCHHRPEVTGGVLAEEAGALLRDFFRARR |
| 117 | MRAALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPEVVGGVGAAEAGALLRDFFRARR |
| 118 | MREALAEARAAAAAGEVPVGAVVVRDGAVLARAGNASIRARDPTAHAEVLALRAA |
| | ARALGNHRLTGCTLYVTLEPCAMCAGAIAHARLARLVYGAADPKGGAVEHGPRFFA |
| | QPTCHHRPEVTGGVGAAEAGALLRAFFAARR |
| 119 | MSDEQFMRRAIELAKKGEELGEVPVGAVLVKDGEIIAEGWNQSISTHDATAHAEIMA |
| | LRAAGEKLQNYRLLDTTLYVTLEPCPMCAGALLHSRVKRVVFGAPDLKAGAAGTVL |
| | NLFESQASYHYADVESGLLEEECREQLQAFFKRRRKEIKALKKAKKEAEQK |
| 120 | MREALAEARAAAAAGEVPIGAVVVRDGAVLARAGNRTVRDCDPTAHAEIVALREA |
| | ARALGNYRLDGCTLYVTLEPCAMCAGAMLHARLARVVYGAADPKTGAAGSVLDLF |
| | AERRLNHQTEVAGGVLAEECGALLRDFFRARRG |
| 121 | MTEALAEARKAAALGEVPVGAVVVRDGAVIARGHNRTVTDTDPTAHAEIVALREAA |
| | RALGNHRLTGCTLYVTLEPCAMCAGAIVHARLDRVVYGAADPKAGAAGSVLDVLG |
| | HPRLNHRPEVAGGVLAAESAALLRDFFRARR |
| 122 | MTDEYFMRQALREARRAYDEGEVPVGAVVVRDGRVIARGRNQVERLKDPTAHAEM |
| | LALTAAAAHLGSKRLEGCTLYVTLEPCAMCAGAIVLARIPRLVFGAFDPKAGACGTL |
| | YDIVRDRRLNHRAEVVSGVLEEECGALLKEFFARLR |
| 123 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGADDPKGGAVAHGPRFF |
| | AQPTCHHRPEVVGGVGEAEAAALLRGFFAARR |
| 124 | MREALAEARAAAAAGEVPVGAVVVRDGAVLARAGNRTVRDADPTAHAELVAIRAA |
| | ARALGSERLTGCTLYVTLEPCAMCAGAISHARVARLVYGAADPKGGAVAHGPRFFE |
| | QPTCHHRPEVTGGVLAEEAGALLRDFFRARR |
| 125 | MTDEYFMRQALREARRAYEEDEVPVGAVVVREGRVIARGRNQVERLKDPTAHAEMI |
| | ALTSAANYLGSKRLEGCTVYVTLEPCPMCAGALVLARVERLVFGAFDPKAGACGTL |
| | YDIPGDRRLNHRIEVVGGVLEEESAALLREFFRRRR |
| 126 | MSEALAEARKAAALGEVPVGAVVVRDGAVIARGHNRTVADADPTAHAEIVALREA |
| | ARALGNHRLTGCTLYVTLEPCAMCAGAIVHARLDRLVYGAADPKAGAAGSVLDVL |
| | GHPRLNHRPEVEGGVLAAESAALLRDFFRARR |
| 127 | MSDEQYMRRALELARQAEQEGEVPVGAVLVKDGEVIAEGWNRSIGSHDATGHAEIM |
| | ALRAAGEKLGNYRLLDTTLYVTLEPCPMCAGALLHSRVKRVVFGAPDLKAGAAGTV |
| | LNLFESQASYHYADVESGLLEEECREQLQAFFKRRRKEIKAARKAKREAEEK |
| 128 | MREALAEARAAAAAGEVPVGAVVVRDGAVLARAGNASIRARDPTAHAEMLALRAA |
| | ARALGNHRLTGCTLYVTLEPCAMCAGAIAHARVARLVYGAADPKGGAVAHGPRFFE |
| | QPTCHHRPEVTGGVLAEEAGALLRAFFRARR |
| 129 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGAADPKAGAAG |
| | SVLDVLGHPRLNHRMEVEGGVLAAECGALLRDFFRARR |
| 130 | MRAALDEARAAAAAGEVPVGAVVVRDGAVLARAGNASIRARDPTAHAEILALRAA |
| | ARALGNHRLTGCTLYVTLEPCAMCAGAIAHARVARLVYGAADPKGGAVEHGPRFFA |
| | QPTCHHRPEVTGGVLAAEAGALLRDFFRARR |
| 131 | MTEALAEARKAAAAGEVPVGAVVVRDGEILARAHNRTVADHDPTAHAEILALREAA |
| | RRLGNHRLTGCELYVTLEPCAMCAGAISHARIARLVYGAADPKGGAVEHGPRFFAQP |
| | TCHHRPEVVGGVGASEAAALLRDFFRARR |
| 132 | MRAALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVAHGPRFF |
| | EQPTCHHRPEVTGGVLAEEAGALLRAFFRARR |
| 133 | MDDAGFMRLALEEARAAAAAGEVPVGAVVVRDGEVLARAGNRTVRDCDPTAHAE |
| | VVALREAARKLGNHRLTGCTLYVTLEPCAMCAGAISHARVARLVYGADDPKGGAV |
| | AHGPRFFAQPTCHHRPEVAGGLLAGEAAALLRDFFAARR |
| 134 | MRDALAEARAAAAAGEVPVGAVVVRDGAVLARAGNASIRARDPTAHAEILALRAA |
| | ARALGNHRLTGCTLYVTLEPCAMCAGAIAHARIARLVYGAADPKGGAVAHGPRFFE |
| | QPTCHHRPEVTGGVLAEEAGALLREFFRARR |
| 135 | MTEALAEARKAAAAGEVPVGAVLVRDGAVLARGGNRTIRDCDPTAHAEIVALRAA |
| | ARALGNYRLPGTTLYVTLEPCAMCAGAMIHARVDRLVYGAADPRAGAAGSVEDVIR |
| | HPALNHRMEVEGGVLAEECGALLRDFFRARR |
| 136 | MREALAEARAAAAAGEVPVGAVVVRDGAVLARAGNRTVRDADPTAHAEIVALREA |
| | ARALGNHRLTGCTLYVTLEPCAMCAGAISHARVARLVYGADDPKGGAVAHGPRFFA |
| | QPTCHHRPEVVGGVGAEEAGALLRDFFRARR |
| 137 | MTDEYFMRQALREARRAYDEGEVPVGAVVVRDGRVIARGRNQVERLKDPTAHAEM |
| | LALTAAAAHLGSKRLEGCTLYVTLEPCAMCAGAIVLARIPRLVFGAFDPKAGACGTL |
| | YDIVRDRRLNHRVEVVSGVLEEESAALLREFFARLRA |
| 138 | MSDEQYMRRALELARQAEEEGEVPVGAVLVKDGEIVAEGWNRSIGSHDATGHAEIM |
| | ALRAAGEKLENYRLLDTTLYVTLEPCPMCAGALLHSRVKRVVFGAPDLKAGAAGTV |
| | LNLFESQASYHYADVESGLLEEECREQLQAFFKRRRKEIKAQRDAEKAAAE |
| 139 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPEVVGGVGAAEAGALLRAFFAARR |
| 140 | MRDALAEARAAAAAGEVPVGAVVVRDGAILARAGNASIRARDPTAHAEVLALRAA |
| | ARALGNHRLDGCTLYVTLEPCAMCAGAIAHARVARLVYGAADPKGGAVEHGPRFF |
| | AQPTCHHRPDVTGGVGAAEAAALLRDFFRARR |
| 141 | MRDALAEARAAAAAGEVPVGAVVVRDGAVLARAGNRTVRDCDPTAHAEIVALRAA |
| | ARALGNHRLDGCTLYVTLEPCAMCAGAIAQARVARLVYGADDPKGGAVAHGPRLF |
| | AQPTCHHRPEVVGGVGAEEAAALLRGFFAARR |
| 142 | MTDEYFMRQALREARRAYDEDEVPVGAVVVRDGKVIARGRNQVERLKDPTAHAEM |
| | IALTSAANHLGSKRLEGCTVYVTLEPCAMCAGALVLARVDRLVFGAFDPKAGACGT |
| | LYDIPRDRRLNHRVEVVGGVLEEESAALLREFFRRRR |
| 143 | MTDEYFMRQALREARRAYDEGEVPVGAVVVRDGKVIARGRNQVERLKDPTAHAEM |
| | IALTAAANHLGAKRLEGCTVYVTLEPCAMCAGALVLARVDRLVFGAFDPKAGACGT |
| | LYDIPRDARLNHRVEVVSGVLEEESAALLREFFARLR |
| 144 | MSEALAEARKAAAAGEVPVGAVLVRDGAVLARGGNRTIRDCDPTAHAEIVALRAAA |
| | RALGNYRLPGTTLYVTLEPCAMCAGAMIHARVDRLVYGAADPRAGAAGSVFDILRH |
| | PALNHRMEVEGGVLADECGALLRDFFRARR |
| 145 | MRAALAEARAAAAAGEVPVGAVVVRDGAILARAGNASIRARDPTAHAEVLALRAA |
| | ARALGNHRLTGCTLYVTLEPCAMCAGAIAHARIARLVYGAADPKGGAVAHGPRFFA |
| | QPTCHHRPEVTGGLGEAEAAALLRAFFAARR |
| 146 | MRAALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVAHGPRFF |
| | EQPTCHHRPEVTGGVLAAEAGALLRDFFRARR |
| 147 | MTEALAEARKAAALGEVPVGAVVVRDGAVIARGHNRTVADADPTAHAEIVALREA |
| | ARALGNHRLTGCTLYVTLEPCAMCAGAIVHARLDRVVYGAADPKAGAAGSVLDVL |
| | GHPRLNHRPEVAGGVLAEECGALLRDFFRARR |
| 148 | MREALAEARAAAAAGEVPVGAVVVRDGAVLARAGNRTVRDADPTAHAEIVALREA |
| | ARALGNHRLGGCTLYVTLEPCAMCAGAIAQARVARLVYGAADPKGGAVAHGPRLF |
| | EQPTCHHRPEVVGGVLAEEAGALLRGFFAARR |
| 149 | MTDEYFMRQALREARRAYEEGEVPVGAVVVRDGRVIARGRNQVERLKDPTAHAEM |
| | IALTAAAAHLGNKRLEGCTLYVTLEPCAMCAGAIVLARIPRLVFGAFDPKAGACGTL |
| | YDIVRDRRLNHRAEVVSGVLEEECGALLKEFFARLR |
| 150 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGAADPKGGAVAHGPRLF |
| | AQPTCHHRPEVVGGVGEAEAAALLRDFFAARR |
| 151 | MDDEFYMRRALELAALAEEHNEVPVGAVLVLNGEIIGEGWNRSIGHHDATAHAEIM |
| | ALRQAGKKLENYRLLDTTLYVTLEPCPMCAGALLHSRVKRVVFGAPDLKAGAAGTV |
| | LNLFESQASYHYADVESGLLEQECREQLQAFFKRRRKEIKALKKAKREAE |
| 152 | MREALAEARAAAAAGEVPVGAVVVRDGAILARAGNASIRARDPTAHAEVLALRAA |
| | ARALGNHRLTGCTLYVTLEPCAMCAGAIAHARVARLVYGAADPKGGAVEHGPRFFA |
| | QPTCHHRPEVTGGVLAAEAGALLRDFFRARR |
| 153 | MTDEYFMRQALREARRAYDEGEVPVGAVVVRDGRVIARGRNQVERLKDPTAHAEM |
| | IALTAAANHLGAKRLEGCTVYVTLEPCAMCAGALVLARVDRLVFGAFDPKAGACGT |
| | LYDIPRDPRLNHRVEVVSGVLAEESAALLREFFAARR |
| 154 | MTDEYFMRQALREARRAYEEDEVPVGAVVVREGRVIARGRNQVERLKDPTAHAEMI |
| | ALTAAANHLGSKRLEGCTVYVTLEPCPMCAGALVLARVRRLVFGAFDPKAGACGTL |
| | YDIPRDRRLNHRVEVVGGVLEEESAALLREFFRRRR |
| 155 | MSDEQYMRRALELARQAEQEGEVPVGAVLVKDGEVVAEGWNRSIGSHDATGHAEI |
| | MALRAAGQVLQNYRLLDTTLYVTLEPCPMCAGALLHSRVKRVVYGAPDLKAGAAG |
| | TVLNLFESQASYHYADVESGLLEEECREQLQAFFKRRRKEIKAARQAKRDAE |
| 156 | MREALAEARAAAAAGEVPVGAVVVRDGAVLARAGNRTVRDADPTAHAEVVALRA |
| | AARALGNHRLGGCTLYVTLEPCAMCAGAIAQARVARLVYGADDPKGGAVAHGPRL |
| | FAQPTCHHRPEVVGGVGAAEAAALLRDFFAARR |
| 157 | MDDAGFMRLALAEARAAAAAGEVPVGAVVVRDGAVLAAAGNRTLRDCDPTAHAE |
| | VVALRAAARALGNYRLDGCTLYVTLEPCAMCAGAMLHARLARVVYGAADPKTGA |
| | AGSVLDLFAQPRLNHHTQVEGGVLAAECGALLQDFFRARRG |
| 158 | MSDEQFMRRAIELARKGEELGEVPVGAVLVKDGEIIAEGWNQSISTHDATAHAEIMA |
| | LRAAGEKLQNYRLLDTTLYVTLEPCPMCAGALLHSRVKRVVFGAPDLKAGAAGTVL |
| | NLFESQASYHYADVESGLLEEECREQLQAFFKRRRKEIKALKKAKREAEEK |
| 159 | MREALAEARAAAAAGEVPVGAVVVRDGVVLARAGNRTVRDADPTAHAEVVALRA |
| | AARALGNHRLGGCTLYVTLEPCAMCAGAIAQARVARLVYGAADPKGGAVAHGPRL |
| | FEQPTCHHRPEVVGGVLAEEAGALLRDFFAARR |
| 160 | MRDALAEARAAAAAGEVPVGAVVVRDGAVLARAGNASIRARDPTAHAEILALRAA |
| | ARALGNHRLDGCTLYVTLEPCAMCAGAIAHARIARLVYGAADPKGGAVEHGPRFFA |
| | QPTCHHRPEVVGGVGAAEAGALLRDFFRARR |
| 161 | MSDEDYMRLALEEARAAAAAGEVPVGAVVVRDGAVLARAGNRTVRDCDPTAHAEI |
| | VALREAARALGNHRLTGCTLYVTLEPCAMCAGAISHARVARLVYGADDPKGGAVA |
| | HGPRFFAQPTCHHRPEVTGGVLAEEAGALLRGFFAARR |
| 162 | MTDEYFMRQALREARRAYDEDEVPVGAVVVRDGRVIARGRNQVERLKDPTAHAEM |
| | IALTSAANYLGSKRLEGCTVYVTLEPCAMCAGALVLARVDRLVFGAFDPKAGACGT |
| | LYDIPGDRRLNHRVAVTGGVLEEESAALLREFFRERR |
| 163 | MSDEQYMRRALELARQAEQEGEVPVGAVLVKDGEIVAEGWNRSIGSHDATAHAEIE |
| | TLRKAGQALENYRLLDTTLYVTLEPCPMCAGALLHSRVKRVVFGAPDLKAGAAGTV |
| | LNLFESQASYHYAEVESGLLEEECREQLQAFFKRRRKEIKALRQAEKEAAEK |
| 164 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVAHGPRFF |
| | EQPTCHHRPEVTGGVLAEEAGALLRDFFRARR |
| 165 | MRDALAEARAAAARGEVPVGAVVVRDGAVLARAGNASIAARDPTAHAEILALRAA |
| | ARALGNHRLAGCTLYVTLEPCAMCAGAIAHARIARLVYGAADPKGGAVAHGPRFFE |
| | QPTCHHRPEVTGGVGAAEAGALLRDFFAARR |
| 166 | MREALAEARAAAAAGEVPVGAVVVRDGAVLARAGNRTVRDADPTAHAELVAIRAA |
| | ARALGSERLTGCTLYVTLEPCAMCAGAISHARVARLVYGADDPKGGAVAHGPRFFA |
| | QPTCHHRPDVTGGVGEAEAGALLRAFFAARR |
| 167 | MDDAGFMRLALAEARKAAAAGEVPVGAVVVRDGAVLAAAGNRTLRDCDPTAHAE |
| | VVALREAARALGNHRLGGCDLYVTLEPCAMCAGAISHARIARLYYGADDPKGGAV |
| | AHGARVFDQPTCHHRPEVYGGIGAAEAAALLRGFFAARR |
| 168 | MTDEYFMRQALREARRAYDEGEVPVGAVVVRDGRVIARGRNQVERLKDPTAHAEM |
| | LALTAAAAHLGSKRLEGCTLYVTLEPCAMCAGAIVLARLPRLVFGAFDPKAGACGTL |
| | YDIVRDRRLNHRVEVVSGVLEEECGALLKEFFARLR |
| 169 | MSDEQYMRRALELARQAEQEGEVPVGAVLVKDGEVIAEGWNRSIGSHDATGHAEIM |
| | ALRAAGEKLQNYRLLDTTLYVTLEPCPMCAGALLHSRVKRVVFGAPDLKAGAAGTV |
| | LNLFESQASYHYADVESGLLEEECREQLQAFFKRRRKEIKALRQAQKAAE |
| 170 | MRDALAEARAAAAAGEVPVGAVVVRDGAILARAGNASIRARDPTAHAEILALRAAA |
| | RALGNHRLTGCTLYVTLEPCAMCAGAIAHARVARLVYGAADPKGGAVAHGPRFFEQ |
| | PTCHHRPEVTGGVGAAEAGALLRDFFAARR |
| 171 | MTEALAEARKAAAAGEVPVGAVLVRDGAVLARGGNRTIRDCDPTAHAEIVVLREAA |
| | RALGNYRLEGCTLYVTLEPCAMCAGAMIHARLDRLVYGADDPKAGAAGSVLDVLN |
| | HPRLNHQMEVTAGVLAEECGALLREFFRARR |
| 172 | MREALAEARAAAAAGEVPVGAVVVRDGAVLARAGNRTVRDADPTAHAELVAIRAA |
| | ARALGSERLTGCTLYVTLEPCAMCAGAISHARIARLVYGADDPKGGGVAHGARVFD |
| | HPQCHHRPEVVGGVGAAEAAALLRDFFAARR |
| 173 | MREALAEARAAAAAGEVPVGAVVVRDGAVLARAGNRTVRDADPTAHAELVAIRAA |
| | AAALGSERLTGCTLYVTLEPCAMCAGAISHARIARLVYGAADPKGGAVAHGPRLFA |
| | QPTCHHRPEVVGGVGEAEAAALLRDFFAARR |
| 174 | MREALAEARAAAAAGEVPVGAVVVRDGAVLARAGNASIRARDPTAHAEILALRAA |
| | ARALGNHRLTGCTLYVTLEPCAMCAGAIAHARIARLVYGAADPKGGAVAHGPRFFE |
| | QPTCHHRPEVTGGVLAEEAGALLRAFFRARR |
| 175 | MRDALAEARAAAARGEVPVGAVVVRDGAILARAGNATVAASDPTAHAEILALRAA |
| | ARALGSQRLPGAVLYVTLEPCAMCAGAISHARIARLVYGAADPKGGAVAHGPRFFE |
| | QPTCHHRPEVTGGVLAEEAGALLRDFFRARR |
| 176 | MRDALAEARAAAAAGEVPVGAVVVRDGAILARAGNASIRARDPTAHAEILALRAAA |
| | RALGNHRLDGCTLYVTLEPCAMCAGAIAHARIARLVYGAADPKGGAVAHGPRLFAQ |
| | PTCHHRPEVTGGVGAAEAAALLRDFFRARR |
| 177 | MTDEYFMRQALREARRAYDEGEVPVGAVVVRDGRVIARGRNQVERLKDPTAHAEM |
| | IALTAAAAHLGNKRLEGCTLYVTLEPCAMCAGALVLARVPRLVFGAFDPKAGACGT |
| | LYDIVRDRRLNHRVEVVSGVLEEESAALLKEFFARLR |
| 178 | MDDAGFMRLALEEARAAAAAGEVPVGAVVVRDGEVLARAGNRTVRDCDPTAHAE |
| | VVALREAARKLGNHRVTGATLYVTLEPCAMCAGAISLARVARLVYAADDPKGGGV |
| | AHGARVFDQPTCHHRPEVVSGVLAEESAALLRGFFAARR |
| 179 | MTDEYFMRQALREARRAYDEDEVPVGAVVVREGKVIARGRNQVERLKDPTAHAEM |
| | IALTSAANHLGSKRLEGCTVYVTLEPCAMCAGALVLARVERLVFGAFDPKAGACGT |
| | LYNIPADRRLNHRVEVVGGVLEEESAALLREFFRKRR |
| 180 | MDDEAWMRRAIALAHQAEAEGEVPVGAVLVKDGEVVAEGWNRSIGSHDATAHAEI |
| | ETLRKAGQALENYRLLDTTLYVTLEPCPMCAGALLHSRVKRVVFGAPDLKAGAAGT |
| | VLNLFESQASYHYADVESGLLEEECREQLQAFFKRRRKEIKALRQAQREAE |
| 181 | MDDAGFMRLALAEARAAAAAGEVPVGAVVVRDGAVLAAAGNRTLRDCDPTAHAE |
| | VVALRAAARALGNHRLEGCTLYVTLEPCAMCAGAALQARLARLVYGAAEPKTGAA |
| | GSVLDVFANPALNHHTAVTGGVLAAEAAALLRDFFAARR |
| 182 | MTEALAEARKAAAAGEVPVGAVLVRDGAVLARGGNRTIRDCDPTAHAEIVALRAA |
| | ARAAGNYRLPGATLYVTLEPCAMCAGAMIHARVDRLVYGAADPRAGAAGSVFDVV |
| | RHPALNHRMAVEGGVLAEECGALLRDFFRARR |
| 183 | MTEKDKFFMQRAIELAKLAEENGEVPVGAVLVKDGEIIAEGWNQSIGSHDATAHAEI |
| | MALRQAGKVLENYRLLDTTLYVTLEPCPMCAGALLHSRVKRIVFGAPDLKAGAAGT |
| | VLNLFESQASYHYADVESGLLEEECREQLQAFFKRRRKEIKALKKAKKEAE |
| 184 | MTDEYFMRQALREARRAYDEDEVPVGAVVVRDGKVIARGRNQVERLKDPTAHAEM |
| | IALTSAANYLGSKRLEGCTVYVTLEPCPMCAGALVLARVERLVFGAFDPKAGACGTL |
| | YDIPGDRRLNHRLEVVGGVLEEESAALLREFFRRRR |
| 185 | MREALAEARAAAAAGEVPVGAVVVRDGAVLARAGNASIRARDPTAHAEMLALRAA |
| | ARALGNHRLDGCTLYVTLEPCAMCAGAIAHARVARLVYGAADPKGGAVAHGPRFF |
| | EQPTCHHRPEVTGGVLAAEAGALLRDFFRARR |
| 186 | MTDEYFMRQALREARRAYDEGEVPVGAVVVRDGRVIARGRNQVERLKDPTAHAEM |
| | IALTAAAAHLGNKRLEGCTLYVTLEPCAMCAGAIVLARIPRLVFGAFDPKAGACGTL |
| | YDIVRDRRLNHRAEVVSGVLEEECGALLKEFFARLR |
| 187 | MSDEQYMRRALELARQAEQAGEVPVGAVLVKDGEIIAEGWNQSISSHDATAHAEIM |
| | ALRAAGQALENYRLLDTTLYVTLEPCPMCAGALLHSRVKRVVFGAPDLKAGAAGTV |
| | LNLFESQASYHYAEVESGLLEEECREQLQAFFKRRRKEIKALKQAKREAEQK |
| 188 | MDDAGFMRLALAEARAAAAAGEVPVGAVVVRDGAVLAAAGNRTLRDCDPTAHAE |
| | VVALRAAARALGNYRLDGCTLYVTLEPCAMCAGAMLHARLARVVYGAADPKTGA |
| | AGSVLDLFAEPRLNHHTRVEGGVLAAECGALLRDFFRARRG |
| 189 | MDDAGFMRLALAEARAAAAAGEVPVGAVVVRDGAVLAAAGNRTLRDRDPTAHAE |
| | MVAIRAAAAALGQERLTDCDLYVTLEPCAMCAGAISFARIRRLYFGAADPKGGAVE |
| | HGPRFFAQPTCHHAPEVYGGIGEGEAAALLRDFFRARR |
| 190 | MREALAEARAAAAAGEVPVGAVVVRDGAVLARAGNRTVRDADPTAHAEVVALRA |
| | AARALGNHRLDGCTLYVTLEPCAMCAGAIAQARVARLVYGAADPKGGAVAHGPRL |
| | FEQPTTHHRPEVVGGVLAEEAAALLRGFFAARR |
| 191 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGAADPKGGAVAHGARVF |
| | DQPQTHHRPEVTGGVLAEEAGALLRAFFAARR |
| 192 | MDDAGFMRLALAEARRAAEAGEVPVGAVVVRGGEVLAAAGNRTLRDCDPTAHAEI |
| | VALREAARRVGNYRLADCDLYVTLEPCAMCAGAIVHARVRRLVYGADDPKAGAVR |
| | SALRVLDAPALNHRVEVTAGVLAEECGALLRDFFRARR |
| 193 | MTEALAEARKAAALGEVPVGAVVVRDGAVIARGHNRTVTDADPTAHAEIVALREA |
| | ARALGNHRLTGCTLYVTLEPCAMCAGAMVHARLDRLVYGAADPKAGAAGSVLDVL |
| | GHPRLNHQMEVTAGVLADESAALLRDFFRARR |
| 194 | MREALAEARAAAAAGEVPVGAVVVRDGAVLARAGNASIRARDPTAHAEMLALRAA |
| | ARALGNHRLDGCTLYVTLEPCAMCAGAIAHARIARLVYGAADPKGGAVAHGPRFFE |
| | QPTCHHRPEVVGGVGAAEAGALLRDFFAARR |
| 195 | MTEALAEARKAAALGEVPVGAVVVRDGAVIARGHNRTVTDTDPTAHAEIVALREAA |
| | RALGNHRLTGCTLYVTLEPCAMCAGAIVHARLDRVVYGAADPKAGAAGSVLDVLG |
| | HPRLNHRPEVEGGVLGEECGALLRDFFRARR |
| 196 | MREALAEARAAAAAGEVPVGAVVVRDGAVLARAGNRTVRDADPTAHAEVVALRE |
| | AARALGNHRLGGCTLYVTLEPCAMCAGAISHARVARLVYGADDPKGGAVRHGARV |
| | FDQPTCHHRPEVVGGVLAEESAALLRGFFAARR |
| 197 | MREALAEARAAAAAGEVPVGAVVVRDGAVLARAGNASIRARDPTAHAEILALRAA |
| | ARALGNHRLDGCTLYVTLEPCAMCAGAIAHARVARLVYGAADPKGGAVEHGPRFF |
| | AQPTCHHRPEVVGGVGAAEAGALLRAFFAARR |
| 198 | MDDAGFMRLALAEARAAAAAGEVPVGAVVVRDGAVLAAAGNRTLRDCDPTAHAE |
| | VVALRAAARALGNHRLGGCDLYVTLEPCAMCAGAISHARIARLYYGADDPKGGAV |
| | AHGARVFAHPQCHHRPEVYGGIGAAEAAALLRDFFAARR |
| 199 | MTDEYFMRQALREARRAYDEDEVPVGAVVVRDGKVIARGRNQVERLKDPTAHAEM |
| | IALTSAANHLGSKRLEGCTVYVTLEPCPMCAGALVLARVERLVFGAFDPKAGACGTL |
| | YDIPGDRRLNHRVEVVGGVLEEESAALLREFFRRRR |
| 200 | MDDEFYMRRALELAALAEEHNEVPVGAVLVLNGEIIGEGWNRSIGHHDATAHAEIM |
| | ALRQAGKKLENYRLLDTTLYVTLEPCPMCAGALLHSRVKRVVFGAPDLKAGAAGTV |
| | LNLFESQASYHYADVESGLLEQECREQLQAFFKRRRKEKKALKQAQKEAE |
| 201 | MRDALAEARAAAAAGEVPVGAVVVRDGAILARAGNASIRARDPTAHAEVLALRAA |
| | ARALGNHRLDGCTLYVTLEPCAMCAGAIAHARIARLVYGAADPKGGAVEHGPRFFA |
| | QPTCHHRPEVVGGVGAAEAAALLRDFFRARR |
| 202 | MRAALDEARAAAAAGEVPVGAVVVRDGAILARAGNRTVRDCDPTAHAEIVALREA |
| | ARALGNHRLTGCTLYVTLEPCAMCAGAISHARVARLVYGADDPKGGAVAHGPRFFA |
| | QPTCHHRPEVVGGVGAGEAAALLRDFFAARR |
| 203 | MREALAEARAAAAAGEVPVGAVVVRDGAILARAGNASIRARDPTAHAEVLALRAA |
| | ARALGNHRLTGCTLYVTLEPCAMCAGAIAHARIARLVYGAADPKGGAVAHGPRFFE |
| | QPTCHHRPEVTGGVLAEEAGALLRAFFRARR |
| 204 | MRDALAEARAAAARGEVPVGAVVVRDGAVLARAGNATVAASDPTAHAEILALRAA |
| | ARAAGNHRLPGAVLYVTLEPCAMCAGAISHARIARLVYGAADPKGGAVAHGPRFFE |
| | QPTCHHRPEVTGGVLAEEAGALLRDFFRARR |
| 205 | MREALAEARAAAAAGEVPVGAVVVRDGAILARAGNRTVRDADPTAHAEIVALREA |
| | ARALGNHRLGGCTLYVTLEPCAMCAGAISQARVARLVYGADDPKGGAVAHGPRFF |
| | AQPTCHHRPEVAGGLGEAEAAALLRDFFAARR |
| 206 | MTEALAEARKAAAAGEVPVGAVLVRDGAVLARGGNRTIRDCDPTAHAEIVALRAA |
| | ARALGNYRLPGTTLYVTLEPCAMCAGAMIHARVARLVYGAADPKTGAAGSVEDVL |
| | RHPALNHRMEVEGGVLAEECGALLRDFFRARR |
| 207 | MTEKDKFFMQRAIELAKKGEENGEVPVGAVLVKDGEIIAEGWNQSIGSHDATAHAEI |
| | MTLREAGKVLQNYRLLDTTLYVTLEPCPMCAGALLHSRVKRVVFGAPDLKAGAAGT |
| | VLNLFESQASYHYADVESGLLEEECREQLQAFFKRRRKEKKALKQAKREAEQK |
| 208 | MTDEDYMRLALEEARAAAAAGEVPIGAVVVRDGEVLARAGNRTVRDCDPTAHAEI |
| | VALREAARALGNYRLTGCTLYVTIEPCAMCAGAMIHARVDRLVYGAADPKAGAVR |
| | STLRVLDHPALNHRVEVTAGVLADECAALLQDFFRSRR |
| 209 | MRDALAEARAAAAAGEVPVGAVVVRDGAVLARAGNRTVRDADPTAHAELVAIRA |
| | AARALGSERLTGCTLYVTLEPCAMCAGAISHARIARLVYGAADPKGGAVAHGPRFFA |
| | QPTCHHRPDVTGGVGEAEAGALLRDFFAARR |
| 210 | MREALAEARAAAAAGEVPIGAVVVRDGAVLARAGNRTVRDCDPTAHAEVVALREA |
| | ARALGNHRLTGCTLYVTVEPCAMCAGAISHARVARLVYGADDPKGGAVRHGPRFFE |
| | QPTCHHRPEVVGGVGAEEAAALLRDFFRARR |
| 211 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVAHGPRFF |
| | EQPTCHHRPEVTGGVLAEEAGALLRAFFAARR |
| 212 | MTDEYFMRQALREARRAYDEGEVPVGAVVVRDGRVIARGRNQVERLKDPTAHAEM |
| | LALTAAAAHLGSKRLEGCTLYVTLEPCAMCAGAIVLARVPRLVFGAFDPKAGACGT |
| | LYDIPRDRRLNHRAEVVGGVLEEESAALLREFFAARR |
| 213 | MREALAEARAAAAAGEVPVGAVVVRDGAVLARAGNRTVRDADPTAHAEIVALREA |
| | ARALGNHRLGGCTLYVTLEPCAMCAGAIAQARVARLVYGADDPKGGAVAHGPRFF |
| | AQPTCHHRPEVVGGVGAAEAGALLRGFFAARR |
| 214 | MDDEAWMRRAIALAHKAEQEGEVPVGAVLVKDGEVIAEGWNRSIGHHDATAHAEI |
| | ETLRKAGQVLENYRLLDTTLYVTLEPCPMCAGALLHSRVKRIVFGAPDLKAGAAGT |
| | VLNLFESQASYHYADVESGLLEEECREQLQAFFKRRRKEIKALRQAQKAAE |
| 215 | MREALAEARAAAAAGEVPVGAVVVRDGAVLARAGNRTVRDADPTAHAEIVALREA |
| | ARALGNHRLGGCTLYVTLEPCAMCAGAIAQARVARLVYGAADPKGGAVAHGPRLF |
| | AQPTCHHRPEVVGGVGEAEAAALLRGFFAARR |
| 216 | MRDALAEARAAAAAGEVPVGAVVVRDGAILARAGNASIRARDPTAHAEILALRAAA |
| | RALGNHRLTGCTLYVTLEPCAMCAGAISHARIARLVYGAADPKGGAVAHGPRFFEQP |
| | TCHHRPEVTGGVLAEEAGALLRAFFRARR |
| 217 | MSDEQYMRRAIELARQAEQEGEVPVGAVLVKDGEIIAEGWNRSIGSHDATAHAEIET |
| | LRKAGQVLENYRLLDTTLYVTLEPCPMCAGALLHSRVKRIVYGAPDLKAGAAGTVL |
| | NLFESQASYHYADVESGLLEEECREQLQAFFKRRRKEIKAARKAKREAEQ |
| 218 | MDDAGFMRLALAEARAAAAAGEVPVGAVVVRDGEVLAAAGNRTLRDCDPTAHAE |
| | VVALRAAARALGNHRLDGCDLYVTLEPCAMCAGAISHARIRRLYYGADDPKGGAV |
| | DNGVRFFASPTCHHAPEVYSGLAESEAAALLRDFFRERR |
| 219 | MREALAEARAAAAAGEVPVGAVVVRDGAVLARAGNRTVRDADPTAHAEVVALRA |
| | AARALGNHRLDGCTLYVTLEPCAMCAGAIAQARVARLVYGAADPKGGAVAHGPRL |
| | FEQPTCHHRPEVVGGVGEAEAAALLRGFFAARR |
| 220 | MTDEYFMRQALREARRAYDEDEVPVGAVVVREGKVIARGRNQVERLKDPTAHAEM |
| | IALTSAANYLGSKRLEGCTVYVTLEPCPMCAGALVLARVERLVFGAFDPKAGACGTL |
| | YDIPGDRRLNHRVEVVGGVLEEESAALLREFFRRRR |
| 221 | MREALAEARAAAAAGEVPVGAVVVRDGAILARAGNASIRARDPTAHAEILALRAAA |
| | RALGNHRLTGCTLYVTLEPCAMCAGAIAHARIARLVYGAADPKGGAVAHGPRFFEQ |
| | PTCHHRPEVTGGLGEAEAAALLRDFFRARR |
| 222 | MTDEYFMRQALREARRAYEEDEVPVGAVVVREGRVIARGRNQVERLKDPTAHAEMI |
| | ALTAAANHLGSKRLEGCTVYVTLEPCPMCAGALVLARVERLVFGAFDPKAGACGTL |
| | YDIPGDRRLNHRVEVVGGVLEEEAAALLREFFRRRR |
| 223 | MDDAGFMRLALAEARAAAAAGEVPVGAVVVRDGAVLAAAGNRTLRDCDPTAHAE |
| | VVALRAAARALGNYRLDGCTLYVTLEPCAMCSGAMLHARLARVVYGAADPKTGA |
| | AGSVLDLFAEPRLNHHTRVEGGVLAGECGALLADFFRGRRG |
| 224 | MDDAGFMRLALAEARAAAAAGEVPVGAVVVRDGAVLAAAGNRTLRDCDPTAHAE |
| | VVALRAAARALGNHRLDGCDLYVTLEPCAMCAGAIAHARIRRLYYGAADPKGGAV |
| | EHGARVFDQPTCHHRPEVYGGIGEAEAAALLRDFFRDR |
| 225 | MSDEQYMRRALELARQAEQEGEVPVGAVLVKDGEIIAEGWNRSIGSHDATGHAEIM |
| | ALRAAGEKLQNYRLLDTTLYVTLEPCPMCAGALLHSRVKRVVFGAPDLKAGAAGTV |
| | LNLFESQASYHYADVESGLLEEECREQLQAFFKRRRKEIKALRQAQKAAE |
| 226 | MREALAEARAAAAAGEVPVGAVVVRDGAVLARAGNASIRARDPTAHAEVLALRAA |
| | ARALGNHRLDGCTLYVTLEPCAMCAGAIAHARVARLVYGAADPKGGAVEHGPRFF |
| | AQPTCHHRPDVTGGVGAAEAAALLRDFFRARR |
| 227 | MREALAEARAAAAAGEVPVGAVVVRDGAILARAGNRTVRDADPTAHAEIVALRAA |
| | ARALGNHRLDGCTLYVTLEPCAMCAGAAVLARVARLVFGADDPKGGAVRTGVRLF |
| | DAPTCHHRPEVTGGVLAEEAGALLREFFAARR |
| 228 | MREALAEARAAAAAGEVPVGAVVVRDGAVLARAGNRTVRDADPTAHAELVAIRAA |
| | ARALGSERLTGCTLYVTLEPCAMCAGAISHARIARLVYGAADPKGGAVAHGPRFFAQ |
| | PTCHHRPEVTGGVGEAEAAALLRGFFAARR |
| 229 | MTDEDYMRLALEEARAAAAAGEVPVGAVVVRDGEVIARARNAPVSACDPTAHAEIL |
| | ALREAARRLGNYRLDGCTLYVTLEPCAMCSGAMLHARLARVVYGAADPKTGAAGS |
| | VLDLFAEPRLNHHTAVEGGVLAAECGALLRDFFRARRG |
| 230 | MSDEQYMRRALELARQAEAEGEVPVGAVLVKDGEVVAEGWNRSIGSHDATGHAEI |
| | MALRAAGQALENYRLLDTTLYVTLEPCPMCAGALLHSRVKRVVFGAPDLKAGAAG |
| | TVLNLFESQASYHYAEVESGLLEEECREQLQAFFKRRRKEIKAARKAAKAAAE |
| 231 | MREALAEARAAAAAGEVPVGAVVVRDGAVLARAGNASIRAHDPTAHAEVLALRAA |
| | ARALGNHRLTGCTLYVTLEPCAMCAGAIAHARVARLVYGAADPKGGAVAHGPRFFE |
| | QPTCHHRPEVTGGVLAEEAGALLREFFRARR |
| 232 | MREALAEARAAAAAGEVPVGAVVVRDGAVLARAGNRTVRDADPTAHAEVVALRA |
| | AARALGNHRLDGCTLYVTLEPCAMCAGAIAQARVARLVYGAADPKGGAVAHGPRL |
| | FEQPTCHHRPEVVGGVGEAEAAALLRAFFAARR |
| 233 | MTEALAEARKAAALGEVPVGAVVVRDGAVIARGHNRTIADSDPTAHAEIVALREAA |
| | RVLGNYRLTGCTLYVTVEPCAMCAGAMVHARLDRLVYGAADPKAGAAGSVLDVL |
| | GHPRLNHQMEVTGGVLAEECAALLRDFFRARR |
| 234 | MDDEFYMRRALELAALAEEHNEVPVGAVLVLNGEIIGEGWNRSIGHHDATAHAEIM |
| | ALRQAGKVLQNYRLLDTTLYVTLEPCPMCAGALVHSRVKRVVYGTPDLKTGAAGS |
| | VMNLLSYEGVNHHVEVTSGVLAEECREQLQAFFRRRRAEKKALKKAQREAE |
| 235 | MDDEAWMRRAIALAHQAEAEGEVPVGAVLVKDGEVIAEGWNRSIGHHDATAHAEI |
| | ETLRQAGQVLQNYRLLDTTLYVTLEPCPMCAGALLHSRVKRVVFGAPDLKAGAAGT |
| | VLNLFESQASYHYADVESGLLEQECREQLQAFFKRRRKEIKAQRDAQRAAQE |
| 236 | MREALAEARAAAAAGEVPVGAVVVRDGAVLARAGNASIRARDPTAHAEILALRAA |
| | ARALGNHRLTGCTLYVTLEPCAMCAGAISHARIARLVYGAADPKGGAVAHGPRFFE |
| | QPTCHHRPDVTGGVLAAEAGALLRDFFRARR |
| 237 | MDDAGFMRLALEEARAAAAAGEVPVGAVVVRDGAVLARAGNRTLRDRDPTAHAE |
| | VVALRAAARALGSERLTGCDLYVTLEPCAMCAGAISFARIRRLYFGAADPKGGAVEN |
| | GVRFFASPTCHHAPEVYGGLAESEAAALLRDFFRARR |
| 238 | MDDAGFMRLALAEARAAAAAGEVPVGAVVVRDGAVLAAAGNRTLRDCDPTAHAE |
| | VVALRAAARALGNHRLGGCDLYVTLEPCAMCAGAISHARIARLYYGADDPKGGAV |
| | AHGARVFDQPTCHHRPEVYGGIGAAEAAALLRDFFRARR |
| 239 | MREALAEARAAAAAGEVPVGAVVVRDGAVLARAGNASIRARDPTAHAEILALRAA |
| | ARALGNHRLDGCTLYVTLEPCAMCAGAIAHARVARLVYGAADPKGGAVEHGPRFF |
| | AQPTCHHRPEVTGGVLAAEAGALLRDFFRARR |
| 240 | MREALAEARAAAAAGEVPVGAVVVRDGAVLARAGNASIRARDPTAHAEILALRAA |
| | ARALGNHRLDGCTLYVTLEPCAMCAGAIAHARIARLVYGAADPKGGAVAHGPRFFA |
| | QPTCHHRPDVTGGVLAAEAGALLRDFFRARR |
| 241 | MREALAEARAAAAAGEVPVGAVVVRDGAVLARAGNASIRARDPTAHAEVLALRAA |
| | ARALGNHRLTGCTLYVTLEPCAMCAGAIAHARVARLVYGAADPKGGAVAHGPRFFE |
| | QPTCHHRPEVTGGVLAEEAGALLRGFFRARR |
| 242 | DDDDRRWMREALAEARAAADAGEVPVGAVVVRDGVLLARAGNASIRDRDPTAHA |
| | EMLALRAAARALGNHRLTGCTLYVTLEPCAMCAGAIAHARIARLVYGASDPKAGAV |
| | EHGPRLFAQPTIHHRPEVTAGVLAEECGALLRDFFRARR |
| 243 | DPTDLAFMREALAEARAAAEAGEVPVGAVVVCDGRIVARAHNRPIALSDPTAHAEIL |
| | ALREAARALGNYRLTGCTLYATLEPCAMCAGAILHARIARLVYGAADPKAGACGSV |
| | LDVMNHPRLNHRVEVTGGVLAEECGALLSGFFRARR |
| 244 | DDDHAWMGAALAEARAAAEAGEVPVGAVLVADGRVLARAGNRTIRDRDPTAHAE |
| | MLALRAAARALGNHRLTGTTLYVTLEPCAMCAGAISLARVARLVYAASDPKGGAVE |
| | HGPRFFAQPTCHHRPEVVGGVGEGEAGALLRDFFRARR |
| 245 | MTAEDDRFMRLALAEARAAAEAGEVPVGAVVVAGGRVVARAHNRPIALHDPTAHA |
| | EVLALRAAARELGNYRLTGCTLYATLEPCAMCAGAVLHARIARLVYGAADPKAGAC |
| | GSVLAVMNHPRLNHRVEVTGGVLAEECGALLREFFRARR |
| 246 | MDSDLAFMREALAEARAAAEAGEVPVGAVVVHEGKIVARAANRMRTDRDPTAHAE |
| | LLALRAAARALETTRLTGCTLYVTLEPCAMCAGAISHARVARLVYGASDPKGGAVE |
| | HGPRFFAQPTCHHRPEVVGGVGEAEAGALLRGFFRARR |
| 247 | DPDDARWMREALAEARAAAEAGEVPVGAVVVRDGRLLARAGNRTIRDRDPTAHAE |
| | MLALRAAARALGNHRLEGCTLYVTLEPCAMCAGAMVQARVARLVYGAADPKAGA |
| | AGSVLDVLGDPRLNHRVEVTGGVLAEECGALLREFFRARR |
| 248 | MDSDLEFMREALAEARAAAEAGEVPVGAVVVRDGVILARAGNRPIRDHDPTAHAEI |
| | LALREAARAVGNYRLTGCTLYVTLEPCAMCAGAILHARVERLVYGAADPKAGAAGS |
| | VLDVFGNARLNHHTRVEGGVLAEECGALLSGFFRARRG |
| 249 | DPDAEFMRLALAEAEAAARAGEVPVGAVVVADGRVIARAGNRTIRDRDPTAHAEML |
| | ALRAAARALGSHRLTGCDLYVTLEPCAMCAGAISHARIRRLYYGAADAKGGAVEHG |
| | PRFFAQPTCHHRPEVYGGIGETAAAALLRDFFRARR |
| 250 | DPDAAFMRAALAEARAAAEAGEVPVGAVVVAGGRIVARAHNRPIELNDPTAHAEIL |
| | ALRQAAAALGNYRLTGCTLYATLEPCAMCAGAILHARIERLVYGAADPKAGAVGSV |
| | LDVFANPRLNHRTQVTAGVLADECGALLREFFRARR |
| 251 | DESSDEEFMRAALEEARAAAAEGEVPVGAVVVAGGRIVARAHNRPIALNDPTAHAEI |
| | LALRQAAAALGNYRLTGCTLYATLEPCAMCAGAILHARIARVVYGAADPKAGACGS |
| | VLAVMNHPQLNHRAEVTGGVLAEECGALLSEFFRARR |
| 252 | DDDHRFMRAALAEARAAAEAGEVPVGAVVVHGGEIIARAHNRPIALHDPTAHAEILA |
| | LRAAARRLGNYRLTGCTLYATLEPCAMCAGAILHARIARLVYGAADPKAGACGSVL |
| | AVMNHPRLNHRVEVTAGVLADECGALLSEFFRARR |
| 253 | MNDEDYMRAALEQARQAAAAGEVPVGAVVVCGGEIVARAHNRPISASDPTAHAEIL |
| | ALREAARKLGNYRLTGCTLYATLEPCAMCSGAILHARIARLVYGAADPKAGAVGSV |
| | LDVINHPRLNHRVEVTSGVLADECGALLKEFFRARR |
| 254 | MNDEDYMRAALEQARQAAAAGEVPVGAVVVCDGKIVARAHNRPISANDPTAHAEI |
| | LALREAARQLGNYRLTGCTLYATLEPCAMCSGAILHARIARLVYGAADPKAGACGS |
| | VLNVINHPQLNHQTEVTGGVLADECGALLKDFFRARR |
| 255 | MRAALEEARRAAAAGEVPVGAVVVRDGAIVARAHNRPIALHDPTAHAEILALREAA |
| | RALGNYRLTGCTLYATLEPCAMCAGAILHARIDRLVYGAADPKAGACGSVLSVMNH |
| | PRLNHRVEVTGGVLAEECGALLTGFFRARR |
| 256 | MSEEDRFMRAALAEARAAAEAGEVPVGAVVVCGGRIVARAHNRPIALNDPTAHAEI |
| | LALREAARELGNYRLTGCTLYATLEPCAMCAGAILHARIARLVYGARDPKAGAVESL |
| | YRLLDDTRLNHRVEAVGGVLAGECGALLSGFFRARR |
| 257 | MDEEFMRLALAEARAAAEAGEVPVGAVVVAGGRVVARAHNRPVALHDPTAHAEV |
| | LALRAAARELGNYRLTGCTLYATLEPCAMCAGAILHARLDRVVYGAADPKAGAAGS |
| | VLDVFANPRLNHRTAVTGGVLAEECGALLREFFRARR |
| 258 | MSEEDRFMRAALAEARAAAEAGEVPVGAVVVAGGRIVARAHNRPIALNDPTAHAEI |
| | LALRAAARALGNYRLTGCTLYATLEPCAMCAGAILHARIARLVYGARDPKAGAVES |
| | LYRLLDDRRLNHRVEAVGGVLAAECGALLSGFFRARRG |
| 259 | MREALAEARAAAAAGEVPVGAVVVRDGAVLARAGNRTLRDRDPTAHAEMLAIRAA |
| | ARALGSERLTGCDLYVTLEPCAMCAGAISHARIARLYYGAADPKGGAVEHGPRFFAQ |
| | PTCHHRPEVYGGIGEGEAAALLRGFFRARR |
| 260 | MRAALEEARRAAAAGEVPVGAVVVRDGAIVARAHNRPIALHDPTAHAEILALREAA |
| | RALGNYRLTGCTLYATLEPCAMCSGAILHARLDRVVYGAADPKTGAAGSVLDVFAN |
| | PRLNHHAAVTGGVLAEECGALLSGFFRARR |
| 261 | MREALAEARAAAAAGEVPVGAVVVRDGAIVARAHNRPIALHDPTAHAEVLALREA |
| | ARALGNYRLTGCTLYATLEPCAMCAGAILHARLDRVVYGAADPKAGAAGSVLDVF |
| | ANPRLNHRTAVTGGVLADECGALLSGFFRARR |
| 262 | MRAALEEARRAAAAGEVPVGAVVVRDGAIVARAHNRPIALHDPTAHAEILALREAA |
| | RALGNYRLTGCTLYATLEPCAMCAGAILHARLDRVVYGAADPKAGAAGSVLQVFD |
| | HPRLNHRTAVTGGVLAEECGALLRDFFRARR |
| 263 | MDDDALMREALAEARAAAAAGEVPVGAVVARGGEIVARAANAPRALCDPTAHAEI |
| | LALRAAARALGNYRLTGCTLYATLEPCAMCAGAILHARIARLVYAAPDPKAGACGS |
| | VLAVLNHPQLNHRVEVTAGLLADECGALLTDFFRARRG |
| 264 | MRAALEEARRAAAAGEVPVGAVVVRDGAIVARAHNRPIALHDPTAHAEILALREAA |
| | RALGNYRLTGCTLYATLEPCAMCAGAILHARIDRLVYGAADPKAGACGSVLSVMNH |
| | PQLNHRVEVTGGVLAEECGALLSGFFRARR |
| 265 | MNDEDFMRAALAQAREAAAAGEVPVGAVVVCDGEIVARAHNRPIALNDPTAHAEIL |
| | ALREAARRLGNYRLTGCTLYATLEPCAMCAGAILHARIARLVYGAADPKAGAVGSV |
| | LDVLNHPRLNHRVEVTRGVLAEECGALLSEFFRARRR |
| 266 | MDEEFMRRALELARQAEAAGEVPVGAVLVKDGEIVAEGWNQSIGRHDATAHAEIQV |
| | LRKAGQVLENYRLLDTTLYVTLEPCPMCAGALLHSRVKRIVYGAPDLKAGAAGTVL |
| | NLFEHQAAYHYADVEHGLLEEECREQLQAFFKRRRKEKKALKQAQREAEEK |
| 267 | MSEEDRFMRAALAEARAAAEAGEVPVGAVVVAGGRIVARAHNRPIALNDPTAHAEI |
| | LALREAARALGNYRLTGCTLYATLEPCAMCAGAILHARIARLVYGAADPKAGAVES |
| | LYRLLDDTRLNHRVEATGGVLAGECGALLSEFFRARRR |
| 268 | MSEEDRFMRAALAEARAAAEAGEVPVGAVVVAGGRIIARAHNRPIALNDPTAHAEIL |
| | ALRAAARALGNYRLTGCTLYATLEPCAMCAGAILHARIARLVYGARDAKAGAVESV |
| | LRLFDHPALNHRVEAVGGVLADECGALLSEFFRARRA |
| 269 | MREALAEARAAAAAGEVPVGAVVVRDGAIVARAHNRPIALHDPTAHAEILALREAA |
| | RALGNYRLTGCTLYATLEPCAMCAGAILHARLDRVVYGAADPKAGAAGSVLDVFA |
| | NPRLNHRTAVTGGVLADECGALLRDFFRARR |
| 270 | MNDEDYMRAALEQARQAAAAGEVPVGAVVVCGGEIVARAHNRPISASDPTAHAEIL |
| | ALREAARKLGNYRLTGCTLYATLEPCAMCSGAILHARIARLVYGAADPKAGAVGSV |
| | LDVINHPRLNHRVEVTGGVLAGECGALLSGFFRARR |
| 271 | MREALAEARAAAAAGEVPVGAVVVRDGAIVARAHNRPIALHDPTAHAEVLALRAA |
| | ARELGNYRLTGCTLYATLEPCAMCAGAILHARLDRVVFGARDPKAGAAGSVLDVFA |
| | NPRLNHRTAVTGGVLAEECGALLSGFFRARR |
| 272 | MNDEDYMRAALEQARQAAAAGEVPVGAVVVCDGKIVARAHNRPISANDPTAHAEI |
| | LALREAARQLGNYRLTGCTLYATLEPCAMCSGAILHARIARLVYGASDPKAGACGSV |
| | LNVINHPQLNHRVEVTGGVLADECGALLKDFFRARR |
| 273 | MSEEDRFMRAALAEARAAAAEGEVPVGAVVVAGGRIVARAHNRPVALNDPTAHAEI |
| | LALRAAARALGNYRLTGCTLYATVEPCAMCAGAILHARIARLVYGAADPKAGAVRS |
| | VLRVLDHPALNHRVEVTAGVLAEECAALLQEFFRARR |
| 274 | MNDEDYMRAALEQARQAAAAGEVPVGAVVVCGGEIVARAHNRPISANDPTAHAEIL |
| | ALREAARKLGNYRLTGCTLYATLEPCAMCSGAILHARIARLVYGAADPKAGACGSV |
| | LNVINHPQLNHRVEVTGGILADECGALLKDFFRARR |
| 275 | MRAALEEARRAAAAGEVPVGAVVVRDGAIVARAHNRPIALHDPTAHAEILALREAA |
| | RRLGNYRLTGCTLYATLEPCAMCAGAILHARIDRLVYGAADPKAGACGSVLSVMNH |
| | PRLNHRVEVTSGVLAEECGALLTDFFRARR |
| 276 | MRAALEEARRAAAAGEVPVGAVVVRDGEIVARAHNRPIALHDPTAHAEILALREAA |
| | RRLGNYRLTGCTLYATLEPCAMCAGAILHARLDRVVYGAADPKAGACGSVLSVFDQ |
| | PRLNHRTTVTGGVLAEECGALLSGFFRARRG |
| 277 | MREALAEARAAAAAGEVPVGAVVVRDGAVLARAGNASIRARDPTAHAEVLALRAA |
| | ARALGNHRLTGCTLYVTLEPCAMCAGAIAHARVARLVYGAADPKGGAVAHGPRFFE |
| | QPTCHHRPEVVGGVGAAEAGALLRDFFAARR |
| 278 | MTDEYFMRQALREARRAYDEGEVPVGAVVVRDGRVIARGRNQVERLKDPTAHAEM |
| | IALTAAANYLGSKRLEGCTVYVTLEPCPMCAGALVLARVERLVFGAFDPKAGACGT |
| | LYDIVRDRRLNHRLEVVGGVLEEESAALLREFFEKRR |
| 279 | MSDEQYMRRALELARQAEQEGEVPVGAVLVKDGEVIAEGWNRSIGSHDATGHAEIM |
| | ALRAAGEKLQNYRLLDTTLYVTLEPCPMCAGALLHSRVKRVVFGAPDLKAGAAGTV |
| | LNLFESQASYHYADVESGLLEEECREQLQAFFKRRRKEIKAARQAKRDAE |
| 280 | MREALAEARAAAAAGEVPVGAVVVRDGAVLARAGNRTVRDADPTAHAEIVALRAA |
| | ARALGNHRLGGCTLYVTLEPCAMCAGAIAQARVARLVYGADDPKGGAVAHGPRLF |
| | AQPTCHHRPEVVGGVGAAEAAALLRDFFAARR |
| 281 | MDDEALMGLALDEARAAAAAGEVPIGAVVARGGEVLARAGNRTLRDCDPTAHAEI |
| | VALREAARALGNYRLTGCTLYVTLEPCAMCAGAAIQARLDRVVYGAADPKAGAAG |
| | SVLDVLGHPRLNHRPAVEGGVLAAESAALLREFFAARR |
| 282 | MREALAEARAAAAAGEVPVGAVVVRDGAVLARAGNASIRARDPTAHAEILALRAA |
| | ARALGNHRLTGCTLYVTLEPCAMCAGAIAHARLARLVYGAADPKGGAVAHGPRLF |
| | AQPTCHHRPEVTGGVGAAEAAALLRDFFAARR |
| 283 | MSDEQFMRRAIELAKKGEELGEVPVGAVLVKDGEIIAEGWNQSISTHDATAHAEIMA |
| | IRAAGEKLENYRLLDTTLYVTLEPCPMCAGALLHSRVKRVVFGAPDLKAGAAGTVL |
| | NLFESQASYHYADVESGLLEEECREQLQAFFKRRRKEIKAAKQAKREAEEK |
| 284 | MDDEFYMRRALELAALAEEHNEVPVGAVLVLNGEIIGEGWNRSIGHHDATAHAEIM |
| | ALRQAGKKLENYRLLDTTLYVTLEPCPMCAGALLHSRVKRVVFGAPDLKAGAAGTV |
| | LNLFESQASYHYADVESGLLEQECREQLQAFFKRRRKEIKALKKAQKEAE |
| 285 | MDDAGFMRLALAEARKAAAAGEVPVGAVVVRDGAVLAAAGNRTLRDCDPTAHAE |
| | VVALRAAARALGNHRLGGCDLYVTLEPCAMCAGAISHARIARLYYGADDPKGGAV |
| | AHGARVFDQPTCHHRPEVYGGIGAGEAAALLRDFFAARR |
| 286 | DLDDARFMREALAEARAAAEAGEVPVGAVVVADGRIVARAHNRPVALNDPTAHAEI |
| | LALREAARALGNYRLTGCTLYATLEPCAMCAGAILHARIARLVYGARDAKAGAVES |
| | LYRLLDDARLNHRVEVAAGVLADECGALLTEFFRARR |
| 287 | DESFMREALAEARAAAEAGEVPVGAVVVHGGQIVARAHNRPIELSDPTAHAEILALR |
| | AAARALGNYRLTGCTLYATLEPCAMCAGAILHARIDRVVYGASDPKAGAAGSVLSV |
| | FDHPALNHRVDVTAGVLAEECGALLREFFRARRR |
| 288 | DPDAAFMRAALAEARAAAEAGEVPVGAVVVADGAIVARAHNRPIALNDPTAHAEIL |
| | ALREAARALGNYRLTGATLYATLEPCAMCAGAILHARIARLVYGARDPKAGAVESL |
| | YRLLDDARLNHRVAVTGGVLAAECGALLTAFFRARR |
| 289 | DERFMREALAEARAAAAAGEVPVGAVVVRDGEIVARAANRTVRDNDPTAHAEILAL |
| | REAARALGSYRLTGCTLYATLEPCAMCAGAILHARIARLVYAAADPKAGACGSVLS |
| | VMNHPQLNHRVEVESGLLAEECGALLTEFFRARRG |
| 290 | MTADEQYMRRALELARQAEAEGEVPVGAVLVKDGEIVAEGWNRSIGHHDATAHAEI |
| | QVLRKAGQVLQNYRLLDTTLYVTLEPCPMCAGALLHSRVKRIVYGAPDLKAGAAGT |
| | VLNLFEHQAAYHYADVEHGLLEDECREQLQAFFKRRRKEKKALKQALKESN |
| 291 | RIRSDEDFMRQALAEARAAAAAGEVPVGAVVVAGGRIVARAHNRPIDLVDPTAHAEI |
| | LALREAARALGNYRLTGCTLYATLEPCAMCAGAILHARIARLVYGAADPKAGAVES |
| | VLRLLDHPALNHRVEAVSGVLAGECGALLREFFRARR |
| 292 | DLDEALMREALAEARRAAEAGEVPVGAVVARGGRIVARAHNQPVALHDPTAHAEIL |
| | ALRQAAREAGNYRLEGCTLYATLEPCAMCAGALLHARVARLVYGARDPKAGAVGS |
| | LYDLLRDPRLNHRVEVTAGVLAEECGALLSGFFRARR |
| 293 | MREALAEARAAAAAGEVPVGAVVVDPAGEIVARAGNAPRALCDPTAHAEILALRAA |
| | AAAAGNYRLPGHVLYVTLEPCAMCAGAISHARIARLVYGAADPKGGAVAHGPRFFA |
| | QPTCHHRPEVTGGVLAEECGALLRDFFRARR |
| 294 | RLDEQFMRRALELAAHAEAEGEVPVGAVLVLDGQVIGEGWNRSIGQHDATAHAEIM |
| | ALRQAGQVVQNYRLLDTTLYVTLEPCPMCAGALLHSRVKRIVYGAPDLKAGAAGTV |
| | LNLFEHQAAYHYADVEHGLLEEECREQLQAFFKRRRKEKKALKQAQRAAE |
| 295 | DPDDVAFMREALAEAERAAAAGEVPVGAVVVHDGVIVGRGHNRPIASRDPTAHAEI |
| | VALREAAAALGNYRLTGCSLYVTIEPCAMCAGAILHARIARLVYGAADPKAGACGS |
| | VLDVFAEPRLNHHAEVTGGVLADECAALLRSFFAARR |
| 296 | DPDERFMRAALAQARAAAEAGEVPVGAVVVRDGRIVARAHNQPIALNDPTAHAEIL |
| | ALREAARELGNYRLTGCTLYATLEPCAMCAGAILHARIARLVYGARDAKAGAVESK |
| | LRLFENGSFNHRVEVTGGVLAGECGALLSEFFRARR |
| 297 | MDEEFMREALAEARAAAEAGEVPVGAVVVAGGRIVGRGRNRPVELADPTAHAEILA |
| | LREAARALGNYRLEGCTLYATLEPCAMCAGAILHARIARLVYGAADPKAGAAGSVL |
| | DVFALPRLNHRTAVTGGVLAEECGALLSGFFRARRG |
| 298 | RSSDAALMREALAEARAAAEAGEVPVGAVVVAGGRIVARAHNRPVALSDPTAHAE |
| | VLALREAARELGNYRLEGCTLYATLEPCAMCSGALLHARVARLVYGAADPKAGAV |
| | GSVLDLFANPRLNHRVEVTGGVLAEECGALLSGFFRARRG |
| 299 | DDDERWMREALAEARAAAAAGEVPVGAVVVRDGVLLARAGNASIRERDPTAHAE |
| | MLALRAAARALGNHRLGGCTLYVTLEPCAMCAGAIAHARIARLVYGAADPKAGAV |
| | DHGPRLFAQPTVHHRPEVVAGVGAEESAALLRDFFRARR |
| 300 | DPDSDEAFMRAALAEARAAAEAGEVPVGAVVVADGAIVARAHNRPIALHDPTAHAE |
| | ILALRAAARRLGNYRLTGCTLYATLEPCAMCAGAILHARIARLVYGAADPKAGACGS |
| | VLAVMNHPQLNHRVEVTGGILAEECGALLRDFFRARR |
| 301 | MREALAEARAAAEAGEVPVGAVVVAGGRIVARAHNRPVALHDPTAHAEILALRQA |
| | AAELGNYRLEGCTLYATLEPCAMCAGAMLHARLARVVYGAADPKAGAAGSVLDLF |
| | ANPRLNHRTEVVGGVLAEECGALLSGFFRSRR |
| 302 | DERFMREALAEARAAAAAGEVPVGAVVVRDGVVLARAGNRTLRDRDPTAHAEML |
| | ALRQAARALGSHRLTGCDLYVTLEPCAMCAGAISHARIARLYYGAADAKGGAVEHG |
| | PRFFAQPTCHHRPEVYGGIGETEAAALLRGFFRERR |
| 303 | MDSDLEFMRLALAEARAAAEAGEVPVGAVVVAGGRVVARAHNRPVALHDPTAHAE |
| | ILALRAAARELGNYRLTGCTLYATLEPCAMCAGAILHARIARLVYGAADPKAGACGS |
| | VLDVIHHPRLNHRVEVTGGVLAEECGALLSGFFRARR |
| 304 | MREALAEARAAAEAGEVPVGAVVVAGGRIVARAHNRPVALHDPTAHAEILALREAA |
| | RALGNYRLEGCTLYATLEPCAMCAGALLHARVARLVYGARDPKAGAVGSVLDLLD |
| | HPRLNHRVEVVGGVLAEECGALLSGFFRARRG |
| 305 | DDRRWMREALAEARAAAEAGEVPVGAVVVRDGRLLARAGNASIRDRDPTAHAELL |
| | ALRAAARALGNHRLTGCTLYVTLEPCAMCAGAIAHARIARLVYGAADPKGGAVAH |
| | GPRLFEQPTLHHRPEVTGGVLAAEAGELLRAFFRARR |
| 306 | MNDEDFMRAALAQARLAAEAGEVPVGAVVVCDGEIVARAHNRPISASDPTAHAEIL |
| | ALREAARRLGNYRLTGCTLYATLEPCAMCAGAILHARIARLVYGAADPKAGAVGSV |
| | LDVLNHPRLNHRVEVTGGVLAEECGALLRDFFRARR |
| 307 | DESSDLRFMREALAEARAAAEAGEVPVGAVVVCGGRIVARAHNRPIALSDPTAHAEI |
| | LALREAARELGNYRLTGCTLYATLEPCAMCAGAILHARIARLVYGAADPKAGACGS |
| | VLDVINHPRLNHRVEVTGGVLAGECGALLSGFFRARRG |
| 308 | DDLFFMREALAEARRAAAEGEVPVGAVVVCGGRIIARAHNRPIALNDPTAHAEILAL |
| | RAAARALGNYRLTDCTLYATLEPCAMCAGAILHARIRRLVYGAADPKAGAVRSVLR |
| | LLDAPALNHRVAVTAGVLAEECGALLSEFFRARR |
| 309 | DPPSADEAFMREALAEARAAAEAGEVPVGAVVVAGGRIVARAHNRPIALSDPTAHA |
| | EILALRAAARALGNYRLTGTTLYATVEPCAMCAGAIVQARVARLVYGAADPKAGAV |
| | ESLFRILDHPALNHRVEVTAGVLAEECAALLREFFRARR |
| 310 | DETEAFMRAALAEARAAAEAGEVPVGAVVVADGRIVARAHNRPVALHDPTAHAEIL |
| | ALREAARRLGNYRLTGCTLYATLEPCAMCAGAVLHARLARLVYGARDAKAGAVGS |
| | VLDLLAHPRLNHRTAVTAGVLADECGALLSAFFRARRG |
| 311 | DDDERWMREALAEARAAAAAGEVPVGAVVVRDGVLLARAGNASIRDRDPTAHAE |
| | MLALRAAARALGNHRLTGCTLYVTLEPCAMCAGAIAHARIARLVYGASDPKAGAVD |
| | HGPRLFEQPTIHHRPEVTAGVLADECGALLRDFFRARR |
| 312 | DPTTDEALMREALAEARAAAEAGEVPVGAVVARDGEIVARAANAPRALCDPTAHAE |
| | ILALRAAARALGNYRLTGCTLYATLEPCAMCAGAALHARFARIVYGAADPKAGAAG |
| | SVLDVLDQPRLNHRTRVTGGVLADECGALLTEFFRARR |
| 313 | MNKDEFYMKRALELAQKAEEEGEVPVGAVLVLDDEIIGEGWNRPIGSHDATAHAEIQ |
| | ALRDAGQKLENYRLLDTTLYVTLEPCPMCAGALLHSRVKRVVYGAPDLKAGAAGT |
| | VLNLFEHEAAYHYADVEHGLLEDECREQLQAFFKRRRKEIKAKKEAEKKALE |
| 314 | MNSDEFYMQRALELAQKAEQEDEVPVGAVLVLDGEIIGEGWNRSIGHHDATAHAEM |
| | MALKQGGKQIQNYRLLDATLYVTLEPCPMCAGALLHSRVKRIVYGAPDLKAGAAGT |
| | VLNLFEHQAAYHYADVEQGLMEEECREQLQAFFKRRRKEKKALKQAKREAEE |
| 315 | DERFMREALAEARAAAAAGEVPVGAVVVRDGVVLARAGNRTLRDRDPTAHAEML |
| | ALRQAARALGSWRLTGCTLYVTLEPCAMCAGAMVQARVDRLVYGAADPKAGAAG |
| | SVLDVLGHPRLNHRPEVAAGVLAEECGALLREFFRERR |
| 316 | MRAALEEARRAAAEGEVPVGAVVVRDGAIVARAHNRPIALNDPTAHAEILALREAA |
| | RALGNYRLTGCTLYATLEPCAMCAGAILHARIARLVYGAADPKAGACGSVLSVINHP |
| | RLNHRVEVTGGVLAEECGALLTGFFRARR |
| 317 | DPDAAFMREALAEARAAAAAGEVPVGAVVVRAGRIVARAHNRPVELHDPTAHAEIL |
| | ALRAAARELGNYRLTGCTLYATLEPCAMCAGAILHARIERLVYGARDAKAGAAGSV |
| | LDVFANPRLNHRVAVEGGVLADECAALLREFFRARR |
| 318 | MDDEALMRVALEEARAAAAAGEVPVGAVVARGGEIVARAANAPIAASDPTAHAEIL |
| | ALRAAARALGNYRLTGCTLYATLEPCAMCAGAILHARIARLVYAAPDPKAGACGSV |
| | LAVLDHPALNHRVEVTGGVLAEECGALLREFFRARR |
| 319 | MSDEQYMRRALELARQAEQEGEVPVGAVLVKDGEIIAEGWNRSIGHHDATAHAEM |
| | QVLRKAGQVLENYRLLDTTLYVTLEPCPMCAGALLHSRVKRIVYGAPDLKAGAAGT |
| | VLNLFEHQAAYHYADVEHGLLEEECREQLQAFFKRRRKEIKARKQAEKEAEAR |
| 320 | DPLEQDERWMREALAEARAAAEAGEVPVGAVLVRDGVLLARAGNRTIRDRDPTAH |
| | AEMLALRAAARALGNHRLTGCTLYVTLEPCAMCAGAIAHARIARLVYGASDPKGGA |
| | VAHGPRLFEQPTLHHRPEVTGGVLAEEAGELLRAFFRARR |
| 321 | MREALAEARAAAEAGEVPVGAVVVAGGRIVARAHNQPIALNDPTAHAEVLALRQA |
| | ARELGNYRLTGCTLYATLEPCAMCAGAILHARLDRVVYGAADPKAGAAGSVLDVFA |
| | NPRLNHHAQVTGGVLAEECGALLSGFFRARR |
| 322 | DDLSFMREALAEAEAAAEAGEVPVGAVLVRDGEIVARGRNRVIEDRDPTAHAEIVAL |
| | REAGRALGNYRLEGCTLYVTLEPCAMCAGALVHARLDRLVYGAADPKAGAAGSVL |
| | DVLNHPRLNHRMEVTGGVLADECGAMLRAFFRARR |
| 323 | DRLRADEQFMRAALAEARAAAAAGEVPVGAVVVRDGEIVARAHNRPIALHDPTAH |
| | AEILALRAAARALGNYRLTGCTLYATLEPCAMCAGAILHARLDRVVYGAADPKAGA |
| | CGSVLSVMNHPRLNHRVEVTGGVLADECGALLTEFFRARR |
| 324 | MREALAEARAAAAAGEVPVGAVVVRGGEIVARAHNRPVALSDPTAHAEVLALREA |
| | ARRLGNYRLEGCTLYATLEPCAMCSGAVLHARLARLVYGAPDPKAGAAGSVLDVL |
| | DHPRLNHRTAVTGGVLAEECGALLSGFFRARRG |
| 325 | DDRRWMREALAEARAAGAAGEVPVGAVVVRDGELLARAGNASIRDRDPTAHAEML |
| | ALRAAARALGNHRLTGCTLYVTLEPCAMCAGAIAHARIARLVYGASDPKGGAVAHG |
| | PRLFEQPTLHHRPEVTAGVLADECGALLRDFFRARR |
| 326 | MREALAEARRAAEAGEVPVGAVVVRDGEIVARAHNRPIALHDPTAHAEILALREAA |
| | RRLGNYRLTGCTLYATLEPCAMCAGAILHARLDRVVYGAPDPKAGACGSVLSVFDQ |
| | PRLNHRTRVEGGVLAEECGALLSGFFRARR |
| 327 | DDRDRRWMRAALAEARAAAEAGEVPVGAVVVRDGELLARAGNASIRDRDPTAHAE |
| | MLALRAAARALGNHRLTGCTLYVTLEPCAMCAGAIAHARIARLVYGAADPKGGAV |
| | AHGPRLFEQPTLHHRPEVTAGVLAEEAGELLRAFFRARR |
| 328 | MREALAEARAAAAAGEVPVGAVVVRGGEIVARAHNRPIALNDPTAHAEILALREAA |
| | RVLGNYRLTGCTLYATLEPCAMCAGAILHARIDRLVYGAADPKAGACGSVLSVMNH |
| | PQLNHRVEVTSGVLAEECGALLTDFFRARR |
| 329 | MREALAEARAAAEAGEVPVGAVVVRDGEIVARAHNRPIALNDPTAHAEILALREAA |
| | RRLGNYRLTGCTLYATLEPCAMCAGAILHARLARLVYGAADPKAGACGSVLSVINH |
| | PRLNHRVEVTSGVLADECGALLSDFFRARRRAA |
| 330 | DDRFMRAALAEARAAAEAGEVPVGAVVVVDGRIVARAHNRPVELSDPTAHAEVLA |
| | LREAARALGNYRLEGATLYATLEPCAMCSGAILHARVERVVYGAADSRAGAAGSVL |
| | DVFATRRLNHQTTVTGGVLAEECGALLSGFFRARR |
| 331 | DDDQRFMREALAEARAAAAAGEVPIGAVVVCDGAIVARAGNRTIRDNDPTAHAEIL |
| | ALRQAARALGNYRLTGCTLYVTLEPCAMCAGAIVQARIARLVYGAADPKAGAVDSV |
| | LDVLNHPRLNHRVEVTRGVLAEECGALLRDFFRARR |
| 332 | MSDDERFMRLALEQARLAAEAGEVPVGAVVVCDGRVVARAHNRPIALNDPTAHAEI |
| | LALREAARELGNYRLTGCTLYATLEPCAMCSGAVLHARLARVVYGAADPKTGAAGS |
| | VLDLFAEPRLNHHAEVTGGVLAGECGALLSGFFRARRG |
| 333 | DPTDLAFMREALAEAEAAAAAGEVPVGAVVVHEGRIVGRGRNRMIAASDPTAHAEI |
| | VALREAARALGNYRLTGCTLYVTLEPCAMCAGAILHARIARLVYGAADPKAGACGS |
| | VLDVLNHPRLNHRTEVTAGVLAEECGAMLSGFFRARRAQQRAARVAAS |
| 334 | DPPDAVEMRAALEEARAAAEAGEVPVGAVLVHDGQILARAGNRTIRDRDPTAHAEIL |
| | ALRQAARVLGSHRLTGATLYVTLEPCAMCAGAISHARIARLVYGADDPKGGAVRHG |
| | PRFFEQPTCHHRPEVTGGILAEESAALLRGFFRARR |
| 335 | MRAALEEARRAAEAGEVPVGAVVVRDGAIVARAHNRPIALHDPTAHAEILALREAA |
| | RRLGNYRLTGCTLYATLEPCAMCAGAILHARLDRVVYGAADPKAGACGSVLSVFDQ |
| | PRLNHRTRVEGGVLAEECGALLSGFFRARR |
| 336 | DDDQRFMALALAQAQAAADAGEVPVGAVVVCDGRVVARAHNRPIALNDPTAHAEI |
| | LALREAARALGNYRLTGCTLYATLEPCAMCAGAILHARIARLVYGARDPKAGAVES |
| | LYRLLDDDRLNHRVAVTGGVLAAACGALLSGFFRARRR |
| 337 | DLTDEQLMRAALAEARAAAAAGEVPVGAVVAKDGEIIARAHNRPIAAHDPTAHAEIL |
| | ALRAAARALGNYRLTGCTLYATLEPCAMCSGAILHARIARLVYGAADPKAGACGSV |
| | LAVMNHPQLNHRVEVTGGVLAEECGALLRDFFRARR |
| 338 | DDDERWMREALAEARAAAEAGEVPVGAVVVRDGELLARAGNASIRDRDPTAHAEM |
| | LALRAAARALGNHRLTGCTLYVTLEPCAMCAGAIAHARIARLVYGASDPKGGAVAH |
| | GPRLFEQPTLHHRPEVTGGVLAEEAGALLRAFFRARR |
| 339 | DLEFMREALAEARAAAEAGEVPVGAVVVRDGAIVARAGNRTLRDRDPTAHAEMLA |
| | LRQAARALDSWRLSGCTLYVTLEPCAMCAGAMVQARLDRLVYGAADPKAGAAGS |
| | VLDVLGHPRLNHRVEVTGGVLAEECGALLREFFRARR |
| 340 | RSESDEALMREALAEARRAADAGEVPVGAVVVRDGAILARAGNAPIAASDPTAHAEI |
| | LALRAAARALGNYRLTGCTLYATLEPCAMCAGAILHARIARLVYGARDPKAGAVES |
| | VLDLLDRPALNHRVEVTGGVLAAECGALLSGFFRARR |
| 341 | MDDAGFMRAALAEARAAAEAGEVPVGAVVVCDGRIVARAGNRTLRDRDPTAHAE |
| | MLALRAAARALGSHRLTGCDLYVTLEPCAMCAGAISHARIRRLYYGASDPKGGAVE |
| | NGVRFFAQPTCHHAPEVYGGIGEAEAAALLRGFFRARR |
| 342 | MRDEDYMRAALAQAREAAAAGEVPVGAVVVADGRIVARAHNRPIALNDPTAHAEI |
| | LALREAARQLGNYRLTGCTLYATLEPCAMCAGAILHARIARLVYGAADPKAGAVGS |
| | VLDVLNHPRLNHRVEVTSGVLAAECGALLTEFFRSRRR |
| 343 | DETEAFMRAALAEARAAAAEGEVPVGAVVVADGAIVARAHNRPIALNDPTAHAEIL |
| | ALREAARALGNYRLTGCTLYATLEPCAMCAGAILHARIARLVYGARDPKAGAVESL |
| | YRLLDDTRLNHRVEAVGGVLADECGALLTEFFRARRG |
| 344 | DDDERWMREALAEARAAAAAGEVPVGAVVVRDGVLLARAGNASIRDRDPTAHAE |
| | MLALRAAARALGNHRLGGCTLYVTLEPCAMCAGAIAHARIARLVYGAADPKAGAV |
| | DHGPRLFEQPTCHHRPEVVSGVGAAESAALLRDFFRARR |
| 345 | MRAALEEARRAAEAGEVPVGAVVVRDGAIVARAHNRPIALNDPTAHAEILALREAA |
| | RALGNYRLTGCTLYATLEPCAMCAGAILHARLDRVVYGAADPKAGACGSVLSVFDQ |
| | PRLNHRTTLTGGVLAEECGALLSGFFRARRRAA |
| 346 | MDDDLFMRAALDEARAAAEAGEVPVGAVVVADGRIVARAHNRPIALNDPTAHAEIL |
| | ALRAAAAALGNYRLTGCELFVTLEPCAMCAGAILHARLARVVYGAADPKTGAAGSV |
| | LDVFANPRLNHRTEVRGGVLADECGALLTEFFRARRG |
| 347 | MDDEALMREALAEARAAAEAGEVPVGAVVARGGEIVARGRNRMIADGDPTAHAEI |
| | VALREAARALGNYRLTGCTLYATLEPCAMCAGAILHARIARLVYAAPDPKAGACGS |
| | VLSVMNHPQLNHRVEVASGLLAEECGALLTNFFRARRG |
| 348 | DPEDERFMREALAEAERAREAGEVPVGAVVVLDGRIVGRGHNRPIGAHDPTAHAEIV |
| | ALREAAAALGNYRLTGATLYVTLEPCAMCAGAILHARIARLVYGAADPKTGAVGSL |
| | LDLLAEPRLNHRTEVTGGVLAEECGALLSAFFRARR |
| 349 | DDLSFMREALAEAEAAAAAGEVPVGAVVVHGGRIVGRGRNRMIADHDPTAHAEIVA |
| | LREAARALGNYRLTGCTLYVTLEPCAMCAGAILHARIERLVYGAADPKAGACGSVLS |
| | VMNHPQLNHRVEVTSGVLADECGAMLSGFFRARR |
| 350 | DLEFMRLALEEARAAAAAGEVPVGAVLVRDGEVLARAGNRTIRDRDPTAHAEMLAI |
| | REAARRLDNYRLEGTTLYVTLEPCAMCSGAILHARVPRVVYGAADPKAGAAGSVLD |
| | VLGHPRLNHRTEVVGGVLAEECGALLREFFRARR |
| 351 | DDDHFMREALAEARRAADAGEVPVGAVVVRAGQIIARAHNRPVALHDPTAHAEILA |
| | LRAAARRLGNYRLTGCTLYATLEPCAMCAGAILHARIARLVYGAADPKAGACGSVL |
| | DVLNHPRLNHRVEVTAGVLAEECGALLSAFFRARR |
| 352 | DDDQRFMREALAEARAAAAAGEVPIGAVVVCDGEIVARAGNRTIRDNDPTAHAEIL |
| | ALRQAARALGNYRLTGCTLYVTLEPCAMCAGAIVHARIARLVYGAADPKAGAVNSV |
| | FDLLNHPRLNHRVEVTSGVLADECGALLRDFFRARR |
| 353 | DESFMREALAEARAAAEAGEVPVGAVVVHGGRIVARAHNRPIELSDPTAHAEILALR |
| | AAARALGNYRLTGCTLYATLEPCAMCAGAILHARIDRVVYGAADPKAGAAGSVLDV |
| | FANPRLNHHARVTGGVLADECGALLSGFFRARR |
| 354 | DPESDAAFMRAALAEAQAAAEAGEVPVGAVVVCDGRIVARAHNRPIGLNDPTAHAE |
| | ILALREAARELGNYRLTGCTLYVTLEPCAMCSGAILHARLARVVYGAADPKTGAAGS |
| | VLDLFANRRLNHQTQVEGGVLAEECGALLSGFFRARRG |
| 355 | MREALAEARAAAAAGEVPVGAVVVRGGEIVARAHNRPIALNDPTAHAEILALREAA |
| | RVLGNYRLTGCTLYATLEPCAMCAGAILHARIARLVYGAADPKAGACGSVLSVMNH |
| | PQLNHRVEVTGGVLAGECGALLSDFFRARR |
| 356 | MSDDERFMRLALAQAREAAAEGEVPVGAVVVCGGRVVARAHNRPIALNDPTAHAE |
| | VLALRAAARELGNYRLTGCTLYATLEPCAMCAGAILHARIARVVYGARDPKAGAAG |
| | SVLDVFANPRLNHHATVTGGVLAEECGALLSDFFRARRR |
| 357 | MDDDERWMREALAEARAAAEAGEVPVGAVVVRDGVLLARAGNASIRDRDPTAHA |
| | EMLALRAAARALGNHRLTGCTLYVTLEPCAMCAGAIAHARIARLVYGAADPKAGA |
| | VDHGPRLFEQPTCHHRPEVVSGVGAAESAALLRDFFRARR |
| 358 | DDDERWMRAALAEARAAAEAGEVPVGAVVVAGGRLVARAGNASIRDSDPTAHAEI |
| | LALRAAARALGNHRLAGCTLYVTLEPCAMCAGAIAHARIARLVYGASDPKGGAVAH |
| | GPRLFEQPTLHHRPEVTGGVLAAEAGALLRAFFRARR |
| 359 | MREALAEARRAAAEGEVPVGAVVVRDGAVIARAHNRPIALRDPTAHAEILALREAA |
| | RLLGNYRLTGCTLYVTLEPCAMCAGAIVHARIDRLVYGAADPKAGAVESVLRVLDH |
| | PRLNHRVEVTRGVLAEECGALLREFFRARR |
| 360 | MNDEDFMRAALAQAREAAAAGEVPVGAVVVCGGRIVARAHNRPISARDPTAHAEIL |
| | ALRAAARELGNYRLTGCELYVTLEPCAMCAGAILHARIARLVYGAADPKTGAAGSV |
| | LDVFANPRLNHRTEVTGGVLADECGALLSGFFRARRG |
| 361 | DDDQRFMAAALEEARAAAAEGEVPVGAVVVAGGRIVARAHNRPIALNDPTAHAEIL |
| | ALRAAARELGNYRLTGCTLYATLEPCAMCAGAILHARIDRVVYGARDPKAGAAGSV |
| | LDVFANPRLNHHAEVTGGVLAEECGALLSDFFRARR |
| 362 | MREALAEARAAAEAGEVPVGAVVVAGGRIVARAHNRPIALHDPTAHAEILALREAA |
| | RELGNYRLTGCTLYATLEPCAMCAGAILHARIARLVYGAADPKAGACGSVLAVMNH |
| | PQLNHRVEVVGGVLAEECGALLREFFRARR |
| 363 | MDSDLEFMREALAEARAAAEAGEVPVGAVVVRDGEIVARAANRTIRDGDPTAHAE |
| | MVALRAAARALGNYRLAGCTLYVTLEPCAMCAGAMIHARLDRVVYGAADPKAGA |
| | AGSVLDVLGHPALNHRTAVTAGVLAEECGALLRDFFRARR |
| 364 | DPTTDEALMREALAEARAAAADGEVPVGAVVARDGVIVARAHNAPVALSDPTAHA |
| | EILALREAARAAGNYRLPGCTLYATLEPCAMCCGAALHARLARVVYGARDPKAGAA |
| | GSVLDLLDDPRLNHRAEVVGGVLAEECGALLSGFFRARR |
| 365 | DPLSADDARFMRLALAEARAAAEAGEVPVGAVVVAGGRVVARAHNRPVALHDPTA |
| | HAEILALRAAARALGNYRLTGCTLYATLEPCAMCAGAILHARIARLVYGAADPKAG |
| | ACGSVLAVMNHPQLNHRVEVTGGVLAEECGALLRDFFRARR |
| 366 | MNDEDFMRAALAQAREAAAAGEVPVGAVVVCDGRIVARAHNRPIALNDPTAHAEIL |
| | ALREAARALGNYRLTGCTLYATLEPCAMCAGAILHARIARLVYGAADPKAGAVGSV |
| | LDVMNHPRLNHRVEVTSGVLAEECGALLKEFFRARR |
| 367 | DDDQRFMREALAEARAAAEAGEVPIGAVVVCDGEIVARAGNRTIRDNDPTAHAEILA |
| | LRQAARRLGNYRLTGCTLYVTLEPCAMCAGAIVQARIARLVYGAADPKAGAVDSVL |
| | QVLNHPRLNHRVEVTRGVLAEECGALLRDFFRARR |
| 368 | MREALAEARRAAEAGEVPVGAVVVRDGEIVARAHNRPIALHDPTAHAEILALREAA |
| | RRLGNYRLTGCTLYATLEPCAMCAGAILHARLDRVVYGAPDPKAGACGSVLSVFDQ |
| | PALNHRVAVTGGVLAEECGALLREFFRARR |
| 369 | DPDARFMREALAEAEAAAAAGEVPVGAVVVAGGRIVARAHNRPVELSDPTAHAEIL |
| | ALREAARELGNYRLEGCTLYATLEPCAMCSGAILHARLARVVYGAADPKAGAAGSV |
| | LDVFALPQLNHRTAVTGGVLADECGALLSGFFRARR |
| 370 | MNDEDYMRAALEQARLAASEGEVPVGAVVVCDGKIVARAHNRPIALNDPTAHAEIL |
| | ALREAARKLGNYRLTGCTLYATLEPCAMCSGAILHARIARLVYGAADPKAGAVGSV |
| | LDVINHPRLNHRVEVTSGVLAEECGALLSGFFRARRK |
| 371 | MRAALEEARRAAEAGEVPVGAVVVRDGAIVARAHNRPIALHDPTAHAEILALREAA |
| | RRLGNYRLTGCTLYATLEPCAMCAGAILHARIDRLVYGAADPKAGACGSVLSVMNH |
| | PRLNHRVEVERGVLAEECGALLTGFFRARR |
| 372 | MSTEDERFMRLALAEARRAAAEGEVPVGAVVVAGGEVVAAAHNRPIALADPTAHA |
| | EILALRAAARALGNYRLTGCTLYATLEPCAMCAGAILHARIARLVYGAADPKAGAVE |
| | SVLRLLDAPGLNHRVAVTGGVLAEECGALLREFFRARR |
| 373 | MREALAEARAAAEAGEVPVGAVVVRDGAIVARAHNRPIALNDPTAHAEILALREAA |
| | RALGNYRLTGCTLYATLEPCAMCAGAILHARLDRVVYGAADPKAGAAGSVLQVFD |
| | HPRLNHRTAVTGGVLAAECGALLSGFFRARRG |
| 374 | MREALAEARAAAAAGEVPVGAVVVRGGEIVARAHNRPIALNDPTAHAEILALREAA |
| | RALGNYRLTGCTLYATLEPCAMCAGAILHARIDRVVFGARDPKAGAAGSVLDVFAN |
| | PRLNHHAEVVGGVLADECGALLSGFFRARRG |
| 375 | DERFMREALAEARAAAAAGEVPVGAVVVRDGVVLARAGNRTLRDRDPTAHAEML |
| | ALRQAARALGSWRLAGCTLYVTLEPCAMCAGAMVLARVDRLVYGAADPKAGAAG |
| | SVLDVLGEPRLNHRPEVAAGVLAEECGELLRAFFRERR |
| 376 | DPTSDEAFMRAALAEARAAAEAGEVPVGAVVVCGGRIVARAGNRPIAARDPTAHAE |
| | ILALRAAARELGSYRLTGATLYVTLEPCAMCAGAILHARIERLVFGAADPKTGAAGS |
| | VLDLFAEPRLNHRTAVEGGVLAEECGALLRDFFRARRG |
| 377 | DPSPDEAFMRAALAEARAAAEAGEVPVGAVVVRDGRIVARAGNRPIALHDPTAHAEI |
| | LALRAAARELGNYRLTGCELYVTLEPCAMCAGAILHARLARLVYGAADPKTGAAGS |
| | VLDVFANRRLNHHTAVTGGVLADECGALLAGFFRARR |
| 378 | MDDAAFMRAALAEARAAAEAGEVPVGAVVVADGAIVARAHNRPIALNDPTAHAEIL |
| | ALRAAARALGNYRLTGCTLYATLEPCAMCAGAILHARIARLVYGARDPKAGAVESL |
| | YRLLDDTRLNHRVAVTGGVLADECGALLTEFFRARRR |
| 379 | DPESDAAFMRAALAEARAAAEAGEVPVGAVVVCDGRIVARAHNRPIGLNDPTAHAE |
| | ILALRAAARALGNYRLTGCTLYVTLEPCAMCAGAILHARLDRVVYGAADPKTGAAG |
| | SVLDLFAQPRLNHHARVEGGVLADECGALLRDFFRARR |
| 380 | DPDAAFMRAALAEARAAAEAGEVPVGAVVVAGGRIVARAHNRPIELNDPTAHAEIL |
| | ALRQAAAALGNYRLEGCTLYVTLEPCAMCAGAILHARLARVVYGAADPKTGAAGS |
| | VLDLFANPRLNHHAEVTGGVLAEECGALLRDFFRARRG |
| 381 | MREALAEARRAAEAGEVPVGAVVVRDGEIVARAHNRPIALHDPTAHAEILALREAA |
| | RRLGNYRLTGCTLYATLEPCAMCAGAILHARIDRLVYGAADPKAGACGSVLSVMNH |
| | PRLNHRVEVESGLLAGECGALLTEFFRRRRAAARG |
| 382 | MSTDLAFMREALAEARAAAEAGEVPVGAVVVRDGAILARAGNRTLRDGDPTAHAE |
| | MVALRAAAAALGSERLTDCDLYVTLEPCAMCAGAISHARIRRLYYGAADPKGGAVE |
| | HGVRFFASPTCHHRPEVYGGIGEGEAAALLRDFFRARR |
| 383 | DDRFMREALAEARAAAAAGEVPVGAVVVRDGEIIARAGNRTLRDRDPTAHAEMLAL |
| | RQAARALGSHRLTGCDLYVTLEPCAMCAGAIQHARIRRLVYGAADPKAGAVDHGV |
| | RLFDSPSCHHRPEVVAGVGEGEAAALLRDFFRERR |
| 384 | MREALAEARAAAAAGEVPVGAVVVRGGEIVARAHNRPIALHDPTAHAEILALREAA |
| | RALGNYRLEGCTLYATLEPCAMCSGAILHARLARLVYGAADPKAGAVGSVLDVIGN |
| | PRLNHRVEVTGGVLAEECGALLSGFFRARR |
| 385 | MDDAGFMRAALAEARAAAAAGEVPVGAVVVRDGAIVARAGNRTLRDRDPTAHAE |
| | MLAIRAAARALGSERLTGCDLYVTLEPCAMCAGAISFARIRRLYYGAADPKGGAVEN |
| | GVRFFASPTCHHAPEVYSGIGEAEAAALLRDFFRARR |
| 386 | RPPSDDERWMAEALAQARAAAAEGEVPVGAVVVRDGRLLARAHNRPIALNDPTAH |
| | AEILALRAAAREIGNYRLTGCTLYATLEPCAMCAGAILHARIARLVYGARDPKAGAC |
| | GSVLDVLNHPRLNHRVEVTGGLLADECGALLSAFFRARR |
| 387 | DLEFMREALAEARAAAEAGEVPVGAVVVAGGRIVARAHNRPIELHDPTAHAEILALR |
| | QAATALGNYRLTGCTLYATLEPCPMCAGAALHARLDRIVYGAADPKAGAAGSVLDL |
| | FADTRLNHHAAVAGGVLADECGALLSGFFRARR |
| 388 | DDDERWMRAALAEARAAAAAGEVPVGAVVVRDGELLARAGNASIRERDPTAHAEL |
| | LALRAAARRLGNHRLTGCTLYVTLEPCAMCAGAIAHARIARLVYGAADPKGGAVAH |
| | GPRLFEQPTLHHRPEVTAGVLAEECGALLREFFRARR |
| 389 | MREALAEARAAAAAGEVPVGAVVVRGGEIVARAHNRPIALHDPTAHAEILALREAA |
| | RRLGNYRLTGCTLYATLEPCAMCAGAILHARIDRLVYGAADPKAGACGSVLSVMNH |
| | PQLNHRVEVTSGVLAEECGALLTGFFRERR |
| 390 | DERWMREALAEARAAAAEGEVPVGAVVVRDGRLLARAGNASIRSRDPTAHAEILAL |
| | RRAARRLGNHRLTGCTLYVTLEPCAMCAGAIAHARIARLVYGASDPKGGAVAHGPR |
| | LFEQPTCHHRPEVTGGVLAEECGALLREFFRARR |
| 391 | MDDAGFMRAALAEARAAAEAGEVPVGAVVVADGRIVARAHNRPVALNDPTAHAEI |
| | LALRAAARALGNYRLTGCTLYATLEPCAMCAGAILHARLDRVVYGAADPKAGAAG |
| | SVLDVFANPRLNHRTAVTGGVLADECGALLTGFFRARR |
| 392 | DDRFMRAALAEARAAAEAGEVPVGAVVVCGGRIVARAHNRPIDLCDPTAHAEILAL |
| | REAARELGNYRLTGCTLYATLEPCAMCAGAILHARIARLVYGARDEKAGAVESLYR |
| | LLDDTRLNHRVAVTGGVLAAECGALLSEFFRARRA |
| 393 | DPDAAFMRAALAEARAAAEAGEVPVGAVVVHEGRIVARAHNRPVALNDPTAHAEIL |
| | ALRAAARALGNYRLTGCTLYATLEPCAMCAGAILHARIARLVYGARDPKAGAVESL |
| | YRLLDDTRLNHRVEAVGGVLADECGALLSEFFRARRR |
| 394 | DDRFMRAALAEARAAAAAGEVPVGAVVVAGGRIVARAHNQPVALVDPTAHAEVLA |
| | LRAAARELGNYRLTGCTLYATLEPCAMCAGAILHARIDRLVYGAADPKAGAVGSVL |
| | DVFANPRLNHRTRVTGGVLAAECGALLSEFFRARRR |
| 395 | MTSAPAPSDLDFMRLALAEARLAAAEGEVPVGAVVVCDGEVVARAHNRPIALHDPT |
| | AHAEILALREAARKLGNYRLTGCTLYATLEPCAMCAGAILHARIARLVYGAADPKAG |
| | AVESRLRLLDAPFLNHRVEVTGGVLAEECGALLKDFFRARR |
| 396 | DDRFMRAALAEARAAAEAGEVPVGAVVVCGGEIVARAHNRPIALNDPTAHAEILAL |
| | REAARRLGNYRLTGCTLYATLEPCAMCAGAILHARIARLVYGAADPKAGAVESLYR |
| | LLDDTRLNHRVEAVGGVLAGECGALLSEFFRARRA |
| 397 | MREALAEARAAAAAGEVPVGAVVVAAGRIVARAHNRPVELSDPTAHAEILALREAA |
| | RELGNYRLEGCTLYATLEPCAMCAGALLHARLARLVYGAADPKAGAVGSVLDVLD |
| | HPRLNHRVEVTAGVLAEECGALLRDFFRARR |
| 398 | MDEEFMREALAEARAAAEAGEVPVGAVVVAGGRIVGRGRNRPVELADPTAHAEILA |
| | LREAAAALGNYRLEGCTLYATLEPCAMCAGAILHARIARVVYGARDPKAGAAGSVL |
| | DVFALPRLNHHTRVTGGVLAEECGALLSGFFRARR |
| 399 | RLDEQFMRRALELAAHAEAEGEVPVGAVLVLDGQVIGEGWNRSIGHHDATAHAEM |
| | MAIEQAGKAVENYRLLDATLYVTLEPCPMCAGALLHSRVKRIVYGAPDLKAGAAGT |
| | VLNLFEHQAAYHYADVEHGLLEEECREQLQAFFKRRRKEKKALKQAQRDAEEK |
| 400 | MSTSEADLTFMREALAEARAAAEAGEVPVGAVVVAGGEIVARAHNRPIALHDPTAH |
| | AEILALRAAARRLGNYRLTGCTLYVTLEPCAMCAGAILHARLARLVYGAADPKTGA |
| | AGSVLDLFANRRLNHHTEVTGGVLADECGALLSDFFRARRA |
| 401 | MDDAGFMRLALAEARRAAEAGEVPVGAVVVRGGEVLAAAGNRTVRDCDPTAHAE |
| | VVALREAARKLGNHRLTGCDLYVTIEPCAMCAGAISHARIARLYYGADDPKGGAVE |
| | HGPRFFGQPTCHHRPEVYGGIGAAEAAELLRGFFRARR |
| 402 | MREALAEARAAAEAGEVPVGAVVVRDGEIIARARNRMVADCDPTAHAEIVALREAA |
| | RALGNHRLGGCTLYVTLEPCAMCAGAMIHARVDRLVYGAADPKAGAAGSVLDVIN |
| | HPRLNHRMEVESGVLADECGALLKEFFRSRR |
| 403 | MDAALAEARAAADAGEVPVGAVVVRDGAVLARAGNRTLRDSDPTAHAEIVALRAA |
| | ARALGNHRLPGCTLYVTLEPCAMCAGAISHARLARLHYGAADPKGGAVEHGPRFFA |
| | QPTCHHRPDVVGGLGETEAADLLRAFFAARR |
| 404 | MSDEQYMRRALELARQAEQQGEVPVGAVLVRDGEVIAEGWNQSISSHDATAHAEIM |
| | ALRAAGQALENYRLLDTTLYVTLEPCPMCAGALLHSRVKRVVFGAPDLKAGAAGTV |
| | LNLFESQASYHYADVESGLLEEECREQLQAFFKRRRKEIKAQRDAKRAADE |
| 405 | MDAALAEARAAADAGEVPVGAVVVRDGVVLARAGNRTVRDADPTAHAEIVALRA |
| | AARALGNHRLPGCTLYVTLEPCAMCAGAISHARLARLVYGAADPKGGAVAHGPRLF |
| | AQPTCHHRPEVTGGVGAAEAAALLRAFFAARR |
| 406 | MDDAGFMRLALAEARAAAAAGEVPVGAVVVRDGEVLAAAGNRTLRDRDPTAHAE |
| | VVALRAAARALGSERLPGCDLYVTLEPCAMCAGAISFARIRRLYFGAGDPKGGAVEH |
| | GPRFFAQPTCHHAPEVYGGLGEGEAAGLLRGFFAARR |
| 407 | MRSALDEARRAAAAGEVPVGAVVVRDGAVLAAAGNRTVRDCDPTAHAEIVALRAA |
| | AAALGNYRLDGCDLYVTLEPCAMCAGAMVHARLARLVYGAADPRAGAAGSVLDV |
| | VRHPALNHRMEVTGGVLADECGALLREFFAARR |
| 408 | MDSAAKDLCYMRRALELAALAEAEGEVPVGAVLVKDGEIVGEGWNRSIGSHDATA |
| | HAEIMALRQAGQALQNYRLLDTTLYVTLEPCPMCAGALLHSRVKRVVFGAPDLKAG |
| | AAGTVLNLFESQASYHYAEVESGLLEQECREQLQAFFKRRRKEIKALRQAEKEAQQK |
| 409 | MDSAAKDLCYMRRALELAALAEAEGEVPVGAVLVKDGEIVGEGWNRSIGSHDATA |
| | HAEIMALRQAGQALQNYRLLDTTLYVTLEPCPMCAGALLHSRVKRVVFGAPDLKAG |
| | AAGTVLNLFESQASYHYAEVESGLLEEECREQLQAFFKRRRKEIKAQRDAKRAAD |
| 410 | MTSAPEHEKYMRRALELARQAEEHGEVPVGAVLVKDGEIIAEGWNQSIGSHDATAH |
| | AEIMALRAAGKALENYRLLDTTLYVTLEPCPMCAGALLHSRVKRVVFGAPDLKAGA |
| | AGTVLNLFESQASYHYADVESGLLEEECREQLQAFFKRRRKEIKALKQAKREQEE |
| 411 | DTEFMREALAEARAAAAAGEVPVGAVVVHEGKIIARAGNRTIRDNDPTAHAEIVALR |
| | AAARALGNYRLAGCTLYVTLEPCAMCAGAMIHARLDRLVYGADDPKAGAAGSVLN |
| | VLNHPRLNHQMQVESGVLADECAALLQDFFRARR |
| 412 | MTDADFMALALEEARAAAALGEVPVGAVVVRDGAVIARAGNRTVRDNDPTAHAEI |
| | VALREAARALGNYRLDGCTLYVTLEPCAMCSGAMLHARLARVVYGAADPKTGAAG |
| | SVLDLFATAQLNHQTQVQGGVLAAECGALLQDFFRQRR |
| 413 | DESYMRRALELARQAEQHGEVPVGAVLVKDGEVIAEGWNQSIGSHDATAHAEIMAL |
| | RAAGQVLENYRLLDTTLYVTLEPCPMCAGALLHSRVKRVVFGAPDLKAGAAGTVLN |
| | LFESQASYHYAEVEGGLLEDECREQLQAFFKRRRKEIKAARQAKRAAE |
| 414 | MDEALAEARRAAAAGEVPVGAVLVRDGRVLARGGNRTIRDCDPTAHAEIVALREAA |
| | RAAGNYRLPGTTLYVTLEPCAMCAGAMIHARVARLVYGAADPRAGAAGSVFDVLR |
| | HPALNHRMEVEGGVRAEECGALLREFFRARRA |
| 415 | MTEADLRFMQLALEEARRAGEAGEVPVGAVLVRDGAVLARGGNRTIRDCDPTAHA |
| | EIVALREAARALGNYRLDGCTLYVTLEPCAMCAGAMIHARLDRLVYGADDPRAGAA |
| | GSVLDVLGHPALNHQTRVTAGVLAEECGALLREFFRARR |
| 416 | MTDEEYMRRALELARQAEQQGEVPVGAVLVKDGEVIAEGWNQSIGSHDATAHAEI |
| | MALRAAGQALENYRLLDTTLYVTLEPCPMCAGALLHSRVKRVVFGAPDLKAGAAG |
| | TVLNLFESQASYHYADVESGLLEEECREQLQAFFKRRRKEIKALKQAKRDAEE |
| 417 | MRDALAEARAAAARGEVPVGAVVVRDGAVLARAGNASIAARDPTAHAEILALRAA |
| | ARALGNHRLTGCTLYVTLEPCAMCAGAIAHARIARLVYGAADPKGGAVAHGPRFFA |
| | QPTCHHRPEVAGGLGEAEAAALLRDFFRARR |
| 418 | MTDEDFMRLALAEAQAAAAAGEAPIGAVLVRDGQVLARGQNRVIRDNDPTAHAEIV |
| | ALREAARQLGNYRLDGCELYVTLEPCAMCAGAMIHARLARLVYGAADPKAGAAGS |
| | VLDVINHPRLNHRMQVTAGVLADECGALLRDFFRQRR |
| 419 | MRAALDEARAAADAGEVPVGAVVVRDGAILARAGNRTVRDADPTAHAEIVALRAA |
| | ARALGNHRLGGCTLYVTLEPCAMCAGAIAQARVARLVYGAADPKGGAVAHGPRLF |
| | AQPTCHHRPEVTGGVLADEAGELLRAFFAARR |
| 420 | DETYMRRALELARQAEQHGEVPVGAVLVKDGEVIAEGWNQSIGAHDATAHAEIMA |
| | LRAAGQALQNYRLLDTTLYVTLEPCPMCAGALLHSRVKRVVFGAPDLKAGAAGTVL |
| | NLFESQASYHYAEVESGLLEEECREQLQAFFKRRRKEIKAARQAARDAQ |
| 421 | MNDEFYMRRALELAKMAEERGEVPVGAVLVRDGEVIAEGWNQSIGSHDATAHAEI |
| | MALRAAGQVLQNYRLLDTTLYVTLEPCPMCAGALLHSRVKRVVFGAPDLKAGAAG |
| | TVLNLFESQASYHYAEVESGLLEEECREQLQAFFKRRRKEIKALKQAQREAEEK |
| 422 | DTSPADLRFMRLALAEARAAAAAGEVPVGAVVVRDGAVLARAGNRTLRDRDPTAH |
| | AEIVALRAAARALGSHRLGGCTLYVTLEPCAMCAGAIAQARVARLVYGADDPKGGA |
| | VAHGPRLFAQPTCHHRPEVVGGVGAEEAAALLRDFFAARR |
| 423 | MTAADERFMRRAIELAAQAEAAGEVPVGAVLVKDGEIVAEGWNQSIGSHDATAHAE |
| | IQTLRQAGQALENYRLLDTTLYVTLEPCPMCAGALLHSRVKRVVFGAPDLKAGAAG |
| | TVLNLFESQASYHYADVEGGLLEDECREQLQAFFKRRRKEIKAARQAAKEAE |
| 424 | MDDDGLMRAAIAEAEAAAAAGEVPVGAVLVRDGVVLARGRNRMIADSDPTAHAEI |
| | VALREAARALGNYRLEGCELYVTLEPCAMCAGAMVHARLARLVYGAADPKAGAA |
| | GSVLDVLGHPRLNHRMEVTGGVLADECGAMLRDFFRARRA |
| 425 | DDTFYMRRALELAALAESEGEVPVGAVLVKDGEIIAEGWNRSIGSHDATAHAEIETL |
| | RKAGQALQNYRLLDTTLYVTLEPCPMCAGALLHSRVKRIVFGAPDLKAGAAGTVLN |
| | LFESQASYHYAEVESGLLEEECREQLQAFFKRRRKEIKALRQAEKAAQE |
| 426 | MDDAGFMREALAEARAAAAAGEVPVGAVVVKDGEIIARAGNRTIRDCDPTAHAEIV |
| | ALRAAAQALGNYRLDGCTLYVTLEPCAMCSGAMLHARLPRVVYGAADPKTGAAGS |
| | VLDLFAEPRLNHHTRVEGGVLAAECGALLTDFFRARRG |
| 427 | MDAALEEARRAAAAGEVPVGAVLVRDGVVLARAGNRTVRDCDPTAHAEIVALRAA |
| | AAALGNYRLPGTTLYVTLEPCAMCAGAMIHARVARLVYGAADPKTGAAGSVFDVL |
| | GSPRLNHRVEVEGGVRAAECGALLQDFFRARRAG |
| 428 | MTDDERWMAEALREAEAAAAEGEVPVGAVVVRDGELIARGRNRVVRDCDPTAHAE |
| | IVALREAARALGNYRLTGCTLYVTLEPCAMCAGAMVHARLDRLVYGAADPKAGAA |
| | GSVLAVLNHPRLNHQMEVAGGVLAGESAALLRDFFRARR |
| 429 | DSEFMAEALAEARAAAEAGEVPIGAVVVCDGEIVARAGNRTIRDNDPTAHAEIVALR |
| | EAAAKLGNYRLAGCTLYVTLEPCAMCAGAMIHARLDRLVYGAADPKAGAAGSVLD |
| | VLGHPQLNHQTEVEGGVLAEECGALLKDFFASRR |
| 430 | DPEYMRRALELAAQAEAEGEVPVGAVLVKDGEVVAEGWNRSIGDHDATAHAEIQV |
| | LRKAGQALQNYRLLDTTLYVTLEPCPMCAGALLHSRVKRVVFGAPDLKAGAAGTVL |
| | NLFESQASYHYADVESGLLEDECREQLQAFFKRRRKEIKAARKAAKEAE |
| 431 | DTEMMRLALAEARAAAEAGEVPVGAVVVRDGVVLARAGNRTVRDNDPTAHAEIVA |
| | LREAARALGNHRLEGCTLYVTLEPCAMCAGAIAQARVARLVYGAADPKGGAVAHG |
| | PRLFEQPTTHHRPEVTGGVLAEEAGALLREFFAARR |
| 432 | MTDADFMALALEEARAAAALGEVPVGAVVVRDGAVVARAGNRTVRDCDPTAHAE |
| | VVALREAARVLGNYRVDGATLYVTLEPCAMCAGAMIHARLPRLVYGAADPRAGAA |
| | GSVLDVLGHPALNHRVEVTGGVLADESAALLREFFRERR |
| 433 | DEPLDRDRYFMEIALAEARAAAAAGEVPVGAVVVRDGEVIARAGNRTVRDNDPTAH |
| | AEIVALREAARALGNHRLTGCTLYVTLEPCAMCAGAISHARVARLVYGADDPKGGA |
| | VAHGPRFFEQPTCHHRPEVTGGVLAAESAALLRGFFAARR |
| 434 | MTSDAEFMALALEEARAAAALGEVPVGAVVVRDGAVIARAGNRTIRDCDPTAHAE |
| | MVALREAARALGNYRLAGCDLYVTLEPCAMCAGAIVHARLARLVYGAADPKTGAA |
| | GSVFDVLGSGRLNHRPEVVAGVLADECGALLTDFFRARR |
| 435 | MTEEDKYFMQRAIELAKLAEENGEVPVGAVLVKDGEIIAEGWNQSIGNHDATAHAEI |
| | MTLRQAGQVLQNYRLLDTTLYVTLEPCPMCAGALLHSRVKRIVFGAPDLKAGAAGT |
| | VLNLFESQASYHYADVESGLLEEECREQLQAFFKRRRKEIKALKKQQKLEQE |
| 436 | MSAALDEARRAAAAGEVPVGAVVVRDGVVLAAAGNRTLRDCDPTAHAEIVALRAA |
| | ARALGNYRLDGCDLYVTLEPCAMCAGAMIHARLARLVYGAADPKTGAAGSVLDVL |
| | GHPRLNHRTAVTGGVLADECGALLRDFFAARR |
| 437 | DTSERDERFMRLALEEARAAAEAGEAPIGAVVVRDGAVLARAGNRTVRDNDPTAHA |
| | EVVALREAARALGNHRLTGCTLYVTLEPCAMCAGAISHARVARLVYGADDPKGGA |
| | VAHGPRFFAQPTCHHRPEVTGGVLAEESAELLRGFFRARR |
| 438 | DPDSRFMQAALAEARAAAEAGEVPIGAVVVRDGEIVARAGNRTIRDNDPTAHAEIVA |
| | LREAARVLGNYRLTGCTLYVTLEPCAMCAGAMIHARIDRLVYGAADPKAGAAGSVL |
| | DVLNHPKLNHQMEVEAGVLADECGALLRDFFRARR |
| 439 | MDDDERWMRLALEEARLAEEAGEVPVGAVVVRDGELLARGRNRVLRDCDPTAHAE |
| | IVALREAARRLGNYRLDGCTLYATLEPCAMCAGAMVHARLARLVYGAPDPKAGAA |
| | GSVLDVLNHPRLNHRMEVTAGVLAEECGALLRRFFQARR |
| 440 | MSAALAEARRAAAAGEVPVGAVVVRDGAVLAAAGNRTVRDCDPTAHAEIVALRAA |
| | ARALGNHRLEGCTLYVTLEPCAMCAGAIAQARVARLVYGAADPKGGAVAHGPRLF |
| | AQPTCHHRPEVVGGVGEAEAAALLRDFFAARR |
| 441 | MRSALALAAAAGAAGEVPVGAVVVRDGVVIGRGENRVIRDSDPTAHAEIVALRDAA |
| | RALGNYRLTGCTLYVTLEPCAMCAGAMIHARIDRLVYGAADPKAGAAGSVLDVLN |
| | HPQLNHQMEVEGGVLAEECGALLRDFFRARR |
| 442 | RTADERWMRLALEEARAAEAAGEVPVGAVVVRDGELIARGRNRVLRDCDPTAHAEI |
| | VALREAARALGNYRVEGCTLYVTLEPCAMCAGAMIHARLDRLVYGAADPRAGAAG |
| | SVLDVLGHPALNHQTQVTGGVLAEECGALLREFFRARRG |
| 443 | DDLSFMQEALAEARAAAEAGEVPIGAVVVRDGAILARAGNRTVRDNDPTAHAEIVA |
| | LREAARALGNYRLAGCTLYVTLEPCAMCAGAMIHARVDRLVYGAADPKAGAAGSV |
| | LDVLGHPRLNHRMEVEGGVLAEECGALLREFFRARR |
| 444 | MSSDAGFMAEALAEAEAAAAAGEVPVGAVVVCGGEIVARAGNRTIRDCDPTAHAE |
| | MVALREAARKLGNYRLAGCTLYVTLEPCAMCAGAMIHARLDRLVYGAADPKAGAA |
| | GSVLDVINHPRLNHQMEVEAGVLADECGAMLRRFFQERR |
| 445 | MDDDRWMRLALEEARRAEEEGEVPVGAVVVLGGEVIGRGRNRVIRDSDPTAHAEIV |
| | ALREAARTLGNYRLDGCELYVTLEPCAMCAGAMVHARLARLVYGAADPRAGAAGS |
| | VLDVLGHPRLNHRMEVTGGVLAEECGALLRDFFRRRR |
| 446 | MDDAGFMREALAEAEAAAAAGEVPVGAVVVCDGRIVARAGNRTVRDNDPTAHAEI |
| | VALREAARALGNHRLGGCTLYVTLEPCAMCAGAMIHARIDRLVYGADDPKAGAVR |
| | SVLQVLDHPRLNHRMAVESGVLAGECAAVLQAFFAARR |
| 447 | MDAALEEARRAAAAGEVPVGAVLVRDGEVLARGGNRTIRDCDPTAHAEIVALRAAA |
| | RAAGNYRLPGTTLYVTLEPCAMCAGAMIHARVARLVYGAADPRAGAAGSVFDVLR |
| | HPALNHRMEVEGGVLAEESAALLRGFFRARR |
| 448 | MRDALALARAAAAAGEVPVGAVVVKDGVVVGRGENRVVRDSDPTAHAEIVAMRE |
| | AARALGNYRLTGCTLYVTLEPCAMCAGAMIHARLDRVVYGAADPKTGAAGSVLDV |
| | LGHPRLNHQTQVEGGVLAAECGALLSDFFRARR |
| 449 | MDDAGFMREALAEAEAAAAAGEVPVGAVVVKDGEIIARAGNRTVRDCDPTAHAEIV |
| | ALREAARKLGNYRLAGCTLYVTLEPCAMCAGAMIHARLDRLVYGADDPKAGAAGS |
| | VLNVLNHPRLNHQMQVEAGVLAEECGAMLRRFFETRR |
| 450 | MDDAAFMGEALAEARAAAAAGEVPIGAVVVCDGEIVARAGNRTVRDNDPTAHAEI |
| | VALREAARKLGNYRLAGCTLYVTLEPCAMCAGAMIHARVDRLVYGADDPKAGAAR |
| | SVLNVVDHPALNHRLEVTSGVLAAECAELLRGFFERRR |
| 451 | MTAADAAAMRAALAEARAAAARGEVPVGAVVMRDGVIVARAGNATVAGHDPTAH |
| | AEIVALRRAARALGNHRLTGCALYVTLEPCAMCAGAIAHARIARLVYGAADPKGGA |
| | VAHGPRFFAQPTCHHRPEVTGGVLAEEAGALLRAFFRARR |
| 452 | DTEMMALALAEARAAAEAGEVPVGAVVVRDGEVLARAGNRTVRDCDPTAHAEIVA |
| | LREAARRLGNYRLPGCTLYVTLEPCAMCAGAIMHARLDRVVYGAADPKTGAAGSV |
| | LDLFAEPRLNHHTAVVGGVLADEAGALLRGFFAARR |
| 453 | MTEEDKYFMKRAIELAKLAEENGEVPVGAVLVKDGEIISEGWNQSIGNNDATAHAEI |
| | MALREAGKVLENYRLLDTTLYVTLEPCPMCAGALLHSRVKRIVFGAPDLKAGAAGT |
| | VLNLFESQASYHYADVESGLLEEECREQLQAFFKRRRKEKKELKKLKREEN |
| 454 | MDSAAKDLCYMRRALELAALAEAEGEVPVGAVLVKDGEIVGEGWNRSIGSHDATA |
| | HAEIMALRQAGQALQNYRLLDTTLYVTLEPCPMCAGALLHSRVKRVVFGAPDLKAG |
| | AAGTVLNLFESQASYHYADVESGLLEEECRAQLQAFFKRRRKEIKAQRDAQRAAQE |
| | K |
| 455 | MRTALEEARRAAEAGEVPVGAVVVRDGVVLARAHNRTVADHDPTAHAEILALREA |
| | ARVLGNHRLTGCTLYVTLEPCAMCAGAIVHARLDRLVYGAADPKAGAAGSVLDVL |
| | GHPRLNHRPEVAAGVLAEECGELLRGFFRERR |
| 456 | DDDERWMREALAEARAAEEAGEVPVGAVVVRDGELVGRGRNRVLRDSDPTAHAEI |
| | VALREAARALGNYRLDGCTLYATLEPCAMCAGAMLHARLARLVYGAADPKAGAA |
| | GSVLDVLNHPRLNHRMEVTGGVLAEECGALLRGFFRARRG |
| 457 | RSSPDERFMALALAEARRAAEAGEVPVGAVLVRDGAVLAAAGNRTIRDNDPTAHAE |
| | IVVLREAARRLGNYRLEGCTLYVTLEPCAMCAGAMVNARLGRLVYGAADPRAGAA |
| | GSVLDVLRHPALNHRMEVTGGVLAAECGALLRDFFAARR |
| 458 | MRDALAEARKAAALGEVPVGAVVVKDGAVIARGHNRTVADSDPTAHAEIVAIRAA |
| | ARALGNHRLTGCTLYVTLEPCAMCAGAIAHARIARLVYGAADPKGGAVDHGVRFFE |
| | SPTCHHRPEVTGGVLAAEAAALLRDFFRARR |
| 459 | DETYMRRALELARQAEEHGEVPVGAVLVKDGEIIAEGWNQSIGAHDATAHAEIMAL |
| | RAAGQALENYRLLDTTLYVTLEPCPMCAGALLHSRVKRIVFGAPDLKAGAAGTVLN |
| | LFESQASYHYADVESGLLEEECREQLQAFFKRRRKEIKALKQAQREAEEK |
| 460 | MDEALAEARRAAAAGEVPVGAVVVRDGAVLARAGNRTVRDCDPTAHAEIVALRAA |
| | ARALGNHRLTGCDLYVTLEPCAMCAGAISHARIARLYYGADDPKGGAVAHGPRFFG |
| | QPTCHHRPEVYGGIGEGAAAALLRAFFAARR |
| 461 | DESYMRRALELARQAEAEGEVPVGAVLVKDGEVVAEGWNRSIGAHDATAHAEIETL |
| | RKAGQALENYRLLDTTLYVTLEPCPMCAGALLHSRVKRVVFGAPDLKAGAAGTVLN |
| | LFESQASYHYAEVESGLLEDECREQLQAFFKRRRKEIKAQRQAQKEAEQK |
| 462 | MSEQDRYFMQRAIELAQQAELNGEVPVGAVLVKDGEVIAEGWNQSIGSHDATAHAE |
| | IMALRKAGQVLENYRLLDTTLYVTLEPCPMCAGALLHSRVKRVVFGAPDLKAGAAG |
| | TVLNLFESQASYHYADVESGLLEDECREQLQAFFKRRRKEKKALKQAQKEAAQ |
| 463 | MTDADFMALALEEARAAAALGEVPVGAVVVKDGEVIARAGNRTVRDCDPTAHAEI |
| | VALREAARKLGNYRLDGCTLYVTLEPCAMCAGAMLHARLPRVVYGAADPKTGAAG |
| | SVLDLFAEPRLNHQTAVQGGVLADEAGALLRDFFKARRA |
| 464 | MDAALEEARRAAAAGEVPVGAVLVRDGVVLARAGNRTVRDCDPTAHAEIVALRAA |
| | AAALGNYRLDGCTLYVTLEPCAMCAGAMIHARLARLVYGAADPRTGAAGSVLDVL |
| | GHPALNHRTEVTGGVLAEECGALLREFFRTRRG |
| 465 | MSDKDLRFMQLALEEARAAADAGEVPVGAVLVRDGQVLARGRNRVILDNDPTAHA |
| | EIVALREGARRLGNYRLAGCTLYVTLEPCAMCAGAIVHARLDRLVYGAADPKAGAA |
| | GSVLDVLNHPKLNHQMEVEGGVLAAESGELLRSFFRARR |
| 466 | DTLYMRRALELAAQAEAEGEVPVGAVLVKDGEVIAEGWNRSIGSHDATAHAEIETL |
| | RKAGQVLQNYRLLDTTLYVTLEPCPMCAGALLHSRVKRIVYGAPDLKAGAAGTVLN |
| | LFESQASYHYAEVESGLLEEECREQLQAFFKRRRKEIKALRQAQKEAE |
| 467 | MDAALEEARRAAEAGEVPVGAVLVRDGAVLARAGNRTIRDCDPTAHAEIVALRAAA |
| | RALGNYRLPGTTLYVTLEPCAMCAGAMIHARVDRLVYGAADPRAGAAGSVLDVIGH |
| | PALNHRMEVEGGVLAEECGALLREFFRARRG |
| 468 | DPLMDEFMGEALAEARAAAAAGEVPVGAVVVRDGAVIARAGNRTVRDCDPTAHAE |
| | IVALRAAARALGNHRLDGCDLYVTLEPCAMCAGAISHARIRRLYYGADDPKGGAVA |
| | HGARVFAHPTCHHRPEVYGGIGAAEAAGLLRAFFAARR |
| 469 | DESYMRRALELARQAEQEGEVPVGAVLVKDGEVIAEGWNRSIGAHDATAHAEIETL |
| | RKAGQVLQNYRLLDTTLYVTLEPCPMCAGALLHSRVKRVVFGAPDLKAGAAGTVL |
| | NLFESQASYHYADVESGLLEDECREQLQAFFKRRRKEIKAQRDAEKAAK |
| 470 | MRDALAEARAAAARGEVPVGAVVVRDGAVLARAGNATIADCDPTAHAEMRALRA |
| | AARALGNYRLPGCTLYVTVEPCAMCAGAMIHARLARLVYGAADPKAGAAGSVLDV |
| | LGHPALNHRMEVTGGVLAAECAALLRDFFAARRGR |
| 471 | MTEEDKKFMQRAIELARKGEQEGEVPVGAVLVKDGEIIAEGWNRSIGDHDATAHAEI |
| | ETLRKAGKALENYRLLDTTLYVTLEPCPMCAGALLHSRVKRVVFGAPDLKAGAAGT |
| | VLNLFESQASYHYADVESGLLEEECREQLQAFFKRRRKEKKALKQAKKEAEK |
| 472 | MDDDALMGLALDEARAAAAAGEVPIGAVVARDGAVVARAGNRTVRDCDPTAHAE |
| | VVALREAARALGNYRLAGCTLYVTLEPCAMCAGAMIHARLDRVVYGAADPKTGAA |
| | GSVLDVLGHPALNHQTRVEGGVLAAECGALLRDFFAARR |
| 473 | MTRDEQYMRRALELARQAEQEGEVPVGAVLVKDGEIVAEGWNRSIGDHDATAHAEI |
| | ETLRKAGQVLENYRLLDTTLYVTLEPCPMCAGALLHSRVKRVVFGAPDLKAGAAGT |
| | VLNLFESQASYHYAEVESGLLEEECREQLQAFFKRRRKEIKALRQAEKEAEQK |
| 474 | DSEFMAEALAEARAAAAAGEVPVGAVVVKDGEIIARAGNRTLRDNDPTAHAEIVAL |
| | RAAARALGNQRLEGCDLYVTLEPCAMCAGAISHARIRRLYYGAEDPKGGAVDNGVR |
| | FFASPTCHHRPEVYGGIGETEAAELLRGFFRER |
| 475 | DDDERFMRLALEEARKAEEAGEVPVGAVLVKDGEVIARGRNRVISDSDPTAHAEIVA |
| | LREAGRALGNYRLDGCTLYVTLEPCAMCAGAMVHARLDRLVYGAADPRAGAAGSV |
| | LDVLNHPALNHRMEVEGGVLADECGALLRDFFRRRR |
| 476 | MDAALAEARAAADAGEVPVGAVVVRDGAVLARAGNRTLRDSDPTAHAEIVALRAA |
| | ARALGNHRLGGCTLYVTLEPCAMCAGAISHARLARLHYGADDPKGGAVAHGPRFFA |
| | QPTCHHRPDVYGGIGEGEAAALLRGFFAARR |
| 477 | DTEFMREALAEARAAAAAGEVPVGAVVVKDGEIIARAGNRTLRDNDPTAHAEIVAL |
| | REAARKLGNHRLTGCTLYVTLEPCAMCAGAISHARIARLVYGADDPKGGAVAHGPR |
| | FFAQPTCHHRPEVTGGVLAEEAGELLRGFFRERR |
| 478 | MDAALEEARRAAAAGEVPVGAVLVAGGRVLARAGNRTIRDCDPTAHAEIVALREAA |
| | RALGNYRLAGTTLYVTLEPCAMCAGAMIHARVDRLVYGAADPRAGAAGSVFDVVR |
| | HPALNHRLEVEGGVLAEECGALLRDFFRARRGR |
| 479 | MRAALDEARRAAAAGEVPVGAVVVRDGVVLARAHNRTVADHDPTAHAEILALREA |
| | ARVLGNHRLTGCTLYVTLEPCAMCAGAIVHARLDRLVYGAADPKAGAAGSVLDVL |
| | DHPRLNHRMEVTGGVLAEESAALLRGFFAARR |
| 480 | MSSDADFMRLALAEARAAAAAGEVPVGAVVVRGGEVIARAGNRTVRDCDPTAHAE |
| | VVALREAARKLGNHRLTGCTLYVTLEPCAMCAGAISHARVARLVYGADDPKGGAV |
| | AHGPRFFAQPTCHHRPEVTGGVLAEEAGELLRGFFRARR |
| 481 | MDAALEEARRAAAAGEVPVGAVLVRDGAVLARGGNRTIRDCDPTAHAEIVALRAA |
| | ARALGNYRLPGTTLYVTLEPCAMCAGAMIHARVARLVYGAADPKTGAAGSVFDVL |
| | GSARLNHRVEVEGGVRAAECGALLRDFFAARRGR |
| 482 | MRAALAEARRAAAEGEVPVGAVVVRDGAVLARAGNRTVRDCDPTAHAEVLALREA |
| | ARALGNHRLTGCTLYVTVEPCAMCAGAISHARVARLVYGADDPKGGAVRHGPRVF |
| | DQPTCHHRPEVVGGVLAEEAGALLRDFFAARR |
| 483 | DTSFMQQALDEARAAAAAGEVPVGAVVVKDGEIIARAGNRTLRDNDPTAHAEIVAL |
| | RAAARALGSQRLGGCDLYVTLEPCAMCAGAISHARIARLYYGADDPKGGAVAHGA |
| | RVFSHPQCHHVPEVYDGIGAGEAAALLRDFFAGR |
| 484 | DTEFMREALAEARAAAAAGEVPVGAVVVRDGAIIARAGNRTIRDRDPTAHAEVVAL |
| | REAARALGNHRLTGCTLYVTLEPCAMCAGAISHARVARLVYGAADPKGGAVAHGP |
| | RFFEQPTCHHRPEVTGGVLAEEAGALLRDFFRARR |
| 485 | MSEQDEFYMRRAIELARKGEENGEVPVGAVLVKDGEIIAEGWNQSIGSHDATAHAEI |
| | VTLRKAGQVLENYRLLDTTLYVTLEPCPMCAGALLHSRVKRVVFGAPDLKAGAAGT |
| | VLNLFESQASYHYADVESGLLEEECREQLQAFFKRRRKEKKALKQAQKELE |
| 486 | MTDADFMALALAEARAAAAAGEVPVGAVVVRDGVVLAAAGNRTLRDCDPTAHAE |
| | VVALRAAARALGNYRLDGCTLYVTLEPCAMCAGAMLHARLARVVYGAADPKTGA |
| | AGSVLDLFAQPRLNHHTAVAGGVLAAECGALLADFFRQRRG |
| 487 | MSDEQYMRRALELARQAEQQGEVPVGAVLVRDGEVIAEGWNQSISSHDATAHAEM |
| | MAIRAAGAALENYRLLDTTLYVTLEPCPMCAGALLHSRVKRVVFGAPDLKAGAAGT |
| | VLNLFESQASYHYADVESGLLEEECREQLQAFFKRRRKEIKALRQAEKEAAAK |
| 488 | MSSETSTDAAADAAFMAEALAEARAAAAAGEVPVGAVVVCGGRIVARAGNRTVRD |
| | NDPTAHAEIVALREAARVLGNHRLTGCTLYVTLEPCAMCAGAISHARIARLVYGADD |
| | PKGGAVRHGPRFFAQPTCHHRPDVAGGVGEAEAAELLRAFFRARR |
| 489 | MTDADFMALALEEARAAAALGEVPVGAVVVKDGEVIARAGNRTIRDCDPTAHAEIV |
| | ALREAARKLGNYRLDGCDLYVTLEPCAMCAGAMIHARLARLVYGAADPKTGAAGS |
| | VLDVFANPQLNHHTAVVGGVLADEAGALLREFFAARR |
| 490 | MDDDARFMGEALAEARAAAAAGEVPIGAVVVRDGAVVARAGNRTVRDNDPTAHA |
| | EVLALREAARALGSQRLTGCTLYVTLEPCAMCAGAISHARVARLVYGAADPKGGAV |
| | AHGPRFFAQPTCHHRPEVTGGVGEAEAAALLRDFFRARR |
| 491 | DRLFMEEALAEARAAAAAGEVPIGAVVVRDGEIVARAGNRTVRDCDPTAHAEIVAL |
| | REAARALGNYRLTGCTLYVTIEPCAMCAGAMIHARLDRLVYGADDPKAGAVRSVLQ |
| | VLDHPALNHRVEVESGVLAAECAALLQEFFASRR |
| 492 | DSLTRDELYMRRALELAALAEAEGEVPVGAVLVKDGEIVGEGWNRSIGSHDATAHA |
| | EIMALRQAGQALQNYRLLDTTLYVTLEPCPMCAGALLHSRVKRVVFGASDLKAGAA |
| | GTVLNLFESQASYHYADVESGLLEQECREQLQAFFKRRRKEIKAQRQAEKAAQ |
| 493 | MRAALAEARRAAEAGEVPVGAVVVRDGAVLAAAGNRTVRDCDPTAHAEIVALRAA |
| | ARAAGNHRLTGCTLYVTLEPCAMCAGAISHARVARLVYGAADPKGGAVAHGPRFFE |
| | QPTCHHRPEVTGGVLAEEAGALLRDFFRARR |
| 494 | MSAALEEARRAAAAGEVPVGAVLVRDGAVLARGGNRTIRDCDPTAHAEIVALRAAA |
| | RALGNYRLPGTTLYVTLEPCAMCAGAMIHARVARLVYGAADPKTGAAGSVEDVLR |
| | HPALNHRMEVEGGVLAEECGALLRDFFRARR |
| 495 | DDTEFMRLALAEAEAAAAAGEVPVGAVVVRDGEVIARAGNRTIRDCDPTAHAEVVA |
| | LREAARKLGNYRLAGCTLYVTLEPCAMCAGAMIHARLDRVVYGAADPKTGAAGSV |
| | LDLFATRQLNHHTQVTGGVLAEECGALLRGFFEARR |
| 496 | DTLYMRRALELAAQAEAEGEVPVGAVLVKDGEVIAEGWNRSIGDHDATAHAEIQTL |
| | RKAGQVLQNYRLLDTTLYVTLEPCPMCAGALLHSRVKRIVYGAPDLKAGAAGTVLN |
| | LFESQASYHYADVESGLLEDECREQLQAFFKRRRKEIKALRQAEKAAEQ |
| 497 | DDPYMALALEEARAAAAAGEVPIGAVVVRDGEVVARAGNRTVRDNDPTAHAEVVA |
| | LREAARALGNHRLTGCTLYVTLEPCAMCAGAISHARVARLVYGADDPKGGAVRHG |
| | ARVFEHPQCHHRPEVTGGVGAAEAGELLRGFFRARR |
| 498 | MDSKEKDEFFMRRALELARLGEEKGEVPVGAVLVKDGEIIAEGWNQSIGENDATAH |
| | AEIMALRKAGKALENYRLLDTTLYVTLEPCPMCAGALLHSRVKRIVFGAPDLKAGA |
| | AGTVLNLFESQASYHYADVESGLLEEECREQLQAFFKRRRKEIKEKKKAEKAALEA |
| 499 | MRSALDLAAAAAAAGEVPVGAVVVRDGAIVGRGENRVLRDSDPTAHAEIVALREAA |
| | RALGNYRLTGCTLYVTLEPCAMCAGAMIHARIDRLVYGAADPKAGAAGSVLDVLG |
| | HPALNHQMEVEGGVLAEECGALLRDFFRARR |
| 500 | MDAALAEARAAADAGEVPVGAVVVRDGVVLARAGNRTVRDADPTAHAEIVALRA |
| | AARALGNHRLGGCTLYVTLEPCAMCAGAISHARVARLVYGADDPKGGAVRHGPRL |
| | FASPTCHHRPEVVSGVGEAEAAALLRDFFAARR |
| 501 | MDAALEEARRAAAAGEVPVGAVLVRDGVVLARAGNRTIRDCDPTAHAEIVALREAA |
| | RAAGNYRLPGTTLYVTLEPCAMCAGAMIHARVDRLVYGAADPRAGAAGSVFDVVR |
| | HPALNHRMEVEGGVLADESAALLRGFFRARR |
| 502 | MTSEDEKYMRRALELARQAEQEGEVPVGAVLVKDGEIVAEGWNRSIGDHDATAHA |
| | EIETLRKAGQVLENYRLLDTTLYVTLEPCPMCAGALLHSRVKRVVFGAPDLKAGAA |
| | GTVLNLFESQASYHYADVESGLLEEECREQLQAFFKRRRKEIKALRQAQKEAEQK |
| 503 | DEPYMRLALDEARAAAAAGEVPIGAVVVRDGEVLARAGNRTVRDNDPTAHAEVLA |
| | LREAARALGNHRLTGCTLYVTLEPCAMCAGAISHARVARLVYGADDPKGGAVAHG |
| | PRFFAQPTCHHRPEVTGGVLAEEAGALLRDFFRARR |
| 504 | MDAALAEARAAADAGEVPVGAVVVRDGAVLARAGNRTLRDADPTAHAEIVALRAA |
| | ARALGNHRLGGCTLYVTLEPCAMCAGAISHARLARLVYGADDPKGGAVAHGPRFFG |
| | QPTCHHRPEVVGGVGAAAAGDLLRGFFRARR |
| 505 | RSSPEEAAMDDAGFMRLALAEAEAAAAAGEVPVGAVVVLGGEVIARAGNRTVRDN |
| | DPTAHAEIVALREAARKLGNYRLAGCTLYVTLEPCAMCAGAMIHARLARLVYGAAD |
| | PKTGAAGSVLDLVAHPALNHRMEVEGGVLAAECGALLRDFFAARRG |
| 506 | MTRDEQYMRRALELARQAEAEGEVPVGAVLVRDGEVIAEGWNRSIGAHDATAHAEI |
| | MALRQAGQVLENYRLLDTTLYVTLEPCPMCAGALLHSRVKRVVYGAPDLKAGAAG |
| | TVLNLFESQASYHYAEVEGGLLEEECREQLQAFFKRRRKEIKAARQARRAAEE |
| 507 | MRAALAEARAAAEAGEVPVGAVVVHEGRIIARAQNRVERDHDPTAHAEILALRAAA |
| | AALGATRLGGCTLYVTLEPCAMCAGAIAHARVARLVYGADDPKGGAVAHGPRFFT |
| | QPTCHHRPEVTGGVGEAEAAALLRDFFRARR |
| 508 | MDDAGFMREALAEAEAAAAAGEVPVGAVVVKDGEIIARARNATVARNDPTAHAEIL |
| | ALREAARVLGNYRLTGCTLYATLEPCAMCAGAILHARIARLVYGAADPKAGACGSV |
| | LDVIGHPRLNHRVEVAGGVLAEECGALLREFFRSRR |
| 509 | DETYMRRALELARQAEQAGEVPVGAVLVKDGEIVAEGWNQSIGTHDATAHAEIMAL |
| | RAAGQALENYRLVDTTLYVTLEPCPMCAGALLHSRVKRVVFGAADLKAGAAGTVL |
| | NLFESQASYHYAEVESGLLEEECREQLQAFFKRRRKEIKALKQAEKAAQE |
| 510 | MNSDDLYMRRALELARQAEEEGEVPVGAVLVKDGEIVAEGWNRSIGSHDATAHAEI |
| | ETLRKAGQVLQNYRLLDTTLYVTLEPCPMCAGALLHSRVKRVVFGAPDLKAGAAGT |
| | VLNLFESQASYHYADVESGLLEDECREQLQAFFKRRRKEIKALRQAQKEAEE |
| 511 | MDAALAEARAAADAGEVPVGAVLVRDGVVLARAGNRTVRDCDPTAHAEIVALRAA |
| | ARALGNYRLPGTTLYVTLEPCAMCAGAMIHARVDRLVFGARDPRAGAAGSVFDVLR |
| | HPALNHRVEVVEGVRAEECGALLRDFFRARR |
| 512 | DRLFMRRALELAAQAEAEGEVPVGAVLVKDGEVIAEGWNRSIGSHDATAHAEIETLR |
| | KAGQVLENYRLLDTTLYVTLEPCPMCAGALLHSRVKRIVYGAPDLKAGAAGTVLNL |
| | FESQASYHYADVESGLLEDECREQLQAFFKRRRKEIKAARQAKREQE |
| 513 | MDDARFMAEALAEARAAAAAGEVPVGAVVVRDGAVLARAGNRTVRDCDPTAHAE |
| | IVALREAARALGNYRLDGCTLYVTLEPCAMCAGAMLHARLPRLVYGAADPKTGAA |
| | GSVLDLFAERRLNHQTEVRGGVLAEACGALLTDFFRSRRA |
| 514 | DSEFMAEALAEARAAAEAGEVPIGAVVVCDGEIVARAGNRTIRDNDPTAHAEIVALR |
| | EAARKLGNYRLTGCTVYVTLEPCAMCAGAMIHARLDRLVYGAADPKAGAAGSVLD |
| | VLGHPRLNHQMAVESGVLADECGALLRDFFRSRR |
| 515 | MDSESDLRFMREALAEARAAAAAGEVPVGAVVVRDGAILARAGNRTLRDNDPTAH |
| | AEIVALRAAAAALGNHRLTGCTLYVTLEPCAMCAGAISHARIARLVYGAADPKGGA |
| | VAHGPRFFEQPTCHHRPEVAGGVLAEEAGALLRAFFAARR |
| 516 | MSSDAGFMAEALAEARAAAAAGEVPVGAVVVRDGAIVARAGNRTVRDNDPTAHAE |
| | VVALREAARALGNHRLGGCELYVTLEPCAMCAGAISHARIARLYYGADDPKGGAVA |
| | HGARVFSHPQCHHRPEVYDGIGAGEAAALLRDFFAARR |
| 517 | DRDDRYMRLALEEARAAAEAGEVPIGAVVVRDGEVLARAGNRTVRDCDPTAHAEIV |
| | ALREAARVLGNYRLAGCTLYVTVEPCAMCAGAMIHARVDRLVYGADDPKGGAVRS |
| | CLQVLDHPRLNHRVEVTAGVLAEECAALLQSFFAARR |
| 518 | DSEFMAEALAEARAAAAAGEVPVGAVVVRDGEIVARAGNRTLRDNDPTAHAEIVAL |
| | REAARALGNHRLTGCTLYVTLEPCAMCAGAISHARIARLVYGADDPKGGGVAHGAR |
| | VFEHPQCHHRPEVVGGVGAAEAGELLRGFFAARR |
| 519 | DREEYLMRKALELAAKAEQLGEVPVGAVLVKDGEIIAEGWNQSIGNHDATAHAEIM |
| | TLRQAGQALENYRLLDTTLYVTLEPCPMCAGALLHSRVKRIVFGAPDLKAGAAGTV |
| | LNLFESQASYHYADVESGLLEDECREQLQAFFKRRRKEIKALKQAKRDAEEN |
| 520 | MDAALAEARAAADAGEVPVGAVVVRDGAVLARAGNRTLRDADPTAHAEIVALRAA |
| | ARALGNHRLGGCTLYVTLEPCAMCAGAISHARLARLHYGAADPKGGAVEHGPRFFA |
| | QPTCHHRPDVYGGIGEGEAAALLRGFFAARR |
| 521 | DEPYMRLALDEARAAAAAGEVPIGAVVVKDGEVIARARNRTLADNDPTAHAEIVAL |
| | RAAAAALGNHRLTGCELYVTLEPCAMCAGAISHARIARLVYGADDPKGGAVAHGPR |
| | FFAQPTCHHRPEVVGGVGEGEAAELLRGFFAARR |
| 522 | DRDSRFMREALAEARAAAEAGEVPIGAVVVRDGEIVARAGNRTVRDCDPTAHAEIV |
| | ALREAARALGNYRLTGCTLYVTIEPCAMCAGAMIHARIDRLVYGAADPKAGAAGSV |
| | LDVLGHPRLNHRMEVTAGVLAEECAALLRDFFAARR |
| 523 | MDAALEEARRAAAAGEVPVGAVLVHDGQILARAGNRTIRDNDPTAHAEIVVLREAA |
| | RALGNYRLEGCTLYVTLEPCAMCAGAMIHARLPRLVYGADDPKAGAAGSVLDVLN |
| | HPALNHRMEVEGGVLAAECGALLRDFFRARR |
| 524 | MSAADDIRFMREALAEARAAAAAGEVPVGAVVVRDGAILARAGNRTIRDCDPTAHA |
| | EVVALREAARALGNHRLTGCTLYVTLEPCAMCAGAISQARVARLVYGADDPKGGA |
| | VAHGPRFFAQPTCHHRPEVTGGVLAEEAGALLRDFFRARR |
| 525 | MTDADFMALALEEARAAAALGEVPVGAVVVKDGEVIARAGNRTIRDCDPTAHAEV |
| | VALREAARKLGNYRLPGLTLYVTLEPCAMCAGAMIHARLDRVVYGADDPKGGAAR |
| | SVYRILDDPRLNHQVAVTSGVLAEECGALLRDFFRARR |
| 526 | MDSAAKDLCYMRRALELAALAEAEGEVPVGAVLVRDGEIVGEGWNRSIGSHDATA |
| | HAEIMALRQAGQALQNYRLLDTTLYVTLEPCPMCAGALLHSRVKRVVFGAPDLKAG |
| | AAGTVLNLFESQASYHYADVEGGLLAEECRAQLQAFFKRRRKEQKALRQAQKEAA |
| 527 | DEEFMREALAEAQAAADAGEVPIGAVVVCDGEIVARAGNRTIRDNDPTAHAEIVALR |
| | EAARKLGNYRLAGCTLYVTLEPCAMCAGAMIHARVDRLVYGADDPKAGAAGSVLD |
| | VIGHPRLNHRMEVTSGVLAEECGAMLREFFRRRRA |
| 528 | MDDAGFMRLALAEARAAAAAGEVPVGAVVVRGGEVLAAAGNRTLRDCDPTAHAE |
| | VVALRAAARALGNHRLDGCDLYVTLEPCAMCAGAISHARIARLYYGADDPKGGAV |
| | AHGPRFFAQPTCHHRPEVYGGIGESEAAALLRDFFAARR |
| 529 | MSDEDYMRLALAEAQAAADAGEVPVGAVLVAGGEVVARGRNRMIADSDPTAHAEI |
| | VALREAARRLGNYRLTGCTLYVTLEPCAMCAGAIVHARLDRVVYGAADPKAGAAG |
| | SVLDVLGHPRLNHRTEVTGGVLADECGALLKDFFRARR |
| 530 | MSSEDEKYMRRALELARQAEEEGEVPVGAVLVKDGEIVAEGWNRSIGSHDATAHAE |
| | IETLRKAGQALENYRLLDTTLYVTLEPCPMCAGALLHSRVKRIVFGAPDLKAGAAGT |
| | VLNLFESQASYHYADVESGLLEDECREQLQAFFKRRRKEIKAARKAAKAE |
| 531 | MSDDAAFMGEALAEARAAAAAGEVPIGAVVVCDGAIVARAGNRTVRDNDPTAHAE |
| | IVALREAAARLGNYRLTGCTLYVTLEPCAMCAGAMIHARIDRLVYGAADPKAGAAG |
| | SVLDVIGHPRLNHRMEVEGGVLAAECGALLRDFFASRR |
| 532 | MRAALAEAERAAAAGEVPVGAVVVRDGAVLAAAGNRTVRDCDPTAHAEVLALRA |
| | AARALGNHRLTGCTLYVTLEPCAMCAGAISHARVARLVYGADDPKGGAVAHGPRFF |
| | AQPTCHHRPEVAGGLGAAAAGELLRAFFRARR |
| 533 | MRAALDEARAAAAAGEVPVGAVVVHEGRIVARAQNRMRRDNDPTAHAEIVALRAA |
| | AAALGSMRLTGCTLYVTLEPCAMCAGAISHARIDRLVYGAADPKGGAVAHGPRFFE |
| | QPTCHHRPDVVGGVLAEEAGALLRGFFAARR |
| 534 | MDSAAKDLCYMRRALELAALAEAEGEVPVGAVLVKDGEIVGEGWNRSIGSHDATA |
| | HAEIMALRQAGQALQNYRLLDTTLYVTLEPCPMCAGALLHSRVKRVVFGAPDLKAG |
| | AAGTVLNLFESQASYHYADVESGLLEQECREQLQAFFKRRRKEIKAQRDAQRAADE |
| 535 | MNDEFYMRRALELAALAEEHNEVPVGAVLVLNGEIIGEGWNRSIGQHDATAHAEIM |
| | ALRQAGKVLQNYRLLDTTLYVTLEPCPMCAGALVHSRVKRVVYGTPDLKAGAAGT |
| | VMNLLSYDAVNHHVAITSGVLAEECREQLQAFFRRRRAEKKALKQAQRA |
| 536 | DDTFYMRRALELARQAEAEGEVPVGAVLVRDGEVIAEGWNRSIGDHDATAHAEIQV |
| | LRKAGQVLQNYRLLDTTLYVTLEPCPMCAGALLHSRVKRVVYGAPDLKAGAAGTV |
| | LNLFESQASYHYADVESGLLEEECREQLQAFFKRRRKEIKAARQAKRAAE |
| 537 | MRSALDLAAAAAAAGEVPVGAVVVRDGAIVGRGENRVLRDSDPTAHAEIVAMREA |
| | ARALGNYRLTGCTLYVTLEPCAMCAGAMIHARIDRLVYGAADPKAGAAGSVLDVL |
| | NHPRLNHQMEVEGGVLAAECGAMLRDFFRARR |
| 538 | MDDDALMGLALDEARAAAAAGEVPIGAVVARDGEVVARAGNRTVRDCDPTAHAEI |
| | VALREAARKLGNYRLTGCTLYVTLEPCAMCAGAMIHARLDRLVYGAADPKTGAAG |
| | SVLDVLGHPALNHQTQVEGGVLAEECGALLRDFFRERR |
| 539 | MTEEDKYFMRRAIELAKLAEENGEVPVGAVLVKDGEIIAEGWNQSIGSHDATAHAE |
| | MMAIREAGKVLENYRLLDTTLYVTLEPCPMCAGALLHSRVKRIVFGAPDLKAGAAG |
| | TVLNLFESQASYHYADVESGLLEEECREQLQAFFKRRRKEIKALKKAQKEAEE |
| 540 | DDDYMRLALAEARAAAEAGEVPIGAVVVCGGEVVARAGNRTVADCDPTAHAEIVA |
| | LREAARKLGNYRLTGCTLYVTLEPCAMCAGAMIHARLDRLVYGADDPKAGAAGSV |
| | LDVLGHPALNHQMQVTAGVLADECAALLRDFFRARR |
| 541 | DDSFYMRRALELAALAEREGEVPVGAVLVKDGEIIAEGWNRSIGSHDATAHAEIETL |
| | RKAGQALQNYRLLDTTLYVTLEPCPMCAGALLHSRVKRIVFGAPDLKAGAAGTVLN |
| | LFESQASYHYADVESGLLEEECREQLQAFFKRRRKEIKAQRDAEKAAAEK |
| 542 | MDAALAEARAAADAGEVPVGAVVVRDGAVLARAGNRTLRDSDPTAHAEIVALRAA |
| | ARALGNHRLGGCDLYVTLEPCAMCAGAISHARIARLYYGADDPKGGAVAHGARVF |
| | AHPQCHHRPEVYDGIGAAEAAALLRDFFAARR |
| 543 | MTRDEQYMRRALELARQAEAEGEVPVGAVLVKDGEIIAEGWNRSIGSHDATGHAEI |
| | MALRAAGQALENYRLLDTTLYVTLEPCPMCAGALLHSRVKRVVFGAPDLKAGAAG |
| | TVLNLFESQASYHYADVESGLLEEECREQLQAFFKRRRKEIKALRQAEKAAAE |
| 544 | MTDADFMALALEEARAAAALGEVPVGAVVVKDGEVIARAGNRTIRDCDPTAHAEIV |
| | ALREAARKLGNYRLPGLALYVTLEPCAMCAGAMIHARLARLVYGAADPKTGAAGS |
| | VLDVLGHPALNHQTAVTGGVLAEEAGALLRDFFAARRAAG |
| 545 | MDAALAEARAAADAGEVPVGAVVVRDGAVLARAGNRTLRDADPTAHAEIVALRAA |
| | ARALGNHRLPGCTLYVTLEPCAMCAGAISHARIARLVYGAADPKGGAVAHGPRFFE |
| | QPTCHHRPEVVGGLGETEAAALLRAFFAARR |
| 546 | MDAALEEARRAAAAGEVPVGAVLVAGGRVLARAGNRTIRDCDPTAHAEIVALRAA |
| | ARALGNYRLPGTTLYVTLEPCAMCAGAMIHARVARLVYGAADPKTGAAGSVEDVL |
| | GSGRLNHRVAVEGGVRAEECGALLRDFFRARR |
| 547 | DDDERWMREALAEARAAAEAGEVPVGAVVVRDGELIARGRNRVEADADPSAHAEI |
| | VALREAARRLGNHRLTGCTLYVTLEPCAMCAGAISHARVARLVYGADDPKGGAVR |
| | HGARVFEQPTCHHRPEVVGGVLASESAALLRDFFRARR |
| 548 | MSAADEQFMRRAIELARQAEAEGEVPVGAVLVKDGEIVAEGWNRSIGAHDATAHAE |
| | IETLRKAGQALENYRLLDTTLYVTLEPCPMCAGALLHSRVKRVVFGAPDLKAGAAG |
| | TVLNLFESQASYHYADVESGLLEEECREQLQAFFKRRRKEIKALRQAQKAAEE |
| 549 | MSSAPSDADFMGLALEQARLAAAAGEVPVGAVVVRDGEVIAQAHNRTVADCDPTA |
| | HAEVVALRAAARALGNHRLTGCTLYVTLEPCAMCAGAIAHARVARVVYGAADPKG |
| | GAVEHGPRFFAQPTCHHRPEVVGGVGAAEAGALLRDFFRARR |
| 550 | MDEALAEARRAAAAGEVPVGAVVVRDGVVLARAGNRTVRDADPTAHAEIVALRAA |
| | AAALGNYRLDGCTLYVTLEPCAMCAGAMVHARLARLVYGAADPRAGAAGSVLDV |
| | LGHPALNHRMEVAGGVRAEECAALLRDFFAARRG |
| 551 | MDEALAEARRAAEAGEVPVGAVVVRDGAVLARAGNRTVRDCDPTAHAEVLALREA |
| | ARALGNHRLAGCTLYVTLEPCAMCAGAISHARVARLVYGADDPKGGAVAHGPRFF |
| | GQPTCHHRPEVAAGLGAAEAGALLRDFFAARR |
| 552 | DTSFMRLALDEARAAADAGEVPVGAVVVCDGEVIARAGNRTVRDCDPTAHAEIVAL |
| | REAARRLGNYRLAGCTLYVTLEPCAMCAGAMIHARLDRLVYGAADPKAGAAGSVL |
| | DVLGHPALNHQTQVEGGVLAAECGALLRDFFRARR |
| 553 | DTLFMRRALELAAQAEAEGEVPVGAVLVKDGEVIAEGWNRSIGSHDATAHAEIETLR |
| | KAGQVLQNYRLLDTTLYVTLEPCPMCAGALLHSRVKRIVYGAPDLKAGAAGTVLNL |
| | FESQASYHYAEVESGLLEEECREQLQAFFKRRRKEIKALRQAEKEAEK |
| 554 | MDDEFFMRQALREARKAYDEGEVPVGAVVVRDGKVIARGRNQVERLKDPTAHAEM |
| | IALTAAAAHLGSKWLKGCTLYVTVEPCAMCAGALVLARLERLVFGARDPKAGACGS |
| | VLDIVRHPRLNHRVEVVSGVLEEECGALLKEFFRRLR |
| 555 | MTRDEQYMRRALALARQAEAEGEVPVGAVLVKDGEVIAEGWNRSIGRHDATAHAEI |
| | ETLRKAGQVLGNYRLLDTTLYVTLEPCPMCAGALLHSRVKRVVFGAPDLKAGAAGT |
| | VLNLFESQASYHYADVESGLLEDECREQLQAFFKRRRKEIKAARKAAKEAE |
| 556 | MDAALEEARRAAAAGEVPVGAVLVRDGEVLARAGNRTVRDNDPTAHAEILVIREAA |
| | RRLGNYRLEGCVLYVTLEPCAMCAGAMVHARLPRLVYGAADPKAGAAGSVLDVLG |
| | HPALNHRVEVTGGVLADACGALLRDFFAARR |
| 557 | MDEALAEARRAAAAGEVPVGAVLVRDGEVLARGGNRTIRDCDPTAHAEIVALREAA |
| | RRAGNYRLPGTTLYVTLEPCAMCAGAMIHARVARLVYGAADPRAGAAGSVEDVLR |
| | HPALNHRMEVEGGVRAEECGALLREFFRARR |
| 558 | MTEKDEFYMKRAIELARKGEEEGEVPVGAVLVKDGEIIAEGWNRSIGSHDATAHAEI |
| | ETLRKAGQALENYRLLDTTLYVTLEPCPMCAGALLHSRVKRVVFGAPDLKAGAAGT |
| | VLNLFESQASYHYADVESGLLEEECREQLQAFFKRRRKEIKALKKAKKEEQDK |
| 559 | DDDFMALALEEARAAAEAGEVPIGAVVVCDGEVVARAGNRTVRDNDPTAHAEIVAL |
| | REAARKLGNYRLAGCTLYVTLEPCAMCAGAMIHARLDRVVYGADDPKAGAARSVL |
| | NVLDHPALNHRVAVEGGVLADECGALLREFFRARR |
| 560 | MTEEDKYFMQRAIELARQAELAGEVPVGAVLVKDGEIIAEGWNQSIGSHDATAHAEI |
| | MALRQAGQVLQNYRLLDTTLYVTLEPCPMCAGALLHSRVKRVVFGAPDLKAGAAG |
| | TVLNLFESQASYHYADVESGLLEQECREQLQAFFKRRRKEIKALKQAQKEAEN |
| 561 | MTDEEYMRRALELARQAEQQGEVPVGAVLVKDGEVIAEGWNQSIGSHDATAHAEI |
| | MALRAAGQALENYRLLDTTLYVTLEPCPMCAGALLHSRVKRVVFGAPDLKAGAAG |
| | TVLNLFESQASYHYADVESGLLEEECREQLQAFFKRRRKEIKAARQAKREAE |
| 562 | MSEQDEFYMRRAIELARQAEAEGEVPVGAVLVKDGEIVAEGWNRSIGSHDATAHAEI |
| | ETLRKAGQALQNYRLLDTTLYVTLEPCPMCAGALLHSRVKRVVFGAPDLKAGAAGT |
| | VLNLFESQASYHYAEVESGLLEEECREQLQAFFKRRRKEIKALRQAQKEAEEK |
| 563 | DDTLYMKRALELARQAEAEGEVPVGAVLVKDGEVIAEGWNRSIGSHDATAHAEIET |
| | LRKAGQALQNYRLLDTTLYVTLEPCPMCAGALLHSRVKRIVFGAPDLKAGAAGTVL |
| | NLFESQASYHYAEVESGLLEEECREQLQAFFKRRRKEIKALRQAEKEAE |
| 564 | DETYMRRALELARQAEEHGEVPVGAVLVKDGEIIAEGWNRSIGDHDATAHAEIMAL |
| | REAGKTLENYRLLDTTLYVTLEPCPMCAGALLHSRVKRVVYGAPDLKAGAAGTVLN |
| | LFESQASYHYAEVESGLLEEECREQLQAFFKRRRKEIKAARKARREAEEK |
| 565 | MTDADFMALALEEARAAAALGEVPVGAVVVRDGAVIARAGNRTIRDCDPTAHAEIV |
| | ALRAAARALGNYRLDGCDLYVTLEPCAMCAGAMIHARLARLVYGAADPKTGAAGS |
| | VLDVFANPRLNHHTAVVGGVLADEAAALLRGFFAARR |
| 566 | DDEKWMRYALSLADKAEALGEVPVGAVLVKDNQVIGEGWNQSISGHDATAHAEIM |
| | AIRDAGKNLQNYRLIDCTLYVTLEPCPMCAGAIVHSRIKRVVFGASDYKTGAAGSVF |
| | NLLSNEQLNHQAEVTAGVLAEECGEKISAFFKRRRKEKKAAKKAAKLAE |
| 567 | MTEEDKYFMRRAIELAKLAEENGEVPVGAVLVKDGEIIAEGWNQSIGSHDATAHAE |
| | MMAIRQAGQVLENYRLLDTTLYVTLEPCPMCAGALLHSRVKRIVFGAPDLKAGAAG |
| | TVLNLFESQASYHYADVESGLLEEECREQLQAFFKRRRKEIKALKKAQKEAE |
| 568 | MTDDERWMREALAEARAAEAAGEVPVGAVVVRDGELIARGRNRVLRDSDPTAHAE |
| | IVALREAARALGNYRLDGCTLYATLEPCAMCAGAMLHARLARLVYGAADPKAGAA |
| | GSVLDVLGHPRLNHRMEVTGGVLAEEAGALLREFFRARR |
| 569 | DDSFYMRRALELAALAEAEGEVPVGAVLVKDGEIIAEGWNRSIGDHDATAHAEIQVL |
| | RKAGQALQNYRLLDTTLYVTLEPCPMCAGALLHSRVKRIVYGAPDLKAGAAGTVLN |
| | LFESQASYHYAEVESGLLEEECREQLQAFFKRRRKEIKALRQAEKEAQN |
| 570 | DEEFMREALAEAQAAADAGEVPIGAVVVCDGEIVARAGNRTIRDNDPTAHAEIVALR |
| | EAARKLGNYRLAGCTLYVTLEPCAMCAGAMIHARIDRLVYGADDPKAGAAGSVLD |
| | VLGHPRLNHQMQVERGVLAAECGAMLTRFFQARR |
| 571 | MTEADEKFMRRAIELAREAEEHGEVPVGAVLVKDGEIIAEGWNRSIGEHDATAHAEI |
| | ETLRKAGQVLENYRLLDTTLYVTLEPCPMCAGALLHSRVKRIVFGAPDLKAGAAGT |
| | VLNLFESQASYHYADVESGLLEEECREQLQAFFKRRRKEIKALRQAEKAAG |
| 572 | MRDALALAAAAAAAGEVPVGAVVVHGGEIVGRGENRVLRDSDPTAHAEIVALREA |
| | ARALGNYRLTGCDLYVTLEPCAMCAGAMIHARIARLVYGAADPKAGAAGSVLDVL |
| | NHPRLNHRMEVTGGVLAEECGALLRDFFRARR |
| 573 | MTEEDKYFMQRAIELARLAEENGEVPVGAVLVKDGEIIAEGWNQSIGNHDATAHAEI |
| | MTLRQAGQVLQNYRLLDTTLYVTLEPCPMCAGALLHSRVKRIVFGAPDLKAGAAGT |
| | VLNLFESQASYHYADVESGLLEDECREQLQAFFKRRRKEIKALKKAKRDAEN |
| 574 | MDPTDLAFMREALAEARAAAEAGEFPIGAVVVCDGRIVARAHNRPIALSDPTAHAEI |
| | LALREAARALGNYRLTGCTLYATLEPCAMCAGAILHARIARLVYGVANPGAGACGS |
| | VLDVMNHPRLNHRVEVTGGVLAEECGALLSGFFRAR |
| 575 | MDPTDLAFMREALAEARAAAEAGEVPVGAVVVCDGRIVARAHNRPIALSDPTAHAE |
| | ILALREAARALGNIRLTGCTLYATLEPCAMCAGAILHARIARLVYGVANPGAGACGS |
| | VLDVMNHPRLNHRVEVTGGVLAEECGALLSGFFRAR |
| 576 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPKTGAAG |
| | SVLDVLGHPRLNHRMEVEGGVLAAECGALLRDFFRAR |
| 577 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPGCGAAG |
| | SVLDVLGHPRLNHRMEVEGGVLAAECGALLRDFFRAR |
| 578 | MTDEYFMRQALREARRAYDEGEVPVGAVVVRDGRVIARGRNQVERLKDPTAHAEM |
| | LALTAAAGHLGSGRLTGCTLYVTVEPCAMCAGALVWARVERVVFGVRNPKTGACG |
| | SVFDIPGDRRLNHRPEVVGGVLEAECAALMRDFFRER |
| 579 | MDPTDLAFMREALAEARAAAEAGEVPVGAVVVCDGRIVARAHNRPIALSDPTAHAE |
| | ILALREAARALGNYRLTGCTLYATLEPCAMCAGAILHARIARLVYGVANPGAGACGS |
| | VLDVMNHPRLNHRVEVTGGVLAEECGALLSGFFRAR |
| 580 | MDPTDLAFMREALAEARAAAEAGEVPIGAVVVCDGRIVARAHNRPIALSDPTAHAEI |
| | LALREAARALGNYRLTGCTLYATLEPCAMCAGAILHARIARLVYGVANAGAGACGS |
| | VLDVMNHPRLNHRVEVTGGVLAEECGALLSGFFRAR |
| 581 | MDDAGFMRLALEEARAAAAAGEVPIGAVVVRDGEVLARAGNRTVRDCDPTAHAEI |
| | VALREAARRLGNYRLAGCTLYVTLEPCAMCAGAMIHARLDRLVYGVANPKTGAAG |
| | SVLDVLGHPRLNHRMEVEGGVLAAECGALLRDFFRAR |
| 582 | MDDAGFMRLALEEARAAAAAGEVPIGAVVVRDGEVLARAGNRTVRDCDPTAHAEI |
| | VALREAARRLGNYRLAGCTLYVTLEPCAMCAGAMIHARLDRLVYGVANPGAGAAG |
| | SVLDVLGHPRLNHRMEVEGGVLAAECGALLRDFFRAR |
| 583 | MTDEYFMRQALREARRAYDEGEVPVGAVVVRDGRVIARGRNQVERLKDPTAHAEM |
| | LALTAAAGHLGSSRLTGCTLYVTVEPCAMCAGALVWARVERVVFGVRNPKTGACG |
| | SVFDIPGDRRLNHRPEVVGGVLEAECAALMRDFFRER |
| 584 | MTDEYFMRQALREARRAYDEGEVPVGAVVVRDGRVIARGRNQVRRLKDPTAHAEM |
| | LALTAAAGHLGSPRLTGCTLYVTVEPCAMCAGALVWARVERVVFGVRNPKTGACG |
| | SVFDIPGDRRLNHRPEVVGGVLEAECAALMRDFFRER |
| 585 | MDPTDLAFMREALAEARAAAEAGEVPIGAVVVCDGRIVARAHNRPIALSDPTAHAEI |
| | LALREAARALGNYRLTGCTLYATLEPCAMCAGAILHARIARLVYGVANPGAGACGS |
| | VLDVMNHPRLNHRVEVTGGVLAEECGALLSGFFRAR |
| 586 | MDPTDLAFMREALAEARAAAEAGELPIGAVVVCDGRIVARAHNRPIALSDPTAHAEI |
| | LALREAARALGNYRLTGCTLYATLEPCAMCAGAILHARIARLVYGVANPGTGACGS |
| | VLDVMNHPRLNHRVEVTGGVLAEECGALLSGFFRAR |
| 587 | MDPTDLAFMREALAEARAAAEAGEMPVGAVVVCDGRIVARAHNRPIALSDPTAHAE |
| | ILALREAARALGNYRLTGCTLYATLEPCAMCAGAILHARIARLVYGVANPGAGACGS |
| | VLDVMNHPRLNHRVEVTGGVLAEECGALLSGFFRAR |
| 588 | MDDAGFMRLALEEARAAAAAGEVPIGAVVVRDGEVLARAGNRTVRDCDPTAHAEI |
| | VALREAARRLGNYRLAGCTLYVTLEPCAMCAGAMIHARLDRLVYGVANPKAGAAG |
| | SVLDVLGHPRLNHRMEVEGGVLAAECGALLRDFFRAR |
| 589 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPGRGAAG |
| | SVLDVLGHPRLNHRMEVEGGVLAAECGALLRDFFRAR |
| 590 | MDDAGFMRLALEEARAAAAAGEVPIGAVVVRDGEVLARAGNRTVRDCDPTAHAEI |
| | VALREAARRLGNYRLAGCTLYVTLEPCAMCAGAMIHARLDRLVYGVANPGSGAAG |
| | SVLDVLGHPRLNHRMEVEGGVLAAECGALLRDFFRAR |
| 591 | MTDEYFMRQALREARRAYDEGEVPVGAVVVRDGRVIARGRNQVIRLKDPTAHAEM |
| | LALTAAAGHLGSPRLTGCTLYVTVEPCAMCAGALVWARVERVVFGVRNPKTGACG |
| | SVFDIPGDRRLNHRPEVVGGVLEAECAALMRDFFRER |
| 592 | MDPTDLAFMREALAEARAAAEAGEIPVGAVVVCDGRIVARAHNRPIALSDPTAHAEI |
| | LALREAARALGNYRLTGCTLYATLEPCAMCAGAILHARIARLVYGVANPGAGACGS |
| | VLDVMNHPRLNHRVEVTGGVLAEECGALLSGFFRAR |
| 593 | MDPTDLAFMREALAEARAAAEAGELPIGAVVVCDGRIVARAHNRPIALSDPTAHAEI |
| | LALREAARALGNYRLTGCTLYATLEPCAMCAGAILHARIARLVYGVANPGAGACGS |
| | VLDVMNHPRLNHRVEVTGGVLAEECGALLSGFFRAR |
| 594 | MDPTDLAFMREALAEARAAAEAGEVPVGAVVVCDGRIVARAHNRPIALSDPTAHAE |
| | ILALREAARALGNVRLTGCTLYATLEPCAMCAGAILHARIARLVYGVANPGAGACGS |
| | VLDVMNHPRLNHRVEVTGGVLAEECGALLSGFFRAR |
| 595 | MDPTDLAFMREALAEARAAAEAGEVPIGAVVVCDGRIVARAHNRPIALSDPTAHAEI |
| | LALREAARALGNSRLTGCTLYATLEPCAMCAGAILHARIARLVYGVANPGAGACGS |
| | VLDVMNHPRLNHRVEVTGGVLAEECGALLSGFFRAR |
| 596 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPKRGAAG |
| | SVLDVLGHPRLNHRMEVEGGVLAAECGALLRDFFRAR |
| 597 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPGSGAAG |
| | SVLDVLGHPRLNHRMEVEGGVLAAECGALLRDFFRAR |
| 598 | MDDAGFMRLALEEARAAAAAGEVPVGAVVVRDGEVLARAGNRTVRDCDPTAHAEI |
| | VALREAARRLGNFRLAGCTLYVTLEPCAMCAGAMIHARLDRLVYGVANPGRGAAG |
| | SVLDVLGHPRLNHRMEVEGGVLAAECGALLRDFFRAR |
| 599 | MDPTDLAFMREALAEARAAAEAGEVPVGAVVVCDGRIVARAHNRPIALSDPTAHAE |
| | ILALREAARALGNYRLTGCTLYATLEPCAMCAGAILHARIARLVYGVANDGAGACG |
| | SVLDVMNHPRLNHRVEVTGGVLAEECGALLSGFFRAR |
| 600 | MDPTDLAFMREALAEARAAAEAGEFPVGAVVVCDGRIVARAHNRPIALSDPTAHAEI |
| | LALREAARALGNYRLTGCTLYATLEPCAMCAGAILHARIARLVYGVANPGTGACGS |
| | VLDVMNHPRLNHRVEVTGGVLAEECGALLSGFFRAR |
| 601 | MDPTDLAFMREALAEARAAAEAGEVPIGAVVVCDGRIVARAHNRPIALSDPTAHAEI |
| | LALREAARALGNWRLTGCTLYATLEPCAMCAGAILHARIARLVYGVANPGAGACGS |
| | VLDVMNHPRLNHRVEVTGGVLAEECGALLSGFFRAR |
| 602 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANAGSGAAG |
| | SVLDVLGHPRLNHRMEVEGGVLAAECGALLRDFFRAR |
| 603 | MTDEYFMRQALREARRAYDEGEVPVGAVVVRDGRVIARGRNQVERLKDPTAHAEM |
| | LALTAAAGHLGSGRLTGCTLYVTVEPCAMCAGALVWARVERVVFGVRNPKTGACG |
| | SVFDIPGDRRLSHRPEVVGGVLEAECAALMRDFFRER |
| 604 | MTDEYFMRQALREARRAYDEGEVPVGAVVVRDGRVIARGRNQVRRLKDPTAHAEM |
| | LALTAAAGHLGSRRLTGCTLYVTVEPCAMCAGALVWARVERVVFGVRNPKTGACG |
| | SVFDIPGDRRLNHRPEVVGGVLEAECAALMRDFFRER |
| 605 | MDPTDLAFMREALAEARAAAEAGEIPIGAVVVCDGRIVARAHNRPIALSDPTAHAEIL |
| | ALREAARALGNYRLTGCTLYATLEPCAMCAGAILHARIARLVYGVANPGAGACGSV |
| | LDVMNHPRLNHRVEVTGGVLAEECGALLSGFFRAR |
| 606 | MDPTDLAFMREALAEARAAAEAGEFPVGAVVVCDGRIVARAHNRPIALSDPTAHAEI |
| | LALREAARALGNYRLTGCTLYATLEPCAMCAGAILHARIARLVYGVANPGAGACGS |
| | VLDVMNHPRLNHRVEVTGGVLAEECGALLSGFFRAR |
| 607 | MDPTDLAFMREALAEARAAAEAGEIPVGAVVVCDGRIVARAHNRPIALSDPTAHAEI |
| | LALREAARALGNYRLTGCTLYATLEPCAMCAGAILHARIARLVYGVANAGAGACGS |
| | VLDVMNHPRLNHRVEVTGGVLAEECGALLSGFFRAR |
| 608 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPGTGAAG |
| | SVLDVLGHPRLNHRMEVEGGVLAAECGALLRDFFRAR |
| 609 | MTDEYFMRQALREARRAYDEGEVPVGAVVVRDGRVIARGRNQVERLKDPTAHAEM |
| | LALTAAAGHLGSRSLTGCTLYVTVEPCAMCAGALVWARVERVVFGVRNPRAGACG |
| | SVFDIPGDRRLNHRPEVVGGVLEAECAALMRDFFRER |
| 610 | MTDEYFMRQALREARRAYDEGEVPVGAVVVRDGRVIARGRNQVVRLKDPTAHAEM |
| | LALTAAAGHLGSPRLTGCTLYVTVEPCAMCAGALVWARVERVVFGVRNPKTGACG |
| | SVFDIPGDRRLNHRPEVVGGVLEAECAALMRDFFRER |
| 611 | MDPTDLAFMREALAEARAAAEAGEVPIGAVVVCDGRIVARAHNRPIALSDPTAHAEI |
| | LALREAARALGNYRLTGCTLYATLEPCAMCAGAILHARIARLVYGVANDGAGACGS |
| | VLDVMNHPRLNHRVEVTGGVLAEECGALLSGFFRAR |
| 612 | MDPTDLAFMREALAEARAAAEAGEVPVGAVVVCDGRIVARAHNRPIALSDPTAHAE |
| | ILALREAARALGNYRLTGCTLYATLEPCAMCAGAILHARIARLVYGVANSGAGACGS |
| | VLDVMNHPRLNHRVEVTGGVLAEECGALLSGFFRAR |
| 613 | MDPTDLAFMREALAEARAAAEAGEVPIGAVVVCDGRIVARAHNRPIALSDPTAHAEI |
| | LALREAARALGNTRLTGCTLYATLEPCAMCAGAILHARIARLVYGVANPGAGACGS |
| | VLDVMNHPRLNHRVEVTGGVLAEECGALLSGFFRAR |
| 614 | MTDEYFMRQALREARRAYDEGEVPVGAVVVRDGRVIARGRNQVNRLKDPTAHAEM |
| | LALTAAAGHLGSPRLTGCTLYVTVEPCAMCAGALVWARVERVVFGVRNPKAGACG |
| | SVFDIPGDRRLNHRPEVVGGVLEAECAALMRDFFRER |
| 615 | MTDEYFMRQALREARRAYDEGEVPVGAVVVRDGRVIARGRNQVNRLKDPTAHAEM |
| | LALTAAAGHLGSRSLTGCTLYVTVEPCAMCAGALVWARVERVVFGVRNPKTGACG |
| | SVFDIPGDRRLNHRPEVVGGVLEAECAALMRDFFRER |
| 616 | MDPTDLAFMREALAEARAAAEAGEVPIGAVVVCDGRIVARAHNRPIALSDPTAHAEI |
| | LALREAARALGNYRLTGCTLYATLEPCAMCAGAILHARIARLVYGVANAGTGACGS |
| | VLDVMNHPRLNHRVEVTGGVLAEECGALLSGFFRAR |
| 617 | MDPTDLAFMREALAEARAAAEAGEFPIGAVVVCDGRIVARAHNRPIALSDPTAHAEI |
| | LALREAARALGNYRLTGCTLYATLEPCAMCAGAILHARIARLVYGVANPGTGACGS |
| | VLDVMNHPRLNHRVEVTGGVLAEECGALLSGFFRAR |
| 618 | MDPTDLAFMREALAEARAAAEAGEVPIGAVVVCDGRIVARAHNRPIALSDPTAHAEI |
| | LALREAARALGNIRLTGCTLYATLEPCAMCAGAILHARIARLVYGVANPGTGACGSV |
| | LDVMNHPRLNHRVEVTGGVLAEECGALLSGFFRAR |
| 619 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANPGAGAAG |
| | SVLDVLGHPRLNHRMEVEGGVLAAECGALLRDFFRAR |
| 620 | MDDAGFMRLALEEARAAAAAGEVPVGAVVVRDGEVLARAGNRTVRDCDPTAHAEI |
| | VALREAARRLGNYRLAGCTLYVTLEPCAMCAGAMIHARLDRLVYGVANAGRGAAG |
| | SVLDVLGHPRLNHRMEVEGGVLAAECGALLRDFFRAR |
| 621 | MDPTDLAFMREALAEARAAAEAGEVPVGAVVVCDGRIVARAHNRPIALRDPTAHAE |
| | VLALREAAAALGSYRLTGCTLYATLEPCPMCAGAILHARIARLVYGVANPKAGACGS |
| | VLDVMNHPRLNHRVEVTGGVLAEECGALLSGFFRAR |
| 622 | MDPTDLAFMREALAEARAAAEAGEVPVGAVVVCDGRIVARAHNRPIALKDPTAHAE |
| | VLALREAAAALGSYRLTGCTLYATLEPCPMCAGAILHARIARLVYGVANPKAGACGS |
| | VLDVMNHPRLNHRVEVTGGVLAEECGALLSGFFRAR |
| 623 | MDPTDLAFMREALAEARAAAEAGEVPVGAVVVCDGRIVARAHNRPIALNDPTAHAE |
| | VLALREAGKALGSYRLTGCTLYATLEPCPMCAGAILHARIARLVYGVANPKAGACGS |
| | VLDVMNHPRLNHRVEVTGGVLAEECGALLSGFFRAR |
| 624 | MDPTDLAFMREALAEARAAAEAGEVPVGAVVVCDGRIVARAHNRPIALRDPTAHAE |
| | VLALREAARALGSYRLTGCTLYATLEPCPMCAGAILHARIARLVYGVANPKAGACGT |
| | VLDVMNHPRLNHRVEVTGGVLAEECGALLSGFFRAR |
| 625 | MDDAGFMRLALEEARAAAAAGEVPVGAVVVRDGEVLARAGNRTVRDRDPTAHAEI |
| | VAIREAARRLGSYRLAGCTLYVTLEPCPMCAGAMIHARLDRLVYGVANPKAGAAGT |
| | VLDVLGHPRLNHRMEVEGGVLAAECGALLRDFFRAR |
| 626 | MTDEYFMRQALREARRAYDEGEVPVGAVVVRDGRVIARGRNQVRRLKDPTAHAEM |
| | LALTAAAGHLGSRSLTGCTLYVTVEPCAMCAGALVWARVERVVFGVRNPKAGACG |
| | SVFDIPGDRRLNHRPEVVGGVLEAECAALMRDFFRER |
| 627 | MTDEYFMRQALREARRAYDEGEVPVGAVVVRDGRVIARGRNQVRRLKDPTAHAEM |
| | LALTAAAGHLGSARLTGCTLYVTVEPCAMCAGALVWARVERVVFGVRNPRAGACG |
| | SVFDIPGDRRLNHRPEVVGGVLEAECAALMRDFFRER |
| 628 | MDPTDLAFMREALAEARAAAEAGEVPVGAVVVCDGRIVARAHNRPIALNDPTAHAE |
| | VLALREAARALGDYRLTGCTLYATLEPCPMCAGAILHARIARLVYGVANPKAGACG |
| | SVLDVMNHPRLNHRVEVTGGVLAEECGALLSGFFRAR |
| 629 | MDPTDLAFMREALAEARAAAEAGEVPVGAVVVCDGRIVARAHNRPIALKDPTAHAE |
| | ILALREAAAALGSYRLTGCTLYATLEPCAMCAGAILHARIARLVYGVANPKAGACGS |
| | VLDVMNHPRLNHRVEVTGGVLAEECGALLSGFFRAR |
| 630 | MDPTDLAFMREALAEARAAAEAGEVPVGAVVVCDGRIVARAHNRPIALKDPTAHAE |
| | ILALREAGRALGSYRLTGCTLYATLEPCPMCAGAILHARIARLVYGVANPKAGACGS |
| | VLDVMNHPRLNHRVEVTGGVLAEECGALLSGFFRAR |
| 631 | MDPTDLAFMREALAEARAAAEAGEVPVGAVVVCDGRIVARAHNRPIALADPTAHAE |
| | VLALREAARALGSYRLTGCTLYATLEPCPMCAGAILHARIARLVYGVANPKAGACGS |
| | VLDVMNHPKLNHRVEVTGGVLAEECGALLSGFFRAR |
| 632 | MDPTDLAFMREALAEARAAAEAGEVPVGAVVVCDGRIVARAHNRPIALNDPTAHAE |
| | ILALREAGRALGSYRLTGCTLYATLEPCPMCAGAILHARIARLVYGVANPKAGACGS |
| | VLDVMNHPRLNHRVEVTGGVLAEECGALLSGFFRAR |
| 633 | MDPTDLAFMREALAEARAAAEAGEVPVGAVVVCDGRIVARAHNRPIALRDPTAHAE |
| | ILALREAGRALGNYRLTGCTLYATLEPCPMCAGAILHARIARLVYGVANPKAGACGS |
| | VLDVMNHPKLNHRVEVTGGVLAEECGALLSGFFRAR |
| 634 | MDPTDLAFMREALAEARAAAEAGEVPVGAVVVCDGRIVARAHNRPIALNDPTAHAE |
| | VLALREAAAALGSYRLTGCTLYATLEPCPMCAGAILHARIARLVYGVANPKAGACG |
| | TVLDVMNHPRLNHRVEVTGGVLAEECGALLSGFFRAR |
| 635 | MDPTDLAFMREALAEARAAAEAGEVPVGAVVVCDGRIVARAHNRPIALNDPTAHAE |
| | ILALREAGRALGSYRLTGCTLYATLEPCPMCAGAILHARIARLVYGVANPKAGACGT |
| | VLDVMNHPRLNHRVEVTGGVLAEECGALLSGFFRAR |
| 636 | MDPTDLAFMREALAEARAAAEAGEVPVGAVVVCDGRIVARAHNRPIALADPTAHAE |
| | VLALREAARALGSYRLTGCTLYATLEPCAMCAGAILHARIARLVYGVANPKAGACG |
| | SVLDVMNHPKLNHRVEVTGGVLAEECGALLSGFFRAR |
| 637 | MDDAGFMRLALEEARAAAAAGEVPVGAVVVRDGEVLARAGNRTVRDRDPTAHAE |
| | VVALREAARRLGSYRLAGCTLYVTLEPCAMCAGAMIHARLDRLVYGVANPKAGAA |
| | GTVLDVLGHPRLNHRMEVEGGVLAAECGALLRDFFRAR |
| 638 | MDDAGFMRLALEEARAAAAAGEVPVGAVVVRDGEVLARAGNRTVRDRDPTAHAEI |
| | VAIREAARRLGSYRLAGCTLYVTLEPCAMCAGAMIHARLDRLVYGVANPKAGAAGT |
| | VLDVLGHPRLNHRMEVEGGVLAAECGALLRDFFRAR |
| 639 | MDDAGFMRLALEEARAAAAAGEVPVGAVVVRDGEVLARAGNRTVRDKDPTAHAEI |
| | VALREAARRLGSYRLAGCTLYVTLEPCAMCAGAMIHARLDRLVYGVANPKAGAAG |
| | TVLDVLGHPRLNHRMEVEGGVLAAECGALLRDFFRAR |
| 640 | MTDEYFMRQALREARRAYDEGEVPVGAVVVRDGRVIARGRNQVVRLKDPTAHAEM |
| | LALTAAAGHLGSPRLTGCTLYVTVEPCAMCAGALVWARVERVVFGVRNPKTGACG |
| | SVFDIPGDRRLSHRPEVVGGVLEAECAALMRDFFRER |
| 641 | MDPTDLAFMREALAEARAAAEAGEVPVGAVVVCDGRIVARAHNRPIALNDPTAHAE |
| | ILALREAARALGSYRLTGCTLYATLEPCAMCAGAILHARIARLVYGVANPKAGACGS |
| | VLDVMNHPKLNHRVEVTGGVLAEECGALLSGFFRAR |
| 642 | MDPTDLAFMREALAEARAAAEAGEVPVGAVVVCDGRIVARAHNRPIALRDPTAHAE |
| | ILALREAARALGSYRLTGCTLYATLEPCAMCAGAILHARIARLVYGVANPKAGACGT |
| | VLDVMNHPRLNHRVEVTGGVLAEECGALLSGFFRAR |
| 643 | MDPTDLAFMREALAEARAAAEAGEVPVGAVVVCDGRIVARAHNRPIALNDPTAHAE |
| | VLALREAAAALGSYRLTGCTLYATLEPCAMCAGAILHARIARLVYGVANPKAGACG |
| | TVLDVMNHPRLNHRVEVTGGVLAEECGALLSGFFRAR |
| 644 | MTDEYFMRQALREARRAYDEGEVPVGAVVVRDGRVIARGRNQVVRLKDPTAHAEM |
| | LALTAAAGHLGSSRLTGCTLYVTVEPCAMCAGALVWARVERVVFGVRNPRTGACG |
| | SVFDIPGDRRLNHRPEVVGGVLEAECAALMRDFFRER |
| 645 | MTDEYFMRQALREARRAYDEGEVPVGAVVVRDGRVIARGRNQVRRLKDPTAHAEM |
| | LALTAAAGHLGSPSLTGCTLYVTVEPCAMCAGALVWARVERVVFGVRNPRTGACGS |
| | VFDIPGDRRLNHRPEVVGGVLEAECAALMRDFFRER |
| 646 | MDPTDLAFMREALAEARAAAEAGEVPVGAVVVCDGRIVARAHNRPIALKDPTAHAE |
| | ILALREAARALGSYRLTGCTLYATLEPCPMCAGAILHARIARLVYGVANPKAGACGS |
| | VLDVMNHPRLNHRVEVTGGVLAEECGALLSGFFRAR |
| 647 | MDPTDLAFMREALAEARAAAEAGEVPVGAVVVCDGRIVARAHNRPIALNDPTAHAE |
| | VLALREAGRALGSYRLTGCTLYATLEPCPMCAGAILHARIARLVYGVANPKAGACGS |
| | VLDVMNHPRLNHRVEVTGGVLAEECGALLSGFFRAR |
| 648 | MDPTDLAFMREALAEARAAAEAGEVPVGAVVVCDGRIVARAHNRPIALNDPTAHAE |
| | VLALREAAKALGSYRLTGCTLYATLEPCPMCAGAILHARIARLVYGVANPKAGACGS |
| | VLDVMNHPKLNHRVEVTGGVLAEECGALLSGFFRAR |
| 649 | MDPTDLAFMREALAEARAAAEAGEVPVGAVVVCDGRIVARAHNRPIALKDPTAHAE |
| | ILALREAAAALGDYRLTGCTLYATLEPCPMCAGAILHARIARLVYGVANPKAGACGS |
| | VLDVMNHPKLNHRVEVTGGVLAEECGALLSGFFRAR |
| 650 | MDPTDLAFMREALAEARAAAEAGEVPVGAVVVCDGRIVARAHNRPIALRDPTAHAE |
| | ILALREAGRALGSYRLTGCTLYATLEPCPMCAGAILHARIARLVYGVANPKAGACGS |
| | VLDVMNHPKLNHRVEVTGGVLAEECGALLSGFFRAR |
| 651 | MDDAGFMRLALEEARAAAAAGEVPVGAVVVRDGEVLARAGNRTVRDRDPTAHAEI |
| | VALREAARRLGSYRLAGCTLYVTLEPCPMCAGAMIHARLDRLVYGVANPKAGAAG |
| | TVLDILGHPRLNHRMEVEGGVLAAECGALLRDFFRAR |
| 652 | MDDAGFMRLALEEARAAAAAGEVPVGAVVVRDGEVLARAGNRTVRDRDPTAHAE |
| | VVAIREAARRLGSYRLAGCTLYVTLEPCAMCAGAMIHARLDRLVYGVANPKAGAA |
| | GTVLDVLGHPRLNHRMEVEGGVLAAECGALLRDFFRAR |
| 653 | MTDEYFMRQALREARRAYDEGEVPVGAVVVRDGRVIARGRNQVNRLKDPTAHAEM |
| | LALTAAAGHLGSPRLTGCTLYVTVEPCAMCAGALVWARVERVVFGVRNPRTGACG |
| | SVFDIPGDRRLSHRPEVVGGVLEAECAALMRDFFRER |
| 654 | MTDEYFMRQALREARRAYDEGEVPVGAVVVRDGRVIARGRNQVRRLKDPTAHAEM |
| | LALTAAAGHLGSPQLTGCTLYVTVEPCAMCAGALVWARVERVVFGVRNPKTGACG |
| | SVFDIPGDRRLNHRPEVVGGVLEAECAALMRDFFRER |
| 655 | MDPTDLAFMREALAEARAAAEAGEVPVGAVVVCDGRIVARAHNRPIALNDPTAHAE |
| | ILALREAGRALGNYRLTGCTLYATLEPCPMCAGAILHARIARLVYGVANPKAGACGT |
| | VLDVMNHPRLNHRVEVTGGVLAEECGALLSGFFRAR |
| 656 | MDPTDLAFMREALAEARAAAEAGEVPVGAVVVCDGRIVARAHNRPIALRDPTAHAE |
| | ILALREAARALGSYRLTGCTLYATLEPCPMCAGAILHARIARLVYGVANPKAGACGT |
| | VLDVMNHPRLNHRVEVTGGVLAEECGALLSGFFRAR |
| 657 | MDPTDLAFMREALAEARAAAEAGEVPVGAVVVCDGRIVARAHNRPIALNDPTAHAE |
| | VLALREAARALGSYRLTGCTLYATLEPCAMCAGAILHARIARLVYGVANPKAGACG |
| | TVLDVMNHPRLNHRVEVTGGVLAEECGALLSGFFRAR |
| 658 | MDPTDLAFMREALAEARAAAEAGEVPVGAVVVCDGRIVARAHNRPIALNDPTAHAE |
| | ILALREAARALGSYRLTGCTLYATLEPCAMCAGAILHARIARLVYGVANPKAGACGT |
| | VLDVMNHPKLNHRVEVTGGVLAEECGALLSGFFRAR |
| 659 | MDPTDLAFMREALAEARAAAEAGEVPVGAVVVCDGRIVARAHNRPIALADPTAHAE |
| | ILALREAAKALGSYRLTGCTLYATLEPCPMCAGAILHARIARLVYGVANPKAGACGT |
| | VLDVMNHPRLNHRVEVTGGVLAEECGALLSGFFRAR |
| 660 | MTDEYFMRQALREARRAYDEGEVPVGAVVVRDGRVIARGRNQVRRLKDPTAHAEM |
| | LALTAAAGHLGSPRLTGCTLYVTVEPCAMCAGALVWARVERVVFGVRNPKTGACG |
| | SVFDIPGDRRLSHRPEVVGGVLEAECAALMRDFFRER |
| 661 | MTDEYFMRQALREARRAYDEGEVPVGAVVVRDGRVIARGRNQVRRLKDPTAHAEM |
| | LALTAAAGHLGSARLTGCTLYVTVEPCAMCAGALVWARVERVVFGVRNPRTGACG |
| | SVFDIPGDRRLNHRPEVVGGVLEAECAALMRDFFRER |
| 662 | MTDEYFMRQALREARRAYDEGEVPVGAVVVRDGRVIARGRNQVIRLKDPTAHAEM |
| | LALTAAAGHLGSPSLTGCTLYVTVEPCAMCAGALVWARVERVVFGVRNPRTGACGS |
| | VFDIPGDRRLNHRPEVVGGVLEAECAALMRDFFRER |
| 663 | MDPTDLAFMREALAEARAAAEAGEVPVGAVVVCDGRIVARAHNRPIALNDPTAHAE |
| | ILALREAARALGSYRLTGCTLYATLEPCPMCAGAILHARIARLVYGVANPKAGACGT |
| | VLDVMNHPRLNHRVEVTGGVLAEECGALLSGFFRAR |
| 664 | MDPTDLAFMREALAEARAAAEAGEVPVGAVVVCDGRIVARAHNRPIALNDPTAHAE |
| | VLALREAARALGSYRLTGCTLYATLEPCPMCAGAILHARIARLVYGVANPKAGACGT |
| | VLDVMNHPRLNHRVEVTGGVLAEECGALLSGFFRAR |
| 665 | MDPTDLAFMREALAEARAAAEAGEVPVGAVVVCDGRIVARAHNRPIALNDPTAHAE |
| | VLALREAAKALGSYRLTGCTLYATLEPCPMCAGAILHARIARLVYGVANPKAGACG |
| | TVLDVMNHPRLNHRVEVTGGVLAEECGALLSGFFRAR |
| 666 | MDPTDLAFMREALAEARAAAEAGEVPVGAVVVCDGRIVARAHNRPIALNDPTAHAE |
| | VLALREAGRALGSYRLTGCTLYATLEPCPMCAGAILHARIARLVYGVANPKAGACGS |
| | VLDVMNHPKLNHRVEVTGGVLAEECGALLSGFFRAR |
| 667 | MDDAGFMRLALEEARAAAAAGEVPVGAVVVRDGEVLARAGNRTVRDKDPTAHAEI |
| | VAIREAARRLGSYRLAGCTLYVTLEPCPMCAGAMIHARLDRLVYGVANPKAGAAGT |
| | VLDVLGHPRLNHRMEVEGGVLAAECGALLRDFFRAR |
| 668 | MTDEYFMRQALREARRAYDEGEVPVGAVVVRDGRVIARGRNQVRRLKDPTAHAEM |
| | LALTAAAGHLGSPSLTGCTLYVTVEPCAMCAGALVWARVERVVFGVRNPKTGACGS |
| | VFDIPGDRRLNHRPEVVGGVLEAECAALMRDFFRER |
| 669 | MTDEYFMRQALREARRAYDEGEVPVGAVVVRDGRVIARGRNQVIRLKDPTAHAEM |
| | LALTAAAGHLGSRSLTGCTLYVTVEPCAMCAGALVWARVERVVFGVRNPRAGACG |
| | SVFDIPGDRRLNHRPEVVGGVLEAECAALMRDFFRER |
| 670 | MTDEYFMRQALREARRAYDEGEVPVGAVVVRDGRVIARGRNQVVRLKDPTAHAEM |
| | LALTAAAGHLGSPSLTGCTLYVTVEPCAMCAGALVWARVERVVFGVRNPRTGACGS |
| | VFDIPGDRRLNHRPEVVGGVLEAECAALMRDFFRER |
| 671 | MDPTDLAFMREALAEARAAAEAGEVPVGAVVVCDGRIVARAHNRPIALKDPTAHAE |
| | ILALREAAAALGNYRLTGCTLYATLEPCPMCAGAILHARIARLVYGVANPKAGACGS |
| | VLDVMNHPLLNHRVEVTGGVLAEECGALLSGFFRAR |
| 672 | MDPTDLAFMREALAEARAAAEAGEVPVGAVVVCDGRIVARAHNRPIALRDPTAHAE |
| | ILALREAAAALGSYRLTGCTLYATLEPCPMCAGAILHARIARLVYGVANPKAGACGT |
| | VLDVMNHPRLNHRVEVTGGVLAEECGALLSGFFRAR |
| 673 | MDPTDLAFMREALAEARAAAEAGEVPVGAVVVCDGRIVARAHNRPIALNDPTAHAE |
| | ILALREAARALGSYRLTGCTLYATLEPCPMCAGAILHARIARLVYGVANPKAGACGT |
| | VLDVMNHPKLNHRVEVTGGVLAEECGALLSGFFRAR |
| 674 | MDPTDLAFMREALAEARAAAEAGEVPVGAVVVCDGRIVARAHNRPIALKDPTAHAE |
| | ILALREAARALGSYRLTGCTLYATLEPCPMCAGAILHARIARLVYGVANPKAGACGT |
| | VLDVMNHPRLNHRVEVTGGVLAEECGALLSGFFRAR |
| 675 | MDPTDLAFMREALAEARAAAEAGEVPVGAVVVCDGRIVARAHNRPIALKDPTAHAE |
| | ILALREAAAALGDYRLTGCTLYATLEPCPMCAGAILHARIARLVYGVANPKAGACGT |
| | VLDVMNHPRLNHRVEVTGGVLAEECGALLSGFFRAR |
| 676 | MTDEYFMRQALREARRAYDEGEVPVGAVVVRDGRVIARGRNQVERLKDPTAHAEIL |
| | ALRAACRHLGSKYLTGCTLYVTVEPCAMCAGALVWARVERVVFGVRNPKAGACGS |
| | VLDIPGDRRLNHRPEVVGGVLEAECAALMRDFFRER |
| 677 | MTDEYFMRQALREARRAYDEGEVPVGAVVVRDGRVIARGRNQVERLKDPTAHAEIL |
| | ALSAACRHLGSKYLTGCTLYVTVEPCAMCAGALVWARVERVVFGVRNPKAGACGS |
| | VLDIPGDRRLNHRPEVVGGVLEAECAALMRDFFRER |
| 678 | MTDEYFMRQALREARRAYDEGEVPVGAVVVRDGRVIARGRNQVERLKDATAHAEI |
| | LALRAACAHLGSKYLTGCTLYVTVEPCAMCAGALVWARVERVVFGVRNPKAGACG |
| | SVLDIPGDRRLNHRPEVVGGVLEAECAALMRDFFRER |
| 679 | MTDEYFMRQALREARRAYDEGEVPVGAVVVRDGRVIARGRNQVERLRDATAHAEI |
| | LALRAACEHLGSKYLTGCTLYVTVEPCAMCAGALVWARVERVVFGVRNPKAGACG |
| | SVLDIPGNRRLNHRPEVVGGVLEAECAALMRDFFRER |
| 680 | MDPEDLAFMRKALEEARQARDAGEVPVGAVVVLDGEIVARAHNRTIQLSDPTAHAE |
| | ILALREAARALGNYRLEGCTLYVTLEPCAMCAGAILHARIERLVFGVANPKAGAAGS |
| | VLDVLNHPRLNHRVEVTGGVLAEECGALLRDFFRAR |
| 681 | MDPTDEAFMKKALDEAKKAAEAGEVPVGAVVVYDGKVVARAHNRRIAESDPTAHA |
| | EILALREAARALGNYRLTGCTLYVTLEPCAMCAGAILHARIARLVYGVPNPKAGATG |
| | SVLNVLNHPRLNHRVEVTGGVLAEECGDLLRGFFRAR |
| 682 | MDDEGWMQLALEEARASRAAGEVPVGAVVVRDGEVLARAGNRTRELCDPTAHAEI |
| | VALREAARRLGNYRLPGCTLYVTLEPCAMCAGAMLHARLDRLVYGVPNPKAGAAG |
| | SVLDVLGHPRLNHRLEVTGGVLAAECGQLLRDFFRAR |
| 683 | MTDEYFMRQALREARRAYDEGEVPVGAVVVRDGRVIARGRNQVERLKDPTAHAEIL |
| | ALKAACAHLGSKYLTGCTLYVTVEPCAMCAGALVWARVERVVFGVRNPKAGACGS |
| | VLDIPGNRRLNHRPEVVGGVLEAECAALMRDFFRER |
| 684 | MDPTDVAFMQQALDEARAAREAGEVPVGAVVVYDGRVVARAHNRTIARSDPTAHA |
| | EILALREAARALGNYRLAGCTLYVTLEPCAMCAGAILHARIERLVYGVPNPKAGATG |
| | SVLNVLNHPRLNHRVEVTGGVLAEECGALLRGFFRAR |
| 685 | MDPEDLRFMRKALAEARKAKEAGEVPVGAVVVKDGKIVAQAHNRTIALSDPTAHAE |
| | ILALREAARALGNYRLTGCTLYATLEPCAMCAGAILHARIARLVYGVPNPKAGASGS |
| | VLNVLNHPRLNHQVEVTGGVLAEECGALLRDFFRAR |
| 686 | MDPKDLRFMQKALAEAREAKAAGEVPVGAVVVRNGKIVARAHNRTRALSDPTAHA |
| | EILALREAARALGNYRLEGCTLYVTLEPCAMCAGAILHARIERLVYGVANPKAGAAG |
| | SVLDVLNHPRLNHRVEVTGGVLAEECGALLRGFFRAR |
| 687 | MTDEYFMRQALREARRAYDEGEVPVGAVVVRDGRVIARGRNQVERLKDPTAHAEIL |
| | ALRAACAHLGSKYLTGCTLYVTVEPCAMCAGALVWARVERVVFGVRNPKAGACGS |
| | VLDIPGDRRLNHRPEVVGGVLEAECAALMRDFFRER |
| 688 | MTDEYFMRQALREARRAYDEGEVPVGAVVVRDGRVIARGRNQVERLQDPTAHAEIL |
| | ALRAACAHLGSKYLTGCTLYVTVEPCAMCAGALVWARVERVVFGVRNPKAGACGS |
| | VLDIPGNRRLNHRPEVVGGVLEAECAALMRDFFRER |
| 689 | MTDEYFMRQALREARRAYDEGEVPVGAVVVRDGRVIARGRNQVERLRDPTAHAEV |
| | LALRAACAHLGSKYLTGCTLYVTVEPCAMCAGALVWARVERVVFGVRNPKAGACG |
| | SVLDIPGDRRMNHRPEVVGGVLEAECAALMRDFFRER |
| 690 | MTDEYFMRQALREARRAYDEGEVPVGAVVVRDGRVIARGRNQVERLKDATAHAEI |
| | LALRAACRHLGSKYLTGCTLYVTVEPCAMCAGALVWARVERVVFGVRNPKAGACG |
| | SVLDIPGNRRLNHRPEVVGGVLEAECAALMRDFFRER |
| 691 | MDALDIEFMRKALAEARAAKEAGEVPVGAVVVRDGKIVARAHNRTIALSDPTAHAE |
| | ILALRAAARALGNYRLTGCTLYVTLEPCAMCAGAILHARIERLVYGVPNPKAGATGS |
| | VLNVLNHPRLNHRVEVTGGVLAEECGRLLSDFFRAR |
| 692 | MDPDDIAFMREALAEARRAKEAGEVPVGAVVVKDGRIVARAHNRTIAESDPTAHAEI |
| | LALREAARQLGNYRLTGCTLYATLEPCAMCAGAILHARIARLVYGVPNPKAGATGS |
| | VLDVMNHPRLNHRVEVTGGVLADECGDLLRDFFRAR |
| 693 | MDPEDIAFMEQALAEARAARDAGEVPVGAVVVLDGEIVAQAHNRTRQLSDPTAHAE |
| | ILALRQAARALGNYRLTGCTLYVTLEPCAMCAGAILHARVERLVYGVANPKAGATG |
| | SVLNVLNHPRLNHRVEVTGGVLAEECGDLLRGFFRAR |
| 694 | MDDQDLRFMKMALDEARKAKDAGEVPVGAVVVHEGEVVARAHNRTIARSDPTAH |
| | AEIRALREAARALGNYRLTGCTLYVTLEPCAMCAGAILHARIARLVYGVPNPKAGAT |
| | GSVLNVLNHPRLNHRVEVTGGVLAEECGDLLRDFFRAR |
| 695 | MDDEGWMRLALEEARASRAAGEVPVGAVVVRDGQVLARAGNRTRERCDPTAHAEI |
| | VALREAARKLGNYRLAGCTLYVTLEPCAMCAGAMLHARLDRLVYGVANPKAGAA |
| | GSVLDVLGHPRLNHRLEVTGGVLEAECGQLLRDFFRAR |
| 696 | MTDEYFMRQALREARRAYDEGEVPVGAVVVRDGRVIARGRNQVERLRDPTAHAEL |
| | LALRAACEHLGSKYLTGCTLYVTVEPCAMCAGALVWARVERVVFGVRNPKAGACG |
| | SVLDIPGDRRLNHRPEVVGGVLEAECAALMRDFFRER |
| 697 | MTDEYFMRQALREARRAYDEGEVPVGAVVVRDGRVIARGRNQVERLRDATAHAEI |
| | LALRAAARHLGSKYLTGCTLYVTVEPCAMCAGALVWARVERVVFGVRNPKAGACG |
| | SVLDIPGNRRLNHRPEVVGGVLEAECAALMRDFFRER |
| 698 | MDAEDLAFMRQALAEAREAKEAGEVPVGAVVVHDGRVVARAHNRTIRLSDPTAHA |
| | EILALREAARALGNYRLAGCTLYVTLEPCAMCAGAILHARIQRLVYGVPNPKAGAAG |
| | SVLDVLNHPRLNHRVEVTGGVLAEECAALLTDFFRAR |
| 699 | MDPEDVEFMRKALAEAREARQAGEVPVGAVVVHDGKIVARAHNRPIALSDPTAHAE |
| | ILALREAARALGNYRLEGCTLYVTLEPCAMCAGAILHARIQRLVYGVPNPKAGATGS |
| | VLNVMNHPRLNHRVEVTGGVLAEECGALLSDFFRAR |
| 700 | MDPEDLRFMREALDEAREAREAGEVPVGAVVVRDGRIVARAHNRTKELSDPTAHAE |
| | ILALRAAARALGNYRLAGCTLYCTLEPCAMCAGAILHARIARLVYGVPNPKAGAAGS |
| | VLDVLNHPRLNHRVEVTGGVLAEECGALLRGFFRAR |
| 701 | MDDAGFMRLALEEARAAAAAGEVPVGAVVVRDGEVLARAGNRTVRDADPTAHAE |
| | VVAIREAARRLGSYRLAGCTLYVTLEPCPMCAGAMIHARLDRLVYGVANPKAGAAG |
| | TVLDVLGHPRLNHRMEVEGGVLAAECGALLRDFFRAR |
| 702 | MTDEYFMRQALREARRAYDEGEVPVGAVVVRDGRVIARGRNQVERLRDPTAHAEIL |
| | ALRAACAHLGSKYLTGCTLYVTVEPCAMCAGALVWARVERVVFGVRNPKAGACGS |
| | VLDIPGNRRLNHRPEVVGGVLEAECAALMRDFFRER |
| 703 | MTDEYFMRQALREARRAYDEGEVPVGAVVVRDGRVIARGRNQVERLRDPTAHAEL |
| | LALRAACAHLGSKYLTGCTLYVTVEPCAMCAGALVWARVERVVFGVRNPKAGACG |
| | SVLDIPGDRRLNHRPEVVGGVLEAECAALMRDFFRER |
| 704 | MTDEYFMRQALREARRAYDEGEVPVGAVVVRDGRVIARGRNQVERLRDATAHAEI |
| | LALRAAAAHLGSKYLTGCTLYVTVEPCAMCAGALVWARVERVVFGVRNPKAGACG |
| | SVLDIPGNRRLNHRPEVVGGVLEAECAALMRDFFRER |
| 705 | MTDEYFMRQALREARRAYDEGEVPVGAVVVRDGRVIARGRNQVERLKDATAHAEI |
| | LALRAACAHLGSKYLTGCTLYVTVEPCAMCAGALVWARVERVVFGVRNPKAGACG |
| | SVLDIPGNRRLNHRPEVVGGVLEAECAALMRDFFRER |
| 706 | MDPKDIRFMRKALAEAREARDAGEVPVGAVVVHDGEIVAQAHNRPIAESDPTAHAEI |
| | LALREAARALGNYRLEGCTLYVTLEPCAMCAGAILHARIARLVYGVANPKAGAAGS |
| | VLNVLNHPRLNHRVEVTGGVLADECGDLLRDFFRAR |
| 707 | MDSEDIKFMRQALAEAREAREAGEVPVGAVVVHDGRIVARAHNRRILESDPTAHAEI |
| | LALRQAARALGNYRLTGCTLYATLEPCAMCAGAILHARIERLVYGVANPKAGATGS |
| | VLNVLNHPRLNHRVEVTGGVLEEECGALLRDFFRAR |
| 708 | MDPVDVRFMKKALAEAREAKEAGEVPVGAVVVKDGEVVARAHNRTIARSDPTAHA |
| | EINALRAAARALGNYRLEGCTLYVTLEPCAMCAGAILHARIQRLVYGVPNPKAGAA |
| | GSVLDVLNHPRLNHRVEVTGGVLAEECGALLSDFFRAR |
| 709 | MDPDDIKFMRQALAEARKAREAGEVPVGAVVVKDGRVVARAHNRTIALSDPTAHA |
| | EIRALREAARALGNYRLVGCTLYVTLEPCAMCAGAILHARIARLVYGVPNPKAGAAG |
| | SVLDVMNHPRLNHRVEVTGGVLAEECGALMSDFFRAR |
| 710 | MDDEGWMQLALEEARASRAAGEVPVGAVVVRDGEVLARAGNRTRERCDPTAHAEI |
| | VALREAARRLGNYRLPGCTLYVTLEPCAMCAGAMLHARLDRLVYGVANPKAGAAG |
| | SVLDVLGHPRLNHRMEVTGGVLADECGRLLRDFFRAR |
| 711 | MTDEYFMRQALREARRAYDEGEVPVGAVVVRDGRVIARGRNQVERLRDATAHAEI |
| | LALKAAAAHLGSKYLTGCTLYVTVEPCAMCAGALVWARVERVVFGVRNPKAGACG |
| | SVLDIPGDRRLNHRPEVVGGVLEAECAALMRDFFRER |
| 712 | MDPEDIEFMEQALAEAKAAREAGEVPVGAVVVHDGKIVARAHNRTRALSDPTAHAE |
| | ILALREAARALGNYRLEGCTLYATLEPCAMCAGAILHARIERLVYGVANPKAGATGS |
| | VLDVLNHPRLNHRVEVTGGVLAEECGALLSDFFRAR |
| 713 | MDPEDIAFMRMALAEARKAKEAGEVPVGAVVVLNGEIVARAHNRPIALSDPTAHAEI |
| | LALREAARALGNYRLPGCTLYVTLEPCAMCAGAILHARIERLVYGVANPKAGATGS |
| | VLDVLNHPRLNHKVEVTGGVLAEECGALLRGFFRAR |
| 714 | MDAEDLEFMQQALDEAREAREAGEVPVGAVVVLDGRIVARAHNRPIAESDPTAHAE |
| | ILALRAAARALGNYRLTGCTLYVTLEPCAMCAGAILHARIERLVYGVPNPKAGACGS |
| | VLNVMNHPRLNHRVEVVGGVLAEECGALLTGFFRAR |
| 715 | MDPKDIKFMEKALEEARKAKEAGEVPVGAVVVKDGRIVARAHNRTIELSDPTAHAEI |
| | RALREAARALGNYRLEGCTLYATLEPCAMCAGAILHARIERLVYGVPNPKAGATGSV |
| | LDVLNHPRLNHRVEVTGGVLAEECGALLRGFFRAR |
| 716 | MDDEGWMRLALEEARASRAAGEVPVGAVVVRDGEVLARAGNRTRELCDPTAHAEI |
| | VALREAARRLGNYRLPGCTLYVTLEPCAMCAGAMIHARLDRLVYGVANPKAGAAG |
| | SVLDVLGHPRLNHRMEVTGGVLAEECGALMRDFFRAR |
| 717 | MTDEYFMRQALREARRAYDEGEVPVGAVVVRDGRVIARGRNQVERLKDPTAHAEIL |
| | ALRAACAHLGSKYLTGCTLYVTVEPCAMCAGALVWARVERVVFGVRNPKAGACGS |
| | VLDIPGNRKLNHRPEVVGGVLEAECAALMRDFFRER |
| 718 | MTDEYFMRQALREARRAYDEGEVPVGAVVVRDGRVIARGRNQVERLRDATAHAEI |
| | LALRAACRHLGSKYLTGCTLYVTVEPCAMCAGALVWARVERVVFGVRNPKAGACG |
| | SVLDIPGNRRLNHRPEVVGGVLEAECAALMRDFFRER |
| 719 | MDPEDIAFMRKALAEARKAKEAGEVPVGAVVVKDGKIVARAHNRTIALSDPTAHAE |
| | ILALREAARALGNYRLTGCTLYVTLEPCAMCAGAILHARIEKLVYGVPNPKAGACGS |
| | VLNVLNHPRLNHRVEVTGGVLAEECGALLKDFFRAR |
| 720 | MDPEDVRYMRKALAEARKAKEAGEVPVGAVVVHDGRVVARAHNRTIALSDPTAHA |
| | EILALREAARALGNYRLEGCTLYATLEPCAMCAGAILHARIQRLVYGVANPKAGAAG |
| | SVLDVMNHPRLNHRVEVTGGVLEEECGALLSDFFRAR |
| 721 | MDPEDVAFMKKALAEARAAQEAGEVPVGAVVVKDGEVVARAHNRTRERSDPTAH |
| | AEILALREAARALGNYRLAGCTLYVTLEPCAMCAGAILHARIERLVYGVANPKAGAA |
| | GSVLNVLNHPRLNHRVEVTGGVLAEECGDLLRGFFRAR |
| 722 | MDPTDLEFMRQALAEAREARDAGEVPVGAVVVHDGRIVARAHNRTRELSDPTAHAE |
| | ILALRAAARALGNYRLTGCTLYATLEPCAMCAGAILHARIERLVYGVANPKAGACGS |
| | VLHVLNHPRLNHRVEVTGGVLAEECGRLLRGFFRAR |
| 723 | MDDEGWMRLALEEARAAKAAGEVPVGAVVVRDGEVLARAGNRTRLDCDPTAHAEI |
| | VALREAARQLGNYRLAGCTLYVTLEPCAMCAGAMLHARLDRLVYGVPNPKAGAAG |
| | SVLDVLGHPRLNHRLEVTGGVLAEECGQLMRDFFRAR |
| 724 | MTDEYFMRQALREARRAYDEGEVPVGAVVVRDGRVIARGRNQVERLRDATAHAEI |
| | LALRAAAAHLGSKYLTGCTLYVTVEPCAMCAGALVWARVERVVFGVRNPKAGACG |
| | SVLDIPGDRRLNHRPEVVGGVLEAECAALMRDFFRER |
| 725 | MTDEYFMRQALREARRAYDEGEVPVGAVVVRDGRVIARGRNQVERLRDATAHAEI |
| | LALRAASAHLGSKYLTGCTLYVTVEPCAMCAGALVWARVERVVFGVRNPKAGACG |
| | SVLDIPGDRRLNHRPEVVGGVLEAECAALMRDFFRER |
| 726 | MTDEYFMRQALREARRAYDEGEVPVGAVVVRDGRVIARGRNQVERLRDPTAHAEIL |
| | ALRAACAHLGSKYLTGCTLYVTVEPCAMCAGALVWARVERVVFGVRNPKAGACGS |
| | VLDIPGNRRMNHRPEVVGGVLEAECAALMRDFFRER |
| 727 | MDPEDLAFMRKALAEARRAAEAGEVPVGAVVVHDGRIVARAHNRREALSDPTAHA |
| | EILALREAARALGNYRLAGCTLYATLEPCAMCAGAILHARIARLVYGVPNPKAGACG |
| | SVLDVLNHPRLNHRVEVTGGVLAEECGALLRDFFRAR |
| 728 | MDPYDLAFMQQALAEARQAKAAGEVPVGAVVVHDGRIVARAHNRTIELSDPTAHA |
| | EILALRQAARALGNYRLEGCTLYCTLEPCAMCAGAILHARIERLVYGVPNPKAGAAG |
| | SVLNVMNHPRLNHRVEVTGGVLAEECGALLRDFFRAR |
| 729 | MDPEDIEFMQKALAEAREAREAGEVPVGAVVVKDGEVVARAHNRVNALSDPTAHA |
| | EILALREAARALGNYRLTGCTLYATLEPCAMCAGAILHARIERLVYGVANPKAGATG |
| | SVLNVLNHPRLNHRVEVTGGVLAEECGDLLRDFFRAR |
| 730 | MDDRGWMRLALEEARAARARGEVPVGAVVVRDGQVLARAGNRTRELCDPTAHAEI |
| | VALREAARRLGNYRLAGCTLYVTLEPCAMCSGAMLHARLDRLVYGVANPKAGAAG |
| | SVLDVLGHPRLNHRLEVEGGVLEEECGALLRDFFRAR |
| 731 | MDDRGWMRLALEEARASRAAGEVPVGAVVVRDGEVLARAGNRVRELCDPTAHAEI |
| | VALREAARRLGNYRLPGCTLYVTLEPCAMCAGAMLHARLDRLVYGVANPKAGAAG |
| | SVLDVLGHPRLNHRMEVTGGVLEEECGALMRDFFRAR |
| 732 | MNDEGFMQLALEEARAARAAGEVPVGAVVVRDGEVIASAGNRTRERCDPTAHAEIV |
| | ALRAAARKLGNYRLPGCTLYVTLEPCAMCAGAMIHARLDRLVYGVANPKAGAAGS |
| | VLDVLGHPRLNHRMEVEGGVLAEECGRLLRDFFRAR |
| 733 | MSDEHFMKQALDEARKAKDRGEVPVGAVVVRDGEIIATGRNRVEELKDPTAHAEM |
| | LAITAAAGHLGSKYLTGCTLYVTLEPCVMCAGACIWARVDRVVFGVRNPKAGACGS |
| | VFNVPGDPRLNHHPEVTGGILEDECAKLLRDFFRER |
| 734 | MSEVDPTDLAFMREALAEARAAAEAGEVPVGAVVVCDGRIVARAHNRPIALSDPTA |
| | HAEILALREAARALGNYRLTGCTLYATLEPCAMCAGAILHARIARLVYGVANPKAGA |
| | CGSVLDVMNHPRLNHRVEVTGGVLAEECGALLSGFFRAPRRVFNAQKKAQSSTD |
| 735 | MSEVEFSHEYWMRHALTLAKRAWDEREVPVGAVLVHNNRVIGEGWNRPIGRHDPT |
| | AHAEIMALRQGGLVMQNYRLIDATLYVTLEPCVMCAGAMIHSRIGRVVFGVRNAKT |
| | GAAGSLMDVLHHPGMNHRVEITEGILADECAALLSDFFRMRRQEIKAQKKAQSSTDS |
| | GGSSGGSSGSETPGTSESATPESSGGSSGGSSEVEFTDEYFMRQALREARRAYDEGEV |
| | PVGAVVVRDGRVIARGRNQVERLKDPTAHAEMLALTAAAGHLGSKYLTGCTLYVT |
| | VEPCAMCAGALVWARVERVVFGVRNPKAGACGSVFDIPGDRRLNHRPEVVGGVLE |
| | AECAALMRDFFREPRRVFNAQKKAQSSTD |
| 736 | MDDEGWMRLALEEARAAKAAGEVPVGAVVVRDGQVIARAGNRTRERCDPTAHAEI |
| | RALREAARRLGNYRLEGCTLYVTLEPCAMCAGAMLHARLDRLVYGVANPKAGAAG |
| | SVLDVLGHPRLNHRLEVTGGVLAEECGQLLRDFFRAR |
| 737 | MDDAGWMQLALEEARASRAAGEVPVGAVVVRDGEVLASAGNRTRELCDPTAHAEI |
| | VALREAARRLGNYRLSGCTLYVTLEPCAMCAGAMLHARLDRLVYGVANPKAGAAG |
| | SVLDVLGHPRLNHRMEVTGGVLAEECAALLRDFFRAR |
| 738 | MDDEGWMRLALEEARAARDAGEVPVGAVVVRDGQVIARAGNRTRQLCDPTAHAEI |
| | VALRAAARRLGNYRLPGCTLYVTLEPCAMCAGAMVHARIDRLVYGVPNPKAGAAG |
| | SVLNVLHHPRLNHRMEVTGGVLAEECGALLTDFFRAR |
| 739 | MDDAGWMQLALEEARASKAAGEVPVGAVVVRDGRILARAGNRTRELCDPTAHAEI |
| | VALREAARRLGNYRLPGCTLYVTLEPCAMCAGAMLHARLDRLVYGVANPKAGAAG |
| | SVLDVLGHPRLNHRLEVEGGVLAAECSALLRDFFRAR |
| 740 | MSEVEFDDAGFMRLALEEARAAAAAGEVPVGAVVVRDGEVLARAGNRTVRDCDPT |
| | AHAEIVALREAARRLGNYRLAGCTLYVTLEPCAMCAGAMIHARLDRLVYGVANPKA |
| | GAAGSVLDVLGHPRLNHRMEVEGGVLAAECGALLRDFFRAPRRVFNAQKKAQSSTD |
| 741 | MDDRGWMKLALEEARASRAAGEVPVGAVVVRDGRVLASAGNRTRERCDPTAHAEI |
| | VALREAARKLGNYRLAGCTLYVTLEPCAMCAGAMLHARLDRLVYGVANPKAGAA |
| | GSVLDVLGHPRLNHRMEVTGGVLAEECGALLRDFFRAR |
| 742 | MDDEGWMQLALDEARKARDAGEVPVGAVVVRDGRVLASAGNRTRERCDPTAHAEI |
| | VALREAARKLGNYRLPGCTLYVTLEPCAMCAGAMLHARLDRLVYGVDNPKAGAAG |
| | SVLDVLGHPRLNHRMEVVGGVLAEECGALLRDFFRAR |
| 743 | MTDEYFMKQALAEARKAYDAGEVPVGAVVVRDGEVIARGHNRVERLKDPTAHAE |
| | MLAITAAAGHLGSKYLEGCTLYVTLEPCVMCAGALVWARVDRVVFGVPNPKAGAC |
| | GSVFDIPGDPRLNHRPEVTGGVLEAECAALIRDFFRER |
| 744 | MSEVEFTDEYFMRQALREARRAYDEGEVPVGAVVVRDGRVIARGRNQVERLKDPTA |
| | HAEMLALTAAAGHLGSKYLTGCTLYVTVEPCAMCAGALVWARVERVVFGVRNPKA |
| | GACGSVFDIPGDRRLNHRPEVVGGVLEAECAALMRDFFREPRRVFNAQKKAQSSTD |
| 745 | MSEVEFSHEYWMRHALTLAKRAWDEREVPVGAVLVHNNRVIGEGWNRPIGRHDPT |
| | AHAEIMALRQGGLVMQNYRLIDATLYVTLEPCVMCAGAMIHSRIGRVVFGVRNAKT |
| | GAAGSLMDVLHHPGMNHRVEITEGILADECAALLSDFFRMRRQEIKAQKKAQSSTDS |
| | GGSSGGSSGSETPGTSESATPESSGGSSGGSSEVEFDEKWMRYALSLADKAEALGEVP |
| | VGAVLVKDNQVIGEGWNQSISGHDATAHAEIMAIRDAGKNLQNYRLIDCTLYVTLEP |
| | CPMCAGAIVHSRIKRVVFGVSNYKTGAAGSVENLLSNEQLNHQAEVTAGVLAEECG |
| | EKISAFFKRPRRVFNAQKKAQSSTD |
| 746 | MDDEGWMQLALEEARASRAAGEVPVGAVVVRDGEVVARAGNRTRELCDPTAHAEI |
| | VALREAARARGNYRLTGCTLYVTLEPCAMCAGAMIHARLDRLVYGVPNPKAGAAG |
| | SVLDVLGHPRLNHRMEVTGGVLAEECGALLRDFFRAR |
| 747 | MDDQGWMQLALEEARASRARGEVPVGAVVVRDGEVIARAGNRVRERCDPTAHAEI |
| | VALREAARALGNYRLPGCTLYVTLEPCAMCAGAMLHARLDRLVYGVANPKAGAAG |
| | SVLDVLGHPRLNHRLEVTGGVLEAECGALLRDFFRAR |
| 748 | MDDEGWMRLALEEARASRARGEVPVGAVVVRDGEVLARAGNRTREKCDPTAHAEI |
| | VALREAARRLGNYRLAGCTLYVTLEPCAMCAGAMLHARLDRLVYGVPNPKAGAAG |
| | SVLDVLGHPRLNHRMEVVGGVLEAECGALLRDFFRAR |
| 749 | MTDEHFMREALREAQKAYDRGEVPVGAVVVRDGEIIARGRNQVEKLKDPTAHAEM |
| | LAITAAAGHLGSKYLRGCTLYVTLEPCVMCAGALVWARVERVVFGVDNPKAGAAG |
| | SVFNVPGDPRLNHRPEVVGGVLEAECAALLRDFFRER |
| 750 | MSEVEFSHEYWMRHALTLAKRAWDEREVPVGAVLVHNNRVIGEGWNRPIGRHDPT |
| | AHAEIMALRQGGLVMQNYRLIDATLYVTLEPCVMCAGAMIHSRIGRVVFGVRNAKT |
| | GAAGSLMDVLHHPGMNHRVEITEGILADECAALLSDFFRMRRQEIKAQKKAQSSTDS |
| | GGSSGGSSGSETPGTSESATPESSGGSSGGSSEVEFSDDAGFMREALAEARAAAAAGE |
| | VPVGAVVVKDGEIIARAGNRTLRDNDPTAHAEIVALRAAARALGNHRLTGCTLYVTL |
| | EPCAMCAGAISHARIARLVYGVDNPKGGAVAHGPRFFAQPTCHHRPEVTGGVGAEE |
| | AGALLRDFFRAPRRVFNAQKKAQSSTD |
| 751 | MDDEFYMRRALELAALAEEHNEVPVGAVLVLNGEIIGEGWNRSIGHHDATAHAEIM |
| | ALRQAGKKLENYRLLDTTLYVTLEPCPMCAGALLHSRVKRVVFGVPNLKAGAAGTV |
| | LNLFESQASYHYADVESGLLEQECREQLQAFFKRRRKEKKALKQAQKEAE |
| 752 | MDDRGWMRLALEEARAARAAGEVPVGAVVVRDGEVLARAGNRTRELCDPTAHAEI |
| | RALREAARQLGNYRLAGCTLYVTLEPCAMCAGAMLHARLDRLVYGVPNPKAGAAG |
| | SVLDVLGHPRLNHRLEVEGGVLAAECAQLLRDFFRAR |
| 753 | MDDAGWMQLALEEARAAREAGEVPVGAVVVRDGEVIARAGNRTRELCDPTAHAEI |
| | VALREAARKLGNYRLAGCTLYVTLEPCAMCAGAMLHARLDRLVYGVANPKAGAA |
| | GSVLDVLGHPRLNHRMEVTGGVLAAECGRLLRDFFRAR |
| 754 | MDDEGFMQLALEEARAARAAGEVPVGAVVVKDGEVLARAGNRTRELCDPTAHAEI |
| | VALREAARALGNYRLSGCTLYVTLEPCAMCAGAMVHARLDRLVYGVPNPKAGAAG |
| | SVLDVLHHPRLNHRMEVTGGVLAAECGALLRDFFRAR |
| 755 | MSDEHFMRQALEEARKAYDEGEVPVGAVVVRDGEVIARGRNQVEILKDPTAHAEM |
| | LALTAAAGHLGSKYLRGCTLYVTLEPCVMCAGACIWARVDRVVFGVRNPKAGAAG |
| | SVFDVPGDPRLNHRPEVVGGVLRDECAQLMRDFFRER |
| 756 | MSEVEFTDEYFMRQALREARRAYDEGEVPVGAVVVRDGRVIARGRNQVERLKDPTA |
| | HAEMLALTAAAGHLGSKYLTGCTLYVTVEPCAMCAGALVWARVERVVFGVRNPKA |
| | GACGSVFDIPGDRRLNHRPEVVGGVLEAECAALMRDFFREPRRVFNAQKKAQSSTDS |
| | GGSSGGSSGSETPGTSESATPESSGGSSGGSSEVEFTDEYFMRQALREARRAYDEGEV |
| | PVGAVVVRDGRVIARGRNQVERLKDPTAHAEMLALTAAAGHLGSKYLTGCTLYVT |
| | VEPCAMCAGALVWARVERVVFGVRNPKAGACGSVFDIPGDRRLNHRPEVVGGVLE |
| | AECAALMRDFFREPRRVFNAQKKAQSSTD |
| 757 | MDEKWMRYALSLADKAEALGEVPVGAVLVKDNQVIGEGWNQSISGHDATAHAEIM |
| | AIRDAGKNLQNYRLIDCTLYVTLEPCPMCAGAIVHSRIKRVVFGVSNYKTGAAGSVF |
| | NLLSNEQLNHQAEVTAGVLAEECGEKISAFFKRRRKEKKAAKKAAKLAE |
| 758 | MNDQGWMRLALEEARAARAAGEVPVGAVVVRDGEVLARAGNRVRLLCDPTAHAE |
| | IVALREAARRLGNYRLEGCTLYVTLEPCAMCAGAMIHARLDRLVYGVPNPKAGAAG |
| | SVLDVLGHPRLNHRLEVTGGVLADECGALMRDFFRAR |
| 759 | MDDEGWMRLALEEARASKAAGEVPVGAVVVRDGEVIARAGNRTRELCDPTAHAEI |
| | VALREAARRLGNYRLAGCTLYVTLEPCAMCAGAMIHARLDRLVYGVPNPKAGAAG |
| | SVLDVLGHPRLNHRMEVEGGVLAEECRALLTDFFRAR |
| 760 | MTDEHFMRQALAEARKAYDEGEVPVGAVVVRDGEIVATGRNQVERLKDPTAHAEM |
| | LAITAAAGHLGSKYLRGCTLYVTVEPCVMCAGACVWARVDRVVFGVRNPKAGACG |
| | SVFDIPGDPRLNHHPEVTGGVLEDECRQLLRDFFRER |
| 761 | MTDEHFMRQALEEARKAYDQGEVPVGAVVVRDGEVIARGRNQVERLKDPTAHAEM |
| | LAITAAAGTLGSKYLEGCTLYVTLEPCVMCAGACIWARVERVVFGVRNPKAGACGS |
| | VFDIPGDPRLNHRPEVVGGVLEAECAALMRDFFRER |
| 762 | MSEVEFSDDAGFMREALAEARAAAAAGEVPVGAVVVKDGEIIARAGNRTLRDNDPT |
| | AHAEIVALRAAARALGNHRLTGCTLYVTLEPCAMCAGAISHARIARLVYGVDNPKG |
| | GAVAHGPRFFAQPTCHHRPEVTGGVGAEEAGALLRDFFRAPRRVFNAQKKAQSSTD |
| 763 | MSEVDPTDLAFMREALAEARAAAEAGEVPVGAVVVCDGRIVARAHNRPIALNDPTA |
| | HAEILALREAAKALGNYRLTGCTLYATLEPCPMCAGAILHARIARLVYGVANPKAGA |
| | CGTVLDVMNHPRLNHRVEVTGGVLAEECGALLSGFFRAPRRVFNAQKKAQSSTDSG |
| | GSSGGSSGSETPGTSESATPESSGGSSGGSSEVDPTDLAFMREALAEARAAAEAGEVP |
| | VGAVVVCDGRIVARAHNRPIALNDPTAHAEILALREAAKALGNYRLTGCTLYATLEP |
| | CPMCAGAILHARIARLVYGVANPKAGACGTVLDVMNHPRLNHRVEVTGGVLAEEC |
| | GALLSGFFRAPRRVFNAQKKAQSSTD |
| 764 | MDDAGFMREALAEARAAAAAGEVPVGAVVVKDGEIIARAGNRTLRDNDPTAHAEIV |
| | ALRAAARALGNHRLTGCTLYVTLEPCAMCAGAISHARIARLVYGVDNPKGGAVAHG |
| | PRFFAQPTCHHRPEVTGGVGAEEAGALLRDFFRAR |
| 765 | MDDEGWMRLALEEARKSRAAGEVPVGAVVVRDGEVLATAGNRTRELCDPTAHAEI |
| | VALREAARRLGNYRLPGCTLYVTLEPCAMCAGAMIHARLDRLVYGVANPKAGAAG |
| | SVLDVLGHPRLNHRMEVEGGVLEAECAALLRDFFRAR |
| 766 | MDDEGWMQLALEEARQSRAAGEVPVGAVVVRDGEVIARAGNRTREKCDPTAHAEI |
| | VALREAARKLGNYRLPGCTLYVTLEPCAMCAGAMVHARLDRLVYGVANPKAGAA |
| | GSVLDVLHHPRLNHRMEVTGGVLAEECGALLRDFFRAR |
| 767 | MDDAGWMRLALEEARKSRARGEVPVGAVVVRDGQVLARAGNRTRELCDPTAHAEI |
| | VALRAAARKLGNYRLPGCTLYVTLEPCAMCAGAMVHARLDRLVYGVRNPKAGAA |
| | GSVLDVLGHPRLNHQMEVTGGVLADECAALLRDFFRAR |
| 768 | MSEVEFDDAGFMRLALEEARAAAAAGEVPVGAVVVRDGEVLARAGNRTVRDNDPT |
| | AHAEIVALREAARRLGSYRLAGCTLYVTLEPCPMCAGAMIHARLDRLVYGVANPKA |
| | GAAGTVLDVLGHPRLNHRMEVEGGVLAAECGALLRDFFRAPRRVFNAQKKAQSSTD |
| | SGGSSGGSSGSETPGTSESATPESSGGSSGGSSEVEFDDAGFMRLALEEARAAAAAGE |
| | VPVGAVVVRDGEVLARAGNRTVRDNDPTAHAEIVALREAARRLGSYRLAGCTLYVT |
| | LEPCPMCAGAMIHARLDRLVYGVANPKAGAAGTVLDVLGHPRLNHRMEVEGGVLA |
| | AECGALLRDFFRAPRRVFNAQKKAQSSTD |
| 769 | MDDAGFMELALDEARAARAAGEVPVGAVVVRDGEVLARAGNRTRELCDPTAHAEI |
| | VALREAARKLGNYRLEGCTLYVTLEPCAMCAGAMLHARLDRLVYGVANPKAGAAG |
| | SVLDVLGHPRLNHRMEVTGGVLEDECAALLRDFFRAR |
| 770 | MDDAGWMQLALEEARAARAAGEVPVGAVVVRDGEVLARAGNRTRERCDPTAHAE |
| | IVALRAAARHLGNYRLEGCTLYVTLEPCAMCAGAMLHARLDRLVYGVDNPKAGAA |
| | GSVLDVLGHPRLNHRLEVTGGVLADECGQLLRDFFRAR |
| 771 | MNDKGWMRLALEEARAARAAGEVPVGAVVVRDGEVIAAAGNRRRELCDPTAHAEI |
| | VALREAARRLGNYRLPGCTLYVTLEPCAMCAGAMIHARLDRLVFGVANPKAGAAGS |
| | VLDVLGHPRLNHRLEVTGGVLEEECGQLLRDFFRAR |
| 772 | MSDEHFMQQALAEARKAYDLGEVPVGAVVVRDGRIIARGHNQVEKLKDPTAHAEM |
| | LAITAAAGHLGSKYLEGCTLYVTLEPCIMCAGACVWARVERVVYGVRNPKAGACGS |
| | VFDIPGDPRLNHHPEVTGGVLEAECARLMRDFFRER |
| 773 | MSDEFFMKQALEEARLAYDEGEVPVGAVVVRDGEVIARGRNQIEELKDPTAHAEML |
| | AITAAAGHLGSKYLLGCTLYVTLEPCVMCAGACVWARVDRVVYGVRNPKAGAAGS |
| | VFDIPGDPRLNHHPEVVGGVLQAECAALMRDFFRER |
| 774 | MSEVEFSHEYWMRHALTLAKRAWDEREVPVGAVLVHNNRVIGEGWNRPIGRHDPT |
| | AHAEIMALRQGGLVMQNYRLIDATLYVTLEPCVMCAGAMIHSRIGRVVFGVRNAKT |
| | GAAGSLMDVLHHPGMNHRVEITEGILADECAALLSDFFRMRRQEIKAQKKAQSSTDS |
| | GGSSGGSSGSETPGTSESATPESSGGSSGGSSEVEFDDAGFMRLALEEARAAAAAGEV |
| | PVGAVVVRDGEVLARAGNRTVRDCDPTAHAEIVALREAARRLGNYRLAGCTLYVTL |
| | EPCAMCAGAMIHARLDRLVYGVANPKAGAAGSVLDVLGHPRLNHRMEVEGGVLAA |
| | ECGALLRDFFRAPRRVFNAQKKAQSSTD |
| 775 | MSEVEFTDEYFMRQALREARRAYDEGEVPVGAVVVRDGRVIARGRNQVERLKDPTA |
| | HAEILALRAACRHLGSKYLTGCTLYVTVEPCAMCAGALVWARVERVVFGVRNPKA |
| | GACGSVLDIPGDRRLNHRPEVVGGVLEAECAALMRDFFREPRRVFNAQKKAQSSTDS |
| | GGSSGGSSGSETPGTSESATPESSGGSSGGSSEVEFTDEYFMRQALREARRAYDEGEV |
| | PVGAVVVRDGRVIARGRNQVERLKDPTAHAEILALRAACRHLGSKYLTGCTLYVTV |
| | EPCAMCAGALVWARVERVVFGVRNPKAGACGSVLDIPGDRRLNHRPEVVGGVLEA |
| | ECAALMRDFFREPRRVFNAQKKAQSSTD |
| 776 | MDPTDLAFMREALAEARAAAEAGEVPVGAVVVCDGRIVARAHNRPIALSDPTAHAE |
| | ILALREAARALGNYRLTGCTLYATLEPCAMCAGAILHARIARLVYGVANPGTGACGS |
| | VLDVMNHPRLNHRVEVTGGVLAEECGALLSGFFRAR |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A polypeptide comprising an adenosine deaminase having an amino acid sequence with at least 75% identity to SEQ ID NO: 2.

2. The polypeptide of claim 1, wherein the adenosine deaminase has an amino acid sequence with at least 90% identity to SEQ ID NO: 2.

3. The polypeptide of claim 1, wherein the adenosine deaminase has an amino acid sequence of SEQ ID NO: 2.

4. A fusion protein comprising the polypeptide of claim 1 and a nucleic acid binding domain.

5. The fusion protein of claim 4, wherein the nucleic acid binding domain is a Clustered Regularly Interspaced Short Palindromic Repeats associated (Cas) protein or a fragment or variant thereof capable of nucleic acid binding.

6. The fusion protein of claim 5, wherein the Cas protein is at least partially catalytically inactivated.

7. The fusion protein of claim 5, wherein the Cas protein is a catalytically inactivated Cas9.

8. The fusion protein of claim 4, further comprising a linker separating the polypeptide and the nucleic acid binding domain, a localization sequence, a tag sequence, a protein transduction domain sequence, or a combination thereof.

9. A nucleic acid encoding the polypeptide of claim 1 or a fusion protein thereof.

10. A system comprising a polypeptide of claim 1 and a nucleic acid binding polypeptide, or one or more nucleic acids encoding thereof,
wherein the polypeptide and the nucleic acid binding polypeptide are fused as a single protein or wherein the polypeptide is linked to a first half of a binding pair and the nucleic acid binding polypeptide is linked to a second half of the binding pair.

11. The system of claim 10, wherein the nucleic acid binding polypeptide is a Cas protein.

12. The system of claim 11, wherein the Cas protein is at least partially catalytically inactivated.

13. The system of claim 11, wherein the Cas protein is catalytically inactivated Cas9.

14. The system of claim 11, further comprising at least one guide RNA or a nucleic acid encoding thereof.

15. A cell comprising a polypeptide of claim 1, a fusion protein or system comprising the polypeptide, or one or more nucleic acids encoding the polypeptide or fusion protein.

16. A method of modifying a target nucleic acid comprising contacting the target nucleic acid with a polypeptide of claim 1, or a fusion protein or system comprising the polypeptide.

17. The method of claim 16, wherein the method installs or reverses one or more point mutations in the target nucleic acid.

18. The method of claim 16, wherein the target nucleic acid is in a cell and the contacting comprises introducing the polypeptide, fusion protein or system comprising the polypeptide, or one or more nucleic acids encoding the polypeptide or fusion protein into the cell.

19. The method of claim 18, wherein the cell is in vitro, ex vivo, or in vivo.

20. The method of claim 19, wherein the introducing comprises administering to a subject.


SEQUENCE LISTING (808 sequences)

SEQ ID NO: 1 - protein, 147 aa, synthetic construct
MDDQGWMKLALEEARASRAAGEVPVGAVVVRDGQIVASAGNRTRELCDPTAHAEIVALRQAARALGNYRLPGCTLYVTLEPCAMCAGAMVHARLDRLVYGVPNPKAGAAGSVLDVLHHPRLNHRLEVTGGVLEDECGALLRDFFRAR

SEQ ID NO: 2 - protein, 149 aa, synthetic construct
MDPEDVAFMRKALDEARKAREAGEVPVGAVVVKDGEIVARAHNRTIQKSDPTAHAEILALRKAARALGNYRLTGCTLYATLEPCAMCAGAILHARIERLVYGVPNPKAGATGSVLNVLNHPRLNHRVEVTGGVLAEECGALLRGFFRAR

SEQ ID NO: 3 - protein, 149 aa, synthetic construct
MDPTDLAFMREALAEARAAAEAGEVPVGAVVVCDGRIVARAHNRPIALSDPTAHAEILALREAARALGNYRLTGCTLYATLEPCAMCAGAILHARIARLVYGVANPKAGACGSVLDVMNHPRLNHRVEVTGGVLAEECGALLSGFFRAR

SEQ ID NO: 4 - protein, 147 aa, synthetic construct
MDDAGFMRLALEEARAAAAAGEVPVGAVVVRDGEVLARAGNRTVRDNDPTAHAEIVALREAARRLGSYRLAGCTLYVTLEPCPMCAGAMIHARLDRLVYGVANPKAGAAGTVLDVLGHPRLNHRMEVEGGVLAAECGALLRDFFRAR

SEQ ID NO: 5 - protein, 147 aa, synthetic construct
MDDKGFMQLALEEARAAQAAGEVPVGAVVVRDGEVLARAGNRTRELCDPTAHAEIVALREAARKRGNYRLPGCTLYVTLEPCAMCAGAMLHARLDRLVYGVANPKAGAAGSVLDVLGHPRLNHRLEVTGGVLEAECGALLRDFFRAR

SEQ ID NO: 6 - protein, 147 aa, synthetic construct
MDDKGWMRLALEEARAAKAAGEVPVGAVVVRDGQVLARAGNQVRELCDPTAHAEIVALREAARKLGNYRLPGCTLYVTLEPCAMCAGAMLHARLDRLVYGVANPKAGAAGSVLDVLGHPRLNHRLEVEGGVLADECGQLLRDFFRAR

SEQ ID NO: 7 - protein, 149 aa, synthetic construct
MDPEDLRFMRQALAEARRAAEAGEVPVGAVVVLDGEVVAQAFNRTRAQSDPTAHAEILALREAARATGNYRLTGCTLYVTLEPCAMCAGAILHARIARLVYGVANPKAGAAGSVLDVLNHPRLNHRVEVTGGVLAEECGALLRDFFRAR

SEQ ID NO: 8 - protein, 147 aa, synthetic construct
MNDEGWMQLALEEARKAKAAGEVPVGAVVVRDGEVIAMAGNRTRERCDPTAHAEIVALREAARRLGNYRLPGCTLYVTLEPCAMCAGAMLHARLDRLVYGVPNPKAGAAGSVLDVLGHPRLNHRMEVTGGVLEEECGRLLRDFFRAR

SEQ ID NO: 9 - protein, 147 aa, synthetic construct
MDDEGWMKLALEEARAARAAGEVPVGAVVVKDGEVIARAGNRTRELCDPTAHAEIIALREAARRLGNYRLPGCTLYVTLEPCAMCAGAMLHARLDRLVYGVANPKAGAAGSVLDVLGHPRLNHRMEVTGGVLEAECGQLLRDFFRAR

SEQ ID NO: 10 - protein, 149 aa, synthetic construct
MDPTDLAFMREALAEARAAAEAGEVPVGAVVVCDGRIVARAHNRPIALNDPTAHAEILALREAAKALGNYRLTGCTLYATLEPCPMCAGAILHARIARLVYGVANPKAGACGTVLDVMNHPRLNHRVEVTGGVLAEECGALLSGFFRAR

SEQ ID NO: 11 - protein, 149 aa, synthetic construct
MDPTDIAFMRQALAEARKAKDAGEVPVGAVVVHDGEIVARAHNRTILESDPTAHAEILALRAAARRLGNYRLTGCTLYVTLEPCAMCAGAILHARIERLVYGVANPKAGAAGSVLNVLNHPRLNHRVEVTGGVLADECGALLSGFFRAR

SEQ ID NO: 12 - protein, 147 aa, synthetic construct
MDDEDWMRLALEEARKARAAGEVPVGAVVVRDGQVLARAGNRTRELCDPTAHAEIIALRQAARRRGNYRLAGCTLYVTLEPCAMCAGAMIHARLDRLVYGVPNPKAGAAGSVLDVLHHPRLNHRLEVTGGVLEAECGALLRDFFRAR

SEQ ID NO: 13 - protein, 149 aa, synthetic construct
MDPDDVEFMRQALDEARRARDAGEVPVGAVVVHDGRVVARAHNRVIAESDPTAHAEIRALREAARALGNYRLTGCTLYATLEPCAMCAGAILHARIARLVYGVPNPKAGATGSVLDVLNHPRLNHRVEVTGGVLEEECGRLLSGFFRAR

SEQ ID NO: 14 - protein, 147 aa, synthetic construct
MDDQGWMQLALEEARAARAAGEVPVGAVVVRDGEVLARAGNRTRELCDPTAHAEIRALREAARALGNYRLAGCTLYVTLEPCAMCAGAMIHARLDRLVYGVRNPKAGAAGSVLDVLGHPRLNHRLEVTGGVLEAECGALLRDFFRAR

SEQ ID NO: 15 - protein, 149 aa, synthetic construct
MDPEDIEFMKKALAEARAAKEAGEVPVGAVVVSDGEVVARAHNQTIERSDPTAHAEILALRAAARALGNYRLTGCTLYVTLEPCAMCAGAILHARIERLVYGVANPKAGAAGSVLDVLNHPRLNHRVEVTGGVLAEECGALLSDFFRAR

SEQ ID NO: 16 - protein, 147 aa, synthetic construct
MDDEGWMRLALEEARAARAAGEVPVGAVVVRDGQVLAQAGNRTRELCDPTAHAEIVALREAARALGNYRLAGCTLYVTLEPCAMCAGAMLHARLDRLVYGVANPKAGAAGSVLDVLGHPRLNHRAEVEGGVLADECGALLRDFFRAR

SEQ ID NO: 17 - protein, 149 aa, synthetic construct
MDPEDREFMRKALEEARKAREAGEVPVGAVVVLDGRIVARAHNQTRAESDPTAHAEILALREAARALGNYRLTGCTLYVTLEPCAMCAGAILHARIERLVYGVPNPKAGATGSVLNVLNHPRLNHRVEVTGGVLAEECGALLSGFFRAR

SEQ ID NO: 18 - protein, 147 aa, synthetic construct
MDDEGWMQLALEEARASKAAGEVPVGAVVVRDGRVLARAGNRTRERCDPTAHAEIVALREAARRLGNYRLTGCTLYVTLEPCAMCAGAMLHARLDRLVYGVPNPKAGAAGSVLDVLHHPRLNHRLEVEGGVLEAECGQLLRDFFRAR

SEQ ID NO: 19 - protein, 147 aa, synthetic construct
MDDRGWMKLALEEARAAKAAGEVPVGAVVVRDGEVIARAGNRTRLKCDPTAHAEIVALREAARRLGNYRLPGCTLYVTLEPCAMCAGAMLHARLDRLVYGVPNPKAGAAGSVLDVLGHPRLNHRLEVTGGVLAAECAALLRDFFRAR

SEQ ID NO: 20 - protein, 147 aa, synthetic construct
MDDEGWMRLALEEARKAKAAGEVPVGAVVVRNGEVIARAGNRTRELCDPTAHAEIVALREAARRLGNYRLAGCTLYVTLEPCAMCAGAMIHARLDRLVYGVANPKAGAAGSVLDVLGHPRLNHRLEVEGGVLEAECGALLRDFFRAR

SEQ ID NO: 21 - protein, 149 aa, synthetic construct
MDPMDIAFMQQALDEARKAKEAGEVPVGAVVVHDGQIVARAHNRTIALSDPTAHAEILALREAARALGNYRLQGCTLYATLEPCAMCAGAILHARIARLVYGVPNPKAGACGSVLNVLNHPRLNHRVEVTGGVLAEECGALLRGFFRAR

SEQ ID NO: 22 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPKAGAAGSVLDVLGHPRLNHRMEVEGGVLAAECGALLRDFFRAR

SEQ ID NO: 23 - protein, 147 aa, synthetic construct
MTDEYFMRQALREARRAYDEGEVPVGAVVVRDGRVIARGRNQVERLKDPTAHAEMLALTAAAGHLGSKYLTGCTLYVTVEPCAMCAGALVWARVERVVFGVRNPKAGACGSVFDIPGDRRLNHRPEVVGGVLEAECAALMRDFFRER

SEQ ID NO: 24 - protein, 142 aa, synthetic construct
MDAALEEARRAAAAGEVPVGAVLVRDGAVLARGGNRTIRDCDPTAHAEIVALRAAARAAGNYRLPGATLYVTLEPCAMCAGAMIHARLDRLVYGAADPRAGAAGSVFDVLRHPALNHQMAVEGGVRAAECGALLRDFFRARR

SEQ ID NO: 25 - protein, 148 aa, synthetic construct
MTDADFMALALEEARAAAALGEVPVGAVVVRDGAVIARAGNRTIRDCDPTAHAEIVALREAARALGNYRLAGCTLYVTLEPCAMCAGAMIHARLDRLVYGAADPKTGAAGSVLDVLNHPKLNHQMQVEAGVLAEECGAMLRDFFQQRR

SEQ ID NO: 26 - protein, 142 aa, synthetic construct
MRDALAEARKAADAGEVPVGAVVVRGGEILARAHNRTVADHDPTAHAEILALREAARVLGNHRLTGCTLYVTLEPCAMCAGAIVHARLDRLVYGAADPKAGAAGSVLDVLNHPRLNHRMEVEGGVLAEESGELLRGFFRARR

SEQ ID NO: 27 - protein, 148 aa, synthetic construct
MDDAGFMREALAEARAAAAAGEVPVGAVVVKDGEIIARAGNRTLRDNDPTAHAEIVALRAAARALGNHRLTGCTLYVTLEPCAMCAGAISHARIARLVYGADDPKGGAVAHGPRFFAQPTCHHRPEVTGGVGAEEAGALLRDFFRARR

SEQ ID NO: 28 - protein, 142 aa, synthetic construct
MREALAEARAAAEAGEVPVGAVVVRDGEIIARARNRMVADCDPTAHAEIVALREAARVLGNHRLTGCTLYATLEPCAMCAGAIAHARIARLVYAADDPKGGAVWHGPRFFEQPTCHHRPEVTSGVLADEAAALLRDFFRARR

SEQ ID NO: 29 - protein, 142 aa, synthetic construct
MREALAEARAAAAAGEVPVGAVVVRDGAVLARAGNASIRARDPTAHAEVLALRAAARALGNHRLTGCTLYVTLEPCAMCAGAIAHARVARLVYGAADPKGGAVAHGPRFFEQPTCHHRPEVTGGVLAEEAGALLRDFFRARR

SEQ ID NO: 30 - protein, 142 aa, synthetic construct
MREALAEARAAAAAGEVPVGAVVVRDGAVLARAGNRTVRDADPTAHAEIVALREAARALGNHRLGGCTLYVTLEPCAMCAGAIAQARVARLVYGADDPKGGAVAHGPRLFAQPTCHHRPEVVGGVGAAEAGALLRDFFAARR

SEQ ID NO: 31 - protein, 142 aa, synthetic construct
MTEALAEARKAAAAGEVPVGAVLVRDGAVLARGGNRTIRDCDPTAHAEIVALRAAARALGNYRLPGTTLYVTLEPCAMCAGAMIHARVARLVYGAADPKTGAAGSVFDVLRHPALNHRMEVEGGVLAEECGALLREFFRARR

SEQ ID NO: 32 - protein, 142 aa, synthetic construct
MREALAEARAAAAAGEVPVGAVVVRDGAVLARAGNRTVRDADPTAHAEIVALREAARALGNHRLTGCTLYVTLEPCAMCAGAISHARVARLVYGADDPKGGAVAHGPRFFAQPTCHHRPEVTGGVLAAEAGELLRGFFRARR

SEQ ID NO: 33 - protein, 142 aa, synthetic construct
MRDALAEARAAAAAGEVPVGAVVVRDGAIVARARNRMVADCDPTAHAEIVALRAAARALGNHRVDGCTLYVTLEPCAMCAGAMIHARLARLVYGAADPRAGAAGSVLDVLGHPALNHRMEVTAGVLAEECGALLRDFFAARR

SEQ ID NO: 34 - protein, 142 aa, synthetic construct
MTEALAEARKAAAAGEVPVGAVLVRDGAVLARGGNRTIRDCDPTAHAEIVALRAAARALGNYRLPGTTLYVTLEPCAMCAGAMIHARVARLVYGAADPKTGAAGSVFDILRHPALNHRMEVEGGVLAEECGALLRDFFRARR

SEQ ID NO: 35 - protein, 142 aa, synthetic construct
MREALAEARAAAAAGEVPVGAVVVRDGAVLARAGNRTVRDADPTAHAEIVALRAAARALGNHRLDGCTLYVTLEPCAMCAGAIAQARVARLVYGAADPKGGAVAHGPRLFEQPTTHHRPEVVGGVLAEEAAALLRGFFAARR

SEQ ID NO: 36 - protein, 142 aa, synthetic construct
MRAALAEARAAAAAGEVPVGAVVVRDGAVLARAGNASIRARDPTAHAEILALRAAARALGNHRLTGCTLYVTLEPCAMCAGAIAHARIARLVYGAADPKGGAVAHGPRFFEQPTCHHRPEVTGGVLAEEAGALLRAFFRARR

SEQ ID NO: 37 - protein, 142 aa, synthetic construct
MRDALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPEVVGGVGAAEAGALLRDFFRARR

SEQ ID NO: 38 - protein, 142 aa, synthetic construct
MREALAEARAAAAAGEVPVGAVVVRDGAVLARAGNRTVRDADPTAHAEIVALRAAARALGNHRLDGCTLYVTLEPCAMCAGAMVQARLARLVYGAADPKAGAVDSVLDVLDHPRLNHRMEVTGGVLAEECGALLREFFAARR

SEQ ID NO: 39 - protein, 142 aa, synthetic construct
MRDALAEARAAAAAGEVPVGAVVVRDGAILARAGNASIRARDPTAHAEILALRAAARALGNHRLDGCTLYVTLEPCAMCAGAIAHARIARLVYGAADPKGGAVAHGPRFFEQPTCHHRPDVTGGVGAAEAAALLRDFFRARR

SEQ ID NO: 40 - protein, 149 aa, synthetic construct
MDDAGFMRLALEEARAAAAAGEVPVGAVVVRDGEVLARARNAPVAAHDPTAHAEILALRAAAAALGNYRLDGCTLYVTLEPCAMCSGAMLHARLARVVYGAADPKTGAAGSVLDLFAQPRLNHHTAVEGGVLAAECGALLRDFFRARRG

SEQ ID NO: 41 - protein, 148 aa, synthetic construct
MDDEFFMREALRLAEEAAAAGEVPVGAVVVRDGEIVGRGRNRVLEDRDPTAHAEIVALREAARRLGNYRLEGCELYVTLEPCAMCAGAMVHARLARLVYGAADPKAGAAGSVLDVLGHPRLNHRMEVTGGVLAEECGALLREFFRARR

SEQ ID NO: 42 - protein, 142 aa, synthetic construct
MRDALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPDVTGGVGAAEAAALLRDFFRARR

SEQ ID NO: 43 - protein, 142 aa, synthetic construct
MREALAEARAAAAAGEVPVGAVVVRDGAVLARAGNRTVRDADPTAHAEVVALREAARALGNHRLGGCTLYVTLEPCAMCAGAIAQARVARLVYGADDPKGGAVAHGPRLFAQPTCHHRPEVVGGVGAAEAAALLRGFFAARR

SEQ ID NO: 44 - protein, 148 aa, synthetic construct
MDDAGFMRLALAEARAAAAAGEVPVGAVVVRDGAVLAAAGNRTLRDCDPTAHAEVVALRAAARALGNHRLGGCDLYVTLEPCAMCAGAISFARIRRLYFGADDPKGGAVEHGPRFFAQPTCHHAPEVYGGLAESEAAALLRDFFRARR

SEQ ID NO: 45 - protein, 149 aa, synthetic construct
MDDAGFMRLALAEARAAAAAGEVPVGAVVVRDGAVLAAAGNRTLRDCDPTAHAEVVALRAAARALGNHRLEGCTLYVTLEPCAMCAGAMLHARLARVVYGAADPKTGAAGSVLDLFANPRLNHHTRVEGGVLAEECGALLQDFFRARRG

SEQ ID NO: 46 - protein, 148 aa, synthetic construct
MSDEDYMRLALEEARAAAAAGEVPIGAVVVRDGEVLARAGNRTVRDCDPTAHAEIVALREAARKLGNYRLAGCTLYVTVEPCAMCAGALVWARVDRLVYGADDPKAGAVRSALAVVDHPRLNHRMEVVSGVLAGECAALLQEFFAARR

SEQ ID NO: 47 - protein, 142 aa, synthetic construct
MREALAEARAAAAAGEVPVGAVVVRDGAVLARAGNASIRARDPTAHAEILALRAAARALGNHRLTGCTLYVTLEPCAMCAGAIAHARVARLVYGAADPKGGAVAHGPRFFEQPTCHHRPEVTGGVLAEEAGALLRDFFRARR

SEQ ID NO: 48 - protein, 142 aa, synthetic construct
MREALAEARAAAAAGEVPVGAVVVRDGAVLARAGNASIRARDPTAHAEILALRAAARALGNHRLDGCTLYVTLEPCAMCAGAIAQARVARLVYGAADPKGGAVAHGPRLFAQPTCHHRPEVTGGVGEAEAAALLRDFFAARR

SEQ ID NO: 49 - protein, 148 aa, synthetic construct
MTDEYFMRQALREARKAYDEDEVPVGAVVVRDGKVIARGRNQVERLKDPTAHAEMIALTSAANYLGSKRLEGCTVYVTLEPCPMCAGALVLARVERLVFGAFDPKAGACGTLYDIPGDRRLNHRIEVVGGVLEEESAALLREFFEKRR

SEQ ID NO: 50 - protein, 142 aa, synthetic construct
MREALAEARAAAAAGEVPVGAVVVRDGAVLARAGNASIRARDPTAHAEMLALRAAARALGNHRLDGCTLYVTLEPCAMCAGAIAHARIARLVYGAADPKGGAVAHGPRFFAQPTCHHRPEVTGGVLAEEAGALLRDFFRARR

SEQ ID NO: 51 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPEVVGGVGAGEAAALLRDFFAARR

SEQ ID NO: 52 - protein, 148 aa, synthetic construct
MDDAGFMRLALAEARAAAAAGEVPVGAVVVRDGAVLAAAGNRTLRDCDPTAHAEVVALRAAARALGNHRLEGCDLYVTLEPCAMCAGAISFARIRRLYFGAADPKGGAVEHGPRFFAQPTCHHAPEVYGGLAESEAAALLRGFFAARR

SEQ ID NO: 53 - protein, 142 aa, synthetic construct
MREALAEARAAAAAGEVPVGAVVVRDGAVLARAGNRTVRDADPTAHAEIVALRAAARALGNHRLDGCTLYVTLEPCAMCAGAIAQARVARLVYGAADPKGGAVAHGPRLFEQPTTHHRPEVVGGVLAAEAGALLRAFFAARR

SEQ ID NO: 54 - protein, 142 aa, synthetic construct
MTEALAEARKAAALGEVPVGAVVVRDGAVIARGHNRTVTDADPTAHAEIVALREAARALGNHRLTGCTLYVTLEPCAMCAGAIVHARVDRLVYGAADPKAGAAGSVLDVIGHPRLNHRPEVEGGVLGEECGALLRDFFRARR

SEQ ID NO: 55 - protein, 142 aa, synthetic construct
MRSALDLAAAAAAAGEVPVGAVVVRDGAIVGRGENRVLRDSDPTAHAEIVAMREAARALGNYRLTGCTLYVTLEPCAMCAGAMVHARLDRLVYGAADPKAGAAGSVLDVLGHPRLNHQMEVEGGVLAAESAALLRDFFRARR

SEQ ID NO: 56 - protein, 142 aa, synthetic construct
MREALAEARAAAAAGEVPVGAVVVRDGAVLARAGNASIRARDPTAHAEILALRAAARALGNHRLTGCTLYVTLEPCAMCAGAIAHARIARLVYGAADPKGGAVAHGPRFFEQPTCHHRPEVTGGVLAEEAGALLRDFFRARR

SEQ ID NO: 57 - protein, 148 aa, synthetic construct
MDDAGFMRLALAEAEAAAAAGEVPVGAVVVRDGEVIARAGNRTVRDCDPTAHAEIVALRAAARALGNHRLTGCTLYVTLEPCAMCAGAISHARVARLVYGADDPKGGAVAHGPRFFAQPTCHHRPEVAGGVLAEEAGALLRGFFRARR

SEQ ID NO: 58 - protein, 148 aa, synthetic construct
MDDAGFMRLALAEARAAAAAGEVPVGAVVVRDGAVLAAAGNRTLRDCDPTAHAEIVALRAAARALGNYRLDGCDLYVTLEPCAMCAGAMLHARLRRVVFGAADPKTGAAGSVLDLFAERRLNHRTAVAGGVLADECGALLRDFFRARR

SEQ ID NO: 59 - protein, 147 aa, synthetic construct
MDDAGFMRLALAEARAAAAAGEVPVGAVVVRDGAVLAAAGNRTLRDCDPTAHAEVVALRAAARALGNHRLDGCDLYVTLEPCAMCAGAISHARIRRLYYGADDPKGGAVDNGVRFFASPTCHHAPEVYGGLAEGEAAALLRDFFRER

SEQ ID NO: 60 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGADDPKGGAVAHGPRFFAQPTCHHRPEVVGGVGAEEAGALLRGFFAARR

SEQ ID NO: 61 - protein, 142 aa, synthetic construct
MTEALREARRAAAAGEVPVGAVVVRDGAVLARAGNRTVRDADPTAHAEIVALREAARALGNHRLTGCTLYVTLEPCAMCAGAISHARIARLVYGAADPKGGAVAHGPRFFEQPTCHHRPEVTGGVGEAEAAALLRDFFRARR

SEQ ID NO: 62 - protein, 142 aa, synthetic construct
MREALAEARAAAAAGEVPVGAVVVRDGAVLARAGNRTVRDADPTAHAEVVALRAAARALGNHRLGGCTLYVTLEPCAMCAGAIAQARVARLVYGAADPKGGAVAHGPRLFAQPTCHHRPEVVGGVGEAEAAALLRDFFAARR

SEQ ID NO: 63 - protein, 142 aa, synthetic construct
MREALAEARAAAAAGEVPVGAVVVRDGAVLARAGNASIRARDPTAHAEVLALRAAARALGNHRLTGCTLYVTLEPCAMCAGAIAHARIARLVYGAADPKGGAVAHGPRFFEQPTCHHRPEVTGGVLAEEAGALLRDFFRARR

SEQ ID NO: 64 - protein, 148 aa, synthetic construct
MTDEYFMRQALREARRAYEEDEVPVGAVVVRDGRVIARGRNQVERLKDPTAHAEMIALTAAANHLGSKRLEGCTVYVTLEPCPMCAGALVLARVPRLVFGAFDPKAGACGTLYDIVRDPRLNHRVEVVGGVLEEECGELLKRFFRERR

SEQ ID NO: 65 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVAHGPRLFAQPTCHHRPEVVGGVGAAEAGALLRDFFAARR

SEQ ID NO: 66 - protein, 142 aa, synthetic construct
MTEALAEARKAAAEGEVPVGAVVVRDGVVLARAHNRTVADHDPTAHAELLAIREAARVLGNHRLTGCTLYVTLEPCAMCAGAIVHARLDRLVYGAADPKAGAAGSVLDVLGHPRLNHRPEVEGGVLAAESAALLRDFFRARR

SEQ ID NO: 67 - protein, 148 aa, synthetic construct
MDDAGFMRLALAEAERAAALGEVPVGAVLVRDGEVLAAAGNRTVADCDPTAHAEMLALREGARRLGNYRLTGCTLYVTLEPCAMCAGAMVHARLDRLVYAAADPKAGAAGSVLDVLNHPALNHRMQVEGGVLAEESAALLRGFFRARR

SEQ ID NO: 68 - protein, 142 aa, synthetic construct
MREALAEARAAAAAGEVPVGAVVVRDGAVLARAGNRTVRDADPTAHAEIVALREAARALGNHRVGGATLYVTLEPCAMCAGAISQARVARLVYGADDPKGGAVAHGPRFFEQPTCHHRPEVTGGVLAEESAALLRGFFAARR

SEQ ID NO: 69 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPEVVGGVGAAEAGALLRDFFRARR

SEQ ID NO: 70 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVAHGPRFFEQPTCHHRPDVTGGVLAEEAGALLRDFFRARR

SEQ ID NO: 71 - protein, 148 aa, synthetic construct
MTDEYFMRQALREARRAYDEGEVPVGAVVVRDGRVIARGRNQVERLKDPTAHAEMIALTAAADYLGSKRLEGCTLYVTLEPCAMCAGAIVLARIPRLVFGAFDPKAGACGTLYDIVRDRRLNHRAEVVSGVLEEECGALLKEFFARLR

SEQ ID NO: 72 - protein, 148 aa, synthetic construct
MDDEFFMREALRLAEEAAAAGEVPVGAVVVRDGEIVGRGRNRVLEDRDPTAHAEIVAMREAARRLGNYRLEGCTLYVTLEPCAMCAGAMVHARVARLVYGAADPKAGAAGSVLDVLGHPRLNHRMEVTGGVLAEECGALLREFFRRRR

SEQ ID NO: 73 - protein, 142 aa, synthetic construct
MREALAEARAAAAAGEVPVGAVVVRDGAVLARAGNRTVRDADPTAHAELVAIRAAAAALGQERLTGCTLYVTLEPCAMCAGAISHARIARLVYGAADPKGGAVAHGPRFFEQPTCHHRPEVTGGVLAEEAGALLRDFFRARR

SEQ ID NO: 74 - protein, 142 aa, synthetic construct
MREALAEARAAAAAGEVPVGAVVVRDGAIVARARNRMVADCDPTAHAEIVALREAARALGNHRLDGCTLYVTLEPCAMCAGAMVHARVARLVYGAADPRAGAAGSVLDVLGHPALNHRMEVAGGVLAEECGALLREFFAARR

SEQ ID NO: 75 - protein, 142 aa, synthetic construct
MRAALAEARAAAAAGEVPVGAVVVRDGAVLARAGNASIRARDPTAHAEILALRAAARALGNHRLDGCTLYVTLEPCAMCAGAIAHARVARLVYGAADPKGGAVAHGPRFFEQPTCHHRPEVTGGVLAEEAGALLRDFFRARR

SEQ ID NO: 76 - protein, 142 aa, synthetic construct
MRAALDEARAAAAAGEVPVGAVVVRDGAILARAGNRTVRDCDPTAHAEIVALREAARALGNHRLTGCTLYVTLEPCAMCAGAISHARVARLVYGADDPKGGAVRHGPRFFEQPTCHHRPEVVGGVGAEEAAALLRGFFAARR

SEQ ID NO: 77 - protein, 162 aa, synthetic construct
MDDEAWMRRAIALAHQAEAEGEVPVGAVLVKDGEVIAEGWNRSIGHHDATAHAEIETLRQAGQALQNYRLLDTTLYVTLEPCPMCAGALLHSRVKRVVFGAPDLKAGAAGTVLNLFESQASYHYAEVESGLLEQECREQLQAFFKRRRKEIKALRQAQRDAE

SEQ ID NO: 78 - protein, 148 aa, synthetic construct
MDDAGFMRLALAEARAAAAAGEVPVGAVVVRDGAVLAAAGNRTLRDCDPTAHAEVVALRAAARALGNHRLEGCDLYVTLEPCAMCAGAIAHARIARLYYGAADPKGGAVEHGARVFDQPQCLHRPEVYGGIGEAEAAALLRGFFAARR

SEQ ID NO: 79 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGADDPKGGAVAHGPRFFAQPTCHHRPEVAGGLGEAEAGALLRDFFAARR

SEQ ID NO: 80 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGAADPKGGAVAHGPRLFAQPTCHHRPEVVGGVGEAEAAALLRAFFAARR

SEQ ID NO: 81 - protein, 142 aa, synthetic construct
MRDALAEARAAAAAGEVPVGAVVVRDGAVLARAGNASIRARDPTAHAEILALRAAARALGNHRLDGCTLYVTLEPCAMCAGAIAHARIARLVYGAADPKGGAVAHGPRFFAQPTCHHRPEVTGGVGEAEAAALLRAFFAARR

SEQ ID NO: 82 - protein, 148 aa, synthetic construct
MTDEYFMRQALREARRAYEEDEVPVGAVVVRDGRVIARGRNQVERLKDPTAHAEMIALTAAANHLGSKRLEGCTVYVTLEPCPMCAGALVLARVERLVFGAFDPKAGACGTLYDIPGDRRLNHRLEVVGGVLEEESAALLREFFRKRR

SEQ ID NO: 83 - protein, 148 aa, synthetic construct
MTDEDYMRLALEEARAAAAAGEVPIGAVVVRDGEVLARAGNRTVRDCDPTAHAEIVALREAARVLGNYRLAGCTLYVTVEPCAMCAGAIVHARVARLVYGADDPKGGAVRSCLEVLDHPRLNHRVEVTAGVLAGECAALLQDFFAARR

SEQ ID NO: 84 - protein, 142 aa, synthetic construct
MREALAEARAAAAAGEVPVGAVVVRDGAVLARAGNRTVRDADPTAHAEVLALRAAARALGSERLTGCTLYVTLEPCAMCAGAISHARVARLVYGAADPKGGAVEHGARVFEQPTCHHRPEVVGGVGAAEAGALLRDFFAARR

SEQ ID NO: 85 - protein, 142 aa, synthetic construct
MREALAEARAAAAAGEVPVGAVVVRDGAILARAGNASIRARDPTAHAEILALRAAARALGNHRLTGCTLYVTLEPCAMCAGAIAHARIARLVYGAADPKGGAVAHGPRFFEQPTCHHRPEVTGGVLAEEAGALLRAFFRARR

SEQ ID NO: 86 - protein, 148 aa, synthetic construct
MDDAGFMRLALEEARAAAAAGEVPVGAVVVRDGAVLARAGNRTLRDRDPTAHAEMVALRAAARALGSERLTGCDLYVTLEPCAMCAGAISFARIRRLYFGAADPKGGAVENGVRFFASPTCHHAPEVYGGLAESEAAALLRDFFRARR

SEQ ID NO: 87 - protein, 148 aa, synthetic construct
MTDEYFMRQALREARRAYDEGEVPVGAVVVRDGRVIARGRNQVERLKDPTAHAEMLALTAAAGHLGSKYLTGCTLYVTVEPCAMCAGALVWARVERVVFGARDPKAGACGSVFDIPGDRRLNHRPEVVGGVLEAECAALMRDFFRERR

SEQ ID NO: 88 - protein, 142 aa, synthetic construct
MREALAEARAAAAAGEVPVGAVVVRDGAVLARAGNASIRARDPTAHAEMLALRAAARALGNHRLDGCTLYVTLEPCAMCAGAIAHARVARLVYGAADPKGGAVAHGPRFFEQPTCHHRPEVTGGVLAEEAGALLREFFRARR

SEQ ID NO: 89 - protein, 142 aa, synthetic construct
MREALAEARAAAAAGEVPVGAVVVRDGAILARAGNASIRARDPTAHAEILALRAAARALGNHRLTGCTLYVTLEPCAMCAGAIAHARIARLVYGAADPKGGAVAHGPRFFAQPTCHHRPEVTGGVLAEEAGALLRAFFRARR

SEQ ID NO: 90 - protein, 163 aa, synthetic construct
MSDEQYMRRALELARQAEQAGEVPVGAVLVKDGEIIAEGWNQSISAHDATAHAEIMALRAAGQALENYRLLDTTLYVTLEPCPMCAGALLHSRVKRVVFGAPDLKAGAAGTVLNLFESQASYHYADVESGLLEDECREQLQAFFKRRRKEIKALRQAQREAEE

SEQ ID NO: 91 - protein, 162 aa, synthetic construct
MSEEQYMRRALELARQAEQEGEVPVGAVLVKDGEVIAEGWNRSIGSHDATGHAEIMALRAAGEKLENYRLLDTTLYVTLEPCPMCAGALLHSRVKRVVFGAPDLKAGAAGTVLNLFESQASYHYADVESGLLEEECREQLQAFFKRRRKEIKALRQAQKEAE

SEQ ID NO: 92 - protein, 148 aa, synthetic construct
MDDAGFMRLALAEARRAAEAGEVPVGAVVVRDGAVLAAAGNRTLRDCDPTAHAEVVALREAARALGNHRLGGCDLYVTLEPCAMCAGAISHARIARLYYGADDPKGGAVAHGARVFDQPTCHHRPEVYGGIGAAEAAALLRDFFRARR

SEQ ID NO: 93 - protein, 142 aa, synthetic construct
MREALAEARAAAAAGEVPVGAVVVRDGAVLARAGNASIRARDPTAHAEVLALRAAARALGNHRLTGCTLYVTLEPCAMCAGAIAHARVARLVYGAADPKGGAVAHGPRFFEQPTCHHRPEVTGGVLAAEAGALLRDFFRARR

SEQ ID NO: 94 - protein, 142 aa, synthetic construct
MREALAEARAAAAAGEVPVGAVVVRDGAVLARAGNASIRARDPTAHAEMLALRAAARALGNHRLDGCTLYVTLEPCAMCAGAIAHARIARLVYGAADPKGGAVAHGPRFFAQPTCHHRPDVTGGVLAEEAGALLRDFFRARR

SEQ ID NO: 95 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGADDPKGGAVAHGPRFFAQPTCHHRPEVVGGVGEAEAGALLRDFFAARR

SEQ ID NO: 96 - protein, 148 aa, synthetic construct
MTDEYFMRQALREARRAYDEGEVPVGAVVVRDGRVIARGRNQVERLKDPTAHAEMLALTAAAAHLGSKRLEGCTVYVTLEPCAMCAGALVLARVDRLVFGAFDPKAGACGTVYDIPRDRRLNHRVEVVGGVLEEESAALLRAFFEERR

SEQ ID NO: 97 - protein, 142 aa, synthetic construct
MREALAEARAAAAAGEVPVGAVVVRDGAVLARAGNASIRARDPTAHAEILALRAAARALGNHRLDGCTLYVTLEPCAMCAGAIAQARVARLVYGAADPKGGAVAHGPRLFAQPTCHHRPEVVGGVGAEEAAALLRGFFAARR

SEQ ID NO: 98 - protein, 148 aa, synthetic construct
MTDEYFMRQALREARRAYDEGEVPVGAVVVRDGRVIARGRNQVERLKDPTAHAEMIALTAAADHLGSKRLEGCTLYVTLEPCAMCAGAIVLARIPRLVFGAFDPKAGACGTLYDIVRDRRLNHRVEVVSGVLEEESAALLREFFAERR

SEQ ID NO: 99 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPDVVGGVGAAEAGALLRDFFRARR

SEQ ID NO: 100 - protein, 148 aa, synthetic construct
MTDEYFMRQALREARRAYDEDEVPVGAVVVRDGRVIARGRNQVERLKDPTAHAEMIALTSAANHLGSKRLEGCTVYVTLEPCPMCAGALVLARVPRLVFGAFDPKAGACGTLYDIPRDRRLNHRVEVVGGVLEEESAALLREFFARRR

SEQ ID NO: 101 - protein, 142 aa, synthetic construct
MTEALAEARKAAAAGEVPVGAVLVRDGEILARGGNRTIRDCDPTAHAEIVALREAARKLGNYRLAGCTLYVTLEPCAMCAGAMIHARLDRLVYGADDPKAGAAGSVLDVLNHPRLNHQMEVTRGVLADECGALLRDFFQARR

SEQ ID NO: 102 - protein, 148 aa, synthetic construct
MTDEYFMRQALREARRAFDEDEVPVGAVVVRDGRVIARGRNQVERLKDPTAHAEMIAITSAANYLGSKRLEGCTVYVTLEPCPMCAGALVLARVPRLVFGAFDPKAGACGTLYDIPRDRRLNHRLEVVGGVLEEESAALLREFFARRR

SEQ ID NO: 103 - protein, 148 aa, synthetic construct
MTDEDYMRLALEEARAAAAAGEVPVGAVVVRDGAVLARAGNRTVRDCDPTAHAEIVALRAAARALGNYRLPGCTLYVTVEPCAMCAGAMIHARLDRLVYGADDPKAGAVRSTLRVLDHPALNHRMAVTAGVLADECAALLQDFFRARR

SEQ ID NO: 104 - protein, 142 aa, synthetic construct
MREALAEARAAAAAGEVPIGAVVVRDGAVLARAGNRTVRDCDPTAHAEIVALREAARALGNHRLTGCTLYVTVEPCAMCAGAISWARVARLVYGAADPKGGAVRHGPRLFEQPTCHHAPEVVDGVCAEEAAALLRDFFRARR

SEQ ID NO: 105 - protein, 148 aa, synthetic construct
MDDEALMGLALDEARAAAAAGEAPIGAVVARGGEVLAAAGNRTLRDCDPTAHAEILALREAARKLGNYRLTGCTLYATLEPCAMCAGAISHARIARLVYGADDPKGGAVRHGPRFFEQPTCHHRPEVAGGVGAAEAGALLRDFFRARR

SEQ ID NO: 106 - protein, 148 aa, synthetic construct
MDDEALMGLALDEARAAAAAGEAPIGAVVARGGEVLAAAGNRTLRDCDPTAHAEVLALREAARRLGNYRLTGCTLYVTLEPCAMCAGAISHARVARLVYGAADPKGGAVAHGPRFFEQPTCHHRPEVTGGVGAAEAGALLRDFFRARR

SEQ ID NO: 107 - protein, 164 aa, synthetic construct
MSDEQYMRRALELARQAEAEGEVPVGAVLVRDGEVIAEGWNRSIGSHDATGHAEIMALRAAGQALQNYRLLDTTLYVTLEPCPMCAGALLHSRVKRVVFGAPDLKAGAAGTVLNLFESQASYHYADVESGLLEEECREQLQAFFKRRRKEIKALRQAQREAEEK

SEQ ID NO: 108 - protein, 142 aa, synthetic construct
MTEALAEARKAAAAGEVPVGAVLVRDGAVLARGGNRTIRDCDPTAHAEIVALRAAARALGNYRLPGTTLYVTLEPCAMCAGAMIHARVDRLVYGAADPRAGAAGSVLDVIGHPALNHRMAVEGGVLAEECGALLRDFFRARR

SEQ ID NO: 109 - protein, 148 aa, synthetic construct
MTDEYFMRQALREARRAYDEGEVPVGAVVVRDGRVIARGRNQVERLKDPTAHAEMLALTAAAAHLGSKRLEGCTLYVTLEPCAMCAGAIVLARVPRLVFGAFDPKAGACGTLYDIVRDRRLNHRVEVVSGVLEEESAALLREFFARLR

SEQ ID NO: 110 - protein, 142 aa, synthetic construct
MREALAEARAAAAAGEVPVGAVVVRDGAVLARAGNRTVRDADPTAHAEIVALRAAARALGNHRLDGCTLYVTLEPCAMCAGAIAQARVARLVYGAADPKGGAVAHGPRLFEQPTTHHRPEVVGGVLAEEAGALLRGFFAARR

SEQ ID NO: 111 - protein, 142 aa, synthetic construct
MREALAEARAAAAAGEVPVGAVVVRDGAVLARAGNASIRARDPTAHAEILALRAAARALGNHRLDGCTLYVTLEPCAMCAGAIAHARVARLVYGAADPKGGAVAHGPRFFEQPTCHHRPEVTGGVLAAEAGALLRDFFRARR

SEQ ID NO: 112 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPEVVGGVGAAEAGALLREFFAARR

SEQ ID NO: 113 - protein, 142 aa, synthetic construct
MRDALAEARAAAAAGEVPVGAVVVRDGAILARAGNASIRARDPTAHAEILALRAAARALGNHRLTGCTLYVTLEPCAMCAGAIAHARIARLVYGAADPKGGAVAHGPRFFEQPTCHHRPEVTGGVLAEEAGALLRDFFRARR

SEQ ID NO: 114 - protein, 148 aa, synthetic construct
MDDAGFMRLALAEAEAAAAAGEVPVGAVVVRDGEVIARAGNRTVRDCDPTAHAEIVALREAARKLGNYRLPGCTLYVTLEPCAMCAGAMIHARLDRVVYGAADPKTGAAGSVLDLFADRRLNHHTAVVGGVLAEEAGALLRAFFAARR

SEQ ID NO: 115 - protein, 142 aa, synthetic construct
MREALAEARAAAAAGEVPVGAVVVRDGAVLARAGNASIRARDPTAHAEILALRAAARALGNHRLTGCTLYVTLEPCAMCAGAIAHARVARLVYGAADPKGGAVAHGPRFFEQPTCHHRPEVTGGVLAEEAGALLRAFFRARR

SEQ ID NO: 116 - protein, 142 aa, synthetic construct
MREALAEARAAAAAGEVPVGAVVVRDGAVLARAGNASIRARDPTAHAEMLALRAAARALGNHRLTGCTLYVTLEPCAMCAGAIAHARVARLVYGAADPKGGAVAHGPRFFEQPTCHHRPEVTGGVLAEEAGALLRDFFRARR

SEQ ID NO: 117 - protein, 142 aa, synthetic construct
MRAALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPEVVGGVGAAEAGALLRDFFRARR

SEQ ID NO: 118 - protein, 142 aa, synthetic construct
MREALAEARAAAAAGEVPVGAVVVRDGAVLARAGNASIRARDPTAHAEVLALRAAARALGNHRLTGCTLYVTLEPCAMCAGAIAHARLARLVYGAADPKGGAVEHGPRFFAQPTCHHRPEVTGGVGAAEAGALLRAFFAARR

SEQ ID NO: 119 - protein, 164 aa, synthetic construct
MSDEQFMRRAIELAKKGEELGEVPVGAVLVKDGEIIAEGWNQSISTHDATAHAEIMALRAAGEKLQNYRLLDTTLYVTLEPCPMCAGALLHSRVKRVVFGAPDLKAGAAGTVLNLFESQASYHYADVESGLLEEECREQLQAFFKRRRKEIKALKKAKKEAEQK

SEQ ID NO: 120 - protein, 143 aa, synthetic construct
MREALAEARAAAAAGEVPIGAVVVRDGAVLARAGNRTVRDCDPTAHAEIVALREAARALGNYRLDGCTLYVTLEPCAMCAGAMLHARLARVVYGAADPKTGAAGSVLDLFAERRLNHQTEVAGGVLAEECGALLRDFFRARRG

SEQ ID NO: 121 - protein, 142 aa, synthetic construct
MTEALAEARKAAALGEVPVGAVVVRDGAVIARGHNRTVTDTDPTAHAEIVALREAARALGNHRLTGCTLYVTLEPCAMCAGAIVHARLDRVVYGAADPKAGAAGSVLDVLGHPRLNHRPEVAGGVLAAESAALLRDFFRARR

SEQ ID NO: 122 - protein, 148 aa, synthetic construct
MTDEYFMRQALREARRAYDEGEVPVGAVVVRDGRVIARGRNQVERLKDPTAHAEMLALTAAAAHLGSKRLEGCTLYVTLEPCAMCAGAIVLARIPRLVFGAFDPKAGACGTLYDIVRDRRLNHRAEVVSGVLEEECGALLKEFFARLR

SEQ ID NO: 123 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGADDPKGGAVAHGPRFFAQPTCHHRPEVVGGVGEAEAAALLRGFFAARR

SEQ ID NO: 124 - protein, 142 aa, synthetic construct
MREALAEARAAAAAGEVPVGAVVVRDGAVLARAGNRTVRDADPTAHAELVAIRAAARALGSERLTGCTLYVTLEPCAMCAGAISHARVARLVYGAADPKGGAVAHGPRFFEQPTCHHRPEVTGGVLAEEAGALLRDFFRARR

SEQ ID NO: 125 - protein, 148 aa, synthetic construct
MTDEYFMRQALREARRAYEEDEVPVGAVVVREGRVIARGRNQVERLKDPTAHAEMIALTSAANYLGSKRLEGCTVYVTLEPCPMCAGALVLARVERLVFGAFDPKAGACGTLYDIPGDRRLNHRIEVVGGVLEEESAALLREFFRRRR

SEQ ID NO: 126 - protein, 142 aa, synthetic construct
MSEALAEARKAAALGEVPVGAVVVRDGAVIARGHNRTVADADPTAHAEIVALREAARALGNHRLTGCTLYVTLEPCAMCAGAIVHARLDRLVYGAADPKAGAAGSVLDVLGHPRLNHRPEVEGGVLAAESAALLRDFFRARR

SEQ ID NO: 127 - protein, 164 aa, synthetic construct
MSDEQYMRRALELARQAEQEGEVPVGAVLVKDGEVIAEGWNRSIGSHDATGHAEIMALRAAGEKLGNYRLLDTTLYVTLEPCPMCAGALLHSRVKRVVFGAPDLKAGAAGTVLNLFESQASYHYADVESGLLEEECREQLQAFFKRRRKEIKAARKAKREAEEK

SEQ ID NO: 128 - protein, 142 aa, synthetic construct
MREALAEARAAAAAGEVPVGAVVVRDGAVLARAGNASIRARDPTAHAEMLALRAAARALGNHRLTGCTLYVTLEPCAMCAGAIAHARVARLVYGAADPKGGAVAHGPRFFEQPTCHHRPEVTGGVLAEEAGALLRAFFRARR

SEQ ID NO: 129 - protein, 148 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGAADPKAGAAGSVLDVLGHPRLNHRMEVEGGVLAAECGALLRDFFRARR

SEQ ID NO: 130 - protein, 142 aa, synthetic construct
MRAALDEARAAAAAGEVPVGAVVVRDGAVLARAGNASIRARDPTAHAEILALRAAARALGNHRLTGCTLYVTLEPCAMCAGAIAHARVARLVYGAADPKGGAVEHGPRFFAQPTCHHRPEVTGGVLAAEAGALLRDFFRARR

SEQ ID NO: 131 - protein, 142 aa, synthetic construct
MTEALAEARKAAAAGEVPVGAVVVRDGEILARAHNRTVADHDPTAHAEILALREAARRLGNHRLTGCELYVTLEPCAMCAGAISHARIARLVYGAADPKGGAVEHGPRFFAQPTCHHRPEVVGGVGASEAAALLRDFFRARR

SEQ ID NO: 132 - protein, 142 aa, synthetic construct
MRAALAEARAAAAAGEVPVGAVVVRDGAVLARAGNASIRARDPTAHAEVLALRAAARALGNHRLDGCTLYVTLEPCAMCAGAIAHARVARLVYGAADPKGGAVAHGPRFFEQPTCHHRPEVTGGVLAEEAGALLRAFFRARR

SEQ ID NO: 133 - protein, 148 aa, synthetic construct
MDDAGFMRLALEEARAAAAAGEVPVGAVVVRDGEVLARAGNRTVRDCDPTAHAEVVALREAARKLGNHRLTGCTLYVTLEPCAMCAGAISHARVARLVYGADDPKGGAVAHGPRFFAQPTCHHRPEVAGGLLAGEAAALLRDFFAARR

SEQ ID NO: 134 - protein, 142 aa, synthetic construct
MRDALAEARAAAAAGEVPVGAVVVRDGAVLARAGNASIRARDPTAHAEILALRAAARALGNHRLTGCTLYVTLEPCAMCAGAIAHARIARLVYGAADPKGGAVAHGPRFFEQPTCHHRPEVTGGVLAEEAGALLREFFRARR

SEQ ID NO: 135 - protein, 142 aa, synthetic construct
MTEALAEARKAAAAGEVPVGAVLVRDGAVLARGGNRTIRDCDPTAHAEIVALRAAARALGNYRLPGTTLYVTLEPCAMCAGAMIHARVDRLVYGAADPRAGAAGSVFDVIRHPALNHRMEVEGGVLAEECGALLRDFFRARR

SEQ ID NO: 136 - protein, 142 aa, synthetic construct
MREALAEARAAAAAGEVPVGAVVVRDGAVLARAGNRTVRDADPTAHAEIVALREAARALGNHRLTGCTLYVTLEPCAMCAGAISHARVARLVYGADDPKGGAVAHGPRFFAQPTCHHRPEVVGGVGAEEAGALLRDFFRARR

SEQ ID NO: 137 - protein, 149 aa, synthetic construct
MTDEYFMRQALREARRAYDEGEVPVGAVVVRDGRVIARGRNQVERLKDPTAHAEMLALTAAAAHLGSKRLEGCTLYVTLEPCAMCAGAIVLARIPRLVFGAFDPKAGACGTLYDIVRDRRLNHRVEVVSGVLEEESAALLREFFARLRA

SEQ ID NO: 138 - protein, 163 aa, synthetic construct
MSDEQYMRRALELARQAEEEGEVPVGAVLVKDGEIVAEGWNRSIGSHDATGHAEIMALRAAGEKLENYRLLDTTLYVTLEPCPMCAGALLHSRVKRVVFGAPDLKAGAAGTVLNLFESQASYHYADVESGLLEEECREQLQAFFKRRRKEIKAQRDAEKAAAE

SEQ ID NO: 139 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPEVVGGVGAAEAGALLRAFFAARR

SEQ ID NO: 140 - protein, 142 aa, synthetic construct
MRDALAEARAAAAAGEVPVGAVVVRDGAILARAGNASIRARDPTAHAEVLALRAAARALGNHRLDGCTLYVTLEPCAMCAGAIAHARVARLVYGAADPKGGAVEHGPRFFAQPTCHHRPDVTGGVGAAEAAALLRDFFRARR

SEQ ID NO: 141 - protein, 142 aa, synthetic construct
MRDALAEARAAAAAGEVPVGAVVVRDGAVLARAGNRTVRDCDPTAHAEIVALRAAARALGNHRLDGCTLYVTLEPCAMCAGAIAQARVARLVYGADDPKGGAVAHGPRLFAQPTCHHRPEVVGGVGAEEAAALLRGFFAARR

SEQ ID NO: 142 - protein, 148 aa, synthetic construct
MTDEYFMRQALREARRAYDEDEVPVGAVVVRDGKVIARGRNQVERLKDPTAHAEMIALTSAANHLGSKRLEGCTVYVTLEPCAMCAGALVLARVDRLVFGAFDPKAGACGTLYDIPRDRRLNHRVEVVGGVLEEESAALLREFFRRRR

SEQ ID NO: 143 - protein, 148 aa, synthetic construct
MTDEYFMRQALREARRAYDEGEVPVGAVVVRDGKVIARGRNQVERLKDPTAHAEMIALTAAANHLGAKRLEGCTVYVTLEPCAMCAGALVLARVDRLVFGAFDPKAGACGTLYDIPRDARLNHRVEVVSGVLEEESAALLREFFARLR

SEQ ID NO: 144 - protein, 142 aa, synthetic construct
MSEALAEARKAAAAGEVPVGAVLVRDGAVLARGGNRTIRDCDPTAHAEIVALRAAARALGNYRLPGTTLYVTLEPCAMCAGAMIHARVDRLVYGAADPRAGAAGSVFDILRHPALNHRMEVEGGVLADECGALLRDFFRARR

SEQ ID NO: 145 - protein, 142 aa, synthetic construct
MRAALAEARAAAAAGEVPVGAVVVRDGAILARAGNASIRARDPTAHAEVLALRAAARALGNHRLTGCTLYVTLEPCAMCAGAIAHARIARLVYGAADPKGGAVAHGPRFFAQPTCHHRPEVTGGLGEAEAAALLRAFFAARR

SEQ ID NO: 146 - protein, 142 aa, synthetic construct
MRAALAEARAAAAAGEVPVGAVVVRDGAVLARAGNASIRARDPTAHAEVLALRAAARALGNHRLDGCTLYVTLEPCAMCAGAIAHARVARLVYGAADPKGGAVAHGPRFFEQPTCHHRPEVTGGVLAAEAGALLRDFFRARR

SEQ ID NO: 147 - protein, 142 aa, synthetic construct
MTEALAEARKAAALGEVPVGAVVVRDGAVIARGHNRTVADADPTAHAEIVALREAARALGNHRLTGCTLYVTLEPCAMCAGAIVHARLDRVVYGAADPKAGAAGSVLDVLGHPRLNHRPEVAGGVLAEECGALLRDFFRARR

SEQ ID NO: 148 - protein, 142 aa, synthetic construct
MREALAEARAAAAAGEVPVGAVVVRDGAVLARAGNRTVRDADPTAHAEIVALREAARALGNHRLGGCTLYVTLEPCAMCAGAIAQARVARLVYGAADPKGGAVAHGPRLFEQPTCHHRPEVVGGVLAEEAGALLRGFFAARR

SEQ ID NO: 149 - protein, 148 aa, synthetic construct
MTDEYFMRQALREARRAYEEGEVPVGAVVVRDGRVIARGRNQVERLKDPTAHAEMIALTAAAAHLGNKRLEGCTLYVTLEPCAMCAGAIVLARIPRLVFGAFDPKAGACGTLYDIVRDRRLNHRAEVVSGVLEEECGALLKEFFARLR

SEQ ID NO: 150 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGAADPKGGAVAHGPRLFAQPTCHHRPEVVGGVGEAEAAALLRDFFAARR

SEQ ID NO: 151 - protein, 162 aa, synthetic construct
MDDEFYMRRALELAALAEEHNEVPVGAVLVLNGEIIGEGWNRSIGHHDATAHAEIMALRQAGKKLENYRLLDTTLYVTLEPCPMCAGALLHSRVKRVVFGAPDLKAGAAGTVLNLFESQASYHYADVESGLLEQECREQLQAFFKRRRKEIKALKKAKREAE

SEQ ID NO: 152 - protein, 142 aa, synthetic construct
MREALAEARAAAAAGEVPVGAVVVRDGAILARAGNASIRARDPTAHAEVLALRAAARALGNHRLTGCTLYVTLEPCAMCAGAIAHARVARLVYGAADPKGGAVEHGPRFFAQPTCHHRPEVTGGVLAAEAGALLRDFFRARR

SEQ ID NO: 153 - protein, 148 aa, synthetic construct
MTDEYFMRQALREARRAYDEGEVPVGAVVVRDGRVIARGRNQVERLKDPTAHAEMIALTAAANHLGAKRLEGCTVYVTLEPCAMCAGALVLARVDRLVFGAFDPKAGACGTLYDIPRDPRLNHRVEVVSGVLAEESAALLREFFAARR

SEQ ID NO: 154 - protein, 148 aa, synthetic construct
MTDEYFMRQALREARRAYEEDEVPVGAVVVREGRVIARGRNQVERLKDPTAHAEMIALTAAANHLGSKRLEGCTVYVTLEPCPMCAGALVLARVRRLVFGAFDPKAGACGTLYDIPRDRRLNHRVEVVGGVLEEESAALLREFFRRRR

SEQ ID NO: 155 - protein, 162 aa, synthetic construct
MSDEQYMRRALELARQAEQEGEVPVGAVLVKDGEVVAEGWNRSIGSHDATGHAEIMALRAAGQVLQNYRLLDTTLYVTLEPCPMCAGALLHSRVKRVVYGAPDLKAGAAGTVLNLFESQASYHYADVESGLLEEECREQLQAFFKRRRKEIKAARQAKRDAE

SEQ ID NO: 156 - protein, 142 aa, synthetic construct
MREALAEARAAAAAGEVPVGAVVVRDGAVLARAGNRTVRDADPTAHAEVVALRAAARALGNHRLGGCTLYVTLEPCAMCAGAIAQARVARLVYGADDPKGGAVAHGPRLFAQPTCHHRPEVVGGVGAAEAAALLRDFFAARR

SEQ ID NO: 157 - protein, 149 aa, synthetic construct
MDDAGFMRLALAEARAAAAAGEVPVGAVVVRDGAVLAAAGNRTLRDCDPTAHAEVVALRAAARALGNYRLDGCTLYVTLEPCAMCAGAMLHARLARVVYGAADPKTGAAGSVLDLFAQPRLNHHTQVEGGVLAAECGALLQDFFRARRG

SEQ ID NO: 158 - protein, 164 aa, synthetic construct
MSDEQFMRRAIELARKGEELGEVPVGAVLVKDGEIIAEGWNQSISTHDATAHAEIMALRAAGEKLQNYRLLDTTLYVTLEPCPMCAGALLHSRVKRVVFGAPDLKAGAAGTVLNLFESQASYHYADVESGLLEEECREQLQAFFKRRRKEIKALKKAKREAEEK

SEQ ID NO: 159 - protein, 142 aa, synthetic construct
MREALAEARAAAAAGEVPVGAVVVRDGVVLARAGNRTVRDADPTAHAEVVALRAAARALGNHRLGGCTLYVTLEPCAMCAGAIAQARVARLVYGAADPKGGAVAHGPRLFEQPTCHHRPEVVGGVLAEEAGALLRDFFAARR

SEQ ID NO: 160 - protein, 142 aa, synthetic construct
MRDALAEARAAAAAGEVPVGAVVVRDGAVLARAGNASIRARDPTAHAEILALRAAARALGNHRLDGCTLYVTLEPCAMCAGAIAHARIARLVYGAADPKGGAVEHGPRFFAQPTCHHRPEVVGGVGAAEAGALLRDFFRARR

SEQ ID NO: 161 - protein, 148 aa, synthetic construct
MSDEDYMRLALEEARAAAAAGEVPVGAVVVRDGAVLARAGNRTVRDCDPTAHAEIVALREAARALGNHRLTGCTLYVTLEPCAMCAGAISHARVARLVYGADDPKGGAVAHGPRFFAQPTCHHRPEVTGGVLAEEAGALLRGFFAARR

SEQ ID NO: 162 - protein, 148 aa, synthetic construct
MTDEYFMRQALREARRAYDEDEVPVGAVVVRDGRVIARGRNQVERLKDPTAHAEMIALTSAANYLGSKRLEGCTVYVTLEPCAMCAGALVLARVDRLVFGAFDPKAGACGTLYDIPGDRRLNHRVAVTGGVLEEESAALLREFFRERR

SEQ ID NO: 163 - protein, 164 aa, synthetic construct
MSDEQYMRRALELARQAEQEGEVPVGAVLVKDGEIVAEGWNRSIGSHDATAHAEIETLRKAGQALENYRLLDTTLYVTLEPCPMCAGALLHSRVKRVVFGAPDLKAGAAGTVLNLFESQASYHYAEVESGLLEEECREQLQAFFKRRRKEIKALRQAEKEAAEK

SEQ ID NO: 164 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVAHGPRFFEQPTCHHRPEVTGGVLAEEAGALLRDFFRARR

SEQ ID NO: 165 - protein, 142 aa, synthetic construct
MRDALAEARAAAARGEVPVGAVVVRDGAVLARAGNASIAARDPTAHAEILALRAAARALGNHRLAGCTLYVTLEPCAMCAGAIAHARIARLVYGAADPKGGAVAHGPRFFEQPTCHHRPEVTGGVGAAEAGALLRDFFAARR

SEQ ID NO: 166 - protein, 142 aa, synthetic construct
MREALAEARAAAAAGEVPVGAVVVRDGAVLARAGNRTVRDADPTAHAELVAIRAAARALGSERLTGCTLYVTLEPCAMCAGAISHARVARLVYGADDPKGGAVAHGPRFFAQPTCHHRPDVTGGVGEAEAGALLRAFFAARR

SEQ ID NO: 167 - protein, 148 aa, synthetic construct
MDDAGFMRLALAEARKAAAAGEVPVGAVVVRDGAVLAAAGNRTLRDCDPTAHAEVVALREAARALGNHRLGGCDLYVTLEPCAMCAGAISHARIARLYYGADDPKGGAVAHGARVFDQPTCHHRPEVYGGIGAAEAAALLRGFFAARR

SEQ ID NO: 168 - protein, 148 aa, synthetic construct
MTDEYFMRQALREARRAYDEGEVPVGAVVVRDGRVIARGRNQVERLKDPTAHAEMLALTAAAAHLGSKRLEGCTLYVTLEPCAMCAGAIVLARLPRLVFGAFDPKAGACGTLYDIVRDRRLNHRVEVVSGVLEEECGALLKEFFARLR

SEQ ID NO: 169 - protein, 162 aa, synthetic construct
MSDEQYMRRALELARQAEQEGEVPVGAVLVKDGEVIAEGWNRSIGSHDATGHAEIMALRAAGEKLQNYRLLDTTLYVTLEPCPMCAGALLHSRVKRVVFGAPDLKAGAAGTVLNLFESQASYHYADVESGLLEEECREQLQAFFKRRRKEIKALRQAQKAAE

SEQ ID NO: 170 - protein, 142 aa, synthetic construct
MRDALAEARAAAAAGEVPVGAVVVRDGAILARAGNASIRARDPTAHAEILALRAAARALGNHRLTGCTLYVTLEPCAMCAGAIAHARVARLVYGAADPKGGAVAHGPRFFEQPTCHHRPEVTGGVGAAEAGALLRDFFAARR

SEQ ID NO: 171 - protein, 142 aa, synthetic construct
MTEALAEARKAAAAGEVPVGAVLVRDGAVLARGGNRTIRDCDPTAHAEIVVLREAARALGNYRLEGCTLYVTLEPCAMCAGAMIHARLDRLVYGADDPKAGAAGSVLDVLNHPRLNHQMEVTAGVLAEECGALLREFFRARR

SEQ ID NO: 172 - protein, 142 aa, synthetic construct
MREALAEARAAAAAGEVPVGAVVVRDGAVLARAGNRTVRDADPTAHAELVAIRAAARALGSERLTGCTLYVTLEPCAMCAGAISHARIARLVYGADDPKGGGVAHGARVFDHPQCHHRPEVVGGVGAAEAAALLRDFFAARR

SEQ ID NO: 173 - protein, 142 aa, synthetic construct
MREALAEARAAAAAGEVPVGAVVVRDGAVLARAGNRTVRDADPTAHAELVAIRAAAAALGSERLTGCTLYVTLEPCAMCAGAISHARIARLVYGAADPKGGAVAHGPRLFAQPTCHHRPEVVGGVGEAEAAALLRDFFAARR

SEQ ID NO: 174 - protein, 142 aa, synthetic construct
MREALAEARAAAAAGEVPVGAVVVRDGAVLARAGNASIRARDPTAHAEILALRAAARALGNHRLTGCTLYVTLEPCAMCAGAIAHARIARLVYGAADPKGGAVAHGPRFFEQPTCHHRPEVTGGVLAEEAGALLRAFFRARR

SEQ ID NO: 175 - protein, 142 aa, synthetic construct
MRDALAEARAAAARGEVPVGAVVVRDGAILARAGNATVAASDPTAHAEILALRAAARALGSQRLPGAVLYVTLEPCAMCAGAISHARIARLVYGAADPKGGAVAHGPRFFEQPTCHHRPEVTGGVLAEEAGALLRDFFRARR

SEQ ID NO: 176 - protein, 142 aa, synthetic construct
MRDALAEARAAAAAGEVPVGAVVVRDGAILARAGNASIRARDPTAHAEILALRAAARALGNHRLDGCTLYVTLEPCAMCAGAIAHARIARLVYGAADPKGGAVAHGPRLFAQPTCHHRPEVTGGVGAAEAAALLRDFFRARR

SEQ ID NO: 177 - protein, 148 aa, synthetic construct
MTDEYFMRQALREARRAYDEGEVPVGAVVVRDGRVIARGRNQVERLKDPTAHAEMIALTAAAAHLGNKRLEGCTLYVTLEPCAMCAGALVLARVPRLVFGAFDPKAGACGTLYDIVRDRRLNHRVEVVSGVLEEESAALLKEFFARLR

SEQ ID NO: 178 - protein, 148 aa, synthetic construct
MDDAGFMRLALEEARAAAAAGEVPVGAVVVRDGEVLARAGNRTVRDCDPTAHAEVVALREAARKLGNHRVTGATLYVTLEPCAMCAGAISLARVARLVYAADDPKGGGVAHGARVFDQPTCHHRPEVVSGVLAEESAALLRGFFAARR

SEQ ID NO: 179 - protein, 148 aa, synthetic construct
MTDEYFMRQALREARRAYDEDEVPVGAVVVREGKVIARGRNQVERLKDPTAHAEMIALTSAANHLGSKRLEGCTVYVTLEPCAMCAGALVLARVERLVFGAFDPKAGACGTLYNIPADRRLNHRVEVVGGVLEEESAALLREFFRKRR

SEQ ID NO: 180 - protein, 162 aa, synthetic construct
MDDEAWMRRAIALAHQAEAEGEVPVGAVLVKDGEVVAEGWNRSIGSHDATAHAEIETLRKAGQALENYRLLDTTLYVTLEPCPMCAGALLHSRVKRVVFGAPDLKAGAAGTVLNLFESQASYHYADVESGLLEEECREQLQAFFKRRRKEIKALRQAQREAE

SEQ ID NO: 181 - protein, 148 aa, synthetic construct
MDDAGFMRLALAEARAAAAAGEVPVGAVVVRDGAVLAAAGNRTLRDCDPTAHAEVVALRAAARALGNHRLEGCTLYVTLEPCAMCAGAALQARLARLVYGAAEPKTGAAGSVLDVFANPALNHHTAVTGGVLAAEAAALLRDFFAARR

SEQ ID NO: 182 - protein, 142 aa, synthetic construct
MTEALAEARKAAAAGEVPVGAVLVRDGAVLARGGNRTIRDCDPTAHAEIVALRAAARAAGNYRLPGATLYVTLEPCAMCAGAMIHARVDRLVYGAADPRAGAAGSVFDVVRHPALNHRMAVEGGVLAEECGALLRDFFRARR

SEQ ID NO: 183 - protein, 164 aa, synthetic construct
MTEKDKFFMQRAIELAKLAEENGEVPVGAVLVKDGEIIAEGWNQSIGSHDATAHAEIMALRQAGKVLENYRLLDTTLYVTLEPCPMCAGALLHSRVKRIVFGAPDLKAGAAGTVLNLFESQASYHYADVESGLLEEECREQLQAFFKRRRKEIKALKKAKKEAE

SEQ ID NO: 184 - protein, 148 aa, synthetic construct
MTDEYFMRQALREARRAYDEDEVPVGAVVVRDGKVIARGRNQVERLKDPTAHAEMIALTSAANYLGSKRLEGCTVYVTLEPCPMCAGALVLARVERLVFGAFDPKAGACGTLYDIPGDRRLNHRLEVVGGVLEEESAALLREFFRRRR

SEQ ID NO: 185 - protein, 142 aa, synthetic construct
MREALAEARAAAAAGEVPVGAVVVRDGAVLARAGNASIRARDPTAHAEMLALRAAARALGNHRLDGCTLYVTLEPCAMCAGAIAHARVARLVYGAADPKGGAVAHGPRFFEQPTCHHRPEVTGGVLAAEAGALLRDFFRARR

SEQ ID NO: 186 - protein, 148 aa, synthetic construct
MTDEYFMRQALREARRAYDEGEVPVGAVVVRDGRVIARGRNQVERLKDPTAHAEMIALTAAAAHLGNKRLEGCTLYVTLEPCAMCAGAIVLARIPRLVFGAFDPKAGACGTLYDIVRDRRLNHRAEVVSGVLEEECGALLKEFFARLR

SEQ ID NO: 187 - protein, 164 aa, synthetic construct
MSDEQYMRRALELARQAEQAGEVPVGAVLVKDGEIIAEGWNQSISSHDATAHAEIMALRAAGQALENYRLLDTTLYVTLEPCPMCAGALLHSRVKRVVFGAPDLKAGAAGTVLNLFESQASYHYAEVESGLLEEECREQLQAFFKRRRKEIKALKQAKREAEQK

SEQ ID NO: 188 - protein, 149 aa, synthetic construct
MDDAGFMRLALAEARAAAAAGEVPVGAVVVRDGAVLAAAGNRTLRDCDPTAHAEVVALRAAARALGNYRLDGCTLYVTLEPCAMCAGAMLHARLARVVYGAADPKTGAAGSVLDLFAEPRLNHHTRVEGGVLAAECGALLRDFFRARRG

SEQ ID NO: 189 - protein, 148 aa, synthetic construct
MDDAGFMRLALAEARAAAAAGEVPVGAVVVRDGAVLAAAGNRTLRDRDPTAHAEMVAIRAAAAALGQERLTDCDLYVTLEPCAMCAGAISFARIRRLYFGAADPKGGAVEHGPRFFAQPTCHHAPEVYGGIGEGEAAALLRDFFRARR

SEQ ID NO: 190 - protein, 142 aa, synthetic construct
MREALAEARAAAAAGEVPVGAVVVRDGAVLARAGNRTVRDADPTAHAEVVALRAAARALGNHRLDGCTLYVTLEPCAMCAGAIAQARVARLVYGAADPKGGAVAHGPRLFEQPTTHHRPEVVGGVLAEEAAALLRGFFAARR

SEQ ID NO: 191 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGAADPKGGAVAHGARVFDQPQTHHRPEVTGGVLAEEAGALLRAFFAARR

SEQ ID NO: 192 - protein, 148 aa, synthetic construct
MDDAGFMRLALAEARRAAEAGEVPVGAVVVRGGEVLAAAGNRTLRDCDPTAHAEIVALREAARRVGNYRLADCDLYVTLEPCAMCAGAIVHARVRRLVYGADDPKAGAVRSALRVLDAPALNHRVEVTAGVLAEECGALLRDFFRARR

SEQ ID NO: 193 - protein, 142 aa, synthetic construct
MTEALAEARKAAALGEVPVGAVVVRDGAVIARGHNRTVTDADPTAHAEIVALREAARALGNHRLTGCTLYVTLEPCAMCAGAMVHARLDRLVYGAADPKAGAAGSVLDVLGHPRLNHQMEVTAGVLADESAALLRDFFRARR

SEQ ID NO: 194 - protein, 142 aa, synthetic construct
MREALAEARAAAAAGEVPVGAVVVRDGAVLARAGNASIRARDPTAHAEMLALRAAARALGNHRLDGCTLYVTLEPCAMCAGAIAHARIARLVYGAADPKGGAVAHGPRFFEQPTCHHRPEVVGGVGAAEAGALLRDFFAARR

SEQ ID NO: 195 - protein, 142 aa, synthetic construct
MTEALAEARKAAALGEVPVGAVVVRDGAVIARGHNRTVTDTDPTAHAEIVALREAARALGNHRLTGCTLYVTLEPCAMCAGAIVHARLDRVVYGAADPKAGAAGSVLDVLGHPRLNHRPEVEGGVLGEECGALLRDFFRARR

SEQ ID NO: 196 - protein, 142 aa, synthetic construct
MREALAEARAAAAAGEVPVGAVVVRDGAVLARAGNRTVRDADPTAHAEVVALREAARALGNHRLGGCTLYVTLEPCAMCAGAISHARVARLVYGADDPKGGAVRHGARVFDQPTCHHRPEVVGGVLAEESAALLRGFFAARR

SEQ ID NO: 197 - protein, 142 aa, synthetic construct
MREALAEARAAAAAGEVPVGAVVVRDGAVLARAGNASIRARDPTAHAEILALRAAARALGNHRLDGCTLYVTLEPCAMCAGAIAHARVARLVYGAADPKGGAVEHGPRFFAQPTCHHRPEVVGGVGAAEAGALLRAFFAARR

SEQ ID NO: 198 - protein, 148 aa, synthetic construct
MDDAGFMRLALAEARAAAAAGEVPVGAVVVRDGAVLAAAGNRTLRDCDPTAHAEVVALRAAARALGNHRLGGCDLYVTLEPCAMCAGAISHARIARLYYGADDPKGGAVAHGARVFAHPQCHHRPEVYGGIGAAEAAALLRDFFAARR

SEQ ID NO: 199 - protein, 148 aa, synthetic construct
MTDEYFMRQALREARRAYDEDEVPVGAVVVRDGKVIARGRNQVERLKDPTAHAEMIALTSAANHLGSKRLEGCTVYVTLEPCPMCAGALVLARVERLVFGAFDPKAGACGTLYDIPGDRRLNHRVEVVGGVLEEESAALLREFFRRRR

SEQ ID NO: 200 - protein, 162 aa, synthetic construct
MDDEFYMRRALELAALAEEHNEVPVGAVLVLNGEIIGEGWNRSIGHHDATAHAEIMALRQAGKKLENYRLLDTTLYVTLEPCPMCAGALLHSRVKRVVFGAPDLKAGAAGTVLNLFESQASYHYADVESGLLEQECREQLQAFFKRRRKEKKALKQAQKEAE

SEQ ID NO: 201 - protein, 142 aa, synthetic construct
MRDALAEARAAAAAGEVPVGAVVVRDGAILARAGNASIRARDPTAHAEVLALRAAARALGNHRLDGCTLYVTLEPCAMCAGAIAHARIARLVYGAADPKGGAVEHGPRFFAQPTCHHRPEVVGGVGAAEAAALLRDFFRARR

SEQ ID NO: 202 - protein, 142 aa, synthetic construct
MRAALDEARAAAAAGEVPVGAVVVRDGAILARAGNRTVRDCDPTAHAEIVALREAARALGNHRLTGCTLYVTLEPCAMCAGAISHARVARLVYGADDPKGGAVAHGPRFFAQPTCHHRPEVVGGVGAGEAAALLRDFFAARR

SEQ ID NO: 203 - protein, 142 aa, synthetic construct
MREALAEARAAAAAGEVPVGAVVVRDGAILARAGNASIRARDPTAHAEVLALRAAARALGNHRLTGCTLYVTLEPCAMCAGAIAHARIARLVYGAADPKGGAVAHGPRFFEQPTCHHRPEVTGGVLAEEAGALLRAFFRARR

SEQ ID NO: 204 - protein, 142 aa, synthetic construct
MRDALAEARAAAARGEVPVGAVVVRDGAVLARAGNATVAASDPTAHAEILALRAAARAAGNHRLPGAVLYVTLEPCAMCAGAISHARIARLVYGAADPKGGAVAHGPRFFEQPTCHHRPEVTGGVLAEEAGALLRDFFRARR

SEQ ID NO: 205 - protein, 142 aa, synthetic construct
MREALAEARAAAAAGEVPVGAVVVRDGAILARAGNRTVRDADPTAHAEIVALREAARALGNHRLGGCTLYVTLEPCAMCAGAISQARVARLVYGADDPKGGAVAHGPRFFAQPTCHHRPEVAGGLGEAEAAALLRDFFAARR

SEQ ID NO: 206 - protein, 142 aa, synthetic construct
MTEALAEARKAAAAGEVPVGAVLVRDGAVLARGGNRTIRDCDPTAHAEIVALRAAARALGNYRLPGTTLYVTLEPCAMCAGAMIHARVARLVYGAADPKTGAAGSVFDVLRHPALNHRMEVEGGVLAEECGALLRDFFRARR

SEQ ID NO: 207 - protein, 166 aa, synthetic construct
MTEKDKFFMQRAIELAKKGEENGEVPVGAVLVKDGEIIAEGWNQSIGSHDATAHAEIMTLREAGKVLQNYRLLDTTLYVTLEPCPMCAGALLHSRVKRVVFGAPDLKAGAAGTVLNLFESQASYHYADVESGLLEEECREQLQAFFKRRRKEKKALKQAKREAEQK

SEQ ID NO: 208 - protein, 148 aa, synthetic construct
MTDEDYMRLALEEARAAAAAGEVPIGAVVVRDGEVLARAGNRTVRDCDPTAHAEIVALREAARALGNYRLTGCTLYVTIEPCAMCAGAMIHARVDRLVYGAADPKAGAVRSTLRVLDHPALNHRVEVTAGVLADECAALLQDFFRSRR

SEQ ID NO: 209 - protein, 142 aa, synthetic construct
MRDALAEARAAAAAGEVPVGAVVVRDGAVLARAGNRTVRDADPTAHAELVAIRAAARALGSERLTGCTLYVTLEPCAMCAGAISHARIARLVYGAADPKGGAVAHGPRFFAQPTCHHRPDVTGGVGEAEAGALLRDFFAARR

SEQ ID NO: 210 - protein, 142 aa, synthetic construct
MREALAEARAAAAAGEVPIGAVVVRDGAVLARAGNRTVRDCDPTAHAEVVALREAARALGNHRLTGCTLYVTVEPCAMCAGAISHARVARLVYGADDPKGGAVRHGPRFFEQPTCHHRPEVVGGVGAEEAAALLRDFFRARR

SEQ ID NO: 211 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVAHGPRFFEQPTCHHRPEVTGGVLAEEAGALLRAFFAARR

SEQ ID NO: 212 - protein, 148 aa, synthetic construct
MTDEYFMRQALREARRAYDEGEVPVGAVVVRDGRVIARGRNQVERLKDPTAHAEMLALTAAAAHLGSKRLEGCTLYVTLEPCAMCAGAIVLARVPRLVFGAFDPKAGACGTLYDIPRDRRLNHRAEVVGGVLEEESAALLREFFAARR

SEQ ID NO: 213 - protein, 142 aa, synthetic construct
MREALAEARAAAAAGEVPVGAVVVRDGAVLARAGNRTVRDADPTAHAEIVALREAARALGNHRLGGCTLYVTLEPCAMCAGAIAQARVARLVYGADDPKGGAVAHGPRFFAQPTCHHRPEVVGGVGAAEAGALLRGFFAARR

SEQ ID NO: 214 - protein, 162 aa, synthetic construct
MDDEAWMRRAIALAHKAEQEGEVPVGAVLVKDGEVIAEGWNRSIGHHDATAHAEIETLRKAGQVLENYRLLDTTLYVTLEPCPMCAGALLHSRVKRIVFGAPDLKAGAAGTVLNLFESQASYHYADVESGLLEEECREQLQAFFKRRRKEIKALRQAQKAAE

SEQ ID NO: 215 - protein, 142 aa, synthetic construct
MREALAEARAAAAAGEVPVGAVVVRDGAVLARAGNRTVRDADPTAHAEIVALREAARALGNHRLGGCTLYVTLEPCAMCAGAIAQARVARLVYGAADPKGGAVAHGPRLFAQPTCHHRPEVVGGVGEAEAAALLRGFFAARR

SEQ ID NO: 216 - protein, 142 aa, synthetic construct
MRDALAEARAAAAAGEVPVGAVVVRDGAILARAGNASIRARDPTAHAEILALRAAARALGNHRLTGCTLYVTLEPCAMCAGAISHARIARLVYGAADPKGGAVAHGPRFFEQPTCHHRPEVTGGVLAEEAGALLRAFFRARR

SEQ ID NO: 217 - protein, 163 aa, synthetic construct
MSDEQYMRRAIELARQAEQEGEVPVGAVLVKDGEIIAEGWNRSIGSHDATAHAEIETLRKAGQVLENYRLLDTTLYVTLEPCPMCAGALLHSRVKRIVYGAPDLKAGAAGTVLNLFESQASYHYADVESGLLEEECREQLQAFFKRRRKEIKAARKAKREAEQ

SEQ ID NO: 218 - protein, 148 aa, synthetic construct
MDDAGFMRLALAEARAAAAAGEVPVGAVVVRDGEVLAAAGNRTLRDCDPTAHAEVVALRAAARALGNHRLDGCDLYVTLEPCAMCAGAISHARIRRLYYGADDPKGGAVDNGVRFFASPTCHHAPEVYSGLAESEAAALLRDFFRERR

SEQ ID NO: 219 - protein, 142 aa, synthetic construct
MREALAEARAAAAAGEVPVGAVVVRDGAVLARAGNRTVRDADPTAHAEVVALRAAARALGNHRLDGCTLYVTLEPCAMCAGAIAQARVARLVYGAADPKGGAVAHGPRLFEQPTCHHRPEVVGGVGEAEAAALLRGFFAARR

SEQ ID NO: 220 - protein, 148 aa, synthetic construct
MTDEYFMRQALREARRAYDEDEVPVGAVVVREGKVIARGRNQVERLKDPTAHAEMIALTSAANYLGSKRLEGCTVYVTLEPCPMCAGALVLARVERLVFGAFDPKAGACGTLYDIPGDRRLNHRVEVVGGVLEEESAALLREFFRRRR

SEQ ID NO: 221 - protein, 142 aa, synthetic construct
MREALAEARAAAAAGEVPVGAVVVRDGAILARAGNASIRARDPTAHAEILALRAAARALGNHRLTGCTLYVTLEPCAMCAGAIAHARIARLVYGAADPKGGAVAHGPRFFEQPTCHHRPEVTGGLGEAEAAALLRDFFRARR

SEQ ID NO: 222 - protein, 148 aa, synthetic construct
MTDEYFMRQALREARRAYEEDEVPVGAVVVREGRVIARGRNQVERLKDPTAHAEMIALTAAANHLGSKRLEGCTVYVTLEPCPMCAGALVLARVERLVFGAFDPKAGACGTLYDIPGDRRLNHRVEVVGGVLEEEAAALLREFFRRRR

SEQ ID NO: 223 - protein, 149 aa, synthetic construct
MDDAGFMRLALAEARAAAAAGEVPVGAVVVRDGAVLAAAGNRTLRDCDPTAHAEVVALRAAARALGNYRLDGCTLYVTLEPCAMCSGAMLHARLARVVYGAADPKTGAAGSVLDLFAEPRLNHHTRVEGGVLAGECGALLADFFRGRRG

SEQ ID NO: 224 - protein, 147 aa, synthetic construct
MDDAGFMRLALAEARAAAAAGEVPVGAVVVRDGAVLAAAGNRTLRDCDPTAHAEVVALRAAARALGNHRLDGCDLYVTLEPCAMCAGAIAHARIRRLYYGAADPKGGAVEHGARVFDQPTCHHRPEVYGGIGEAEAAALLRDFFRDR

SEQ ID NO: 225 - protein, 162 aa, synthetic construct
MSDEQYMRRALELARQAEQEGEVPVGAVLVKDGEIIAEGWNRSIGSHDATGHAEIMALRAAGEKLQNYRLLDTTLYVTLEPCPMCAGALLHSRVKRVVFGAPDLKAGAAGTVLNLFESQASYHYADVESGLLEEECREQLQAFFKRRRKEIKALRQAQKAAE

SEQ ID NO: 226 - protein, 142 aa, synthetic construct
MREALAEARAAAAAGEVPVGAVVVRDGAVLARAGNASIRARDPTAHAEVLALRAAARALGNHRLDGCTLYVTLEPCAMCAGAIAHARVARLVYGAADPKGGAVEHGPRFFAQPTCHHRPDVTGGVGAAEAAALLRDFFRARR

SEQ ID NO: 227 - protein, 142 aa, synthetic construct
MREALAEARAAAAAGEVPVGAVVVRDGAILARAGNRTVRDADPTAHAEIVALRAAARALGNHRLDGCTLYVTLEPCAMCAGAAVLARVARLVFGADDPKGGAVRTGVRLFDAPTCHHRPEVTGGVLAEEAGALLREFFAARR

SEQ ID NO: 228 - protein, 142 aa, synthetic construct
MREALAEARAAAAAGEVPVGAVVVRDGAVLARAGNRTVRDADPTAHAELVAIRAAARALGSERLTGCTLYVTLEPCAMCAGAISHARIARLVYGAADPKGGAVAHGPRFFAQPTCHHRPEVTGGVGEAEAAALLRGFFAARR

SEQ ID NO: 229 - protein, 149 aa, synthetic construct
MTDEDYMRLALEEARAAAAAGEVPVGAVVVRDGEVIARARNAPVSACDPTAHAEILALREAARRLGNYRLDGCTLYVTLEPCAMCSGAMLHARLARVVYGAADPKTGAAGSVLDLFAEPRLNHHTAVEGGVLAAECGALLRDFFRARRG

SEQ ID NO: 230 - protein, 163 aa, synthetic construct
MSDEQYMRRALELARQAEAEGEVPVGAVLVKDGEVVAEGWNRSIGSHDATGHAEIMALRAAGQALENYRLLDTTLYVTLEPCPMCAGALLHSRVKRVVFGAPDLKAGAAGTVLNLFESQASYHYAEVESGLLEEECREQLQAFFKRRRKEIKAARKAAKAAAE

SEQ ID NO: 231 - protein, 142 aa, synthetic construct
MREALAEARAAAAAGEVPVGAVVVRDGAVLARAGNASIRAHDPTAHAEVLALRAAARALGNHRLTGCTLYVTLEPCAMCAGAIAHARVARLVYGAADPKGGAVAHGPRFFEQPTCHHRPEVTGGVLAEEAGALLREFFRARR

SEQ ID NO: 232 - protein, 142 aa, synthetic construct
MREALAEARAAAAAGEVPVGAVVVRDGAVLARAGNRTVRDADPTAHAEVVALRAAARALGNHRLDGCTLYVTLEPCAMCAGAIAQARVARLVYGAADPKGGAVAHGPRLFEQPTCHHRPEVVGGVGEAEAAALLRAFFAARR

SEQ ID NO: 233 - protein, 142 aa, synthetic construct
MTEALAEARKAAALGEVPVGAVVVRDGAVIARGHNRTIADSDPTAHAEIVALREAARVLGNYRLTGCTLYVTVEPCAMCAGAMVHARLDRLVYGAADPKAGAAGSVLDVLGHPRLNHQMEVTGGVLAEECAALLRDFFRARR

SEQ ID NO: 234 - protein, 162 aa, synthetic construct
MDDEFYMRRALELAALAEEHNEVPVGAVLVLNGEIIGEGWNRSIGHHDATAHAEIMALRQAGKVLQNYRLLDTTLYVTLEPCPMCAGALVHSRVKRVVYGTPDLKTGAAGSVMNLLSYEGVNHHVEVTSGVLAEECREQLQAFFRRRRAEKKALKKAQREAE

SEQ ID NO: 235 - protein, 163 aa, synthetic construct
MDDEAWMRRAIALAHQAEAEGEVPVGAVLVKDGEVIAEGWNRSIGHHDATAHAEIETLRQAGQVLQNYRLLDTTLYVTLEPCPMCAGALLHSRVKRVVFGAPDLKAGAAGTVLNLFESQASYHYADVESGLLEQECREQLQAFFKRRRKEIKAQRDAQRAAQE

SEQ ID NO: 236 - protein, 142 aa, synthetic construct
MREALAEARAAAAAGEVPVGAVVVRDGAVLARAGNASIRARDPTAHAEILALRAAARALGNHRLTGCTLYVTLEPCAMCAGAISHARIARLVYGAADPKGGAVAHGPRFFEQPTCHHRPDVTGGVLAAEAGALLRDFFRARR

SEQ ID NO: 237 - protein, 148 aa, synthetic construct
MDDAGFMRLALEEARAAAAAGEVPVGAVVVRDGAVLARAGNRTLRDRDPTAHAEVVALRAAARALGSERLTGCDLYVTLEPCAMCAGAISFARIRRLYFGAADPKGGAVENGVRFFASPTCHHAPEVYGGLAESEAAALLRDFFRARR

SEQ ID NO: 238 - protein, 148 aa, synthetic construct
MDDAGFMRLALAEARAAAAAGEVPVGAVVVRDGAVLAAAGNRTLRDCDPTAHAEVVALRAAARALGNHRLGGCDLYVTLEPCAMCAGAISHARIARLYYGADDPKGGAVAHGARVFDQPTCHHRPEVYGGIGAAEAAALLRDFFRARR

SEQ ID NO: 239 - protein, 142 aa, synthetic construct
MREALAEARAAAAAGEVPVGAVVVRDGAVLARAGNASIRARDPTAHAEILALRAAARALGNHRLDGCTLYVTLEPCAMCAGAIAHARVARLVYGAADPKGGAVEHGPRFFAQPTCHHRPEVTGGVLAAEAGALLRDFFRARR

SEQ ID NO: 240 - protein, 142 aa, synthetic construct
MREALAEARAAAAAGEVPVGAVVVRDGAVLARAGNASIRARDPTAHAEILALRAAARALGNHRLDGCTLYVTLEPCAMCAGAIAHARIARLVYGAADPKGGAVAHGPRFFAQPTCHHRPDVTGGVLAAEAGALLRDFFRARR

SEQ ID NO: 241 - protein, 142 aa, synthetic construct
MREALAEARAAAAAGEVPVGAVVVRDGAVLARAGNASIRARDPTAHAEVLALRAAARALGNHRLTGCTLYVTLEPCAMCAGAIAHARVARLVYGAADPKGGAVAHGPRFFEQPTCHHRPEVTGGVLAEEAGALLRGFFRARR

SEQ ID NO: 242 - protein, 149 aa, synthetic construct
DDDDRRWMREALAEARAAADAGEVPVGAVVVRDGVLLARAGNASIRDRDPTAHAEMLALRAAARALGNHRLTGCTLYVTLEPCAMCAGAIAHARIARLVYGASDPKAGAVEHGPRLFAQPTIHHRPEVTAGVLAEECGALLRDFFRARR

SEQ ID NO: 243 - protein, 149 aa, synthetic construct
DPTDLAFMREALAEARAAAEAGEVPVGAVVVCDGRIVARAHNRPIALSDPTAHAEILALREAARALGNYRLTGCTLYATLEPCAMCAGAILHARIARLVYGAADPKAGACGSVLDVMNHPRLNHRVEVTGGVLAEECGALLSGFFRARR

SEQ ID NO: 244 - protein, 148 aa, synthetic construct
DDDHAWMGAALAEARAAAEAGEVPVGAVLVADGRVLARAGNRTIRDRDPTAHAEMLALRAAARALGNHRLTGTTLYVTLEPCAMCAGAISLARVARLVYAASDPKGGAVEHGPRFFAQPTCHHRPEVVGGVGEGEAGALLRDFFRARR

SEQ ID NO: 245 - protein, 150 aa, synthetic construct
MTAEDDRFMRLALAEARAAAEAGEVPVGAVVVAGGRVVARAHNRPIALHDPTAHAEVLALRAAARELGNYRLTGCTLYATLEPCAMCAGAVLHARIARLVYGAADPKAGACGSVLAVMNHPRLNHRVEVTGGVLAEECGALLREFFRARR

SEQ ID NO: 246 - protein, 149 aa, synthetic construct
MDSDLAFMREALAEARAAAEAGEVPVGAVVVHEGKIVARAANRMRTDRDPTAHAELLALRAAARALETTRLTGCTLYVTLEPCAMCAGAISHARVARLVYGASDPKGGAVEHGPRFFAQPTCHHRPEVVGGVGEAEAGALLRGFFRARR

SEQ ID NO: 247 - protein, 149 aa, synthetic construct
DPDDARWMREALAEARAAAEAGEVPVGAVVVRDGRLLARAGNRTIRDRDPTAHAEMLALRAAARALGNHRLEGCTLYVTLEPCAMCAGAMVQARVARLVYGAADPKAGAAGSVLDVLGDPRLNHRVEVTGGVLAEECGALLREFFRARR

SEQ ID NO: 248 - protein, 150 aa, synthetic construct
MDSDLEFMREALAEARAAAEAGEVPVGAVVVRDGVILARAGNRPIRDHDPTAHAEILALREAARAVGNYRLTGCTLYVTLEPCAMCAGAILHARVERLVYGAADPKAGAAGSVLDVFGNARLNHHTRVEGGVLAEECGALLSGFFRARRG

SEQ ID NO: 249 - protein, 148 aa, synthetic construct
DPDAEFMRLALAEAEAAARAGEVPVGAVVVADGRVIARAGNRTIRDRDPTAHAEMLALRAAARALGSHRLTGCDLYVTLEPCAMCAGAISHARIRRLYYGAADAKGGAVEHGPRFFAQPTCHHRPEVYGGIGETAAAALLRDFFRARR

SEQ ID NO: 250 - protein, 148 aa, synthetic construct
DPDAAFMRAALAEARAAAEAGEVPVGAVVVAGGRIVARAHNRPIELNDPTAHAEILALRQAAAALGNYRLTGCTLYATLEPCAMCAGAILHARIERLVYGAADPKAGAVGSVLDVFANPRLNHRTQVTAGVLADECGALLREFFRARR

SEQ ID NO: 251 - protein, 150 aa, synthetic construct
DESSDEEFMRAALEEARAAAAEGEVPVGAVVVAGGRIVARAHNRPIALNDPTAHAEILALRQAAAALGNYRLTGCTLYATLEPCAMCAGAILHARIARVVYGAADPKAGACGSVLAVMNHPQLNHRAEVTGGVLAEECGALLSEFFRARR

SEQ ID NO: 252 - protein, 148 aa, synthetic construct
DDDHRFMRAALAEARAAAEAGEVPVGAVVVHGGEIIARAHNRPIALHDPTAHAEILALRAAARRLGNYRLTGCTLYATLEPCAMCAGAILHARIARLVYGAADPKAGACGSVLAVMNHPRLNHRVEVTAGVLADECGALLSEFFRARR

SEQ ID NO: 253 - protein, 148 aa, synthetic construct
MNDEDYMRAALEQARQAAAAGEVPVGAVVVCGGEIVARAHNRPISASDPTAHAEILALREAARKLGNYRLTGCTLYATLEPCAMCSGAILHARIARLVYGAADPKAGAVGSVLDVINHPRLNHRVEVTSGVLADECGALLKEFFRARR

SEQ ID NO: 254 - protein, 148 aa, synthetic construct
MNDEDYMRAALEQARQAAAAGEVPVGAVVVCDGKIVARAHNRPISANDPTAHAEILALREAARQLGNYRLTGCTLYATLEPCAMCSGAILHARIARLVYGAADPKAGACGSVLNVINHPQLNHQTEVTGGVLADECGALLKDFFRARR

SEQ ID NO: 255 - protein, 142 aa, synthetic construct
MRAALEEARRAAAAGEVPVGAVVVRDGAIVARAHNRPIALHDPTAHAEILALREAARALGNYRLTGCTLYATLEPCAMCAGAILHARIDRLVYGAADPKAGACGSVLSVMNHPRLNHRVEVTGGVLAEECGALLTGFFRARR

SEQ ID NO: 256 - protein, 149 aa, synthetic construct
MSEEDRFMRAALAEARAAAEAGEVPVGAVVVCGGRIVARAHNRPIALNDPTAHAEILALREAARELGNYRLTGCTLYATLEPCAMCAGAILHARIARLVYGARDPKAGAVESLYRLLDDTRLNHRVEAVGGVLAGECGALLSGFFRARR

SEQ ID NO: 257 - protein, 147 aa, synthetic construct
MDEEFMRLALAEARAAAEAGEVPVGAVVVAGGRVVARAHNRPVALHDPTAHAEVLALRAAARELGNYRLTGCTLYATLEPCAMCAGAILHARLDRVVYGAADPKAGAAGSVLDVFANPRLNHRTAVTGGVLAEECGALLREFFRARR

SEQ ID NO: 258 - protein, 150 aa, synthetic construct
MSEEDRFMRAALAEARAAAEAGEVPVGAVVVAGGRIVARAHNRPIALNDPTAHAEILALRAAARALGNYRLTGCTLYATLEPCAMCAGAILHARIARLVYGARDPKAGAVESLYRLLDDRRLNHRVEAVGGVLAAECGALLSGFFRARRG

SEQ ID NO: 259 - protein, 142 aa, synthetic construct
MREALAEARAAAAAGEVPVGAVVVRDGAVLARAGNRTLRDRDPTAHAEMLAIRAAARALGSERLTGCDLYVTLEPCAMCAGAISHARIARLYYGAADPKGGAVEHGPRFFAQPTCHHRPEVYGGIGEGEAAALLRGFFRARR

SEQ ID NO: 260 - protein, 142 aa, synthetic construct
MRAALEEARRAAAAGEVPVGAVVVRDGAIVARAHNRPIALHDPTAHAEILALREAARALGNYRLTGCTLYATLEPCAMCSGAILHARLDRVVYGAADPKTGAAGSVLDVFANPRLNHHAAVTGGVLAEECGALLSGFFRARR

SEQ ID NO: 261 - protein, 142 aa, synthetic construct
MREALAEARAAAAAGEVPVGAVVVRDGAIVARAHNRPIALHDPTAHAEVLALREAARALGNYRLTGCTLYATLEPCAMCAGAILHARLDRVVYGAADPKAGAAGSVLDVFANPRLNHRTAVTGGVLADECGALLSGFFRARR

SEQ ID NO: 262 - protein, 142 aa, synthetic construct
MRAALEEARRAAAAGEVPVGAVVVRDGAIVARAHNRPIALHDPTAHAEILALREAARALGNYRLTGCTLYATLEPCAMCAGAILHARLDRVVYGAADPKAGAAGSVLQVFDHPRLNHRTAVTGGVLAEECGALLRDFFRARR

SEQ ID NO: 263 - protein, 149 aa, synthetic construct
MDDDALMREALAEARAAAAAGEVPVGAVVARGGEIVARAANAPRALCDPTAHAEILALRAAARALGNYRLTGCTLYATLEPCAMCAGAILHARIARLVYAAPDPKAGACGSVLAVLNHPQLNHRVEVTAGLLADECGALLTDFFRARRG

SEQ ID NO: 264 - protein, 142 aa, synthetic construct
MRAALEEARRAAAAGEVPVGAVVVRDGAIVARAHNRPIALHDPTAHAEILALREAARALGNYRLTGCTLYATLEPCAMCAGAILHARIDRLVYGAADPKAGACGSVLSVMNHPQLNHRVEVTGGVLAEECGALLSGFFRARR

SEQ ID NO: 265 - protein, 149 aa, synthetic construct
MNDEDFMRAALAQAREAAAAGEVPVGAVVVCDGEIVARAHNRPIALNDPTAHAEILALREAARRLGNYRLTGCTLYATLEPCAMCAGAILHARIARLVYGAADPKAGAVGSVLDVLNHPRLNHRVEVTRGVLAEECGALLSEFFRARRR

SEQ ID NO: 266 - protein, 163 aa, synthetic construct
MDEEFMRRALELARQAEAAGEVPVGAVLVKDGEIVAEGWNQSIGRHDATAHAEIQVLRKAGQVLENYRLLDTTLYVTLEPCPMCAGALLHSRVKRIVYGAPDLKAGAAGTVLNLFEHQAAYHYADVEHGLLEEECREQLQAFFKRRRKEKKALKQAQREAEEK

SEQ ID NO: 267 - protein, 150 aa, synthetic construct
MSEEDRFMRAALAEARAAAEAGEVPVGAVVVAGGRIVARAHNRPIALNDPTAHAEILALREAARALGNYRLTGCTLYATLEPCAMCAGAILHARIARLVYGAADPKAGAVESLYRLLDDTRLNHRVEATGGVLAGECGALLSEFFRARRR

SEQ ID NO: 268 - protein, 150 aa, synthetic construct
MSEEDRFMRAALAEARAAAEAGEVPVGAVVVAGGRIIARAHNRPIALNDPTAHAEILALRAAARALGNYRLTGCTLYATLEPCAMCAGAILHARIARLVYGARDAKAGAVESVLRLFDHPALNHRVEAVGGVLADECGALLSEFFRARRA

SEQ ID NO: 269 - protein, 142 aa, synthetic construct
MREALAEARAAAAAGEVPVGAVVVRDGAIVARAHNRPIALHDPTAHAEILALREAARALGNYRLTGCTLYATLEPCAMCAGAILHARLDRVVYGAADPKAGAAGSVLDVFANPRLNHRTAVTGGVLADECGALLRDFFRARR

SEQ ID NO: 270 - protein, 148 aa, synthetic construct
MNDEDYMRAALEQARQAAAAGEVPVGAVVVCGGEIVARAHNRPISASDPTAHAEILALREAARKLGNYRLTGCTLYATLEPCAMCSGAILHARIARLVYGAADPKAGAVGSVLDVINHPRLNHRVEVTGGVLAGECGALLSGFFRARR

SEQ ID NO: 271 - protein, 142 aa, synthetic construct
MREALAEARAAAAAGEVPVGAVVVRDGAIVARAHNRPIALHDPTAHAEVLALRAAARELGNYRLTGCTLYATLEPCAMCAGAILHARLDRVVFGARDPKAGAAGSVLDVFANPRLNHRTAVTGGVLAEECGALLSGFFRARR

SEQ ID NO: 272 - protein, 148 aa, synthetic construct
MNDEDYMRAALEQARQAAAAGEVPVGAVVVCDGKIVARAHNRPISANDPTAHAEILALREAARQLGNYRLTGCTLYATLEPCAMCSGAILHARIARLVYGASDPKAGACGSVLNVINHPQLNHRVEVTGGVLADECGALLKDFFRARR

SEQ ID NO: 273 - protein, 149 aa, synthetic construct
MSEEDRFMRAALAEARAAAAEGEVPVGAVVVAGGRIVARAHNRPVALNDPTAHAEILALRAAARALGNYRLTGCTLYATVEPCAMCAGAILHARIARLVYGAADPKAGAVRSVLRVLDHPALNHRVEVTAGVLAEECAALLQEFFRARR

SEQ ID NO: 274 - protein, 148 aa, synthetic construct
MNDEDYMRAALEQARQAAAAGEVPVGAVVVCGGEIVARAHNRPISANDPTAHAEILALREAARKLGNYRLTGCTLYATLEPCAMCSGAILHARIARLVYGAADPKAGACGSVLNVINHPQLNHRVEVTGGILADECGALLKDFFRARR

SEQ ID NO: 275 - protein, 142 aa, synthetic construct
MRAALEEARRAAAAGEVPVGAVVVRDGAIVARAHNRPIALHDPTAHAEILALREAARRLGNYRLTGCTLYATLEPCAMCAGAILHARIDRLVYGAADPKAGACGSVLSVMNHPRLNHRVEVTSGVLAEECGALLTDFFRARR

SEQ ID NO: 276 - protein, 143 aa, synthetic construct
MRAALEEARRAAAAGEVPVGAVVVRDGEIVARAHNRPIALHDPTAHAEILALREAARRLGNYRLTGCTLYATLEPCAMCAGAILHARLDRVVYGAADPKAGACGSVLSVFDQPRLNHRTTVTGGVLAEECGALLSGFFRARRG

SEQ ID NO: 277 - protein, 142 aa, synthetic construct
MREALAEARAAAAAGEVPVGAVVVRDGAVLARAGNASIRARDPTAHAEVLALRAAARALGNHRLTGCTLYVTLEPCAMCAGAIAHARVARLVYGAADPKGGAVAHGPRFFEQPTCHHRPEVVGGVGAAEAGALLRDFFAARR

SEQ ID NO: 278 - protein, 148 aa, synthetic construct
MTDEYFMRQALREARRAYDEGEVPVGAVVVRDGRVIARGRNQVERLKDPTAHAEMIALTAAANYLGSKRLEGCTVYVTLEPCPMCAGALVLARVERLVFGAFDPKAGACGTLYDIVRDRRLNHRLEVVGGVLEEESAALLREFFEKRR

SEQ ID NO: 279 - protein, 162 aa, synthetic construct
MSDEQYMRRALELARQAEQEGEVPVGAVLVKDGEVIAEGWNRSIGSHDATGHAEIMALRAAGEKLQNYRLLDTTLYVTLEPCPMCAGALLHSRVKRVVFGAPDLKAGAAGTVLNLFESQASYHYADVESGLLEEECREQLQAFFKRRRKEIKAARQAKRDAE

SEQ ID NO: 280 - protein, 142 aa, synthetic construct
MREALAEARAAAAAGEVPVGAVVVRDGAVLARAGNRTVRDADPTAHAEIVALRAAARALGNHRLGGCTLYVTLEPCAMCAGAIAQARVARLVYGADDPKGGAVAHGPRLFAQPTCHHRPEVVGGVGAAEAAALLRDFFAARR

SEQ ID NO: 281 - protein, 148 aa, synthetic construct
MDDEALMGLALDEARAAAAAGEVPIGAVVARGGEVLARAGNRTLRDCDPTAHAEIVALREAARALGNYRLTGCTLYVTLEPCAMCAGAAIQARLDRVVYGAADPKAGAAGSVLDVLGHPRLNHRPAVEGGVLAAESAALLREFFAARR

SEQ ID NO: 282 - protein, 142 aa, synthetic construct
MREALAEARAAAAAGEVPVGAVVVRDGAVLARAGNASIRARDPTAHAEILALRAAARALGNHRLTGCTLYVTLEPCAMCAGAIAHARLARLVYGAADPKGGAVAHGPRLFAQPTCHHRPEVTGGVGAAEAAALLRDFFAARR

SEQ ID NO: 283 - protein, 164 aa, synthetic construct
MSDEQFMRRAIELAKKGEELGEVPVGAVLVKDGEIIAEGWNQSISTHDATAHAEIMAIRAAGEKLENYRLLDTTLYVTLEPCPMCAGALLHSRVKRVVFGAPDLKAGAAGTVLNLFESQASYHYADVESGLLEEECREQLQAFFKRRRKEIKAAKQAKREAEEK

SEQ ID NO: 284 - protein, 162 aa, synthetic construct
MDDEFYMRRALELAALAEEHNEVPVGAVLVLNGEIIGEGWNRSIGHHDATAHAEIMALRQAGKKLENYRLLDTTLYVTLEPCPMCAGALLHSRVKRVVFGAPDLKAGAAGTVLNLFESQASYHYADVESGLLEQECREQLQAFFKRRRKEIKALKKAQKEAE

SEQ ID NO: 285 - protein, 148 aa, synthetic construct
MDDAGFMRLALAEARKAAAAGEVPVGAVVVRDGAVLAAAGNRTLRDCDPTAHAEVVALRAAARALGNHRLGGCDLYVTLEPCAMCAGAISHARIARLYYGADDPKGGAVAHGARVFDQPTCHHRPEVYGGIGAGEAAALLRDFFAARR

SEQ ID NO: 286 - protein, 149 aa, synthetic construct
DLDDARFMREALAEARAAAEAGEVPVGAVVVADGRIVARAHNRPVALNDPTAHAEILALREAARALGNYRLTGCTLYATLEPCAMCAGAILHARIARLVYGARDAKAGAVESLYRLLDDARLNHRVEVAAGVLADECGALLTEFFRARR

SEQ ID NO: 287 - protein, 147 aa, synthetic construct
DESFMREALAEARAAAEAGEVPVGAVVVHGGQIVARAHNRPIELSDPTAHAEILALRAAARALGNYRLTGCTLYATLEPCAMCAGAILHARIDRVVYGASDPKAGAAGSVLSVFDHPALNHRVDVTAGVLAEECGALLREFFRARRR

SEQ ID NO: 288 - protein, 148 aa, synthetic construct
DPDAAFMRAALAEARAAAEAGEVPVGAVVVADGAIVARAHNRPIALNDPTAHAEILALREAARALGNYRLTGATLYATLEPCAMCAGAILHARIARLVYGARDPKAGAVESLYRLLDDARLNHRVAVTGGVLAAECGALLTAFFRARR

SEQ ID NO: 289 - protein, 147 aa, synthetic construct
DERFMREALAEARAAAAAGEVPVGAVVVRDGEIVARAANRTVRDNDPTAHAEILALREAARALGSYRLTGCTLYATLEPCAMCAGAILHARIARLVYAAADPKAGACGSVLSVMNHPQLNHRVEVESGLLAEECGALLTEFFRARRG

SEQ ID NO: 290 - protein, 163 aa, synthetic construct
MTADEQYMRRALELARQAEAEGEVPVGAVLVKDGEIVAEGWNRSIGHHDATAHAEIQVLRKAGQVLQNYRLLDTTLYVTLEPCPMCAGALLHSRVKRIVYGAPDLKAGAAGTVLNLFEHQAAYHYADVEHGLLEDECREQLQAFFKRRRKEKKALKQALKESN

SEQ ID NO: 291 - protein, 150 aa, synthetic construct
RIRSDEDFMRQALAEARAAAAAGEVPVGAVVVAGGRIVARAHNRPIDLVDPTAHAEILALREAARALGNYRLTGCTLYATLEPCAMCAGAILHARIARLVYGAADPKAGAVESVLRLLDHPALNHRVEAVSGVLAGECGALLREFFRARR

SEQ ID NO: 292 - protein, 148 aa, synthetic construct
DLDEALMREALAEARRAAEAGEVPVGAVVARGGRIVARAHNQPVALHDPTAHAEILALRQAAREAGNYRLEGCTLYATLEPCAMCAGALLHARVARLVYGARDPKAGAVGSLYDLLRDPRLNHRVEVTAGVLAEECGALLSGFFRARR

SEQ ID NO: 293 - protein, 143 aa, synthetic construct
MREALAEARAAAAAGEVPVGAVVVDPAGEIVARAGNAPRALCDPTAHAEILALRAAAAAAGNYRLPGHVLYVTLEPCAMCAGAISHARIARLVYGAADPKGGAVAHGPRFFAQPTCHHRPEVTGGVLAEECGALLRDFFRARR

SEQ ID NO: 294 - protein, 162 aa, synthetic construct
RLDEQFMRRALELAAHAEAEGEVPVGAVLVLDGQVIGEGWNRSIGQHDATAHAEIMALRQAGQVVQNYRLLDTTLYVTLEPCPMCAGALLHSRVKRIVYGAPDLKAGAAGTVLNLFEHQAAYHYADVEHGLLEEECREQLQAFFKRRRKEKKALKQAQRAAE

SEQ ID NO: 295 - protein, 149 aa, synthetic construct
DPDDVAFMREALAEAERAAAAGEVPVGAVVVHDGVIVGRGHNRPIASRDPTAHAEIVALREAAAALGNYRLTGCSLYVTIEPCAMCAGAILHARIARLVYGAADPKAGACGSVLDVFAEPRLNHHAEVTGGVLADECAALLRSFFAARR

SEQ ID NO: 296 - protein, 148 aa, synthetic construct
DPDERFMRAALAQARAAAEAGEVPVGAVVVRDGRIVARAHNQPIALNDPTAHAEILALREAARELGNYRLTGCTLYATLEPCAMCAGAILHARIARLVYGARDAKAGAVESKLRLFENGSFNHRVEVTGGVLAGECGALLSEFFRARR

SEQ ID NO: 297 - protein, 148 aa, synthetic construct
MDEEFMREALAEARAAAEAGEVPVGAVVVAGGRIVGRGRNRPVELADPTAHAEILALREAARALGNYRLEGCTLYATLEPCAMCAGAILHARIARLVYGAADPKAGAAGSVLDVFALPRLNHRTAVTGGVLAEECGALLSGFFRARRG

SEQ ID NO: 298 - protein, 150 aa, synthetic construct
RSSDAALMREALAEARAAAEAGEVPVGAVVVAGGRIVARAHNRPVALSDPTAHAEVLALREAARELGNYRLEGCTLYATLEPCAMCSGALLHARVARLVYGAADPKAGAVGSVLDLFANPRLNHRVEVTGGVLAEECGALLSGFFRARRG

SEQ ID NO: 299 - protein, 148 aa, synthetic construct
DDDERWMREALAEARAAAAAGEVPVGAVVVRDGVLLARAGNASIRERDPTAHAEMLALRAAARALGNHRLGGCTLYVTLEPCAMCAGAIAHARIARLVYGAADPKAGAVDHGPRLFAQPTVHHRPEVVAGVGAEESAALLRDFFRARR

SEQ ID NO: 300 - protein, 150 aa, synthetic construct
DPDSDEAFMRAALAEARAAAEAGEVPVGAVVVADGAIVARAHNRPIALHDPTAHAEILALRAAARRLGNYRLTGCTLYATLEPCAMCAGAILHARIARLVYGAADPKAGACGSVLAVMNHPQLNHRVEVTGGILAEECGALLRDFFRARR

SEQ ID NO: 301 - protein, 142 aa, synthetic construct
MREALAEARAAAEAGEVPVGAVVVAGGRIVARAHNRPVALHDPTAHAEILALRQAAAELGNYRLEGCTLYATLEPCAMCAGAMLHARLARVVYGAADPKAGAAGSVLDLFANPRLNHRTEVVGGVLAEECGALLSGFFRSRR

SEQ ID NO: 302 - protein, 146 aa, synthetic construct
DERFMREALAEARAAAAAGEVPVGAVVVRDGVVLARAGNRTLRDRDPTAHAEMLALRQAARALGSHRLTGCDLYVTLEPCAMCAGAISHARIARLYYGAADAKGGAVEHGPRFFAQPTCHHRPEVYGGIGETEAAALLRGFFRERR

SEQ ID NO: 303 - protein, 149 aa, synthetic construct
MDSDLEFMRLALAEARAAAEAGEVPVGAVVVAGGRVVARAHNRPVALHDPTAHAEILALRAAARELGNYRLTGCTLYATLEPCAMCAGAILHARIARLVYGAADPKAGACGSVLDVIHHPRLNHRVEVTGGVLAEECGALLSGFFRARR

SEQ ID NO: 304 - protein, 143 aa, synthetic construct
MREALAEARAAAEAGEVPVGAVVVAGGRIVARAHNRPVALHDPTAHAEILALREAARALGNYRLEGCTLYATLEPCAMCAGALLHARVARLVYGARDPKAGAVGSVLDLLDHPRLNHRVEVVGGVLAEECGALLSGFFRARRG

SEQ ID NO: 305 - protein, 147 aa, synthetic construct
DDRRWMREALAEARAAAEAGEVPVGAVVVRDGRLLARAGNASIRDRDPTAHAELLALRAAARALGNHRLTGCTLYVTLEPCAMCAGAIAHARIARLVYGAADPKGGAVAHGPRLFEQPTLHHRPEVTGGVLAAEAGELLRAFFRARR

SEQ ID NO: 306 - protein, 148 aa, synthetic construct
MNDEDFMRAALAQARLAAEAGEVPVGAVVVCDGEIVARAHNRPISASDPTAHAEILALREAARRLGNYRLTGCTLYATLEPCAMCAGAILHARIARLVYGAADPKAGAVGSVLDVLNHPRLNHRVEVTGGVLAEECGALLRDFFRARR

SEQ ID NO: 307 - protein, 151 aa, synthetic construct
DESSDLRFMREALAEARAAAEAGEVPVGAVVVCGGRIVARAHNRPIALSDPTAHAEILALREAARELGNYRLTGCTLYATLEPCAMCAGAILHARIARLVYGAADPKAGACGSVLDVINHPRLNHRVEVTGGVLAGECGALLSGFFRARRG

SEQ ID NO: 308 - protein, 147 aa, synthetic construct
DDLFFMREALAEARRAAAEGEVPVGAVVVCGGRIIARAHNRPIALNDPTAHAEILALRAAARALGNYRLTDCTLYATLEPCAMCAGAILHARIRRLVYGAADPKAGAVRSVLRLLDAPALNHRVAVTAGVLAEECGALLSEFFRARR

SEQ ID NO: 309 - protein, 151 aa, synthetic construct
DPPSADEAFMREALAEARAAAEAGEVPVGAVVVAGGRIVARAHNRPIALSDPTAHAEILALRAAARALGNYRLTGTTLYATVEPCAMCAGAIVQARVARLVYGAADPKAGAVESLFRILDHPALNHRVEVTAGVLAEECAALLREFFRARR

SEQ ID NO: 310 - protein, 149 aa, synthetic construct
DETEAFMRAALAEARAAAEAGEVPVGAVVVADGRIVARAHNRPVALHDPTAHAEILALREAARRLGNYRLTGCTLYATLEPCAMCAGAVLHARLARLVYGARDAKAGAVGSVLDLLAHPRLNHRTAVTAGVLADECGALLSAFFRARRG

SEQ ID NO: 311 - protein, 148 aa, synthetic construct
DDDERWMREALAEARAAAAAGEVPVGAVVVRDGVLLARAGNASIRDRDPTAHAEMLALRAAARALGNHRLTGCTLYVTLEPCAMCAGAIAHARIARLVYGASDPKAGAVDHGPRLFEQPTIHHRPEVTAGVLADECGALLRDFFRARR

SEQ ID NO: 312 - protein, 150 aa, synthetic construct
DPTTDEALMREALAEARAAAEAGEVPVGAVVARDGEIVARAANAPRALCDPTAHAEILALRAAARALGNYRLTGCTLYATLEPCAMCAGAALHARFARIVYGAADPKAGAAGSVLDVLDQPRLNHRTRVTGGVLADECGALLTEFFRARR

SEQ ID NO: 313 - protein, 164 aa, synthetic construct
MNKDEFYMKRALELAQKAEEEGEVPVGAVLVLDDEIIGEGWNRPIGSHDATAHAEIQALRDAGQKLENYRLLDTTLYVTLEPCPMCAGALLHSRVKRVVYGAPDLKAGAAGTVLNLFEHEAAYHYADVEHGLLEDECREQLQAFFKRRRKEIKAKKEAEKKALE

SEQ ID NO: 314 - protein, 164 aa, synthetic construct
MNSDEFYMQRALELAQKAEQEDEVPVGAVLVLDGEIIGEGWNRSIGHHDATAHAEMMALKQGGKQIQNYRLLDATLYVTLEPCPMCAGALLHSRVKRIVYGAPDLKAGAAGTVLNLFEHQAAYHYADVEQGLMEEECREQLQAFFKRRRKEKKALKQAKREAEE

SEQ ID NO: 315 - protein, 146 aa, synthetic construct
DERFMREALAEARAAAAAGEVPVGAVVVRDGVVLARAGNRTLRDRDPTAHAEMLALRQAARALGSWRLTGCTLYVTLEPCAMCAGAMVQARVDRLVYGAADPKAGAAGSVLDVLGHPRLNHRPEVAAGVLAEECGALLREFFRERR

SEQ ID NO: 316 - protein, 142 aa, synthetic construct
MRAALEEARRAAAEGEVPVGAVVVRDGAIVARAHNRPIALNDPTAHAEILALREAARALGNYRLTGCTLYATLEPCAMCAGAILHARIARLVYGAADPKAGACGSVLSVINHPRLNHRVEVTGGVLAEECGALLTGFFRARR

SEQ ID NO: 317 - protein, 148 aa, synthetic construct
DPDAAFMREALAEARAAAAAGEVPVGAVVVRAGRIVARAHNRPVELHDPTAHAEILALRAAARELGNYRLTGCTLYATLEPCAMCAGAILHARIERLVYGARDAKAGAAGSVLDVFANPRLNHRVAVEGGVLADECAALLREFFRARR

SEQ ID NO: 318 - protein, 148 aa, synthetic construct
MDDEALMRVALEEARAAAAAGEVPVGAVVARGGEIVARAANAPIAASDPTAHAEILALRAAARALGNYRLTGCTLYATLEPCAMCAGAILHARIARLVYAAPDPKAGACGSVLAVLDHPALNHRVEVTGGVLAEECGALLREFFRARR

SEQ ID NO: 319 - protein, 164 aa, synthetic construct
MSDEQYMRRALELARQAEQEGEVPVGAVLVKDGEIIAEGWNRSIGHHDATAHAEMQVLRKAGQVLENYRLLDTTLYVTLEPCPMCAGALLHSRVKRIVYGAPDLKAGAAGTVLNLFEHQAAYHYADVEHGLLEEECREQLQAFFKRRRKEIKARKQAEKEAEAR

SEQ ID NO: 320 - protein, 151 aa, synthetic construct
DPLEQDERWMREALAEARAAAEAGEVPVGAVLVRDGVLLARAGNRTIRDRDPTAHAEMLALRAAARALGNHRLTGCTLYVTLEPCAMCAGAIAHARIARLVYGASDPKGGAVAHGPRLFEQPTLHHRPEVTGGVLAEEAGELLRAFFRARR

SEQ ID NO: 321 - protein, 142 aa, synthetic construct
MREALAEARAAAEAGEVPVGAVVVAGGRIVARAHNQPIALNDPTAHAEVLALRQAARELGNYRLTGCTLYATLEPCAMCAGAILHARLDRVVYGAADPKAGAAGSVLDVFANPRLNHHAQVTGGVLAEECGALLSGFFRARR

SEQ ID NO: 322 - protein, 147 aa, synthetic construct
DDLSFMREALAEAEAAAEAGEVPVGAVLVRDGEIVARGRNRVIEDRDPTAHAEIVALREAGRALGNYRLEGCTLYVTLEPCAMCAGALVHARLDRLVYGAADPKAGAAGSVLDVLNHPRLNHRMEVTGGVLADECGAMLRAFFRARR

SEQ ID NO: 323 - protein, 151 aa, synthetic construct
DRLRADEQFMRAALAEARAAAAAGEVPVGAVVVRDGEIVARAHNRPIALHDPTAHAEILALRAAARALGNYRLTGCTLYATLEPCAMCAGAILHARLDRVVYGAADPKAGACGSVLSVMNHPRLNHRVEVTGGVLADECGALLTEFFRARR

SEQ ID NO: 324 - protein, 143 aa, synthetic construct
MREALAEARAAAAAGEVPVGAVVVRGGEIVARAHNRPVALSDPTAHAEVLALREAARRLGNYRLEGCTLYATLEPCAMCSGAVLHARLARLVYGAPDPKAGAAGSVLDVLDHPRLNHRTAVTGGVLAEECGALLSGFFRARRG

SEQ ID NO: 325 - protein, 147 aa, synthetic construct
DDRRWMREALAEARAAGAAGEVPVGAVVVRDGELLARAGNASIRDRDPTAHAEMLALRAAARALGNHRLTGCTLYVTLEPCAMCAGAIAHARIARLVYGASDPKGGAVAHGPRLFEQPTLHHRPEVTAGVLADECGALLRDFFRARR

SEQ ID NO: 326 - protein, 142 aa, synthetic construct
MREALAEARRAAEAGEVPVGAVVVRDGEIVARAHNRPIALHDPTAHAEILALREAARRLGNYRLTGCTLYATLEPCAMCAGAILHARLDRVVYGAPDPKAGACGSVLSVFDQPRLNHRTRVEGGVLAEECGALLSGFFRARR

SEQ ID NO: 327 - protein, 149 aa, synthetic construct
DDRDRRWMRAALAEARAAAEAGEVPVGAVVVRDGELLARAGNASIRDRDPTAHAEMLALRAAARALGNHRLTGCTLYVTLEPCAMCAGAIAHARIARLVYGAADPKGGAVAHGPRLFEQPTLHHRPEVTAGVLAEEAGELLRAFFRARR

SEQ ID NO: 328 - protein, 142 aa, synthetic construct
MREALAEARAAAAAGEVPVGAVVVRGGEIVARAHNRPIALNDPTAHAEILALREAARVLGNYRLTGCTLYATLEPCAMCAGAILHARIDRLVYGAADPKAGACGSVLSVMNHPQLNHRVEVTSGVLAEECGALLTDFFRARR

SEQ ID NO: 329 - protein, 145 aa, synthetic construct
MREALAEARAAAEAGEVPVGAVVVRDGEIVARAHNRPIALNDPTAHAEILALREAARRLGNYRLTGCTLYATLEPCAMCAGAILHARLARLVYGAADPKAGACGSVLSVINHPRLNHRVEVTSGVLADECGALLSDFFRARRRAA

SEQ ID NO: 330 - protein, 146 aa, synthetic construct
DDRFMRAALAEARAAAEAGEVPVGAVVVVDGRIVARAHNRPVELSDPTAHAEVLALREAARALGNYRLEGATLYATLEPCAMCSGAILHARVERVVYGAADSRAGAAGSVLDVFATRRLNHQTTVTGGVLAEECGALLSGFFRARR

SEQ ID NO: 331 - protein, 148 aa, synthetic construct
DDDQRFMREALAEARAAAAAGEVPIGAVVVCDGAIVARAGNRTIRDNDPTAHAEILALRQAARALGNYRLTGCTLYVTLEPCAMCAGAIVQARIARLVYGAADPKAGAVDSVLDVLNHPRLNHRVEVTRGVLAEECGALLRDFFRARR

SEQ ID NO: 332 - protein, 150 aa, synthetic construct
MSDDERFMRLALEQARLAAEAGEVPVGAVVVCDGRVVARAHNRPIALNDPTAHAEILALREAARELGNYRLTGCTLYATLEPCAMCSGAVLHARLARVVYGAADPKTGAAGSVLDLFAEPRLNHHAEVTGGVLAGECGALLSGFFRARRG

SEQ ID NO: 333 - protein, 160 aa, synthetic construct
DPTDLAFMREALAEAEAAAAAGEVPVGAVVVHEGRIVGRGRNRMIAASDPTAHAEIVALREAARALGNYRLTGCTLYVTLEPCAMCAGAILHARIARLVYGAADPKAGACGSVLDVLNHPRLNHRTEVTAGVLAEECGAMLSGFFRARRAQQRAARVAAS

SEQ ID NO: 334 - protein, 149 aa, synthetic construct
DPPDAVFMRAALEEARAAAEAGEVPVGAVLVHDGQILARAGNRTIRDRDPTAHAEILALRQAARVLGSHRLTGATLYVTLEPCAMCAGAISHARIARLVYGADDPKGGAVRHGPRFFEQPTCHHRPEVTGGILAEESAALLRGFFRARR

SEQ ID NO: 335 - protein, 142 aa, synthetic construct
MRAALEEARRAAEAGEVPVGAVVVRDGAIVARAHNRPIALHDPTAHAEILALREAARRLGNYRLTGCTLYATLEPCAMCAGAILHARLDRVVYGAADPKAGACGSVLSVFDQPRLNHRTRVEGGVLAEECGALLSGFFRARR

SEQ ID NO: 336 - protein, 149 aa, synthetic construct
DDDQRFMALALAQAQAAADAGEVPVGAVVVCDGRVVARAHNRPIALNDPTAHAEILALREAARALGNYRLTGCTLYATLEPCAMCAGAILHARIARLVYGARDPKAGAVESLYRLLDDDRLNHRVAVTGGVLAAACGALLSGFFRARRR

SEQ ID NO: 337 - protein, 149 aa, synthetic construct
DLTDEQLMRAALAEARAAAAAGEVPVGAVVAKDGEIIARAHNRPIAAHDPTAHAEILALRAAARALGNYRLTGCTLYATLEPCAMCSGAILHARIARLVYGAADPKAGACGSVLAVMNHPQLNHRVEVTGGVLAEECGALLRDFFRARR

SEQ ID NO: 338 - protein, 148 aa, synthetic construct
DDDERWMREALAEARAAAEAGEVPVGAVVVRDGELLARAGNASIRDRDPTAHAEMLALRAAARALGNHRLTGCTLYVTLEPCAMCAGAIAHARIARLVYGASDPKGGAVAHGPRLFEQPTLHHRPEVTGGVLAEEAGALLRAFFRARR

SEQ ID NO: 339 - protein, 146 aa, synthetic construct
DLEFMREALAEARAAAEAGEVPVGAVVVRDGAIVARAGNRTLRDRDPTAHAEMLALRQAARALDSWRLSGCTLYVTLEPCAMCAGAMVQARLDRLVYGAADPKAGAAGSVLDVLGHPRLNHRVEVTGGVLAEECGALLREFFRARR

SEQ ID NO: 340 - protein, 150 aa, synthetic construct
RSESDEALMREALAEARRAADAGEVPVGAVVVRDGAILARAGNAPIAASDPTAHAEILALRAAARALGNYRLTGCTLYATLEPCAMCAGAILHARIARLVYGARDPKAGAVESVLDLLDRPALNHRVEVTGGVLAAECGALLSGFFRARR

SEQ ID NO: 341 - protein, 148 aa, synthetic construct
MDDAGFMRAALAEARAAAEAGEVPVGAVVVCDGRIVARAGNRTLRDRDPTAHAEMLALRAAARALGSHRLTGCDLYVTLEPCAMCAGAISHARIRRLYYGASDPKGGAVENGVRFFAQPTCHHAPEVYGGIGEAEAAALLRGFFRARR

SEQ ID NO: 342 - protein, 149 aa, synthetic construct
MRDEDYMRAALAQAREAAAAGEVPVGAVVVADGRIVARAHNRPIALNDPTAHAEILALREAARQLGNYRLTGCTLYATLEPCAMCAGAILHARIARLVYGAADPKAGAVGSVLDVLNHPRLNHRVEVTSGVLAAECGALLTEFFRSRRR

SEQ ID NO: 343 - protein, 149 aa, synthetic construct
DETEAFMRAALAEARAAAAEGEVPVGAVVVADGAIVARAHNRPIALNDPTAHAEILALREAARALGNYRLTGCTLYATLEPCAMCAGAILHARIARLVYGARDPKAGAVESLYRLLDDTRLNHRVEAVGGVLADECGALLTEFFRARRG

SEQ ID NO: 344 - protein, 148 aa, synthetic construct
DDDERWMREALAEARAAAAAGEVPVGAVVVRDGVLLARAGNASIRDRDPTAHAEMLALRAAARALGNHRLGGCTLYVTLEPCAMCAGAIAHARIARLVYGAADPKAGAVDHGPRLFEQPTCHHRPEVVSGVGAAESAALLRDFFRARR

SEQ ID NO: 345 - protein, 145 aa, synthetic construct
MRAALEEARRAAEAGEVPVGAVVVRDGAIVARAHNRPIALNDPTAHAEILALREAARALGNYRLTGCTLYATLEPCAMCAGAILHARLDRVVYGAADPKAGACGSVLSVFDQPRLNHRTTLTGGVLAEECGALLSGFFRARRRAA

SEQ ID NO: 346 - protein, 149 aa, synthetic construct
MDDDLFMRAALDEARAAAEAGEVPVGAVVVADGRIVARAHNRPIALNDPTAHAEILALRAAAAALGNYRLTGCELFVTLEPCAMCAGAILHARLARVVYGAADPKTGAAGSVLDVFANPRLNHRTEVRGGVLADECGALLTEFFRARRG

SEQ ID NO: 347 - protein, 149 aa, synthetic construct
MDDEALMREALAEARAAAEAGEVPVGAVVARGGEIVARGRNRMIADGDPTAHAEIVALREAARALGNYRLTGCTLYATLEPCAMCAGAILHARIARLVYAAPDPKAGACGSVLSVMNHPQLNHRVEVASGLLAEECGALLTNFFRARRG

SEQ ID NO: 348 - protein, 149 aa, synthetic construct
DPEDERFMREALAEAERAREAGEVPVGAVVVLDGRIVGRGHNRPIGAHDPTAHAEIVALREAAAALGNYRLTGATLYVTLEPCAMCAGAILHARIARLVYGAADPKTGAVGSLLDLLAEPRLNHRTEVTGGVLAEECGALLSAFFRARR

SEQ ID NO: 349 - protein, 147 aa, synthetic construct
DDLSFMREALAEAEAAAAAGEVPVGAVVVHGGRIVGRGRNRMIADHDPTAHAEIVALREAARALGNYRLTGCTLYVTLEPCAMCAGAILHARIERLVYGAADPKAGACGSVLSVMNHPQLNHRVEVTSGVLADECGAMLSGFFRARR

SEQ ID NO: 350 - protein, 146 aa, synthetic construct
DLEFMRLALEEARAAAAAGEVPVGAVLVRDGEVLARAGNRTIRDRDPTAHAEMLAIREAARRLDNYRLEGTTLYVTLEPCAMCSGAILHARVPRVVYGAADPKAGAAGSVLDVLGHPRLNHRTEVVGGVLAEECGALLREFFRARR

SEQ ID NO: 351 - protein, 147 aa, synthetic construct
DDDHFMREALAEARRAADAGEVPVGAVVVRAGQIIARAHNRPVALHDPTAHAEILALRAAARRLGNYRLTGCTLYATLEPCAMCAGAILHARIARLVYGAADPKAGACGSVLDVLNHPRLNHRVEVTAGVLAEECGALLSAFFRARR

SEQ ID NO: 352 - protein, 148 aa, synthetic construct
DDDQRFMREALAEARAAAAAGEVPIGAVVVCDGEIVARAGNRTIRDNDPTAHAEILALRQAARALGNYRLTGCTLYVTLEPCAMCAGAIVHARIARLVYGAADPKAGAVNSVFDLLNHPRLNHRVEVTSGVLADECGALLRDFFRARR

SEQ ID NO: 353 - protein, 146 aa, synthetic construct
DESFMREALAEARAAAEAGEVPVGAVVVHGGRIVARAHNRPIELSDPTAHAEILALRAAARALGNYRLTGCTLYATLEPCAMCAGAILHARIDRVVYGAADPKAGAAGSVLDVFANPRLNHHARVTGGVLADECGALLSGFFRARR

SEQ ID NO: 354 - protein, 151 aa, synthetic construct
DPESDAAFMRAALAEAQAAAEAGEVPVGAVVVCDGRIVARAHNRPIGLNDPTAHAEILALREAARELGNYRLTGCTLYVTLEPCAMCSGAILHARLARVVYGAADPKTGAAGSVLDLFANRRLNHQTQVEGGVLAEECGALLSGFFRARRG

SEQ ID NO: 355 - protein, 142 aa, synthetic construct
MREALAEARAAAAAGEVPVGAVVVRGGEIVARAHNRPIALNDPTAHAEILALREAARVLGNYRLTGCTLYATLEPCAMCAGAILHARIARLVYGAADPKAGACGSVLSVMNHPQLNHRVEVTGGVLAGECGALLSDFFRARR

SEQ ID NO: 356 - protein, 150 aa, synthetic construct
MSDDERFMRLALAQAREAAAEGEVPVGAVVVCGGRVVARAHNRPIALNDPTAHAEVLALRAAARELGNYRLTGCTLYATLEPCAMCAGAILHARIARVVYGARDPKAGAAGSVLDVFANPRLNHHATVTGGVLAEECGALLSDFFRARRR

SEQ ID NO: 357 - protein, 149 aa, synthetic construct
MDDDERWMREALAEARAAAEAGEVPVGAVVVRDGVLLARAGNASIRDRDPTAHAEMLALRAAARALGNHRLTGCTLYVTLEPCAMCAGAIAHARIARLVYGAADPKAGAVDHGPRLFEQPTCHHRPEVVSGVGAAESAALLRDFFRARR

SEQ ID NO: 358 - protein, 148 aa, synthetic construct
DDDERWMRAALAEARAAAEAGEVPVGAVVVAGGRLVARAGNASIRDSDPTAHAEILALRAAARALGNHRLAGCTLYVTLEPCAMCAGAIAHARIARLVYGASDPKGGAVAHGPRLFEQPTLHHRPEVTGGVLAAEAGALLRAFFRARR

SEQ ID NO: 359 - protein, 142 aa, synthetic construct
MREALAEARRAAAEGEVPVGAVVVRDGAVIARAHNRPIALRDPTAHAEILALREAARLLGNYRLTGCTLYVTLEPCAMCAGAIVHARIDRLVYGAADPKAGAVESVLRVLDHPRLNHRVEVTRGVLAEECGALLREFFRARR

SEQ ID NO: 360 - protein, 149 aa, synthetic construct
MNDEDFMRAALAQAREAAAAGEVPVGAVVVCGGRIVARAHNRPISARDPTAHAEILALRAAARELGNYRLTGCELYVTLEPCAMCAGAILHARIARLVYGAADPKTGAAGSVLDVFANPRLNHRTEVTGGVLADECGALLSGFFRARRG

SEQ ID NO: 361 - protein, 148 aa, synthetic construct
DDDQRFMAAALEEARAAAAEGEVPVGAVVVAGGRIVARAHNRPIALNDPTAHAEILALRAAARELGNYRLTGCTLYATLEPCAMCAGAILHARIDRVVYGARDPKAGAAGSVLDVFANPRLNHHAEVTGGVLAEECGALLSDFFRARR

SEQ ID NO: 362 - protein, 142 aa, synthetic construct
MREALAEARAAAEAGEVPVGAVVVAGGRIVARAHNRPIALHDPTAHAEILALREAARELGNYRLTGCTLYATLEPCAMCAGAILHARIARLVYGAADPKAGACGSVLAVMNHPQLNHRVEVVGGVLAEECGALLREFFRARR

SEQ ID NO: 363 - protein, 149 aa, synthetic construct
MDSDLEFMREALAEARAAAEAGEVPVGAVVVRDGEIVARAANRTIRDGDPTAHAEMVALRAAARALGNYRLAGCTLYVTLEPCAMCAGAMIHARLDRVVYGAADPKAGAAGSVLDVLGHPALNHRTAVTAGVLAEECGALLRDFFRARR

SEQ ID NO: 364 - protein, 150 aa, synthetic construct
DPTTDEALMREALAEARAAAADGEVPVGAVVARDGVIVARAHNAPVALSDPTAHAEILALREAARAAGNYRLPGCTLYATLEPCAMCCGAALHARLARVVYGARDPKAGAAGSVLDLLDDPRLNHRAEVVGGVLAEECGALLSGFFRARR

SEQ ID NO: 365 - protein, 152 aa, synthetic construct
DPLSADDARFMRLALAEARAAAEAGEVPVGAVVVAGGRVVARAHNRPVALHDPTAHAEILALRAAARALGNYRLTGCTLYATLEPCAMCAGAILHARIARLVYGAADPKAGACGSVLAVMNHPQLNHRVEVTGGVLAEECGALLRDFFRARR

SEQ ID NO: 366 - protein, 148 aa, synthetic construct
MNDEDFMRAALAQAREAAAAGEVPVGAVVVCDGRIVARAHNRPIALNDPTAHAEILALREAARALGNYRLTGCTLYATLEPCAMCAGAILHARIARLVYGAADPKAGAVGSVLDVMNHPRLNHRVEVTSGVLAEECGALLKEFFRARR

SEQ ID NO: 367 - protein, 148 aa, synthetic construct
DDDQRFMREALAEARAAAEAGEVPIGAVVVCDGEIVARAGNRTIRDNDPTAHAEILALRQAARRLGNYRLTGCTLYVTLEPCAMCAGAIVQARIARLVYGAADPKAGAVDSVLQVLNHPRLNHRVEVTRGVLAEECGALLRDFFRARR

SEQ ID NO: 368 - protein, 142 aa, synthetic construct
MREALAEARRAAEAGEVPVGAVVVRDGEIVARAHNRPIALHDPTAHAEILALREAARRLGNYRLTGCTLYATLEPCAMCAGAILHARLDRVVYGAPDPKAGACGSVLSVFDQPALNHRVAVTGGVLAEECGALLREFFRARR

SEQ ID NO: 369 - protein, 148 aa, synthetic construct
DPDARFMREALAEAEAAAAAGEVPVGAVVVAGGRIVARAHNRPVELSDPTAHAEILALREAARELGNYRLEGCTLYATLEPCAMCSGAILHARLARVVYGAADPKAGAAGSVLDVFALPQLNHRTAVTGGVLADECGALLSGFFRARR

SEQ ID NO: 370 - protein, 149 aa, synthetic construct
MNDEDYMRAALEQARLAASEGEVPVGAVVVCDGKIVARAHNRPIALNDPTAHAEILALREAARKLGNYRLTGCTLYATLEPCAMCSGAILHARIARLVYGAADPKAGAVGSVLDVINHPRLNHRVEVTSGVLAEECGALLSGFFRARRK

SEQ ID NO: 371 - protein, 142 aa, synthetic construct
MRAALEEARRAAEAGEVPVGAVVVRDGAIVARAHNRPIALHDPTAHAEILALREAARRLGNYRLTGCTLYATLEPCAMCAGAILHARIDRLVYGAADPKAGACGSVLSVMNHPRLNHRVEVERGVLAEECGALLTGFFRARR

SEQ ID NO: 372 - protein, 150 aa, synthetic construct
MSTEDERFMRLALAEARRAAAEGEVPVGAVVVAGGEVVAAAHNRPIALADPTAHAEILALRAAARALGNYRLTGCTLYATLEPCAMCAGAILHARIARLVYGAADPKAGAVESVLRLLDAPGLNHRVAVTGGVLAEECGALLREFFRARR

SEQ ID NO: 373 - protein, 143 aa, synthetic construct
MREALAEARAAAEAGEVPVGAVVVRDGAIVARAHNRPIALNDPTAHAEILALREAARALGNYRLTGCTLYATLEPCAMCAGAILHARLDRVVYGAADPKAGAAGSVLQVFDHPRLNHRTAVTGGVLAAECGALLSGFFRARRG

SEQ ID NO: 374 - protein, 143 aa, synthetic construct
MREALAEARAAAAAGEVPVGAVVVRGGEIVARAHNRPIALNDPTAHAEILALREAARALGNYRLTGCTLYATLEPCAMCAGAILHARIDRVVFGARDPKAGAAGSVLDVFANPRLNHHAEVVGGVLADECGALLSGFFRARRG

SEQ ID NO: 375 - protein, 146 aa, synthetic construct
DERFMREALAEARAAAAAGEVPVGAVVVRDGVVLARAGNRTLRDRDPTAHAEMLALRQAARALGSWRLAGCTLYVTLEPCAMCAGAMVLARVDRLVYGAADPKAGAAGSVLDVLGEPRLNHRPEVAAGVLAEECGELLRAFFRERR

SEQ ID NO: 376 - protein, 151 aa, synthetic construct
DPTSDEAFMRAALAEARAAAEAGEVPVGAVVVCGGRIVARAGNRPIAARDPTAHAEILALRAAARELGSYRLTGATLYVTLEPCAMCAGAILHARIERLVFGAADPKTGAAGSVLDLFAEPRLNHRTAVEGGVLAEECGALLRDFFRARRG

SEQ ID NO: 377 - protein, 150 aa, synthetic construct
DPSPDEAFMRAALAEARAAAEAGEVPVGAVVVRDGRIVARAGNRPIALHDPTAHAEILALRAAARELGNYRLTGCELYVTLEPCAMCAGAILHARLARLVYGAADPKTGAAGSVLDVFANRRLNHHTAVTGGVLADECGALLAGFFRARR

SEQ ID NO: 378 - protein, 149 aa, synthetic construct
MDDAAFMRAALAEARAAAEAGEVPVGAVVVADGAIVARAHNRPIALNDPTAHAEILALRAAARALGNYRLTGCTLYATLEPCAMCAGAILHARIARLVYGARDPKAGAVESLYRLLDDTRLNHRVAVTGGVLADECGALLTEFFRARRR

SEQ ID NO: 379 - protein, 150 aa, synthetic construct
DPESDAAFMRAALAEARAAAEAGEVPVGAVVVCDGRIVARAHNRPIGLNDPTAHAEILALRAAARALGNYRLTGCTLYVTLEPCAMCAGAILHARLDRVVYGAADPKTGAAGSVLDLFAQPRLNHHARVEGGVLADECGALLRDFFRARR

SEQ ID NO: 380 - protein, 149 aa, synthetic construct
DPDAAFMRAALAEARAAAEAGEVPVGAVVVAGGRIVARAHNRPIELNDPTAHAEILALRQAAAALGNYRLEGCTLYVTLEPCAMCAGAILHARLARVVYGAADPKTGAAGSVLDLFANPRLNHHAEVTGGVLAEECGALLRDFFRARRG

SEQ ID NO: 381 - protein, 147 aa, synthetic construct
MREALAEARRAAEAGEVPVGAVVVRDGEIVARAHNRPIALHDPTAHAEILALREAARRLGNYRLTGCTLYATLEPCAMCAGAILHARIDRLVYGAADPKAGACGSVLSVMNHPRLNHRVEVESGLLAGECGALLTEFFRRRRAAARG

SEQ ID NO: 382 - protein, 149 aa, synthetic construct
MSTDLAFMREALAEARAAAEAGEVPVGAVVVRDGAILARAGNRTLRDGDPTAHAEMVALRAAAAALGSERLTDCDLYVTLEPCAMCAGAISHARIRRLYYGAADPKGGAVEHGVRFFASPTCHHRPEVYGGIGEGEAAALLRDFFRARR

SEQ ID NO: 383 - protein, 146 aa, synthetic construct
DDRFMREALAEARAAAAAGEVPVGAVVVRDGEIIARAGNRTLRDRDPTAHAEMLALRQAARALGSHRLTGCDLYVTLEPCAMCAGAIQHARIRRLVYGAADPKAGAVDHGVRLFDSPSCHHRPEVVAGVGEGEAAALLRDFFRERR

SEQ ID NO: 384 - protein, 142 aa, synthetic construct
MREALAEARAAAAAGEVPVGAVVVRGGEIVARAHNRPIALHDPTAHAEILALREAARALGNYRLEGCTLYATLEPCAMCSGAILHARLARLVYGAADPKAGAVGSVLDVIGNPRLNHRVEVTGGVLAEECGALLSGFFRARR

SEQ ID NO: 385 - protein, 148 aa, synthetic construct
MDDAGFMRAALAEARAAAAAGEVPVGAVVVRDGAIVARAGNRTLRDRDPTAHAEMLAIRAAARALGSERLTGCDLYVTLEPCAMCAGAISFARIRRLYYGAADPKGGAVENGVRFFASPTCHHAPEVYSGIGEAEAAALLRDFFRARR

SEQ ID NO: 386 - protein, 151 aa, synthetic construct
RPPSDDERWMAEALAQARAAAAEGEVPVGAVVVRDGRLLARAHNRPIALNDPTAHAEILALRAAAREIGNYRLTGCTLYATLEPCAMCAGAILHARIARLVYGARDPKAGACGSVLDVLNHPRLNHRVEVTGGLLADECGALLSAFFRARR

SEQ ID NO: 387 - protein, 146 aa, synthetic construct
DLEFMREALAEARAAAEAGEVPVGAVVVAGGRIVARAHNRPIELHDPTAHAEILALRQAATALGNYRLTGCTLYATLEPCPMCAGAALHARLDRIVYGAADPKAGAAGSVLDLFADTRLNHHAAVAGGVLADECGALLSGFFRARR

SEQ ID NO: 388 - protein, 148 aa, synthetic construct
DDDERWMRAALAEARAAAAAGEVPVGAVVVRDGELLARAGNASIRERDPTAHAELLALRAAARRLGNHRLTGCTLYVTLEPCAMCAGAIAHARIARLVYGAADPKGGAVAHGPRLFEQPTLHHRPEVTAGVLAEECGALLREFFRARR

SEQ ID NO: 389 - protein, 142 aa, synthetic construct
MREALAEARAAAAAGEVPVGAVVVRGGEIVARAHNRPIALHDPTAHAEILALREAARRLGNYRLTGCTLYATLEPCAMCAGAILHARIDRLVYGAADPKAGACGSVLSVMNHPQLNHRVEVTSGVLAEECGALLTGFFRERR

SEQ ID NO: 390 - protein, 146 aa, synthetic construct
DERWMREALAEARAAAAEGEVPVGAVVVRDGRLLARAGNASIRSRDPTAHAEILALRRAARRLGNHRLTGCTLYVTLEPCAMCAGAIAHARIARLVYGASDPKGGAVAHGPRLFEQPTCHHRPEVTGGVLAEECGALLREFFRARR

SEQ ID NO: 391 - protein, 148 aa, synthetic construct
MDDAGFMRAALAEARAAAEAGEVPVGAVVVADGRIVARAHNRPVALNDPTAHAEILALRAAARALGNYRLTGCTLYATLEPCAMCAGAILHARLDRVVYGAADPKAGAAGSVLDVFANPRLNHRTAVTGGVLADECGALLTGFFRARR

SEQ ID NO: 392 - protein, 147 aa, synthetic construct
DDRFMRAALAEARAAAEAGEVPVGAVVVCGGRIVARAHNRPIDLCDPTAHAEILALREAARELGNYRLTGCTLYATLEPCAMCAGAILHARIARLVYGARDEKAGAVESLYRLLDDTRLNHRVAVTGGVLAAECGALLSEFFRARRA

SEQ ID NO: 393 - protein, 149 aa, synthetic construct
DPDAAFMRAALAEARAAAEAGEVPVGAVVVHEGRIVARAHNRPVALNDPTAHAEILALRAAARALGNYRLTGCTLYATLEPCAMCAGAILHARIARLVYGARDPKAGAVESLYRLLDDTRLNHRVEAVGGVLADECGALLSEFFRARRR

SEQ ID NO: 394 - protein, 147 aa, synthetic construct
DDRFMRAALAEARAAAAAGEVPVGAVVVAGGRIVARAHNQPVALVDPTAHAEVLALRAAARELGNYRLTGCTLYATLEPCAMCAGAILHARIDRLVYGAADPKAGAVGSVLDVFANPRLNHRTRVTGGVLAAECGALLSEFFRARRR

SEQ ID NO: 395 - protein, 154 aa, synthetic construct
MTSAPAPSDLDFMRLALAEARLAAAEGEVPVGAVVVCDGEVVARAHNRPIALHDPTAHAEILALREAARKLGNYRLTGCTLYATLEPCAMCAGAILHARIARLVYGAADPKAGAVESRLRLLDAPFLNHRVEVTGGVLAEECGALLKDFFRARR

SEQ ID NO: 396 - protein, 147 aa, synthetic construct
DDRFMRAALAEARAAAEAGEVPVGAVVVCGGEIVARAHNRPIALNDPTAHAEILALREAARRLGNYRLTGCTLYATLEPCAMCAGAILHARIARLVYGAADPKAGAVESLYRLLDDTRLNHRVEAVGGVLAGECGALLSEFFRARRA

SEQ ID NO: 397 - protein, 142 aa, synthetic construct
MREALAEARAAAAAGEVPVGAVVVAAGRIVARAHNRPVELSDPTAHAEILALREAARELGNYRLEGCTLYATLEPCAMCAGALLHARLARLVYGAADPKAGAVGSVLDVLDHPRLNHRVEVTAGVLAEECGALLRDFFRARR

SEQ ID NO: 398 - protein, 147 aa, synthetic construct
MDEEFMREALAEARAAAEAGEVPVGAVVVAGGRIVGRGRNRPVELADPTAHAEILALREAAAALGNYRLEGCTLYATLEPCAMCAGAILHARIARVVYGARDPKAGAAGSVLDVFALPRLNHHTRVTGGVLAEECGALLSGFFRARR

SEQ ID NO: 399 - protein, 164 aa, synthetic construct
RLDEQFMRRALELAAHAEAEGEVPVGAVLVLDGQVIGEGWNRSIGHHDATAHAEMMAIEQAGKAVENYRLLDATLYVTLEPCPMCAGALLHSRVKRIVYGAPDLKAGAAGTVLNLFEHQAAYHYADVEHGLLEEECREQLQAFFKRRRKEKKALKQAQRDAEEK

SEQ ID NO: 400 - protein, 153 aa, synthetic construct
MSTSEADLTFMREALAEARAAAEAGEVPVGAVVVAGGEIVARAHNRPIALHDPTAHAEILALRAAARRLGNYRLTGCTLYVTLEPCAMCAGAILHARLARLVYGAADPKTGAAGSVLDLFANRRLNHHTEVTGGVLADECGALLSDFFRARRA

SEQ ID NO: 401 - protein, 148 aa, synthetic construct
MDDAGFMRLALAEARRAAEAGEVPVGAVVVRGGEVLAAAGNRTVRDCDPTAHAEVVALREAARKLGNHRLTGCDLYVTIEPCAMCAGAISHARIARLYYGADDPKGGAVEHGPRFFGQPTCHHRPEVYGGIGAAEAAELLRGFFRARR

SEQ ID NO: 402 - protein, 142 aa, synthetic construct
MREALAEARAAAEAGEVPVGAVVVRDGEIIARARNRMVADCDPTAHAEIVALREAARALGNHRLGGCTLYVTLEPCAMCAGAMIHARVDRLVYGAADPKAGAAGSVLDVINHPRLNHRMEVESGVLADECGALLKEFFRSRR

SEQ ID NO: 403 - protein, 142 aa, synthetic construct
MDAALAEARAAADAGEVPVGAVVVRDGAVLARAGNRTLRDSDPTAHAEIVALRAAARALGNHRLPGCTLYVTLEPCAMCAGAISHARLARLHYGAADPKGGAVEHGPRFFAQPTCHHRPDVVGGLGETEAADLLRAFFAARR

SEQ ID NO: 404 - protein, 163 aa, synthetic construct
MSDEQYMRRALELARQAEQQGEVPVGAVLVRDGEVIAEGWNQSISSHDATAHAEIMALRAAGQALENYRLLDTTLYVTLEPCPMCAGALLHSRVKRVVFGAPDLKAGAAGTVLNLFESQASYHYADVESGLLEEECREQLQAFFKRRRKEIKAQRDAKRAADE

SEQ ID NO: 405 - protein, 142 aa, synthetic construct
MDAALAEARAAADAGEVPVGAVVVRDGVVLARAGNRTVRDADPTAHAEIVALRAAARALGNHRLPGCTLYVTLEPCAMCAGAISHARLARLVYGAADPKGGAVAHGPRLFAQPTCHHRPEVTGGVGAAEAAALLRAFFAARR

SEQ ID NO: 406 - protein, 148 aa, synthetic construct
MDDAGFMRLALAEARAAAAAGEVPVGAVVVRDGEVLAAAGNRTLRDRDPTAHAEVVALRAAARALGSERLPGCDLYVTLEPCAMCAGAISFARIRRLYFGAGDPKGGAVEHGPRFFAQPTCHHAPEVYGGLGEGEAAGLLRGFFAARR

SEQ ID NO: 407 - protein, 142 aa, synthetic construct
MRSALDEARRAAAAGEVPVGAVVVRDGAVLAAAGNRTVRDCDPTAHAEIVALRAAAAALGNYRLDGCDLYVTLEPCAMCAGAMVHARLARLVYGAADPRAGAAGSVLDVVRHPALNHRMEVTGGVLADECGALLREFFAARR

SEQ ID NO: 408 - protein, 168 aa, synthetic construct
MDSAAKDLCYMRRALELAALAEAEGEVPVGAVLVKDGEIVGEGWNRSIGSHDATAHAEIMALRQAGQALQNYRLLDTTLYVTLEPCPMCAGALLHSRVKRVVFGAPDLKAGAAGTVLNLFESQASYHYAEVESGLLEQECREQLQAFFKRRRKEIKALRQAEKEAQQK

SEQ ID NO: 409 - protein, 166 aa, synthetic construct
MDSAAKDLCYMRRALELAALAEAEGEVPVGAVLVKDGEIVGEGWNRSIGSHDATAHAEIMALRQAGQALQNYRLLDTTLYVTLEPCPMCAGALLHSRVKRVVFGAPDLKAGAAGTVLNLFESQASYHYAEVESGLLEEECREQLQAFFKRRRKEIKAQRDAKRAAD

SEQ ID NO: 410 - protein, 167 aa, synthetic construct
MTSAPEHEKYMRRALELARQAEEHGEVPVGAVLVKDGEIIAEGWNQSIGSHDATAHAEIMALRAAGKALENYRLLDTTLYVTLEPCPMCAGALLHSRVKRVVFGAPDLKAGAAGTVLNLFESQASYHYADVESGLLEEECREQLQAFFKRRRKEIKALKQAKREQEE

SEQ ID NO: 411 - protein, 146 aa, synthetic construct
DTEFMREALAEARAAAAAGEVPVGAVVVHEGKIIARAGNRTIRDNDPTAHAEIVALRAAARALGNYRLAGCTLYVTLEPCAMCAGAMIHARLDRLVYGADDPKAGAAGSVLNVLNHPRLNHQMQVESGVLADECAALLQDFFRARR

SEQ ID NO: 412 - protein, 148 aa, synthetic construct
MTDADFMALALEEARAAAALGEVPVGAVVVRDGAVIARAGNRTVRDNDPTAHAEIVALREAARALGNYRLDGCTLYVTLEPCAMCSGAMLHARLARVVYGAADPKTGAAGSVLDLFATAQLNHQTQVQGGVLAAECGALLQDFFRQRR

SEQ ID NO: 413 - protein, 160 aa, synthetic construct
DESYMRRALELARQAEQHGEVPVGAVLVKDGEVIAEGWNQSIGSHDATAHAEIMALRAAGQVLENYRLLDTTLYVTLEPCPMCAGALLHSRVKRVVFGAPDLKAGAAGTVLNLFESQASYHYAEVEGGLLEDECREQLQAFFKRRRKEIKAARQAKRAAE

SEQ ID NO: 414 - protein, 143 aa, synthetic construct
MDEALAEARRAAAAGEVPVGAVLVRDGRVLARGGNRTIRDCDPTAHAEIVALREAARAAGNYRLPGTTLYVTLEPCAMCAGAMIHARVARLVYGAADPRAGAAGSVFDVLRHPALNHRMEVEGGVRAEECGALLREFFRARRA

SEQ ID NO: 415 - protein, 150 aa, synthetic construct
MTEADLRFMQLALEEARRAGEAGEVPVGAVLVRDGAVLARGGNRTIRDCDPTAHAEIVALREAARALGNYRLDGCTLYVTLEPCAMCAGAMIHARLDRLVYGADDPRAGAAGSVLDVLGHPALNHQTRVTAGVLAEECGALLREFFRARR

SEQ ID NO: 416 - protein, 163 aa, synthetic construct
MTDEEYMRRALELARQAEQQGEVPVGAVLVKDGEVIAEGWNQSIGSHDATAHAEIMALRAAGQALENYRLLDTTLYVTLEPCPMCAGALLHSRVKRVVFGAPDLKAGAAGTVLNLFESQASYHYADVESGLLEEECREQLQAFFKRRRKEIKALKQAKRDAEE

SEQ ID NO: 417 - protein, 142 aa, synthetic construct
MRDALAEARAAAARGEVPVGAVVVRDGAVLARAGNASIAARDPTAHAEILALRAAARALGNHRLTGCTLYVTLEPCAMCAGAIAHARIARLVYGAADPKGGAVAHGPRFFAQPTCHHRPEVAGGLGEAEAAALLRDFFRARR

SEQ ID NO: 418 - protein, 148 aa, synthetic construct
MTDEDFMRLALAEAQAAAAAGEAPIGAVLVRDGQVLARGQNRVIRDNDPTAHAEIVALREAARQLGNYRLDGCELYVTLEPCAMCAGAMIHARLARLVYGAADPKAGAAGSVLDVINHPRLNHRMQVTAGVLADECGALLRDFFRQRR

SEQ ID NO: 419 - protein, 142 aa, synthetic construct
MRAALDEARAAADAGEVPVGAVVVRDGAILARAGNRTVRDADPTAHAEIVALRAAARALGNHRLGGCTLYVTLEPCAMCAGAIAQARVARLVYGAADPKGGAVAHGPRLFAQPTCHHRPEVTGGVLADEAGELLRAFFAARR

SEQ ID NO: 420 - protein, 160 aa, synthetic construct
DETYMRRALELARQAEQHGEVPVGAVLVKDGEVIAEGWNQSIGAHDATAHAEIMALRAAGQALQNYRLLDTTLYVTLEPCPMCAGALLHSRVKRVVFGAPDLKAGAAGTVLNLFESQASYHYAEVESGLLEEECREQLQAFFKRRRKEIKAARQAARDAQ

SEQ ID NO: 421 - protein, 164 aa, synthetic construct
MNDEFYMRRALELAKMAEERGEVPVGAVLVRDGEVIAEGWNQSIGSHDATAHAEIMALRAAGQVLQNYRLLDTTLYVTLEPCPMCAGALLHSRVKRVVFGAPDLKAGAAGTVLNLFESQASYHYAEVESGLLEEECREQLQAFFKRRRKEIKALKQAQREAEEK

SEQ ID NO: 422 - protein, 151 aa, synthetic construct
DTSPADLRFMRLALAEARAAAAAGEVPVGAVVVRDGAVLARAGNRTLRDRDPTAHAEIVALRAAARALGSHRLGGCTLYVTLEPCAMCAGAIAQARVARLVYGADDPKGGAVAHGPRLFAQPTCHHRPEVVGGVGAEEAAALLRDFFAARR

SEQ ID NO: 423 - protein, 164 aa, synthetic construct
MTAADERFMRRAIELAAQAEAAGEVPVGAVLVKDGEIVAEGWNQSIGSHDATAHAEIQTLRQAGQALENYRLLDTTLYVTLEPCPMCAGALLHSRVKRVVFGAPDLKAGAAGTVLNLFESQASYHYADVEGGLLEDECREQLQAFFKRRRKEIKAARQAAKEAE

SEQ ID NO: 424 - protein, 149 aa, synthetic construct
MDDDGLMRAAIAEAEAAAAAGEVPVGAVLVRDGVVLARGRNRMIADSDPTAHAEIVALREAARALGNYRLEGCELYVTLEPCAMCAGAMVHARLARLVYGAADPKAGAAGSVLDVLGHPRLNHRMEVTGGVLADECGAMLRDFFRARRA

SEQ ID NO: 425 - protein, 162 aa, synthetic construct
DDTFYMRRALELAALAESEGEVPVGAVLVKDGEIIAEGWNRSIGSHDATAHAEIETLRKAGQALQNYRLLDTTLYVTLEPCPMCAGALLHSRVKRIVFGAPDLKAGAAGTVLNLFESQASYHYAEVESGLLEEECREQLQAFFKRRRKEIKALRQAEKAAQE

SEQ ID NO: 426 - protein, 149 aa, synthetic construct
MDDAGFMREALAEARAAAAAGEVPVGAVVVKDGEIIARAGNRTIRDCDPTAHAEIVALRAAAQALGNYRLDGCTLYVTLEPCAMCSGAMLHARLPRVVYGAADPKTGAAGSVLDLFAEPRLNHHTRVEGGVLAAECGALLTDFFRARRG

SEQ ID NO: 427 - protein, 144 aa, synthetic construct
MDAALEEARRAAAAGEVPVGAVLVRDGVVLARAGNRTVRDCDPTAHAEIVALRAAAAALGNYRLPGTTLYVTLEPCAMCAGAMIHARVARLVYGAADPKTGAAGSVFDVLGSPRLNHRVEVEGGVRAAECGALLQDFFRARRAG

SEQ ID NO: 428 - protein, 149 aa, synthetic construct
MTDDERWMAEALREAEAAAAEGEVPVGAVVVRDGELIARGRNRVVRDCDPTAHAEIVALREAARALGNYRLTGCTLYVTLEPCAMCAGAMVHARLDRLVYGAADPKAGAAGSVLAVLNHPRLNHQMEVAGGVLAGESAALLRDFFRARR

SEQ ID NO: 429 - protein, 146 aa, synthetic construct
DSEFMAEALAEARAAAEAGEVPIGAVVVCDGEIVARAGNRTIRDNDPTAHAEIVALREAAAKLGNYRLAGCTLYVTLEPCAMCAGAMIHARLDRLVYGAADPKAGAAGSVLDVLGHPQLNHQTEVEGGVLAEECGALLKDFFASRR

SEQ ID NO: 430 - protein, 160 aa, synthetic construct
DPEYMRRALELAAQAEAEGEVPVGAVLVKDGEVVAEGWNRSIGDHDATAHAEIQVLRKAGQALQNYRLLDTTLYVTLEPCPMCAGALLHSRVKRVVFGAPDLKAGAAGTVLNLFESQASYHYADVESGLLEDECREQLQAFFKRRRKEIKAARKAAKEAE

SEQ ID NO: 431 - protein, 146 aa, synthetic construct
DTEMMRLALAEARAAAEAGEVPVGAVVVRDGVVLARAGNRTVRDNDPTAHAEIVALREAARALGNHRLEGCTLYVTLEPCAMCAGAIAQARVARLVYGAADPKGGAVAHGPRLFEQPTTHHRPEVTGGVLAEEAGALLREFFAARR

SEQ ID NO: 432 - protein, 148 aa, synthetic construct
MTDADFMALALEEARAAAALGEVPVGAVVVRDGAVVARAGNRTVRDCDPTAHAEVVALREAARVLGNYRVDGATLYVTLEPCAMCAGAMIHARLPRLVYGAADPRAGAAGSVLDVLGHPALNHRVEVTGGVLADESAALLREFFRERR

SEQ ID NO: 433 - protein, 152 aa, synthetic construct
DEPLDRDRYFMEIALAEARAAAAAGEVPVGAVVVRDGEVIARAGNRTVRDNDPTAHAEIVALREAARALGNHRLTGCTLYVTLEPCAMCAGAISHARVARLVYGADDPKGGAVAHGPRFFEQPTCHHRPEVTGGVLAAESAALLRGFFAARR

SEQ ID NO: 434 - protein, 149 aa, synthetic construct
MTSDAEFMALALEEARAAAALGEVPVGAVVVRDGAVIARAGNRTIRDCDPTAHAEMVALREAARALGNYRLAGCDLYVTLEPCAMCAGAIVHARLARLVYGAADPKTGAAGSVFDVLGSGRLNHRPEVVAGVLADECGALLTDFFRARR

SEQ ID NO: 435 - protein, 165 aa, synthetic construct
MTEEDKYFMQRAIELAKLAEENGEVPVGAVLVKDGEIIAEGWNQSIGNHDATAHAEIMTLRQAGQVLQNYRLLDTTLYVTLEPCPMCAGALLHSRVKRIVFGAPDLKAGAAGTVLNLFESQASYHYADVESGLLEEECREQLQAFFKRRRKEIKALKKQQKLEQE

SEQ ID NO: 436 - protein, 142 aa, synthetic construct
MSAALDEARRAAAAGEVPVGAVVVRDGVVLAAAGNRTLRDCDPTAHAEIVALRAAARALGNYRLDGCDLYVTLEPCAMCAGAMIHARLARLVYGAADPKTGAAGSVLDVLGHPRLNHRTAVTGGVLADECGALLRDFFAARR

SEQ ID NO: 437 - protein, 151 aa, synthetic construct
DTSERDERFMRLALEEARAAAEAGEAPIGAVVVRDGAVLARAGNRTVRDNDPTAHAEVVALREAARALGNHRLTGCTLYVTLEPCAMCAGAISHARVARLVYGADDPKGGAVAHGPRFFAQPTCHHRPEVTGGVLAEESAELLRGFFRARR

SEQ ID NO: 438 - protein, 148 aa, synthetic construct
DPDSRFMQAALAEARAAAEAGEVPIGAVVVRDGEIVARAGNRTIRDNDPTAHAEIVALREAARVLGNYRLTGCTLYVTLEPCAMCAGAMIHARIDRLVYGAADPKAGAAGSVLDVLNHPKLNHQMEVEAGVLADECGALLRDFFRARR

SEQ ID NO: 439 - protein, 149 aa, synthetic construct
MDDDERWMRLALEEARLAEEAGEVPVGAVVVRDGELLARGRNRVLRDCDPTAHAEIVALREAARRLGNYRLDGCTLYATLEPCAMCAGAMVHARLARLVYGAPDPKAGAAGSVLDVLNHPRLNHRMEVTAGVLAEECGALLRRFFQARR

SEQ ID NO: 440 - protein, 142 aa, synthetic construct
MSAALAEARRAAAAGEVPVGAVVVRDGAVLAAAGNRTVRDCDPTAHAEIVALRAAARALGNHRLEGCTLYVTLEPCAMCAGAIAQARVARLVYGAADPKGGAVAHGPRLFAQPTCHHRPEVVGGVGEAEAAALLRDFFAARR

SEQ ID NO: 441 - protein, 142 aa, synthetic construct
MRSALALAAAAGAAGEVPVGAVVVRDGVVIGRGENRVIRDSDPTAHAEIVALRDAARALGNYRLTGCTLYVTLEPCAMCAGAMIHARIDRLVYGAADPKAGAAGSVLDVLNHPQLNHQMEVEGGVLAEECGALLRDFFRARR

SEQ ID NO: 442 - protein, 150 aa, synthetic construct
RTADERWMRLALEEARAAEAAGEVPVGAVVVRDGELIARGRNRVLRDCDPTAHAEIVALREAARALGNYRVEGCTLYVTLEPCAMCAGAMIHARLDRLVYGAADPRAGAAGSVLDVLGHPALNHQTQVTGGVLAEECGALLREFFRARRG

SEQ ID NO: 443 - protein, 147 aa, synthetic construct
DDLSFMQEALAEARAAAEAGEVPIGAVVVRDGAILARAGNRTVRDNDPTAHAEIVALREAARALGNYRLAGCTLYVTLEPCAMCAGAMIHARVDRLVYGAADPKAGAAGSVLDVLGHPRLNHRMEVEGGVLAEECGALLREFFRARR

SEQ ID NO: 444 - protein, 149 aa, synthetic construct
MSSDAGFMAEALAEAEAAAAAGEVPVGAVVVCGGEIVARAGNRTIRDCDPTAHAEMVALREAARKLGNYRLAGCTLYVTLEPCAMCAGAMIHARLDRLVYGAADPKAGAAGSVLDVINHPRLNHQMEVEAGVLADECGAMLRRFFQERR

SEQ ID NO: 445 - protein, 148 aa, synthetic construct
MDDDRWMRLALEEARRAEEEGEVPVGAVVVLGGEVIGRGRNRVIRDSDPTAHAEIVALREAARTLGNYRLDGCELYVTLEPCAMCAGAMVHARLARLVYGAADPRAGAAGSVLDVLGHPRLNHRMEVTGGVLAEECGALLRDFFRRRR

SEQ ID NO: 446 - protein, 148 aa, synthetic construct
MDDAGFMREALAEAEAAAAAGEVPVGAVVVCDGRIVARAGNRTVRDNDPTAHAEIVALREAARALGNHRLGGCTLYVTLEPCAMCAGAMIHARIDRLVYGADDPKAGAVRSVLQVLDHPRLNHRMAVESGVLAGECAAVLQAFFAARR

SEQ ID NO: 447 - protein, 142 aa, synthetic construct
MDAALEEARRAAAAGEVPVGAVLVRDGEVLARGGNRTIRDCDPTAHAEIVALRAAARAAGNYRLPGTTLYVTLEPCAMCAGAMIHARVARLVYGAADPRAGAAGSVFDVLRHPALNHRMEVEGGVLAEESAALLRGFFRARR

SEQ ID NO: 448 - protein, 142 aa, synthetic construct
MRDALALARAAAAAGEVPVGAVVVKDGVVVGRGENRVVRDSDPTAHAEIVAMREAARALGNYRLTGCTLYVTLEPCAMCAGAMIHARLDRVVYGAADPKTGAAGSVLDVLGHPRLNHQTQVEGGVLAAECGALLSDFFRARR

SEQ ID NO: 449 - protein, 148 aa, synthetic construct
MDDAGFMREALAEAEAAAAAGEVPVGAVVVKDGEIIARAGNRTVRDCDPTAHAEIVALREAARKLGNYRLAGCTLYVTLEPCAMCAGAMIHARLDRLVYGADDPKAGAAGSVLNVLNHPRLNHQMQVEAGVLAEECGAMLRRFFETRR

SEQ ID NO: 450 - protein, 148 aa, synthetic construct
MDDAAFMGEALAEARAAAAAGEVPIGAVVVCDGEIVARAGNRTVRDNDPTAHAEIVALREAARKLGNYRLAGCTLYVTLEPCAMCAGAMIHARVDRLVYGADDPKAGAARSVLNVVDHPALNHRLEVTSGVLAAECAELLRGFFERRR

SEQ ID NO: 451 - protein, 150 aa, synthetic construct
MTAADAAAMRAALAEARAAAARGEVPVGAVVMRDGVIVARAGNATVAGHDPTAHAEIVALRRAARALGNHRLTGCALYVTLEPCAMCAGAIAHARIARLVYGAADPKGGAVAHGPRFFAQPTCHHRPEVTGGVLAEEAGALLRAFFRARR

SEQ ID NO: 452 - protein, 146 aa, synthetic construct
DTEMMALALAEARAAAEAGEVPVGAVVVRDGEVLARAGNRTVRDCDPTAHAEIVALREAARRLGNYRLPGCTLYVTLEPCAMCAGAIMHARLDRVVYGAADPKTGAAGSVLDLFAEPRLNHHTAVVGGVLADEAGALLRGFFAARR

SEQ ID NO: 453 - protein, 164 aa, synthetic construct
MTEEDKYFMKRAIELAKLAEENGEVPVGAVLVKDGEIISEGWNQSIGNNDATAHAEIMALREAGKVLENYRLLDTTLYVTLEPCPMCAGALLHSRVKRIVFGAPDLKAGAAGTVLNLFESQASYHYADVESGLLEEECREQLQAFFKRRRKEKKELKKLKREEN

SEQ ID NO: 454 - protein, 168 aa, synthetic construct
MDSAAKDLCYMRRALELAALAEAEGEVPVGAVLVKDGEIVGEGWNRSIGSHDATAHAEIMALRQAGQALQNYRLLDTTLYVTLEPCPMCAGALLHSRVKRVVFGAPDLKAGAAGTVLNLFESQASYHYADVESGLLEEECRAQLQAFFKRRRKEIKAQRDAQRAAQEK

SEQ ID NO: 455 - protein, 142 aa, synthetic construct
MRTALEEARRAAEAGEVPVGAVVVRDGVVLARAHNRTVADHDPTAHAEILALREAARVLGNHRLTGCTLYVTLEPCAMCAGAIVHARLDRLVYGAADPKAGAAGSVLDVLGHPRLNHRPEVAAGVLAEECGELLRGFFRERR

SEQ ID NO: 456 - protein, 149 aa, synthetic construct
DDDERWMREALAEARAAEEAGEVPVGAVVVRDGELVGRGRNRVLRDSDPTAHAEIVALREAARALGNYRLDGCTLYATLEPCAMCAGAMLHARLARLVYGAADPKAGAAGSVLDVLNHPRLNHRMEVTGGVLAEECGALLRGFFRARRG

SEQ ID NO: 457 - protein, 150 aa, synthetic construct
RSSPDERFMALALAEARRAAEAGEVPVGAVLVRDGAVLAAAGNRTIRDNDPTAHAEIVVLREAARRLGNYRLEGCTLYVTLEPCAMCAGAMVNARLGRLVYGAADPRAGAAGSVLDVLRHPALNHRMEVTGGVLAAECGALLRDFFAARR

SEQ ID NO: 458 - protein, 142 aa, synthetic construct
MRDALAEARKAAALGEVPVGAVVVKDGAVIARGHNRTVADSDPTAHAEIVAIRAAARALGNHRLTGCTLYVTLEPCAMCAGAIAHARIARLVYGAADPKGGAVDHGVRFFESPTCHHRPEVTGGVLAAEAAALLRDFFRARR

SEQ ID NO: 459 - protein, 162 aa, synthetic construct
DETYMRRALELARQAEEHGEVPVGAVLVKDGEIIAEGWNQSIGAHDATAHAEIMALRAAGQALENYRLLDTTLYVTLEPCPMCAGALLHSRVKRIVFGAPDLKAGAAGTVLNLFESQASYHYADVESGLLEEECREQLQAFFKRRRKEIKALKQAQREAEEK

SEQ ID NO: 460 - protein, 142 aa, synthetic construct
MDEALAEARRAAAAGEVPVGAVVVRDGAVLARAGNRTVRDCDPTAHAEIVALRAAARALGNHRLTGCDLYVTLEPCAMCAGAISHARIARLYYGADDPKGGAVAHGPRFFGQPTCHHRPEVYGGIGEGAAAALLRAFFAARR

SEQ ID NO: 461 - protein, 162 aa, synthetic construct
DESYMRRALELARQAEAEGEVPVGAVLVKDGEVVAEGWNRSIGAHDATAHAEIETLRKAGQALENYRLLDTTLYVTLEPCPMCAGALLHSRVKRVVFGAPDLKAGAAGTVLNLFESQASYHYAEVESGLLEDECREQLQAFFKRRRKEIKAQRQAQKEAEQK

SEQ ID NO: 462 - protein, 165 aa, synthetic construct
MSEQDRYFMQRAIELAQQAELNGEVPVGAVLVKDGEVIAEGWNQSIGSHDATAHAEIMALRKAGQVLENYRLLDTTLYVTLEPCPMCAGALLHSRVKRVVFGAPDLKAGAAGTVLNLFESQASYHYADVESGLLEDECREQLQAFFKRRRKEKKALKQAQKEAAQ

SEQ ID NO: 463 - protein, 149 aa, synthetic construct
MTDADFMALALEEARAAAALGEVPVGAVVVKDGEVIARAGNRTVRDCDPTAHAEIVALREAARKLGNYRLDGCTLYVTLEPCAMCAGAMLHARLPRVVYGAADPKTGAAGSVLDLFAEPRLNHQTAVQGGVLADEAGALLRDFFKARRA

SEQ ID NO: 464 - protein, 143 aa, synthetic construct
MDAALEEARRAAAAGEVPVGAVLVRDGVVLARAGNRTVRDCDPTAHAEIVALRAAAAALGNYRLDGCTLYVTLEPCAMCAGAMIHARLARLVYGAADPRTGAAGSVLDVLGHPALNHRTEVTGGVLAEECGALLREFFRTRRG

SEQ ID NO: 465 - protein, 150 aa, synthetic construct
MSDKDLRFMQLALEEARAAADAGEVPVGAVLVRDGQVLARGRNRVILDNDPTAHAEIVALREGARRLGNYRLAGCTLYVTLEPCAMCAGAIVHARLDRLVYGAADPKAGAAGSVLDVLNHPKLNHQMEVEGGVLAAESGELLRSFFRARR

SEQ ID NO: 466 - protein, 160 aa, synthetic construct
DTLYMRRALELAAQAEAEGEVPVGAVLVKDGEVIAEGWNRSIGSHDATAHAEIETLRKAGQVLQNYRLLDTTLYVTLEPCPMCAGALLHSRVKRIVYGAPDLKAGAAGTVLNLFESQASYHYAEVESGLLEEECREQLQAFFKRRRKEIKALRQAQKEAE

SEQ ID NO: 467 - protein, 143 aa, synthetic construct
MDAALEEARRAAEAGEVPVGAVLVRDGAVLARAGNRTIRDCDPTAHAEIVALRAAARALGNYRLPGTTLYVTLEPCAMCAGAMIHARVDRLVYGAADPRAGAAGSVLDVIGHPALNHRMEVEGGVLAEECGALLREFFRARRG

SEQ ID NO: 468 - protein, 149 aa, synthetic construct
DPLMDEFMGEALAEARAAAAAGEVPVGAVVVRDGAVIARAGNRTVRDCDPTAHAEIVALRAAARALGNHRLDGCDLYVTLEPCAMCAGAISHARIRRLYYGADDPKGGAVAHGARVFAHPTCHHRPEVYGGIGAAEAAGLLRAFFAARR

SEQ ID NO: 469 - protein, 160 aa, synthetic construct
DESYMRRALELARQAEQEGEVPVGAVLVKDGEVIAEGWNRSIGAHDATAHAEIETLRKAGQVLQNYRLLDTTLYVTLEPCPMCAGALLHSRVKRVVFGAPDLKAGAAGTVLNLFESQASYHYADVESGLLEDECREQLQAFFKRRRKEIKAQRDAEKAAK

SEQ ID NO: 470 - protein, 144 aa, synthetic construct
MRDALAEARAAAARGEVPVGAVVVRDGAVLARAGNATIADCDPTAHAEMRALRAAARALGNYRLPGCTLYVTVEPCAMCAGAMIHARLARLVYGAADPKAGAAGSVLDVLGHPALNHRMEVTGGVLAAECAALLRDFFAARRGR

SEQ ID NO: 471 - protein, 165 aa, synthetic construct
MTEEDKKFMQRAIELARKGEQEGEVPVGAVLVKDGEIIAEGWNRSIGDHDATAHAEIETLRKAGKALENYRLLDTTLYVTLEPCPMCAGALLHSRVKRVVFGAPDLKAGAAGTVLNLFESQASYHYADVESGLLEEECREQLQAFFKRRRKEKKALKQAKKEAEK

SEQ ID NO: 472 - protein, 148 aa, synthetic construct
MDDDALMGLALDEARAAAAAGEVPIGAVVARDGAVVARAGNRTVRDCDPTAHAEVVALREAARALGNYRLAGCTLYVTLEPCAMCAGAMIHARLDRVVYGAADPKTGAAGSVLDVLGHPALNHQTRVEGGVLAAECGALLRDFFAARR

SEQ ID NO: 473 - protein, 165 aa, synthetic construct
MTRDEQYMRRALELARQAEQEGEVPVGAVLVKDGEIVAEGWNRSIGDHDATAHAEIETLRKAGQVLENYRLLDTTLYVTLEPCPMCAGALLHSRVKRVVFGAPDLKAGAAGTVLNLFESQASYHYAEVESGLLEEECREQLQAFFKRRRKEIKALRQAEKEAEQK

SEQ ID NO: 474 - protein, 145 aa, synthetic construct
DSEFMAEALAEARAAAAAGEVPVGAVVVKDGEIIARAGNRTLRDNDPTAHAEIVALRAAARALGNQRLEGCDLYVTLEPCAMCAGAISHARIRRLYYGAEDPKGGAVDNGVRFFASPTCHHRPEVYGGIGETEAAELLRGFFRER

SEQ ID NO: 475 - protein, 148 aa, synthetic construct
DDDERFMRLALEEARKAEEAGEVPVGAVLVKDGEVIARGRNRVISDSDPTAHAEIVALREAGRALGNYRLDGCTLYVTLEPCAMCAGAMVHARLDRLVYGAADPRAGAAGSVLDVLNHPALNHRMEVEGGVLADECGALLRDFFRRRR

SEQ ID NO: 476 - protein, 142 aa, synthetic construct
MDAALAEARAAADAGEVPVGAVVVRDGAVLARAGNRTLRDSDPTAHAEIVALRAAARALGNHRLGGCTLYVTLEPCAMCAGAISHARLARLHYGADDPKGGAVAHGPRFFAQPTCHHRPDVYGGIGEGEAAALLRGFFAARR

SEQ ID NO: 477 - protein, 146 aa, synthetic construct
DTEFMREALAEARAAAAAGEVPVGAVVVKDGEIIARAGNRTLRDNDPTAHAEIVALREAARKLGNHRLTGCTLYVTLEPCAMCAGAISHARIARLVYGADDPKGGAVAHGPRFFAQPTCHHRPEVTGGVLAEEAGELLRGFFRERR

SEQ ID NO: 478 - protein, 144 aa, synthetic construct
MDAALEEARRAAAAGEVPVGAVLVAGGRVLARAGNRTIRDCDPTAHAEIVALREAARALGNYRLAGTTLYVTLEPCAMCAGAMIHARVDRLVYGAADPRAGAAGSVFDVVRHPALNHRLEVEGGVLAEECGALLRDFFRARRGR

SEQ ID NO: 479 - protein, 142 aa, synthetic construct
MRAALDEARRAAAAGEVPVGAVVVRDGVVLARAHNRTVADHDPTAHAEILALREAARVLGNHRLTGCTLYVTLEPCAMCAGAIVHARLDRLVYGAADPKAGAAGSVLDVLDHPRLNHRMEVTGGVLAEESAALLRGFFAARR

SEQ ID NO: 480 - protein, 149 aa, synthetic construct
MSSDADFMRLALAEARAAAAAGEVPVGAVVVRGGEVIARAGNRTVRDCDPTAHAEVVALREAARKLGNHRLTGCTLYVTLEPCAMCAGAISHARVARLVYGADDPKGGAVAHGPRFFAQPTCHHRPEVTGGVLAEEAGELLRGFFRARR

SEQ ID NO: 481 - protein, 144 aa, synthetic construct
MDAALEEARRAAAAGEVPVGAVLVRDGAVLARGGNRTIRDCDPTAHAEIVALRAAARALGNYRLPGTTLYVTLEPCAMCAGAMIHARVARLVYGAADPKTGAAGSVFDVLGSARLNHRVEVEGGVRAAECGALLRDFFAARRGR

SEQ ID NO: 482 - protein, 142 aa, synthetic construct
MRAALAEARRAAAEGEVPVGAVVVRDGAVLARAGNRTVRDCDPTAHAEVLALREAARALGNHRLTGCTLYVTVEPCAMCAGAISHARVARLVYGADDPKGGAVRHGPRVFDQPTCHHRPEVVGGVLAEEAGALLRDFFAARR

SEQ ID NO: 483 - protein, 145 aa, synthetic construct
DTSFMQQALDEARAAAAAGEVPVGAVVVKDGEIIARAGNRTLRDNDPTAHAEIVALRAAARALGSQRLGGCDLYVTLEPCAMCAGAISHARIARLYYGADDPKGGAVAHGARVFSHPQCHHVPEVYDGIGAGEAAALLRDFFAGR

SEQ ID NO: 484 - protein, 146 aa, synthetic construct
DTEFMREALAEARAAAAAGEVPVGAVVVRDGAIIARAGNRTIRDRDPTAHAEVVALREAARALGNHRLTGCTLYVTLEPCAMCAGAISHARVARLVYGAADPKGGAVAHGPRFFEQPTCHHRPEVTGGVLAEEAGALLRDFFRARR

SEQ ID NO: 485 - protein, 164 aa, synthetic construct
MSEQDEFYMRRAIELARKGEENGEVPVGAVLVKDGEIIAEGWNQSIGSHDATAHAEIVTLRKAGQVLENYRLLDTTLYVTLEPCPMCAGALLHSRVKRVVFGAPDLKAGAAGTVLNLFESQASYHYADVESGLLEEECREQLQAFFKRRRKEKKALKQAQKELE

SEQ ID NO: 486 - protein, 149 aa, synthetic construct
MTDADFMALALAEARAAAAAGEVPVGAVVVRDGVVLAAAGNRTLRDCDPTAHAEVVALRAAARALGNYRLDGCTLYVTLEPCAMCAGAMLHARLARVVYGAADPKTGAAGSVLDLFAQPRLNHHTAVAGGVLAAECGALLADFFRQRRG

SEQ ID NO: 487 - protein, 164 aa, synthetic construct
MSDEQYMRRALELARQAEQQGEVPVGAVLVRDGEVIAEGWNQSISSHDATAHAEMMAIRAAGAALENYRLLDTTLYVTLEPCPMCAGALLHSRVKRVVFGAPDLKAGAAGTVLNLFESQASYHYADVESGLLEEECREQLQAFFKRRRKEIKALRQAEKEAAAK

SEQ ID NO: 488 - protein, 157 aa, synthetic construct
MSSETSTDAAADAAFMAEALAEARAAAAAGEVPVGAVVVCGGRIVARAGNRTVRDNDPTAHAEIVALREAARVLGNHRLTGCTLYVTLEPCAMCAGAISHARIARLVYGADDPKGGAVRHGPRFFAQPTCHHRPDVAGGVGEAEAAELLRAFFRARR

SEQ ID NO: 489 - protein, 148 aa, synthetic construct
MTDADFMALALEEARAAAALGEVPVGAVVVKDGEVIARAGNRTIRDCDPTAHAEIVALREAARKLGNYRLDGCDLYVTLEPCAMCAGAMIHARLARLVYGAADPKTGAAGSVLDVFANPQLNHHTAVVGGVLADEAGALLREFFAARR

SEQ ID NO: 490 - protein, 149 aa, synthetic construct
MDDDARFMGEALAEARAAAAAGEVPIGAVVVRDGAVVARAGNRTVRDNDPTAHAEVLALREAARALGSQRLTGCTLYVTLEPCAMCAGAISHARVARLVYGAADPKGGAVAHGPRFFAQPTCHHRPEVTGGVGEAEAAALLRDFFRARR

SEQ ID NO: 491 - protein, 146 aa, synthetic construct
DRLFMEEALAEARAAAAAGEVPIGAVVVRDGEIVARAGNRTVRDCDPTAHAEIVALREAARALGNYRLTGCTLYVTIEPCAMCAGAMIHARLDRLVYGADDPKAGAVRSVLQVLDHPALNHRVEVESGVLAAECAALLQEFFASRR

SEQ ID NO: 492 - protein, 165 aa, synthetic construct
DSLTRDELYMRRALELAALAEAEGEVPVGAVLVKDGEIVGEGWNRSIGSHDATAHAEIMALRQAGQALQNYRLLDTTLYVTLEPCPMCAGALLHSRVKRVVFGASDLKAGAAGTVLNLFESQASYHYADVESGLLEQECREQLQAFFKRRRKEIKAQRQAEKAAQ

SEQ ID NO: 493 - protein, 142 aa, synthetic construct
MRAALAEARRAAEAGEVPVGAVVVRDGAVLAAAGNRTVRDCDPTAHAEIVALRAAARAAGNHRLTGCTLYVTLEPCAMCAGAISHARVARLVYGAADPKGGAVAHGPRFFEQPTCHHRPEVTGGVLAEEAGALLRDFFRARR

SEQ ID NO: 494 - protein, 142 aa, synthetic construct
MSAALEEARRAAAAGEVPVGAVLVRDGAVLARGGNRTIRDCDPTAHAEIVALRAAARALGNYRLPGTTLYVTLEPCAMCAGAMIHARVARLVYGAADPKTGAAGSVFDVLRHPALNHRMEVEGGVLAEECGALLRDFFRARR

SEQ ID NO: 495 - protein, 147 aa, synthetic construct
DDTEFMRLALAEAEAAAAAGEVPVGAVVVRDGEVIARAGNRTIRDCDPTAHAEVVALREAARKLGNYRLAGCTLYVTLEPCAMCAGAMIHARLDRVVYGAADPKTGAAGSVLDLFATRQLNHHTQVTGGVLAEECGALLRGFFEARR

SEQ ID NO: 496 - protein, 161 aa, synthetic construct
DTLYMRRALELAAQAEAEGEVPVGAVLVKDGEVIAEGWNRSIGDHDATAHAEIQTLRKAGQVLQNYRLLDTTLYVTLEPCPMCAGALLHSRVKRIVYGAPDLKAGAAGTVLNLFESQASYHYADVESGLLEDECREQLQAFFKRRRKEIKALRQAEKAAEQ

SEQ ID NO: 497 - protein, 146 aa, synthetic construct
DDPYMALALEEARAAAAAGEVPIGAVVVRDGEVVARAGNRTVRDNDPTAHAEVVALREAARALGNHRLTGCTLYVTLEPCAMCAGAISHARVARLVYGADDPKGGAVRHGARVFEHPQCHHRPEVTGGVGAAEAGELLRGFFRARR

SEQ ID NO: 498 - protein, 168 aa, synthetic construct
MDSKEKDEFFMRRALELARLGEEKGEVPVGAVLVKDGEIIAEGWNQSIGENDATAHAEIMALRKAGKALENYRLLDTTLYVTLEPCPMCAGALLHSRVKRIVFGAPDLKAGAAGTVLNLFESQASYHYADVESGLLEEECREQLQAFFKRRRKEIKEKKKAEKAALEA

SEQ ID NO: 499 - protein, 142 aa, synthetic construct
MRSALDLAAAAAAAGEVPVGAVVVRDGAIVGRGENRVLRDSDPTAHAEIVALREAARALGNYRLTGCTLYVTLEPCAMCAGAMIHARIDRLVYGAADPKAGAAGSVLDVLGHPALNHQMEVEGGVLAEECGALLRDFFRARR

SEQ ID NO: 500 - protein, 142 aa, synthetic construct
MDAALAEARAAADAGEVPVGAVVVRDGVVLARAGNRTVRDADPTAHAEIVALRAAARALGNHRLGGCTLYVTLEPCAMCAGAISHARVARLVYGADDPKGGAVRHGPRLFASPTCHHRPEVVSGVGEAEAAALLRDFFAARR

SEQ ID NO: 501 - protein, 142 aa, synthetic construct
MDAALEEARRAAAAGEVPVGAVLVRDGVVLARAGNRTIRDCDPTAHAEIVALREAARAAGNYRLPGTTLYVTLEPCAMCAGAMIHARVDRLVYGAADPRAGAAGSVFDVVRHPALNHRMEVEGGVLADESAALLRGFFRARR

SEQ ID NO: 502 - protein, 166 aa, synthetic construct
MTSEDEKYMRRALELARQAEQEGEVPVGAVLVKDGEIVAEGWNRSIGDHDATAHAEIETLRKAGQVLENYRLLDTTLYVTLEPCPMCAGALLHSRVKRVVFGAPDLKAGAAGTVLNLFESQASYHYADVESGLLEEECREQLQAFFKRRRKEIKALRQAQKEAEQK

SEQ ID NO: 503 - protein, 146 aa, synthetic construct
DEPYMRLALDEARAAAAAGEVPIGAVVVRDGEVLARAGNRTVRDNDPTAHAEVLALREAARALGNHRLTGCTLYVTLEPCAMCAGAISHARVARLVYGADDPKGGAVAHGPRFFAQPTCHHRPEVTGGVLAEEAGALLRDFFRARR

SEQ ID NO: 504 - protein, 142 aa, synthetic construct
MDAALAEARAAADAGEVPVGAVVVRDGAVLARAGNRTLRDADPTAHAEIVALRAAARALGNHRLGGCTLYVTLEPCAMCAGAISHARLARLVYGADDPKGGAVAHGPRFFGQPTCHHRPEVVGGVGAAAAGDLLRGFFRARR

SEQ ID NO: 505 - protein, 157 aa, synthetic construct
RSSPEEAAMDDAGFMRLALAEAEAAAAAGEVPVGAVVVLGGEVIARAGNRTVRDNDPTAHAEIVALREAARKLGNYRLAGCTLYVTLEPCAMCAGAMIHARLARLVYGAADPKTGAAGSVLDLVAHPALNHRMEVEGGVLAAECGALLRDFFAARRG

SEQ ID NO: 506 - protein, 164 aa, synthetic construct
MTRDEQYMRRALELARQAEAEGEVPVGAVLVRDGEVIAEGWNRSIGAHDATAHAEIMALRQAGQVLENYRLLDTTLYVTLEPCPMCAGALLHSRVKRVVYGAPDLKAGAAGTVLNLFESQASYHYAEVEGGLLEEECREQLQAFFKRRRKEIKAARQARRAAEE

SEQ ID NO: 507 - protein, 142 aa, synthetic construct
MRAALAEARAAAEAGEVPVGAVVVHEGRIIARAQNRVERDHDPTAHAEILALRAAAAALGATRLGGCTLYVTLEPCAMCAGAIAHARVARLVYGADDPKGGAVAHGPRFFTQPTCHHRPEVTGGVGEAEAAALLRDFFRARR

SEQ ID NO: 508 - protein, 148 aa, synthetic construct
MDDAGFMREALAEAEAAAAAGEVPVGAVVVKDGEIIARARNATVARNDPTAHAEILALREAARVLGNYRLTGCTLYATLEPCAMCAGAILHARIARLVYGAADPKAGACGSVLDVIGHPRLNHRVEVAGGVLAEECGALLREFFRSRR

SEQ ID NO: 509 - protein, 161 aa, synthetic construct
DETYMRRALELARQAEQAGEVPVGAVLVKDGEIVAEGWNQSIGTHDATAHAEIMALRAAGQALENYRLVDTTLYVTLEPCPMCAGALLHSRVKRVVFGAADLKAGAAGTVLNLFESQASYHYAEVESGLLEEECREQLQAFFKRRRKEIKALKQAEKAAQE

SEQ ID NO: 510 - protein, 164 aa, synthetic construct
MNSDDLYMRRALELARQAEEEGEVPVGAVLVKDGEIVAEGWNRSIGSHDATAHAEIETLRKAGQVLQNYRLLDTTLYVTLEPCPMCAGALLHSRVKRVVFGAPDLKAGAAGTVLNLFESQASYHYADVESGLLEDECREQLQAFFKRRRKEIKALRQAQKEAEE

SEQ ID NO: 511 - protein, 142 aa, synthetic construct
MDAALAEARAAADAGEVPVGAVLVRDGVVLARAGNRTVRDCDPTAHAEIVALRAAARALGNYRLPGTTLYVTLEPCAMCAGAMIHARVDRLVFGARDPRAGAAGSVFDVLRHPALNHRVEVVEGVRAEECGALLRDFFRARR

SEQ ID NO: 512 - protein, 160 aa, synthetic construct
DRLFMRRALELAAQAEAEGEVPVGAVLVKDGEVIAEGWNRSIGSHDATAHAEIETLRKAGQVLENYRLLDTTLYVTLEPCPMCAGALLHSRVKRIVYGAPDLKAGAAGTVLNLFESQASYHYADVESGLLEDECREQLQAFFKRRRKEIKAARQAKREQE

SEQ ID NO: 513 - protein, 149 aa, synthetic construct
MDDARFMAEALAEARAAAAAGEVPVGAVVVRDGAVLARAGNRTVRDCDPTAHAEIVALREAARALGNYRLDGCTLYVTLEPCAMCAGAMLHARLPRLVYGAADPKTGAAGSVLDLFAERRLNHQTEVRGGVLAEACGALLTDFFRSRRA

SEQ ID NO: 514 - protein, 146 aa, synthetic construct
DSEFMAEALAEARAAAEAGEVPIGAVVVCDGEIVARAGNRTIRDNDPTAHAEIVALREAARKLGNYRLTGCTVYVTLEPCAMCAGAMIHARLDRLVYGAADPKAGAAGSVLDVLGHPRLNHQMAVESGVLADECGALLRDFFRSRR

SEQ ID NO: 515 - protein, 151 aa, synthetic construct
MDSESDLRFMREALAEARAAAAAGEVPVGAVVVRDGAILARAGNRTLRDNDPTAHAEIVALRAAAAALGNHRLTGCTLYVTLEPCAMCAGAISHARIARLVYGAADPKGGAVAHGPRFFEQPTCHHRPEVAGGVLAEEAGALLRAFFAARR

SEQ ID NO: 516 - protein, 149 aa, synthetic construct
MSSDAGFMAEALAEARAAAAAGEVPVGAVVVRDGAIVARAGNRTVRDNDPTAHAEVVALREAARALGNHRLGGCELYVTLEPCAMCAGAISHARIARLYYGADDPKGGAVAHGARVFSHPQCHHRPEVYDGIGAGEAAALLRDFFAARR

SEQ ID NO: 517 - protein, 148 aa, synthetic construct
DRDDRYMRLALEEARAAAEAGEVPIGAVVVRDGEVLARAGNRTVRDCDPTAHAEIVALREAARVLGNYRLAGCTLYVTVEPCAMCAGAMIHARVDRLVYGADDPKGGAVRSCLQVLDHPRLNHRVEVTAGVLAEECAALLQSFFAARR

SEQ ID NO: 518 - protein, 146 aa, synthetic construct
DSEFMAEALAEARAAAAAGEVPVGAVVVRDGEIVARAGNRTLRDNDPTAHAEIVALREAARALGNHRLTGCTLYVTLEPCAMCAGAISHARIARLVYGADDPKGGGVAHGARVFEHPQCHHRPEVVGGVGAAEAGELLRGFFAARR

SEQ ID NO: 519 - protein, 164 aa, synthetic construct
DREEYLMRKALELAAKAEQLGEVPVGAVLVKDGEIIAEGWNQSIGNHDATAHAEIMTLRQAGQALENYRLLDTTLYVTLEPCPMCAGALLHSRVKRIVFGAPDLKAGAAGTVLNLFESQASYHYADVESGLLEDECREQLQAFFKRRRKEIKALKQAKRDAEEN

SEQ ID NO: 520 - protein, 142 aa, synthetic construct
MDAALAEARAAADAGEVPVGAVVVRDGAVLARAGNRTLRDADPTAHAEIVALRAAARALGNHRLGGCTLYVTLEPCAMCAGAISHARLARLHYGAADPKGGAVEHGPRFFAQPTCHHRPDVYGGIGEGEAAALLRGFFAARR

SEQ ID NO: 521 - protein, 146 aa, synthetic construct
DEPYMRLALDEARAAAAAGEVPIGAVVVKDGEVIARARNRTLADNDPTAHAEIVALRAAAAALGNHRLTGCELYVTLEPCAMCAGAISHARIARLVYGADDPKGGAVAHGPRFFAQPTCHHRPEVVGGVGEGEAAELLRGFFAARR

SEQ ID NO: 522 - protein, 148 aa, synthetic construct
DRDSRFMREALAEARAAAEAGEVPIGAVVVRDGEIVARAGNRTVRDCDPTAHAEIVALREAARALGNYRLTGCTLYVTIEPCAMCAGAMIHARIDRLVYGAADPKAGAAGSVLDVLGHPRLNHRMEVTAGVLAEECAALLRDFFAARR

SEQ ID NO: 523 - protein, 142 aa, synthetic construct
MDAALEEARRAAAAGEVPVGAVLVHDGQILARAGNRTIRDNDPTAHAEIVVLREAARALGNYRLEGCTLYVTLEPCAMCAGAMIHARLPRLVYGADDPKAGAAGSVLDVLNHPALNHRMEVEGGVLAAECGALLRDFFRARR

SEQ ID NO: 524 - protein, 151 aa, synthetic construct
MSAADDIRFMREALAEARAAAAAGEVPVGAVVVRDGAILARAGNRTIRDCDPTAHAEVVALREAARALGNHRLTGCTLYVTLEPCAMCAGAISQARVARLVYGADDPKGGAVAHGPRFFAQPTCHHRPEVTGGVLAEEAGALLRDFFRARR

SEQ ID NO: 525 - protein, 148 aa, synthetic construct
MTDADFMALALEEARAAAALGEVPVGAVVVKDGEVIARAGNRTIRDCDPTAHAEVVALREAARKLGNYRLPGLTLYVTLEPCAMCAGAMIHARLDRVVYGADDPKGGAARSVYRILDDPRLNHQVAVTSGVLAEECGALLRDFFRARR

SEQ ID NO: 526 - protein, 166 aa, synthetic construct
MDSAAKDLCYMRRALELAALAEAEGEVPVGAVLVRDGEIVGEGWNRSIGSHDATAHAEIMALRQAGQALQNYRLLDTTLYVTLEPCPMCAGALLHSRVKRVVFGAPDLKAGAAGTVLNLFESQASYHYADVEGGLLAEECRAQLQAFFKRRRKEQKALRQAQKEAA

SEQ ID NO: 527 - protein, 147 aa, synthetic construct
DEEFMREALAEAQAAADAGEVPIGAVVVCDGEIVARAGNRTIRDNDPTAHAEIVALREAARKLGNYRLAGCTLYVTLEPCAMCAGAMIHARVDRLVYGADDPKAGAAGSVLDVIGHPRLNHRMEVTSGVLAEECGAMLREFFRRRRA

SEQ ID NO: 528 - protein, 148 aa, synthetic construct
MDDAGFMRLALAEARAAAAAGEVPVGAVVVRGGEVLAAAGNRTLRDCDPTAHAEVVALRAAARALGNHRLDGCDLYVTLEPCAMCAGAISHARIARLYYGADDPKGGAVAHGPRFFAQPTCHHRPEVYGGIGESEAAALLRDFFAARR

SEQ ID NO: 529 - protein, 148 aa, synthetic construct
MSDEDYMRLALAEAQAAADAGEVPVGAVLVAGGEVVARGRNRMIADSDPTAHAEIVALREAARRLGNYRLTGCTLYVTLEPCAMCAGAIVHARLDRVVYGAADPKAGAAGSVLDVLGHPRLNHRTEVTGGVLADECGALLKDFFRARR

SEQ ID NO: 530 - protein, 163 aa, synthetic construct
MSSEDEKYMRRALELARQAEEEGEVPVGAVLVKDGEIVAEGWNRSIGSHDATAHAEIETLRKAGQALENYRLLDTTLYVTLEPCPMCAGALLHSRVKRIVFGAPDLKAGAAGTVLNLFESQASYHYADVESGLLEDECREQLQAFFKRRRKEIKAARKAAKAE

SEQ ID NO: 531 - protein, 149 aa, synthetic construct
MSDDAAFMGEALAEARAAAAAGEVPIGAVVVCDGAIVARAGNRTVRDNDPTAHAEIVALREAAARLGNYRLTGCTLYVTLEPCAMCAGAMIHARIDRLVYGAADPKAGAAGSVLDVIGHPRLNHRMEVEGGVLAAECGALLRDFFASRR

SEQ ID NO: 532 - protein, 142 aa, synthetic construct
MRAALAEAERAAAAGEVPVGAVVVRDGAVLAAAGNRTVRDCDPTAHAEVLALRAAARALGNHRLTGCTLYVTLEPCAMCAGAISHARVARLVYGADDPKGGAVAHGPRFFAQPTCHHRPEVAGGLGAAAAGELLRAFFRARR

SEQ ID NO: 533 - protein, 142 aa, synthetic construct
MRAALDEARAAAAAGEVPVGAVVVHEGRIVARAQNRMRRDNDPTAHAEIVALRAAAAALGSMRLTGCTLYVTLEPCAMCAGAISHARIDRLVYGAADPKGGAVAHGPRFFEQPTCHHRPDVVGGVLAEEAGALLRGFFAARR

SEQ ID NO: 534 - protein, 167 aa, synthetic construct
MDSAAKDLCYMRRALELAALAEAEGEVPVGAVLVKDGEIVGEGWNRSIGSHDATAHAEIMALRQAGQALQNYRLLDTTLYVTLEPCPMCAGALLHSRVKRVVFGAPDLKAGAAGTVLNLFESQASYHYADVESGLLEQECREQLQAFFKRRRKEIKAQRDAQRAADE

SEQ ID NO: 535 - protein, 160 aa, synthetic construct
MNDEFYMRRALELAALAEEHNEVPVGAVLVLNGEIIGEGWNRSIGQHDATAHAEIMALRQAGKVLQNYRLLDTTLYVTLEPCPMCAGALVHSRVKRVVYGTPDLKAGAAGTVMNLLSYDAVNHHVAITSGVLAEECREQLQAFFRRRRAEKKALKQAQRA

SEQ ID NO: 536 - protein, 161 aa, synthetic construct
DDTFYMRRALELARQAEAEGEVPVGAVLVRDGEVIAEGWNRSIGDHDATAHAEIQVLRKAGQVLQNYRLLDTTLYVTLEPCPMCAGALLHSRVKRVVYGAPDLKAGAAGTVLNLFESQASYHYADVESGLLEEECREQLQAFFKRRRKEIKAARQAKRAAE

SEQ ID NO: 537 - protein, 142 aa, synthetic construct
MRSALDLAAAAAAAGEVPVGAVVVRDGAIVGRGENRVLRDSDPTAHAEIVAMREAARALGNYRLTGCTLYVTLEPCAMCAGAMIHARIDRLVYGAADPKAGAAGSVLDVLNHPRLNHQMEVEGGVLAAECGAMLRDFFRARR

SEQ ID NO: 538 - protein, 148 aa, synthetic construct
MDDDALMGLALDEARAAAAAGEVPIGAVVARDGEVVARAGNRTVRDCDPTAHAEIVALREAARKLGNYRLTGCTLYVTLEPCAMCAGAMIHARLDRLVYGAADPKTGAAGSVLDVLGHPALNHQTQVEGGVLAEECGALLRDFFRERR

SEQ ID NO: 539 - protein, 165 aa, synthetic construct
MTEEDKYFMRRAIELAKLAEENGEVPVGAVLVKDGEIIAEGWNQSIGSHDATAHAEMMAIREAGKVLENYRLLDTTLYVTLEPCPMCAGALLHSRVKRIVFGAPDLKAGAAGTVLNLFESQASYHYADVESGLLEEECREQLQAFFKRRRKEIKALKKAQKEAEE

SEQ ID NO: 540 - protein, 146 aa, synthetic construct
DDDYMRLALAEARAAAEAGEVPIGAVVVCGGEVVARAGNRTVADCDPTAHAEIVALREAARKLGNYRLTGCTLYVTLEPCAMCAGAMIHARLDRLVYGADDPKAGAAGSVLDVLGHPALNHQMQVTAGVLADECAALLRDFFRARR

SEQ ID NO: 541 - protein, 163 aa, synthetic construct
DDSFYMRRALELAALAEREGEVPVGAVLVKDGEIIAEGWNRSIGSHDATAHAEIETLRKAGQALQNYRLLDTTLYVTLEPCPMCAGALLHSRVKRIVFGAPDLKAGAAGTVLNLFESQASYHYADVESGLLEEECREQLQAFFKRRRKEIKAQRDAEKAAAEK

SEQ ID NO: 542 - protein, 142 aa, synthetic construct
MDAALAEARAAADAGEVPVGAVVVRDGAVLARAGNRTLRDSDPTAHAEIVALRAAARALGNHRLGGCDLYVTLEPCAMCAGAISHARIARLYYGADDPKGGAVAHGARVFAHPQCHHRPEVYDGIGAAEAAALLRDFFAARR

SEQ ID NO: 543 - protein, 164 aa, synthetic construct
MTRDEQYMRRALELARQAEAEGEVPVGAVLVKDGEIIAEGWNRSIGSHDATGHAEIMALRAAGQALENYRLLDTTLYVTLEPCPMCAGALLHSRVKRVVFGAPDLKAGAAGTVLNLFESQASYHYADVESGLLEEECREQLQAFFKRRRKEIKALRQAEKAAAE

SEQ ID NO: 544 - protein, 151 aa, synthetic construct
MTDADFMALALEEARAAAALGEVPVGAVVVKDGEVIARAGNRTIRDCDPTAHAEIVALREAARKLGNYRLPGLALYVTLEPCAMCAGAMIHARLARLVYGAADPKTGAAGSVLDVLGHPALNHQTAVTGGVLAEEAGALLRDFFAARRAAG

SEQ ID NO: 545 - protein, 142 aa, synthetic construct
MDAALAEARAAADAGEVPVGAVVVRDGAVLARAGNRTLRDADPTAHAEIVALRAAARALGNHRLPGCTLYVTLEPCAMCAGAISHARIARLVYGAADPKGGAVAHGPRFFEQPTCHHRPEVVGGLGETEAAALLRAFFAARR

SEQ ID NO: 546 - protein, 142 aa, synthetic construct
MDAALEEARRAAAAGEVPVGAVLVAGGRVLARAGNRTIRDCDPTAHAEIVALRAAARALGNYRLPGTTLYVTLEPCAMCAGAMIHARVARLVYGAADPKTGAAGSVFDVLGSGRLNHRVAVEGGVRAEECGALLRDFFRARR

SEQ ID NO: 547 - protein, 148 aa, synthetic construct
DDDERWMREALAEARAAAEAGEVPVGAVVVRDGELIARGRNRVEADADPSAHAEIVALREAARRLGNHRLTGCTLYVTLEPCAMCAGAISHARVARLVYGADDPKGGAVRHGARVFEQPTCHHRPEVVGGVLASESAALLRDFFRARR

SEQ ID NO: 548 - protein, 165 aa, synthetic construct
MSAADEQFMRRAIELARQAEAEGEVPVGAVLVKDGEIVAEGWNRSIGAHDATAHAEIETLRKAGQALENYRLLDTTLYVTLEPCPMCAGALLHSRVKRVVFGAPDLKAGAAGTVLNLFESQASYHYADVESGLLEEECREQLQAFFKRRRKEIKALRQAQKAAEE

SEQ ID NO: 549 - protein, 152 aa, synthetic construct
MSSAPSDADFMGLALEQARLAAAAGEVPVGAVVVRDGEVIAQAHNRTVADCDPTAHAEVVALRAAARALGNHRLTGCTLYVTLEPCAMCAGAIAHARVARVVYGAADPKGGAVEHGPRFFAQPTCHHRPEVVGGVGAAEAGALLRDFFRARR

SEQ ID NO: 550 - protein, 143 aa, synthetic construct
MDEALAEARRAAAAGEVPVGAVVVRDGVVLARAGNRTVRDADPTAHAEIVALRAAAAALGNYRLDGCTLYVTLEPCAMCAGAMVHARLARLVYGAADPRAGAAGSVLDVLGHPALNHRMEVAGGVRAEECAALLRDFFAARRG

SEQ ID NO: 551 - protein, 142 aa, synthetic construct
MDEALAEARRAAEAGEVPVGAVVVRDGAVLARAGNRTVRDCDPTAHAEVLALREAARALGNHRLAGCTLYVTLEPCAMCAGAISHARVARLVYGADDPKGGAVAHGPRFFGQPTCHHRPEVAAGLGAAEAGALLRDFFAARR

SEQ ID NO: 552 - protein, 146 aa, synthetic construct
DTSFMRLALDEARAAADAGEVPVGAVVVCDGEVIARAGNRTVRDCDPTAHAEIVALREAARRLGNYRLAGCTLYVTLEPCAMCAGAMIHARLDRLVYGAADPKAGAAGSVLDVLGHPALNHQTQVEGGVLAAECGALLRDFFRARR

SEQ ID NO: 553 - protein, 161 aa, synthetic construct
DTLFMRRALELAAQAEAEGEVPVGAVLVKDGEVIAEGWNRSIGSHDATAHAEIETLRKAGQVLQNYRLLDTTLYVTLEPCPMCAGALLHSRVKRIVYGAPDLKAGAAGTVLNLFESQASYHYAEVESGLLEEECREQLQAFFKRRRKEIKALRQAEKEAEK

SEQ ID NO: 554 - protein, 148 aa, synthetic construct
MDDEFFMRQALREARKAYDEGEVPVGAVVVRDGKVIARGRNQVERLKDPTAHAEMIALTAAAAHLGSKWLKGCTLYVTVEPCAMCAGALVLARLERLVFGARDPKAGACGSVLDIVRHPRLNHRVEVVSGVLEEECGALLKEFFRRLR

SEQ ID NO: 555 - protein, 163 aa, synthetic construct
MTRDEQYMRRALALARQAEAEGEVPVGAVLVKDGEVIAEGWNRSIGRHDATAHAEIETLRKAGQVLGNYRLLDTTLYVTLEPCPMCAGALLHSRVKRVVFGAPDLKAGAAGTVLNLFESQASYHYADVESGLLEDECREQLQAFFKRRRKEIKAARKAAKEAE

SEQ ID NO: 556 - protein, 142 aa, synthetic construct
MDAALEEARRAAAAGEVPVGAVLVRDGEVLARAGNRTVRDNDPTAHAEILVIREAARRLGNYRLEGCVLYVTLEPCAMCAGAMVHARLPRLVYGAADPKAGAAGSVLDVLGHPALNHRVEVTGGVLADACGALLRDFFAARR

SEQ ID NO: 557 - protein, 142 aa, synthetic construct
MDEALAEARRAAAAGEVPVGAVLVRDGEVLARGGNRTIRDCDPTAHAEIVALREAARRAGNYRLPGTTLYVTLEPCAMCAGAMIHARVARLVYGAADPRAGAAGSVFDVLRHPALNHRMEVEGGVRAEECGALLREFFRARR

SEQ ID NO: 558 - protein, 166 aa, synthetic construct
MTEKDEFYMKRAIELARKGEEEGEVPVGAVLVKDGEIIAEGWNRSIGSHDATAHAEIETLRKAGQALENYRLLDTTLYVTLEPCPMCAGALLHSRVKRVVFGAPDLKAGAAGTVLNLFESQASYHYADVESGLLEEECREQLQAFFKRRRKEIKALKKAKKEEQDK

SEQ ID NO: 559 - protein, 146 aa, synthetic construct
DDDFMALALEEARAAAEAGEVPIGAVVVCDGEVVARAGNRTVRDNDPTAHAEIVALREAARKLGNYRLAGCTLYVTLEPCAMCAGAMIHARLDRVVYGADDPKAGAARSVLNVLDHPALNHRVAVEGGVLADECGALLREFFRARR

SEQ ID NO: 560 - protein, 165 aa, synthetic construct
MTEEDKYFMQRAIELARQAELAGEVPVGAVLVKDGEIIAEGWNQSIGSHDATAHAEIMALRQAGQVLQNYRLLDTTLYVTLEPCPMCAGALLHSRVKRVVFGAPDLKAGAAGTVLNLFESQASYHYADVESGLLEQECREQLQAFFKRRRKEIKALKQAQKEAEN

SEQ ID NO: 561 - protein, 162 aa, synthetic construct
MTDEEYMRRALELARQAEQQGEVPVGAVLVKDGEVIAEGWNQSIGSHDATAHAEIMALRAAGQALENYRLLDTTLYVTLEPCPMCAGALLHSRVKRVVFGAPDLKAGAAGTVLNLFESQASYHYADVESGLLEEECREQLQAFFKRRRKEIKAARQAKREAE

SEQ ID NO: 562 - protein, 166 aa, synthetic construct
MSEQDEFYMRRAIELARQAEAEGEVPVGAVLVKDGEIVAEGWNRSIGSHDATAHAEIETLRKAGQALQNYRLLDTTLYVTLEPCPMCAGALLHSRVKRVVFGAPDLKAGAAGTVLNLFESQASYHYAEVESGLLEEECREQLQAFFKRRRKEIKALRQAQKEAEEK

SEQ ID NO: 563 - protein, 161 aa, synthetic construct
DDTLYMKRALELARQAEAEGEVPVGAVLVKDGEVIAEGWNRSIGSHDATAHAEIETLRKAGQALQNYRLLDTTLYVTLEPCPMCAGALLHSRVKRIVFGAPDLKAGAAGTVLNLFESQASYHYAEVESGLLEEECREQLQAFFKRRRKEIKALRQAEKEAE

SEQ ID NO: 564 - protein, 162 aa, synthetic construct
DETYMRRALELARQAEEHGEVPVGAVLVKDGEIIAEGWNRSIGDHDATAHAEIMALREAGKTLENYRLLDTTLYVTLEPCPMCAGALLHSRVKRVVYGAPDLKAGAAGTVLNLFESQASYHYAEVESGLLEEECREQLQAFFKRRRKEIKAARKARREAEEK

SEQ ID NO: 565 - protein, 148 aa, synthetic construct
MTDADFMALALEEARAAAALGEVPVGAVVVRDGAVIARAGNRTIRDCDPTAHAEIVALRAAARALGNYRLDGCDLYVTLEPCAMCAGAMIHARLARLVYGAADPKTGAAGSVLDVFANPRLNHHTAVVGGVLADEAAALLRGFFAARR

SEQ ID NO: 566 - protein, 161 aa, synthetic construct
DDEKWMRYALSLADKAEALGEVPVGAVLVKDNQVIGEGWNQSISGHDATAHAEIMAIRDAGKNLQNYRLIDCTLYVTLEPCPMCAGAIVHSRIKRVVFGASDYKTGAAGSVFNLLSNEQLNHQAEVTAGVLAEECGEKISAFFKRRRKEKKAAKKAAKLAE

SEQ ID NO: 567 - protein, 164 aa, synthetic construct
MTEEDKYFMRRAIELAKLAEENGEVPVGAVLVKDGEIIAEGWNQSIGSHDATAHAEMMAIRQAGQVLENYRLLDTTLYVTLEPCPMCAGALLHSRVKRIVFGAPDLKAGAAGTVLNLFESQASYHYADVESGLLEEECREQLQAFFKRRRKEIKALKKAQKEAE

SEQ ID NO: 568 - protein, 149 aa, synthetic construct
MTDDERWMREALAEARAAEAAGEVPVGAVVVRDGELIARGRNRVLRDSDPTAHAEIVALREAARALGNYRLDGCTLYATLEPCAMCAGAMLHARLARLVYGAADPKAGAAGSVLDVLGHPRLNHRMEVTGGVLAEEAGALLREFFRARR

SEQ ID NO: 569 - protein, 162 aa, synthetic construct
DDSFYMRRALELAALAEAEGEVPVGAVLVKDGEIIAEGWNRSIGDHDATAHAEIQVLRKAGQALQNYRLLDTTLYVTLEPCPMCAGALLHSRVKRIVYGAPDLKAGAAGTVLNLFESQASYHYAEVESGLLEEECREQLQAFFKRRRKEIKALRQAEKEAQN

SEQ ID NO: 570 - protein, 146 aa, synthetic construct
DEEFMREALAEAQAAADAGEVPIGAVVVCDGEIVARAGNRTIRDNDPTAHAEIVALREAARKLGNYRLAGCTLYVTLEPCAMCAGAMIHARIDRLVYGADDPKAGAAGSVLDVLGHPRLNHQMQVERGVLAAECGAMLTRFFQARR

SEQ ID NO: 571 - protein, 164 aa, synthetic construct
MTEADEKFMRRAIELAREAEEHGEVPVGAVLVKDGEIIAEGWNRSIGEHDATAHAEIETLRKAGQVLENYRLLDTTLYVTLEPCPMCAGALLHSRVKRIVFGAPDLKAGAAGTVLNLFESQASYHYADVESGLLEEECREQLQAFFKRRRKEIKALRQAEKAAG

SEQ ID NO: 572 - protein, 142 aa, synthetic construct
MRDALALAAAAAAAGEVPVGAVVVHGGEIVGRGENRVLRDSDPTAHAEIVALREAARALGNYRLTGCDLYVTLEPCAMCAGAMIHARIARLVYGAADPKAGAAGSVLDVLNHPRLNHRMEVTGGVLAEECGALLRDFFRARR

SEQ ID NO: 573 - protein, 165 aa, synthetic construct
MTEEDKYFMQRAIELARLAEENGEVPVGAVLVKDGEIIAEGWNQSIGNHDATAHAEIMTLRQAGQVLQNYRLLDTTLYVTLEPCPMCAGALLHSRVKRIVFGAPDLKAGAAGTVLNLFESQASYHYADVESGLLEDECREQLQAFFKRRRKEIKALKKAKRDAEN

SEQ ID NO: 574 - protein, 149 aa, synthetic construct
MDPTDLAFMREALAEARAAAEAGEFPIGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 575 - protein, 149 aa, synthetic construct
MDPTDLAFMREALAEARAAAEAGEVPVGAVVVCDGRIVARAHNRPIALSDPTAHAEILALREAARALGNIRLTGCTLYATLEPCAMCAGAILHARIARLVYGVANPGAGACGSVLDVMNHPRLNHRVEVTGGVLAEECGALLSGFFRAR

SEQ ID NO: 576 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPKTGAAGSVLDVLGHPRLNHRMEVEGGVLAAECGALLRDFFRAR

SEQ ID NO: 577 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPGCGAAGSVLDVLGHPRLNHRMEVEGGVLAAECGALLRDFFRAR

SEQ ID NO: 578 - protein, 147 aa, synthetic construct
MTDEYFMRQALREARRAYDEGEVPVGAVVVRDGRVIARGRNQVERLKDPTAHAEMLALTAAAGHLGSGRLTGCTLYVTVEPCAMCAGALVWARVERVVFGVRNPKTGACGSVFDIPGDRRLNHRPEVVGGVLEAECAALMRDFFRER

SEQ ID NO: 579 - protein, 149 aa, synthetic construct
MDPTDLAFMREALAEARAAAEAGEVPVGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 580 - protein, 149 aa, synthetic construct
MDPTDLAFMREALAEARAAAEAGEVPIGAVVVCDGRIVARAHNRPIALSDPTAHAEILALREAARALGNYRLTGCTLYATLEPCAMCAGAILHARIARLVYGVANAGAGACGSVLDVMNHPRLNHRVEVTGGVLAEECGALLSGFFRAR

SEQ ID NO: 581 - protein, 147 aa, synthetic construct
MDDAGFMRLALEEARAAAAAGEVPIGAVVVRDGEVLARAGNRTVRDCDPTAHAEIVALREAARRLGNYRLAGCTLYVTLEPCAMCAGAMIHARLDRLVYGVANPKTGAAGSVLDVLGHPRLNHRMEVEGGVLAAECGALLRDFFRAR

SEQ ID NO: 582 - protein, 147 aa, synthetic construct
MDDAGFMRLALEEARAAAAAGEVPIGAVVVRDGEVLARAGNRTVRDCDPTAHAEIVALREAARRLGNYRLAGCTLYVTLEPCAMCAGAMIHARLDRLVYGVANPGAGAAGSVLDVLGHPRLNHRMEVEGGVLAAECGALLRDFFRAR

SEQ ID NO: 583 - protein, 147 aa, synthetic construct
MTDEYFMRQALREARRAYDEGEVPVGAVVVRDGRVIARGRNQVERLKDPTAHAEMLALTAAAGHLGSSRLTGCTLYVTVEPCAMCAGALVWARVERVVFGVRNPKTGACGSVFDIPGDRRLNHRPEVVGGVLEAECAALMRDFFRER

SEQ ID NO: 584 - protein, 147 aa, synthetic construct
MTDEYFMRQALREARRAYDEGEVPVGAVVVRDGRVIARGRNQVRRLKDPTAHAEMLALTAAAGHLGSPRLTGCTLYVTVEPCAMCAGALVWARVERVVFGVRNPKTGACGSVFDIPGDRRLNHRPEVVGGVLEAECAALMRDFFRER

SEQ ID NO: 585 - protein, 149 aa, synthetic construct
MDPTDLAFMREALAEARAAAEAGEVPIGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 586 - protein, 149 aa, synthetic construct
MDPTDLAFMREALAEARAAAEAGELPIGAVVVCDGRIVARAHNRPIALSDPTAHAEILALREAARALGNYRLTGCTLYATLEPCAMCAGAILHARIARLVYGVANPGTGACGSVLDVMNHPRLNHRVEVTGGVLAEECGALLSGFFRAR

SEQ ID NO: 587 - protein, 149 aa, synthetic construct
MDPTDLAFMREALAEARAAAEAGEMPVGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 588 - protein, 147 aa, synthetic construct
MDDAGFMRLALEEARAAAAAGEVPIGAVVVRDGEVLARAGNRTVRDCDPTAHAEIVALREAARRLGNYRLAGCTLYVTLEPCAMCAGAMIHARLDRLVYGVANPKAGAAGSVLDVLGHPRLNHRMEVEGGVLAAECGALLRDFFRAR

SEQ ID NO: 589 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPGRGAAGSVLDVLGHPRLNHRMEVEGGVLAAECGALLRDFFRAR

SEQ ID NO: 590 - protein, 147 aa, synthetic construct
MDDAGFMRLALEEARAAAAAGEVPIGAVVVRDGEVLARAGNRTVRDCDPTAHAEIVALREAARRLGNYRLAGCTLYVTLEPCAMCAGAMIHARLDRLVYGVANPGSGAAGSVLDVLGHPRLNHRMEVEGGVLAAECGALLRDFFRAR

SEQ ID NO: 591 - protein, 147 aa, synthetic construct
MTDEYFMRQALREARRAYDEGEVPVGAVVVRDGRVIARGRNQVIRLKDPTAHAEMLALTAAAGHLGSPRLTGCTLYVTVEPCAMCAGALVWARVERVVFGVRNPKTGACGSVFDIPGDRRLNHRPEVVGGVLEAECAALMRDFFRER

SEQ ID NO: 592 - protein, 149 aa, synthetic construct
MDPTDLAFMREALAEARAAAEAGEIPVGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 593 - protein, 149 aa, synthetic construct
MDPTDLAFMREALAEARAAAEAGELPIGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 594 - protein, 149 aa, synthetic construct
MDPTDLAFMREALAEARAAAEAGEVPVGAVVVCDGRIVARAHNRPIALSDPTAHAEILALREAARALGNVRLTGCTLYATLEPCAMCAGAILHARIARLVYGVANPGAGACGSVLDVMNHPRLNHRVEVTGGVLAEECGALLSGFFRAR

SEQ ID NO: 595 - protein, 149 aa, synthetic construct
MDPTDLAFMREALAEARAAAEAGEVPIGAVVVCDGRIVARAHNRPIALSDPTAHAEILALREAARALGNSRLTGCTLYATLEPCAMCAGAILHARIARLVYGVANPGAGACGSVLDVMNHPRLNHRVEVTGGVLAEECGALLSGFFRAR

SEQ ID NO: 596 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPKRGAAGSVLDVLGHPRLNHRMEVEGGVLAAECGALLRDFFRAR

SEQ ID NO: 597 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPGSGAAGSVLDVLGHPRLNHRMEVEGGVLAAECGALLRDFFRAR

SEQ ID NO: 598 - protein, 147 aa, synthetic construct
MDDAGFMRLALEEARAAAAAGEVPVGAVVVRDGEVLARAGNRTVRDCDPTAHAEIVALREAARRLGNFRLAGCTLYVTLEPCAMCAGAMIHARLDRLVYGVANPGRGAAGSVLDVLGHPRLNHRMEVEGGVLAAECGALLRDFFRAR

SEQ ID NO: 599 - protein, 149 aa, synthetic construct
MDPTDLAFMREALAEARAAAEAGEVPVGAVVVCDGRIVARAHNRPIALSDPTAHAEILALREAARALGNYRLTGCTLYATLEPCAMCAGAILHARIARLVYGVANDGAGACGSVLDVMNHPRLNHRVEVTGGVLAEECGALLSGFFRAR

SEQ ID NO: 600 - protein, 149 aa, synthetic construct
MDPTDLAFMREALAEARAAAEAGEFPVGAVVVCDGRIVARAHNRPIALSDPTAHAEILALREAARALGNYRLTGCTLYATLEPCAMCAGAILHARIARLVYGVANPGTGACGSVLDVMNHPRLNHRVEVTGGVLAEECGALLSGFFRAR

SEQ ID NO: 601 - protein, 149 aa, synthetic construct
MDPTDLAFMREALAEARAAAEAGEVPIGAVVVCDGRIVARAHNRPIALSDPTAHAEILALREAARALGNWRLTGCTLYATLEPCAMCAGAILHARIARLVYGVANPGAGACGSVLDVMNHPRLNHRVEVTGGVLAEECGALLSGFFRAR

SEQ ID NO: 602 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANAGSGAAGSVLDVLGHPRLNHRMEVEGGVLAAECGALLRDFFRAR

SEQ ID NO: 603 - protein, 147 aa, synthetic construct
MTDEYFMRQALREARRAYDEGEVPVGAVVVRDGRVIARGRNQVERLKDPTAHAEMLALTAAAGHLGSGRLTGCTLYVTVEPCAMCAGALVWARVERVVFGVRNPKTGACGSVFDIPGDRRLSHRPEVVGGVLEAECAALMRDFFRER

SEQ ID NO: 604 - protein, 147 aa, synthetic construct
MTDEYFMRQALREARRAYDEGEVPVGAVVVRDGRVIARGRNQVRRLKDPTAHAEMLALTAAAGHLGSRRLTGCTLYVTVEPCAMCAGALVWARVERVVFGVRNPKTGACGSVFDIPGDRRLNHRPEVVGGVLEAECAALMRDFFRER

SEQ ID NO: 605 - protein, 149 aa, synthetic construct
MDPTDLAFMREALAEARAAAEAGEIPIGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 606 - protein, 149 aa, synthetic construct
MDPTDLAFMREALAEARAAAEAGEFPVGAVVVCDGRIVARAHNRPIALSDPTAHAEILALREAARALGNYRLTGCTLYATLEPCAMCAGAILHARIARLVYGVANPGAGACGSVLDVMNHPRLNHRVEVTGGVLAEECGALLSGFFRAR

SEQ ID NO: 607 - protein, 149 aa, synthetic construct
MDPTDLAFMREALAEARAAAEAGEIPVGAVVVCDGRIVARAHNRPIALSDPTAHAEILALREAARALGNYRLTGCTLYATLEPCAMCAGAILHARIARLVYGVANAGAGACGSVLDVMNHPRLNHRVEVTGGVLAEECGALLSGFFRAR

SEQ ID NO: 608 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPGTGAAGSVLDVLGHPRLNHRMEVEGGVLAAECGALLRDFFRAR

SEQ ID NO: 609 - protein, 147 aa, synthetic construct
MTDEYFMRQALREARRAYDEGEVPVGAVVVRDGRVIARGRNQVERLKDPTAHAEMLALTAAAGHLGSRSLTGCTLYVTVEPCAMCAGALVWARVERVVFGVRNPRAGACGSVFDIPGDRRLNHRPEVVGGVLEAECAALMRDFFRER

SEQ ID NO: 610 - protein, 147 aa, synthetic construct
MTDEYFMRQALREARRAYDEGEVPVGAVVVRDGRVIARGRNQVVRLKDPTAHAEMLALTAAAGHLGSPRLTGCTLYVTVEPCAMCAGALVWARVERVVFGVRNPKTGACGSVFDIPGDRRLNHRPEVVGGVLEAECAALMRDFFRER

SEQ ID NO: 611 - protein, 149 aa, synthetic construct
MDPTDLAFMREALAEARAAAEAGEVPIGAVVVCDGRIVARAHNRPIALSDPTAHAEILALREAARALGNYRLTGCTLYATLEPCAMCAGAILHARIARLVYGVANDGAGACGSVLDVMNHPRLNHRVEVTGGVLAEECGALLSGFFRAR

SEQ ID NO: 612 - protein, 149 aa, synthetic construct
MDPTDLAFMREALAEARAAAEAGEVPVGAVVVCDGRIVARAHNRPIALSDPTAHAEILALREAARALGNYRLTGCTLYATLEPCAMCAGAILHARIARLVYGVANSGAGACGSVLDVMNHPRLNHRVEVTGGVLAEECGALLSGFFRAR

SEQ ID NO: 613 - protein, 149 aa, synthetic construct
MDPTDLAFMREALAEARAAAEAGEVPIGAVVVCDGRIVARAHNRPIALSDPTAHAEILALREAARALGNTRLTGCTLYATLEPCAMCAGAILHARIARLVYGVANPGAGACGSVLDVMNHPRLNHRVEVTGGVLAEECGALLSGFFRAR

SEQ ID NO: 614 - protein, 147 aa, synthetic construct
MTDEYFMRQALREARRAYDEGEVPVGAVVVRDGRVIARGRNQVNRLKDPTAHAEMLALTAAAGHLGSPRLTGCTLYVTVEPCAMCAGALVWARVERVVFGVRNPKAGACGSVFDIPGDRRLNHRPEVVGGVLEAECAALMRDFFRER

SEQ ID NO: 615 - protein, 147 aa, synthetic construct
MTDEYFMRQALREARRAYDEGEVPVGAVVVRDGRVIARGRNQVNRLKDPTAHAEMLALTAAAGHLGSRSLTGCTLYVTVEPCAMCAGALVWARVERVVFGVRNPKTGACGSVFDIPGDRRLNHRPEVVGGVLEAECAALMRDFFRER

SEQ ID NO: 616 - protein, 149 aa, synthetic construct
MDPTDLAFMREALAEARAAAEAGEVPIGAVVVCDGRIVARAHNRPIALSDPTAHAEILALREAARALGNYRLTGCTLYATLEPCAMCAGAILHARIARLVYGVANAGTGACGSVLDVMNHPRLNHRVEVTGGVLAEECGALLSGFFRAR

SEQ ID NO: 617 - protein, 149 aa, synthetic construct
MDPTDLAFMREALAEARAAAEAGEFPIGAVVVCDGRIVARAHNRPIALSDPTAHAEILALREAARALGNYRLTGCTLYATLEPCAMCAGAILHARIARLVYGVANPGTGACGSVLDVMNHPRLNHRVEVTGGVLAEECGALLSGFFRAR

SEQ ID NO: 618 - protein, 149 aa, synthetic construct
MDPTDLAFMREALAEARAAAEAGEVPIGAVVVCDGRIVARAHNRPIALSDPTAHAEILALREAARALGNIRLTGCTLYATLEPCAMCAGAILHARIARLVYGVANPGTGACGSVLDVMNHPRLNHRVEVTGGVLAEECGALLSGFFRAR

SEQ ID NO: 619 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPGAGAAGSVLDVLGHPRLNHRMEVEGGVLAAECGALLRDFFRAR

SEQ ID NO: 620 - protein, 147 aa, synthetic construct
MDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANAGRGAAGSVLDVLGHPRLNHRMEVEGGVLAAECGALLRDFFRAR

SEQ ID NO: 621 - protein, 149 aa, synthetic construct
MDPTDLAFMREALAEARAAAEAGEVPVGAVVVCDGRIVARAHNRPIALRDPTAHAEVLALREAAAALGSYRLTGCTLYATLEPCPMCAGAILHARIARLVYGVANPKAGACGSVLDVMNHPRLNHRVEVTGGVLAEECGALLSGFFRAR

SEQ ID NO: 622 - protein, 149 aa, synthetic construct
MDPTDLAFMREALAEARAAAEAGEVPVGAVVVCDGRIVARAHNRPIALKDPTAHAEVLALREAAAALGSYRLTGCTLYATLEPCPMCAGAILHARIARLVYGVANPKAGACGSVLDVMNHPRLNHRVEVTGGVLAEECGALLSGFFRAR

SEQ ID NO: 623 - protein, 149 aa, synthetic construct
MDPTDLAFMREALAEARAAAEAGEVPVGAVVVCDGRIVARAHNRPIALNDPTAHAEVLALREAGKALGSYRLTGCTLYATLEPCPMCAGAILHARIARLVYGVANPKAGACGSVLDVMNHPRLNHRVEVTGGVLAEECGALLSGFFRAR

SEQ ID NO: 624 - protein, 149 aa, synthetic construct
MDPTDLAFMREALAEARAAAEAGEVPVGAVVVCDGRIVARAHNRPIALRDPTAHAEVLALREAARALGSYRLTGCTLYATLEPCPMCAGAILHARIARLVYGVANPKAGACGTVLDVMNHPRLNHRVEVTGGVLAEECGALLSGFFRAR

SEQ ID NO: 625 - protein, 147 aa, synthetic construct
MDDAGFMRLALEEARAAAAAGEVPVGAVVVRDGEVLARAGNRTVRDRDPTAHAEIVAIREAARRLGSYRLAGCTLYVTLEPCPMCAGAMIHARLDRLVYGVANPKAGAAGTVLDVLGHPRLNHRMEVEGGVLAAECGALLRDFFRAR

SEQ ID NO: 626 - protein, 147 aa, synthetic construct
MTDEYFMRQALREARRAYDEGEVPVGAVVVRDGRVIARGRNQVRRLKDPTAHAEMLALTAAAGHLGSRSLTGCTLYVTVEPCAMCAGALVWARVERVVFGVRNPKAGACGSVFDIPGDRRLNHRPEVVGGVLEAECAALMRDFFRER

SEQ ID NO: 627 - protein, 147 aa, synthetic construct
MTDEYFMRQALREARRAYDEGEVPVGAVVVRDGRVIARGRNQVRRLKDPTAHAEMLALTAAAGHLGSARLTGCTLYVTVEPCAMCAGALVWARVERVVFGVRNPRAGACGSVFDIPGDRRLNHRPEVVGGVLEAECAALMRDFFRER

SEQ ID NO: 628 - protein, 149 aa, synthetic construct
MDPTDLAFMREALAEARAAAEAGEVPVGAVVVCDGRIVARAHNRPIALNDPTAHAEVLALREAARALGDYRLTGCTLYATLEPCPMCAGAILHARIARLVYGVANPKAGACGSVLDVMNHPRLNHRVEVTGGVLAEECGALLSGFFRAR

SEQ ID NO: 629 - protein, 149 aa, synthetic construct
MDPTDLAFMREALAEARAAAEAGEVPVGAVVVCDGRIVARAHNRPIALKDPTAHAEILALREAAAALGSYRLTGCTLYATLEPCAMCAGAILHARIARLVYGVANPKAGACGSVLDVMNHPRLNHRVEVTGGVLAEECGALLSGFFRAR

SEQ ID NO: 630 - protein, 149 aa, synthetic construct
MDPTDLAFMREALAEARAAAEAGEVPVGAVVVCDGRIVARAHNRPIALKDPTAHAEILALREAGRALGSYRLTGCTLYATLEPCPMCAGAILHARIARLVYGVANPKAGACGSVLDVMNHPRLNHRVEVTGGVLAEECGALLSGFFRAR

SEQ ID NO: 631 - protein, 149 aa, synthetic construct
MDPTDLAFMREALAEARAAAEAGEVPVGAVVVCDGRIVARAHNRPIALADPTAHAEVLALREAARALGSYRLTGCTLYATLEPCPMCAGAILHARIARLVYGVANPKAGACGSVLDVMNHPKLNHRVEVTGGVLAEECGALLSGFFRAR

SEQ ID NO: 632 - protein, 149 aa, synthetic construct
MDPTDLAFMREALAEARAAAEAGEVPVGAVVVCDGRIVARAHNRPIALNDPTAHAEILALREAGRALGSYRLTGCTLYATLEPCPMCAGAILHARIARLVYGVANPKAGACGSVLDVMNHPRLNHRVEVTGGVLAEECGALLSGFFRAR

SEQ ID NO: 633 - protein, 149 aa, synthetic construct
MDPTDLAFMREALAEARAAAEAGEVPVGAVVVCDGRIVARAHNRPIALRDPTAHAEILALREAGRALGNYRLTGCTLYATLEPCPMCAGAILHARIARLVYGVANPKAGACGSVLDVMNHPKLNHRVEVTGGVLAEECGALLSGFFRAR

SEQ ID NO: 634 - protein, 149 aa, synthetic construct
MDPTDLAFMREALAEARAAAEAGEVPVGAVVVCDGRIVARAHNRPIALNDPTAHAEVLALREAAAALGSYRLTGCTLYATLEPCPMCAGAILHARIARLVYGVANPKAGACGTVLDVMNHPRLNHRVEVTGGVLAEECGALLSGFFRAR

SEQ ID NO: 635 - protein, 149 aa, synthetic construct
MDPTDLAFMREALAEARAAAEAGEVPVGAVVVCDGRIVARAHNRPIALNDPTAHAEILALREAGRALGSYRLTGCTLYATLEPCPMCAGAILHARIARLVYGVANPKAGACGTVLDVMNHPRLNHRVEVTGGVLAEECGALLSGFFRAR

SEQ ID NO: 636 - protein, 149 aa, synthetic construct
MDPTDLAFMREALAEARAAAEAGEVPVGAVVVCDGRIVARAHNRPIALADPTAHAEVLALREAARALGSYRLTGCTLYATLEPCAMCAGAILHARIARLVYGVANPKAGACGSVLDVMNHPKLNHRVEVTGGVLAEECGALLSGFFRAR

SEQ ID NO: 637 - protein, 147 aa, synthetic construct
MDDAGFMRLALEEARAAAAAGEVPVGAVVVRDGEVLARAGNRTVRDRDPTAHAEVVALREAARRLGSYRLAGCTLYVTLEPCAMCAGAMIHARLDRLVYGVANPKAGAAGTVLDVLGHPRLNHRMEVEGGVLAAECGALLRDFFRAR

SEQ ID NO: 638 - protein, 147 aa, synthetic construct
MDDAGFMRLALEEARAAAAAGEVPVGAVVVRDGEVLARAGNRTVRDRDPTAHAEIVAIREAARRLGSYRLAGCTLYVTLEPCAMCAGAMIHARLDRLVYGVANPKAGAAGTVLDVLGHPRLNHRMEVEGGVLAAECGALLRDFFRAR

SEQ ID NO: 639 - protein, 147 aa, synthetic construct
MDDAGFMRLALEEARAAAAAGEVPVGAVVVRDGEVLARAGNRTVRDKDPTAHAEIVALREAARRLGSYRLAGCTLYVTLEPCAMCAGAMIHARLDRLVYGVANPKAGAAGTVLDVLGHPRLNHRMEVEGGVLAAECGALLRDFFRAR

SEQ ID NO: 640 - protein, 147 aa, synthetic construct
MTDEYFMRQALREARRAYDEGEVPVGAVVVRDGRVIARGRNQVVRLKDPTAHAEMLALTAAAGHLGSPRLTGCTLYVTVEPCAMCAGALVWARVERVVFGVRNPKTGACGSVFDIPGDRRLSHRPEVVGGVLEAECAALMRDFFRER

SEQ ID NO: 641 - protein, 149 aa, synthetic construct
MDPTDLAFMREALAEARAAAEAGEVPVGAVVVCDGRIVARAHNRPIALNDPTAHAEILALREAARALGSYRLTGCTLYATLEPCAMCAGAILHARIARLVYGVANPKAGACGSVLDVMNHPKLNHRVEVTGGVLAEECGALLSGFFRAR

SEQ ID NO: 642 - protein, 149 aa, synthetic construct
MDPTDLAFMREALAEARAAAEAGEVPVGAVVVCDGRIVARAHNRPIALRDPTAHAEILALREAARALGSYRLTGCTLYATLEPCAMCAGAILHARIARLVYGVANPKAGACGTVLDVMNHPRLNHRVEVTGGVLAEECGALLSGFFRAR

SEQ ID NO: 643 - protein, 149 aa, synthetic construct
MDPTDLAFMREALAEARAAAEAGEVPVGAVVVCDGRIVARAHNRPIALNDPTAHAEVLALREAAAALGSYRLTGCTLYATLEPCAMCAGAILHARIARLVYGVANPKAGACGTVLDVMNHPRLNHRVEVTGGVLAEECGALLSGFFRAR

SEQ ID NO: 644 - protein, 147 aa, synthetic construct
MTDEYFMRQALREARRAYDEGEVPVGAVVVRDGRVIARGRNQVVRLKDPTAHAEMLALTAAAGHLGSSRLTGCTLYVTVEPCAMCAGALVWARVERVVFGVRNPRTGACGSVFDIPGDRRLNHRPEVVGGVLEAECAALMRDFFRER

SEQ ID NO: 645 - protein, 147 aa, synthetic construct
MTDEYFMRQALREARRAYDEGEVPVGAVVVRDGRVIARGRNQVRRLKDPTAHAEMLALTAAAGHLGSPSLTGCTLYVTVEPCAMCAGALVWARVERVVFGVRNPRTGACGSVFDIPGDRRLNHRPEVVGGVLEAECAALMRDFFRER

SEQ ID NO: 646 - protein, 149 aa, synthetic construct
MDPTDLAFMREALAEARAAAEAGEVPVGAVVVCDGRIVARAHNRPIALKDPTAHAEILALREAARALGSYRLTGCTLYATLEPCPMCAGAILHARIARLVYGVANPKAGACGSVLDVMNHPRLNHRVEVTGGVLAEECGALLSGFFRAR

SEQ ID NO: 647 - protein, 149 aa, synthetic construct
MDPTDLAFMREALAEARAAAEAGEVPVGAVVVCDGRIVARAHNRPIALNDPTAHAEVLALREAGRALGSYRLTGCTLYATLEPCPMCAGAILHARIARLVYGVANPKAGACGSVLDVMNHPRLNHRVEVTGGVLAEECGALLSGFFRAR

SEQ ID NO: 648 - protein, 149 aa, synthetic construct
MDPTDLAFMREALAEARAAAEAGEVPVGAVVVCDGRIVARAHNRPIALNDPTAHAEVLALREAAKALGSYRLTGCTLYATLEPCPMCAGAILHARIARLVYGVANPKAGACGSVLDVMNHPKLNHRVEVTGGVLAEECGALLSGFFRAR

SEQ ID NO: 649 - protein, 149 aa, synthetic construct
MDPTDLAFMREALAEARAAAEAGEVPVGAVVVCDGRIVARAHNRPIALKDPTAHAEILALREAAAALGDYRLTGCTLYATLEPCPMCAGAILHARIARLVYGVANPKAGACGSVLDVMNHPKLNHRVEVTGGVLAEECGALLSGFFRAR

SEQ ID NO: 650 - protein, 149 aa, synthetic construct
MDPTDLAFMREALAEARAAAEAGEVPVGAVVVCDGRIVARAHNRPIALRDPTAHAEILALREAGRALGSYRLTGCTLYATLEPCPMCAGAILHARIARLVYGVANPKAGACGSVLDVMNHPKLNHRVEVTGGVLAEECGALLSGFFRAR

SEQ ID NO: 651 - protein, 147 aa, synthetic construct
MDDAGFMRLALEEARAAAAAGEVPVGAVVVRDGEVLARAGNRTVRDRDPTAHAEIVALREAARRLGSYRLAGCTLYVTLEPCPMCAGAMIHARLDRLVYGVANPKAGAAGTVLDILGHPRLNHRMEVEGGVLAAECGALLRDFFRAR

SEQ ID NO: 652 - protein, 147 aa, synthetic construct
MDDAGFMRLALEEARAAAAAGEVPVGAVVVRDGEVLARAGNRTVRDRDPTAHAEVVAIREAARRLGSYRLAGCTLYVTLEPCAMCAGAMIHARLDRLVYGVANPKAGAAGTVLDVLGHPRLNHRMEVEGGVLAAECGALLRDFFRAR

SEQ ID NO: 653 - protein, 147 aa, synthetic construct
MTDEYFMRQALREARRAYDEGEVPVGAVVVRDGRVIARGRNQVNRLKDPTAHAEMLALTAAAGHLGSPRLTGCTLYVTVEPCAMCAGALVWARVERVVFGVRNPRTGACGSVFDIPGDRRLSHRPEVVGGVLEAECAALMRDFFRER

SEQ ID NO: 654 - protein, 147 aa, synthetic construct
MTDEYFMRQALREARRAYDEGEVPVGAVVVRDGRVIARGRNQVRRLKDPTAHAEMLALTAAAGHLGSPQLTGCTLYVTVEPCAMCAGALVWARVERVVFGVRNPKTGACGSVFDIPGDRRLNHRPEVVGGVLEAECAALMRDFFRER

SEQ ID NO: 655 - protein, 149 aa, synthetic construct
MDPTDLAFMREALAEARAAAEAGEVPVGAVVVCDGRIVARAHNRPIALNDPTAHAEILALREAGRALGNYRLTGCTLYATLEPCPMCAGAILHARIARLVYGVANPKAGACGTVLDVMNHPRLNHRVEVTGGVLAEECGALLSGFFRAR

SEQ ID NO: 656 - protein, 149 aa, synthetic construct
MDPTDLAFMREALAEARAAAEAGEVPVGAVVVCDGRIVARAHNRPIALRDPTAHAEILALREAARALGSYRLTGCTLYATLEPCPMCAGAILHARIARLVYGVANPKAGACGTVLDVMNHPRLNHRVEVTGGVLAEECGALLSGFFRAR

SEQ ID NO: 657 - protein, 149 aa, synthetic construct
MDPTDLAFMREALAEARAAAEAGEVPVGAVVVCDGRIVARAHNRPIALNDPTAHAEVLALREAARALGSYRLTGCTLYATLEPCAMCAGAILHARIARLVYGVANPKAGACGTVLDVMNHPRLNHRVEVTGGVLAEECGALLSGFFRAR

SEQ ID NO: 658 - protein, 149 aa, synthetic construct
MDPTDLAFMREALAEARAAAEAGEVPVGAVVVCDGRIVARAHNRPIALNDPTAHAEILALREAARALGSYRLTGCTLYATLEPCAMCAGAILHARIARLVYGVANPKAGACGTVLDVMNHPKLNHRVEVTGGVLAEECGALLSGFFRAR

SEQ ID NO: 659 - protein, 149 aa, synthetic construct
MDPTDLAFMREALAEARAAAEAGEVPVGAVVVCDGRIVARAHNRPIALADPTAHAEILALREAAKALGSYRLTGCTLYATLEPCPMCAGAILHARIARLVYGVANPKAGACGTVLDVMNHPRLNHRVEVTGGVLAEECGALLSGFFRAR

SEQ ID NO: 660 - protein, 147 aa, synthetic construct
MTDEYFMRQALREARRAYDEGEVPVGAVVVRDGRVIARGRNQVRRLKDPTAHAEMLALTAAAGHLGSPRLTGCTLYVTVEPCAMCAGALVWARVERVVFGVRNPKTGACGSVFDIPGDRRLSHRPEVVGGVLEAECAALMRDFFRER

SEQ ID NO: 661 - protein, 147 aa, synthetic construct
MTDEYFMRQALREARRAYDEGEVPVGAVVVRDGRVIARGRNQVRRLKDPTAHAEMLALTAAAGHLGSARLTGCTLYVTVEPCAMCAGALVWARVERVVFGVRNPRTGACGSVFDIPGDRRLNHRPEVVGGVLEAECAALMRDFFRER

SEQ ID NO: 662 - protein, 147 aa, synthetic construct
MTDEYFMRQALREARRAYDEGEVPVGAVVVRDGRVIARGRNQVIRLKDPTAHAEMLALTAAAGHLGSPSLTGCTLYVTVEPCAMCAGALVWARVERVVFGVRNPRTGACGSVFDIPGDRRLNHRPEVVGGVLEAECAALMRDFFRER

SEQ ID NO: 663 - protein, 149 aa, synthetic construct
MDPTDLAFMREALAEARAAAEAGEVPVGAVVVCDGRIVARAHNRPIALNDPTAHAEILALREAARALGSYRLTGCTLYATLEPCPMCAGAILHARIARLVYGVANPKAGACGTVLDVMNHPRLNHRVEVTGGVLAEECGALLSGFFRAR

SEQ ID NO: 664 - protein, 149 aa, synthetic construct
MDPTDLAFMREALAEARAAAEAGEVPVGAVVVCDGRIVARAHNRPIALNDPTAHAEVLALREAARALGSYRLTGCTLYATLEPCPMCAGAILHARIARLVYGVANPKAGACGTVLDVMNHPRLNHRVEVTGGVLAEECGALLSGFFRAR

SEQ ID NO: 665 - protein, 149 aa, synthetic construct
MDPTDLAFMREALAEARAAAEAGEVPVGAVVVCDGRIVARAHNRPIALNDPTAHAEVLALREAAKALGSYRLTGCTLYATLEPCPMCAGAILHARIARLVYGVANPKAGACGTVLDVMNHPRLNHRVEVTGGVLAEECGALLSGFFRAR

SEQ ID NO: 666 - protein, 149 aa, synthetic construct
MDPTDLAFMREALAEARAAAEAGEVPVGAVVVCDGRIVARAHNRPIALNDPTAHAEVLALREAGRALGSYRLTGCTLYATLEPCPMCAGAILHARIARLVYGVANPKAGACGSVLDVMNHPKLNHRVEVTGGVLAEECGALLSGFFRAR

SEQ ID NO: 667 - protein, 147 aa, synthetic construct
MDDAGFMRLALEEARAAAAAGEVPVGAVVVRDGEVLARAGNRTVRDKDPTAHAEIVAIREAARRLGSYRLAGCTLYVTLEPCPMCAGAMIHARLDRLVYGVANPKAGAAGTVLDVLGHPRLNHRMEVEGGVLAAECGALLRDFFRAR

SEQ ID NO: 668 - protein, 147 aa, synthetic construct
MTDEYFMRQALREARRAYDEGEVPVGAVVVRDGRVIARGRNQVRRLKDPTAHAEMLALTAAAGHLGSPSLTGCTLYVTVEPCAMCAGALVWARVERVVFGVRNPKTGACGSVFDIPGDRRLNHRPEVVGGVLEAECAALMRDFFRER

SEQ ID NO: 669 - protein, 147 aa, synthetic construct
MTDEYFMRQALREARRAYDEGEVPVGAVVVRDGRVIARGRNQVIRLKDPTAHAEMLALTAAAGHLGSRSLTGCTLYVTVEPCAMCAGALVWARVERVVFGVRNPRAGACGSVFDIPGDRRLNHRPEVVGGVLEAECAALMRDFFRER

SEQ ID NO: 670 - protein, 147 aa, synthetic construct
MTDEYFMRQALREARRAYDEGEVPVGAVVVRDGRVIARGRNQVVRLKDPTAHAEMLALTAAAGHLGSPSLTGCTLYVTVEPCAMCAGALVWARVERVVFGVRNPRTGACGSVFDIPGDRRLNHRPEVVGGVLEAECAALMRDFFRER

SEQ ID NO: 671 - protein, 149 aa, synthetic construct
MDPTDLAFMREALAEARAAAEAGEVPVGAVVVCDGRIVARAHNRPIALKDPTAHAEILALREAAAALGNYRLTGCTLYATLEPCPMCAGAILHARIARLVYGVANPKAGACGSVLDVMNHPLLNHRVEVTGGVLAEECGALLSGFFRAR

SEQ ID NO: 672 - protein, 149 aa, synthetic construct
MDPTDLAFMREALAEARAAAEAGEVPVGAVVVCDGRIVARAHNRPIALRDPTAHAEILALREAAAALGSYRLTGCTLYATLEPCPMCAGAILHARIARLVYGVANPKAGACGTVLDVMNHPRLNHRVEVTGGVLAEECGALLSGFFRAR

SEQ ID NO: 673 - protein, 149 aa, synthetic construct
MDPTDLAFMREALAEARAAAEAGEVPVGAVVVCDGRIVARAHNRPIALNDPTAHAEILALREAARALGSYRLTGCTLYATLEPCPMCAGAILHARIARLVYGVANPKAGACGTVLDVMNHPKLNHRVEVTGGVLAEECGALLSGFFRAR

SEQ ID NO: 674 - protein, 149 aa, synthetic construct
MDPTDLAFMREALAEARAAAEAGEVPVGAVVVCDGRIVARAHNRPIALKDPTAHAEILALREAARALGSYRLTGCTLYATLEPCPMCAGAILHARIARLVYGVANPKAGACGTVLDVMNHPRLNHRVEVTGGVLAEECGALLSGFFRAR

SEQ ID NO: 675 - protein, 149 aa, synthetic construct
MDPTDLAFMREALAEARAAAEAGEVPVGAVVVCDGRIVARAHNRPIALKDPTAHAEILALREAAAALGDYRLTGCTLYATLEPCPMCAGAILHARIARLVYGVANPKAGACGTVLDVMNHPRLNHRVEVTGGVLAEECGALLSGFFRAR

SEQ ID NO: 676 - protein, 147 aa, synthetic construct
MTDEYFMRQALREARRAYDEGEVPVGAVVVRDGRVIARGRNQVERLKDPTAHAEILALRAACRHLGSKYLTGCTLYVTVEPCAMCAGALVWARVERVVFGVRNPKAGACGSVLDIPGDRRLNHRPEVVGGVLEAECAALMRDFFRER

SEQ ID NO: 677 - protein, 147 aa, synthetic construct
MTDEYFMRQALREARRAYDEGEVPVGAVVVRDGRVIARGRNQVERLKDPTAHAEILALSAACRHLGSKYLTGCTLYVTVEPCAMCAGALVWARVERVVFGVRNPKAGACGSVLDIPGDRRLNHRPEVVGGVLEAECAALMRDFFRER

SEQ ID NO: 678 - protein, 147 aa, synthetic construct
MTDEYFMRQALREARRAYDEGEVPVGAVVVRDGRVIARGRNQVERLKDATAHAEILALRAACAHLGSKYLTGCTLYVTVEPCAMCAGALVWARVERVVFGVRNPKAGACGSVLDIPGDRRLNHRPEVVGGVLEAECAALMRDFFRER

SEQ ID NO: 679 - protein, 147 aa, synthetic construct
MTDEYFMRQALREARRAYDEGEVPVGAVVVRDGRVIARGRNQVERLRDATAHAEILALRAACEHLGSKYLTGCTLYVTVEPCAMCAGALVWARVERVVFGVRNPKAGACGSVLDIPGNRRLNHRPEVVGGVLEAECAALMRDFFRER

SEQ ID NO: 680 - protein, 149 aa, synthetic construct
MDPEDLAFMRKALEEARQARDAGEVPVGAVVVLDGEIVARAHNRTIQLSDPTAHAEILALREAARALGNYRLEGCTLYVTLEPCAMCAGAILHARIERLVFGVANPKAGAAGSVLDVLNHPRLNHRVEVTGGVLAEECGALLRDFFRAR

SEQ ID NO: 681 - protein, 149 aa, synthetic construct
MDPTDEAFMKKALDEAKKAAEAGEVPVGAVVVYDGKVVARAHNRRIAESDPTAHAEILALREAARALGNYRLTGCTLYVTLEPCAMCAGAILHARIARLVYGVPNPKAGATGSVLNVLNHPRLNHRVEVTGGVLAEECGDLLRGFFRAR

SEQ ID NO: 682 - protein, 147 aa, synthetic construct
MDDEGWMQLALEEARASRAAGEVPVGAVVVRDGEVLARAGNRTRELCDPTAHAEIVALREAARRLGNYRLPGCTLYVTLEPCAMCAGAMLHARLDRLVYGVPNPKAGAAGSVLDVLGHPRLNHRLEVTGGVLAAECGQLLRDFFRAR

SEQ ID NO: 683 - protein, 147 aa, synthetic construct
MTDEYFMRQALREARRAYDEGEVPVGAVVVRDGRVIARGRNQVERLKDPTAHAEILALKAACAHLGSKYLTGCTLYVTVEPCAMCAGALVWARVERVVFGVRNPKAGACGSVLDIPGNRRLNHRPEVVGGVLEAECAALMRDFFRER

SEQ ID NO: 684 - protein, 149 aa, synthetic construct
MDPTDVAFMQQALDEARAAREAGEVPVGAVVVYDGRVVARAHNRTIARSDPTAHAEILALREAARALGNYRLAGCTLYVTLEPCAMCAGAILHARIERLVYGVPNPKAGATGSVLNVLNHPRLNHRVEVTGGVLAEECGALLRGFFRAR

SEQ ID NO: 685 - protein, 149 aa, synthetic construct
MDPEDLRFMRKALAEARKAKEAGEVPVGAVVVKDGKIVAQAHNRTIALSDPTAHAEILALREAARALGNYRLTGCTLYATLEPCAMCAGAILHARIARLVYGVPNPKAGASGSVLNVLNHPRLNHQVEVTGGVLAEECGALLRDFFRAR

SEQ ID NO: 686 - protein, 149 aa, synthetic construct
MDPKDLRFMQKALAEAREAKAAGEVPVGAVVVRNGKIVARAHNRTRALSDPTAHAEILALREAARALGNYRLEGCTLYVTLEPCAMCAGAILHARIERLVYGVANPKAGAAGSVLDVLNHPRLNHRVEVTGGVLAEECGALLRGFFRAR

SEQ ID NO: 687 - protein, 147 aa, synthetic construct
MTDEYFMRQALREARRAYDEGEVPVGAVVVRDGRVIARGRNQVERLKDPTAHAEILALRAACAHLGSKYLTGCTLYVTVEPCAMCAGALVWARVERVVFGVRNPKAGACGSVLDIPGDRRLNHRPEVVGGVLEAECAALMRDFFRER

SEQ ID NO: 688 - protein, 147 aa, synthetic construct
MTDEYFMRQALREARRAYDEGEVPVGAVVVRDGRVIARGRNQVERLQDPTAHAEILALRAACAHLGSKYLTGCTLYVTVEPCAMCAGALVWARVERVVFGVRNPKAGACGSVLDIPGNRRLNHRPEVVGGVLEAECAALMRDFFRER

SEQ ID NO: 689 - protein, 147 aa, synthetic construct
MTDEYFMRQALREARRAYDEGEVPVGAVVVRDGRVIARGRNQVERLRDPTAHAEVLALRAACAHLGSKYLTGCTLYVTVEPCAMCAGALVWARVERVVFGVRNPKAGACGSVLDIPGDRRMNHRPEVVGGVLEAECAALMRDFFRER

SEQ ID NO: 690 - protein, 147 aa, synthetic construct
MTDEYFMRQALREARRAYDEGEVPVGAVVVRDGRVIARGRNQVERLKDATAHAEILALRAACRHLGSKYLTGCTLYVTVEPCAMCAGALVWARVERVVFGVRNPKAGACGSVLDIPGNRRLNHRPEVVGGVLEAECAALMRDFFRER

SEQ ID NO: 691 - protein, 149 aa, synthetic construct
MDALDIEFMRKALAEARAAKEAGEVPVGAVVVRDGKIVARAHNRTIALSDPTAHAEILALRAAARALGNYRLTGCTLYVTLEPCAMCAGAILHARIERLVYGVPNPKAGATGSVLNVLNHPRLNHRVEVTGGVLAEECGRLLSDFFRAR

SEQ ID NO: 692 - protein, 149 aa, synthetic construct
MDPDDIAFMREALAEARRAKEAGEVPVGAVVVKDGRIVARAHNRTIAESDPTAHAEILALREAARQLGNYRLTGCTLYATLEPCAMCAGAILHARIARLVYGVPNPKAGATGSVLDVMNHPRLNHRVEVTGGVLADECGDLLRDFFRAR

SEQ ID NO: 693 - protein, 149 aa, synthetic construct
MDPEDIAFMEQALAEARAARDAGEVPVGAVVVLDGEIVAQAHNRTRQLSDPTAHAEILALRQAARALGNYRLTGCTLYVTLEPCAMCAGAILHARVERLVYGVANPKAGATGSVLNVLNHPRLNHRVEVTGGVLAEECGDLLRGFFRAR

SEQ ID NO: 694 - protein, 149 aa, synthetic construct
MDDQDLRFMKMALDEARKAKDAGEVPVGAVVVHEGEVVARAHNRTIARSDPTAHAEIRALREAARALGNYRLTGCTLYVTLEPCAMCAGAILHARIARLVYGVPNPKAGATGSVLNVLNHPRLNHRVEVTGGVLAEECGDLLRDFFRAR

SEQ ID NO: 695 - protein, 147 aa, synthetic construct
MDDEGWMRLALEEARASRAAGEVPVGAVVVRDGQVLARAGNRTRERCDPTAHAEIVALREAARKLGNYRLAGCTLYVTLEPCAMCAGAMLHARLDRLVYGVANPKAGAAGSVLDVLGHPRLNHRLEVTGGVLEAECGQLLRDFFRAR

SEQ ID NO: 696 - protein, 147 aa, synthetic construct
MTDEYFMRQALREARRAYDEGEVPVGAVVVRDGRVIARGRNQVERLRDPTAHAELLALRAACEHLGSKYLTGCTLYVTVEPCAMCAGALVWARVERVVFGVRNPKAGACGSVLDIPGDRRLNHRPEVVGGVLEAECAALMRDFFRER

SEQ ID NO: 697 - protein, 147 aa, synthetic construct
MTDEYFMRQALREARRAYDEGEVPVGAVVVRDGRVIARGRNQVERLRDATAHAEILALRAAARHLGSKYLTGCTLYVTVEPCAMCAGALVWARVERVVFGVRNPKAGACGSVLDIPGNRRLNHRPEVVGGVLEAECAALMRDFFRER

SEQ ID NO: 698 - protein, 149 aa, synthetic construct
MDAEDLAFMRQALAEAREAKEAGEVPVGAVVVHDGRVVARAHNRTIRLSDPTAHAEILALREAARALGNYRLAGCTLYVTLEPCAMCAGAILHARIQRLVYGVPNPKAGAAGSVLDVLNHPRLNHRVEVTGGVLAEECAALLTDFFRAR

SEQ ID NO: 699 - protein, 149 aa, synthetic construct
MDPEDVEFMRKALAEAREARQAGEVPVGAVVVHDGKIVARAHNRPIALSDPTAHAEILALREAARALGNYRLEGCTLYVTLEPCAMCAGAILHARIQRLVYGVPNPKAGATGSVLNVMNHPRLNHRVEVTGGVLAEECGALLSDFFRAR

SEQ ID NO: 700 - protein, 149 aa, synthetic construct
MDPEDLRFMREALDEAREAREAGEVPVGAVVVRDGRIVARAHNRTKELSDPTAHAEILALRAAARALGNYRLAGCTLYCTLEPCAMCAGAILHARIARLVYGVPNPKAGAAGSVLDVLNHPRLNHRVEVTGGVLAEECGALLRGFFRAR

SEQ ID NO: 701 - protein, 147 aa, synthetic construct
MDDAGFMRLALEEARAAAAAGEVPVGAVVVRDGEVLARAGNRTVRDADPTAHAEVVAIREAARRLGSYRLAGCTLYVTLEPCPMCAGAMIHARLDRLVYGVANPKAGAAGTVLDVLGHPRLNHRMEVEGGVLAAECGALLRDFFRAR

SEQ ID NO: 702 - protein, 147 aa, synthetic construct
MTDEYFMRQALREARRAYDEGEVPVGAVVVRDGRVIARGRNQVERLRDPTAHAEILALRAACAHLGSKYLTGCTLYVTVEPCAMCAGALVWARVERVVFGVRNPKAGACGSVLDIPGNRRLNHRPEVVGGVLEAECAALMRDFFRER

SEQ ID NO: 703 - protein, 147 aa, synthetic construct
MTDEYFMRQALREARRAYDEGEVPVGAVVVRDGRVIARGRNQVERLRDPTAHAELLALRAACAHLGSKYLTGCTLYVTVEPCAMCAGALVWARVERVVFGVRNPKAGACGSVLDIPGDRRLNHRPEVVGGVLEAECAALMRDFFRER

SEQ ID NO: 704 - protein, 147 aa, synthetic construct
MTDEYFMRQALREARRAYDEGEVPVGAVVVRDGRVIARGRNQVERLRDATAHAEILALRAAAAHLGSKYLTGCTLYVTVEPCAMCAGALVWARVERVVFGVRNPKAGACGSVLDIPGNRRLNHRPEVVGGVLEAECAALMRDFFRER

SEQ ID NO: 705 - protein, 147 aa, synthetic construct
MTDEYFMRQALREARRAYDEGEVPVGAVVVRDGRVIARGRNQVERLKDATAHAEILALRAACAHLGSKYLTGCTLYVTVEPCAMCAGALVWARVERVVFGVRNPKAGACGSVLDIPGNRRLNHRPEVVGGVLEAECAALMRDFFRER

SEQ ID NO: 706 - protein, 149 aa, synthetic construct
MDPKDIRFMRKALAEAREARDAGEVPVGAVVVHDGEIVAQAHNRPIAESDPTAHAEILALREAARALGNYRLEGCTLYVTLEPCAMCAGAILHARIARLVYGVANPKAGAAGSVLNVLNHPRLNHRVEVTGGVLADECGDLLRDFFRAR

SEQ ID NO: 707 - protein, 149 aa, synthetic construct
MDSEDIKFMRQALAEAREAREAGEVPVGAVVVHDGRIVARAHNRRILESDPTAHAEILALRQAARALGNYRLTGCTLYATLEPCAMCAGAILHARIERLVYGVANPKAGATGSVLNVLNHPRLNHRVEVTGGVLEEECGALLRDFFRAR

SEQ ID NO: 708 - protein, 149 aa, synthetic construct
MDPVDVRFMKKALAEAREAKEAGEVPVGAVVVKDGEVVARAHNRTIARSDPTAHAEINALRAAARALGNYRLEGCTLYVTLEPCAMCAGAILHARIQRLVYGVPNPKAGAAGSVLDVLNHPRLNHRVEVTGGVLAEECGALLSDFFRAR

SEQ ID NO: 709 - protein, 149 aa, synthetic construct
MDPDDIKFMRQALAEARKAREAGEVPVGAVVVKDGRVVARAHNRTIALSDPTAHAEIRALREAARALGNYRLVGCTLYVTLEPCAMCAGAILHARIARLVYGVPNPKAGAAGSVLDVMNHPRLNHRVEVTGGVLAEECGALMSDFFRAR

SEQ ID NO: 710 - protein, 147 aa, synthetic construct
MDDEGWMQLALEEARASRAAGEVPVGAVVVRDGEVLARAGNRTRERCDPTAHAEIVALREAARRLGNYRLPGCTLYVTLEPCAMCAGAMLHARLDRLVYGVANPKAGAAGSVLDVLGHPRLNHRMEVTGGVLADECGRLLRDFFRAR

SEQ ID NO: 711 - protein, 147 aa, synthetic construct
MTDEYFMRQALREARRAYDEGEVPVGAVVVRDGRVIARGRNQVERLRDATAHAEILALKAAAAHLGSKYLTGCTLYVTVEPCAMCAGALVWARVERVVFGVRNPKAGACGSVLDIPGDRRLNHRPEVVGGVLEAECAALMRDFFRER

SEQ ID NO: 712 - protein, 149 aa, synthetic construct
MDPEDIEFMEQALAEAKAAREAGEVPVGAVVVHDGKIVARAHNRTRALSDPTAHAEILALREAARALGNYRLEGCTLYATLEPCAMCAGAILHARIERLVYGVANPKAGATGSVLDVLNHPRLNHRVEVTGGVLAEECGALLSDFFRAR

SEQ ID NO: 713 - protein, 149 aa, synthetic construct
MDPEDIAFMRMALAEARKAKEAGEVPVGAVVVLNGEIVARAHNRPIALSDPTAHAEILALREAARALGNYRLPGCTLYVTLEPCAMCAGAILHARIERLVYGVANPKAGATGSVLDVLNHPRLNHKVEVTGGVLAEECGALLRGFFRAR

SEQ ID NO: 714 - protein, 149 aa, synthetic construct
MDAEDLEFMQQALDEAREAREAGEVPVGAVVVLDGRIVARAHNRPIAESDPTAHAEILALRAAARALGNYRLTGCTLYVTLEPCAMCAGAILHARIERLVYGVPNPKAGACGSVLNVMNHPRLNHRVEVVGGVLAEECGALLTGFFRAR

SEQ ID NO: 715 - protein, 149 aa, synthetic construct
MDPKDIKFMEKALEEARKAKEAGEVPVGAVVVKDGRIVARAHNRTIELSDPTAHAEIRALREAARALGNYRLEGCTLYATLEPCAMCAGAILHARIERLVYGVPNPKAGATGSVLDVLNHPRLNHRVEVTGGVLAEECGALLRGFFRAR

SEQ ID NO: 716 - protein, 147 aa, synthetic construct
MDDEGWMRLALEEARASRAAGEVPVGAVVVRDGEVLARAGNRTRELCDPTAHAEIVALREAARRLGNYRLPGCTLYVTLEPCAMCAGAMIHARLDRLVYGVANPKAGAAGSVLDVLGHPRLNHRMEVTGGVLAEECGALMRDFFRAR

SEQ ID NO: 717 - protein, 147 aa, synthetic construct
MTDEYFMRQALREARRAYDEGEVPVGAVVVRDGRVIARGRNQVERLKDPTAHAEILALRAACAHLGSKYLTGCTLYVTVEPCAMCAGALVWARVERVVFGVRNPKAGACGSVLDIPGNRKLNHRPEVVGGVLEAECAALMRDFFRER

SEQ ID NO: 718 - protein, 147 aa, synthetic construct
MTDEYFMRQALREARRAYDEGEVPVGAVVVRDGRVIARGRNQVERLRDATAHAEILALRAACRHLGSKYLTGCTLYVTVEPCAMCAGALVWARVERVVFGVRNPKAGACGSVLDIPGNRRLNHRPEVVGGVLEAECAALMRDFFRER

SEQ ID NO: 719 - protein, 149 aa, synthetic construct
MDPEDIAFMRKALAEARKAKEAGEVPVGAVVVKDGKIVARAHNRTIALSDPTAHAEILALREAARALGNYRLTGCTLYVTLEPCAMCAGAILHARIEKLVYGVPNPKAGACGSVLNVLNHPRLNHRVEVTGGVLAEECGALLKDFFRAR

SEQ ID NO: 720 - protein, 149 aa, synthetic construct
MDPEDVRYMRKALAEARKAKEAGEVPVGAVVVHDGRVVARAHNRTIALSDPTAHAEILALREAARALGNYRLEGCTLYATLEPCAMCAGAILHARIQRLVYGVANPKAGAAGSVLDVMNHPRLNHRVEVTGGVLEEECGALLSDFFRAR

SEQ ID NO: 721 - protein, 149 aa, synthetic construct
MDPEDVAFMKKALAEARAAQEAGEVPVGAVVVKDGEVVARAHNRTRERSDPTAHAEILALREAARALGNYRLAGCTLYVTLEPCAMCAGAILHARIERLVYGVANPKAGAAGSVLNVLNHPRLNHRVEVTGGVLAEECGDLLRGFFRAR

SEQ ID NO: 722 - protein, 149 aa, synthetic construct
MDPTDLEFMRQALAEAREARDAGEVPVGAVVVHDGRIVARAHNRTRELSDPTAHAEILALRAAARALGNYRLTGCTLYATLEPCAMCAGAILHARIERLVYGVANPKAGACGSVLHVLNHPRLNHRVEVTGGVLAEECGRLLRGFFRAR

SEQ ID NO: 723 - protein, 147 aa, synthetic construct
MDDEGWMRLALEEARAAKAAGEVPVGAVVVRDGEVLARAGNRTRLDCDPTAHAEIVALREAARQLGNYRLAGCTLYVTLEPCAMCAGAMLHARLDRLVYGVPNPKAGAAGSVLDVLGHPRLNHRLEVTGGVLAEECGQLMRDFFRAR

SEQ ID NO: 724 - protein, 147 aa, synthetic construct
MTDEYFMRQALREARRAYDEGEVPVGAVVVRDGRVIARGRNQVERLRDATAHAEILALRAAAAHLGSKYLTGCTLYVTVEPCAMCAGALVWARVERVVFGVRNPKAGACGSVLDIPGDRRLNHRPEVVGGVLEAECAALMRDFFRER

SEQ ID NO: 725 - protein, 147 aa, synthetic construct
MTDEYFMRQALREARRAYDEGEVPVGAVVVRDGRVIARGRNQVERLRDATAHAEILALRAASAHLGSKYLTGCTLYVTVEPCAMCAGALVWARVERVVFGVRNPKAGACGSVLDIPGDRRLNHRPEVVGGVLEAECAALMRDFFRER

SEQ ID NO: 726 - protein, 147 aa, synthetic construct
MTDEYFMRQALREARRAYDEGEVPVGAVVVRDGRVIARGRNQVERLRDPTAHAEILALRAACAHLGSKYLTGCTLYVTVEPCAMCAGALVWARVERVVFGVRNPKAGACGSVLDIPGNRRMNHRPEVVGGVLEAECAALMRDFFRER

SEQ ID NO: 727 - protein, 149 aa, synthetic construct
MDPEDLAFMRKALAEARRAAEAGEVPVGAVVVHDGRIVARAHNRREALSDPTAHAEILALREAARALGNYRLAGCTLYATLEPCAMCAGAILHARIARLVYGVPNPKAGACGSVLDVLNHPRLNHRVEVTGGVLAEECGALLRDFFRAR

SEQ ID NO: 728 - protein, 149 aa, synthetic construct
MDPYDLAFMQQALAEARQAKAAGEVPVGAVVVHDGRIVARAHNRTIELSDPTAHAEILALRQAARALGNYRLEGCTLYCTLEPCAMCAGAILHARIERLVYGVPNPKAGAAGSVLNVMNHPRLNHRVEVTGGVLAEECGALLRDFFRAR

SEQ ID NO: 729 - protein, 149 aa, synthetic construct
MDPEDIEFMQKALAEAREAREAGEVPVGAVVVKDGEVVARAHNRVNALSDPTAHAEILALREAARALGNYRLTGCTLYATLEPCAMCAGAILHARIERLVYGVANPKAGATGSVLNVLNHPRLNHRVEVTGGVLAEECGDLLRDFFRAR

SEQ ID NO: 730 - protein, 147 aa, synthetic construct
MDDRGWMRLALEEARAARARGEVPVGAVVVRDGQVLARAGNRTRELCDPTAHAEIVALREAARRLGNYRLAGCTLYVTLEPCAMCSGAMLHARLDRLVYGVANPKAGAAGSVLDVLGHPRLNHRLEVEGGVLEEECGALLRDFFRAR

SEQ ID NO: 731 - protein, 147 aa, synthetic construct
MDDRGWMRLALEEARASRAAGEVPVGAVVVRDGEVLARAGNRVRELCDPTAHAEIVALREAARRLGNYRLPGCTLYVTLEPCAMCAGAMLHARLDRLVYGVANPKAGAAGSVLDVLGHPRLNHRMEVTGGVLEEECGALMRDFFRAR

SEQ ID NO: 732 - protein, 147 aa, synthetic construct
MNDEGFMQLALEEARAARAAGEVPVGAVVVRDGEVIASAGNRTRERCDPTAHAEIVALRAAARKLGNYRLPGCTLYVTLEPCAMCAGAMIHARLDRLVYGVANPKAGAAGSVLDVLGHPRLNHRMEVEGGVLAEECGRLLRDFFRAR

SEQ ID NO: 733 - protein, 147 aa, synthetic construct
MSDEHFMKQALDEARKAKDRGEVPVGAVVVRDGEIIATGRNRVEELKDPTAHAEMLAITAAAGHLGSKYLTGCTLYVTLEPCVMCAGACIWARVDRVVFGVRNPKAGACGSVFNVPGDPRLNHHPEVTGGILEDECAKLLRDFFRER

SEQ ID NO: 734 - protein, 167 aa, synthetic construct
MSEVDPTDLAFMREALAEARAAAEAGEVPVGAVVVCDGRIVARAHNRPIALSDPTAHAEILALREAARALGNYRLTGCTLYATLEPCAMCAGAILHARIARLVYGVANPKAGACGSVLDVMNHPRLNHRVEVTGGVLAEECGALLSGFFRAPRRVFNAQKKAQSSTD

SEQ ID NO: 735 - protein, 365 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSDFFRMRRQEIKAQKKAQSSTDSGGSSGGSSGSETPGTSESATPESSGGSSGGSSEVEFTDEYFMRQALREARRAYDEGEVPVGAVVVRDGRVIARGRNQVERLKDPTAHAEMLALTAAAGHLGSKYLTGCTLYVTVEPCAMCAGALVWARVERVVFGVRNPKAGACGSVFDIPGDRRLNHRPEVVGGVLEAECAALMRDFFREPRRVFNAQKKAQSSTD

SEQ ID NO: 736 - protein, 147 aa, synthetic construct
MDDEGWMRLALEEARAAKAAGEVPVGAVVVRDGQVIARAGNRTRERCDPTAHAEIRALREAARRLGNYRLEGCTLYVTLEPCAMCAGAMLHARLDRLVYGVANPKAGAAGSVLDVLGHPRLNHRLEVTGGVLAEECGQLLRDFFRAR

SEQ ID NO: 737 - protein, 147 aa, synthetic construct
MDDAGWMQLALEEARASRAAGEVPVGAVVVRDGEVLASAGNRTRELCDPTAHAEIVALREAARRLGNYRLSGCTLYVTLEPCAMCAGAMLHARLDRLVYGVANPKAGAAGSVLDVLGHPRLNHRMEVTGGVLAEECAALLRDFFRAR

SEQ ID NO: 738 - protein, 147 aa, synthetic construct
MDDEGWMRLALEEARAARDAGEVPVGAVVVRDGQVIARAGNRTRQLCDPTAHAEIVALRAAARRLGNYRLPGCTLYVTLEPCAMCAGAMVHARIDRLVYGVPNPKAGAAGSVLNVLHHPRLNHRMEVTGGVLAEECGALLTDFFRAR

SEQ ID NO: 739 - protein, 147 aa, synthetic construct
MDDAGWMQLALEEARASKAAGEVPVGAVVVRDGRILARAGNRTRELCDPTAHAEIVALREAARRLGNYRLPGCTLYVTLEPCAMCAGAMLHARLDRLVYGVANPKAGAAGSVLDVLGHPRLNHRLEVEGGVLAAECSALLRDFFRAR

SEQ ID NO: 740 - protein, 167 aa, synthetic construct
MSEVEFDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPKAGAAGSVLDVLGHPRLNHRMEVEGGVLAAECGALLRDFFRAPRRVFNAQKKAQSSTD

SEQ ID NO: 741 - protein, 147 aa, synthetic construct
MDDRGWMKLALEEARASRAAGEVPVGAVVVRDGRVLASAGNRTRERCDPTAHAEIVALREAARKLGNYRLAGCTLYVTLEPCAMCAGAMLHARLDRLVYGVANPKAGAAGSVLDVLGHPRLNHRMEVTGGVLAEECGALLRDFFRAR

SEQ ID NO: 742 - protein, 147 aa, synthetic construct
MDDEGWMQLALDEARKARDAGEVPVGAVVVRDGRVLASAGNRTRERCDPTAHAEIVALREAARKLGNYRLPGCTLYVTLEPCAMCAGAMLHARLDRLVYGVDNPKAGAAGSVLDVLGHPRLNHRMEVVGGVLAEECGALLRDFFRAR

SEQ ID NO: 743 - protein, 147 aa, synthetic construct
MTDEYFMKQALAEARKAYDAGEVPVGAVVVRDGEVIARGHNRVERLKDPTAHAEMLAITAAAGHLGSKYLEGCTLYVTLEPCVMCAGALVWARVDRVVFGVPNPKAGACGSVFDIPGDPRLNHRPEVTGGVLEAECAALIRDFFRER

SEQ ID NO: 744 - protein, 167 aa, synthetic construct
MSEVEFTDEYFMRQALREARRAYDEGEVPVGAVVVRDGRVIARGRNQVERLKDPTAHAEMLALTAAAGHLGSKYLTGCTLYVTVEPCAMCAGALVWARVERVVFGVRNPKAGACGSVFDIPGDRRLNHRPEVVGGVLEAECAALMRDFFREPRRVFNAQKKAQSSTD

SEQ ID NO: 745 - protein, 364 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSDFFRMRRQEIKAQKKAQSSTDSGGSSGGSSGSETPGTSESATPESSGGSSGGSSEVEFDEKWMRYALSLADKAEALGEVPVGAVLVKDNQVIGEGWNQSISGHDATAHAEIMAIRDAGKNLQNYRLIDCTLYVTLEPCPMCAGAIVHSRIKRVVFGVSNYKTGAAGSVFNLLSNEQLNHQAEVTAGVLAEECGEKISAFFKRPRRVFNAQKKAQSSTD

SEQ ID NO: 746 - protein, 147 aa, synthetic construct
MDDEGWMQLALEEARASRAAGEVPVGAVVVRDGEVVARAGNRTRELCDPTAHAEIVALREAARARGNYRLTGCTLYVTLEPCAMCAGAMIHARLDRLVYGVPNPKAGAAGSVLDVLGHPRLNHRMEVTGGVLAEECGALLRDFFRAR

SEQ ID NO: 747 - protein, 147 aa, synthetic construct
MDDQGWMQLALEEARASRARGEVPVGAVVVRDGEVIARAGNRVRERCDPTAHAEIVALREAARALGNYRLPGCTLYVTLEPCAMCAGAMLHARLDRLVYGVANPKAGAAGSVLDVLGHPRLNHRLEVTGGVLEAECGALLRDFFRAR

SEQ ID NO: 748 - protein, 147 aa, synthetic construct
MDDEGWMRLALEEARASRARGEVPVGAVVVRDGEVLARAGNRTREKCDPTAHAEIVALREAARRLGNYRLAGCTLYVTLEPCAMCAGAMLHARLDRLVYGVPNPKAGAAGSVLDVLGHPRLNHRMEVVGGVLEAECGALLRDFFRAR

SEQ ID NO: 749 - protein, 147 aa, synthetic construct
MTDEHFMREALREAQKAYDRGEVPVGAVVVRDGEIIARGRNQVEKLKDPTAHAEMLAITAAAGHLGSKYLRGCTLYVTLEPCVMCAGALVWARVERVVFGVDNPKAGAAGSVFNVPGDPRLNHRPEVVGGVLEAECAALLRDFFRER

SEQ ID NO: 750 - protein, 366 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSDFFRMRRQEIKAQKKAQSSTDSGGSSGGSSGSETPGTSESATPESSGGSSGGSSEVEFSDDAGFMREALAEARAAAAAGEVPVGAVVVKDGEIIARAGNRTLRDNDPTAHAEIVALRAAARALGNHRLTGCTLYVTLEPCAMCAGAISHARIARLVYGVDNPKGGAVAHGPRFFAQPTCHHRPEVTGGVGAEEAGALLRDFFRAPRRVFNAQKKAQSSTD

SEQ ID NO: 751 - protein, 162 aa, synthetic construct
MDDEFYMRRALELAALAEEHNEVPVGAVLVLNGEIIGEGWNRSIGHHDATAHAEIMALRQAGKKLENYRLLDTTLYVTLEPCPMCAGALLHSRVKRVVFGVPNLKAGAAGTVLNLFESQASYHYADVESGLLEQECREQLQAFFKRRRKEKKALKQAQKEAE

SEQ ID NO: 752 - protein, 147 aa, synthetic construct
MDDRGWMRLALEEARAARAAGEVPVGAVVVRDGEVLARAGNRTRELCDPTAHAEIRALREAARQLGNYRLAGCTLYVTLEPCAMCAGAMLHARLDRLVYGVPNPKAGAAGSVLDVLGHPRLNHRLEVEGGVLAAECAQLLRDFFRAR

SEQ ID NO: 753 - protein, 147 aa, synthetic construct
MDDAGWMQLALEEARAAREAGEVPVGAVVVRDGEVIARAGNRTRELCDPTAHAEIVALREAARKLGNYRLAGCTLYVTLEPCAMCAGAMLHARLDRLVYGVANPKAGAAGSVLDVLGHPRLNHRMEVTGGVLAAECGRLLRDFFRAR

SEQ ID NO: 754 - protein, 147 aa, synthetic construct
MDDEGFMQLALEEARAARAAGEVPVGAVVVKDGEVLARAGNRTRELCDPTAHAEIVALREAARALGNYRLSGCTLYVTLEPCAMCAGAMVHARLDRLVYGVPNPKAGAAGSVLDVLHHPRLNHRMEVTGGVLAAECGALLRDFFRAR

SEQ ID NO: 755 - protein, 147 aa, synthetic construct
MSDEHFMRQALEEARKAYDEGEVPVGAVVVRDGEVIARGRNQVEILKDPTAHAEMLALTAAAGHLGSKYLRGCTLYVTLEPCVMCAGACIWARVDRVVFGVRNPKAGAAGSVFDVPGDPRLNHRPEVVGGVLRDECAQLMRDFFRER

SEQ ID NO: 756 - protein, 365 aa, synthetic construct
MSEVEFTDEYFMRQALREARRAYDEGEVPVGAVVVRDGRVIARGRNQVERLKDPTAHAEMLALTAAAGHLGSKYLTGCTLYVTVEPCAMCAGALVWARVERVVFGVRNPKAGACGSVFDIPGDRRLNHRPEVVGGVLEAECAALMRDFFREPRRVFNAQKKAQSSTDSGGSSGGSSGSETPGTSESATPESSGGSSGGSSEVEFTDEYFMRQALREARRAYDEGEVPVGAVVVRDGRVIARGRNQVERLKDPTAHAEMLALTAAAGHLGSKYLTGCTLYVTVEPCAMCAGALVWARVERVVFGVRNPKAGACGSVFDIPGDRRLNHRPEVVGGVLEAECAALMRDFFREPRRVFNAQKKAQSSTD

SEQ ID NO: 757 - protein, 161 aa, synthetic construct
MDEKWMRYALSLADKAEALGEVPVGAVLVKDNQVIGEGWNQSISGHDATAHAEIMAIRDAGKNLQNYRLIDCTLYVTLEPCPMCAGAIVHSRIKRVVFGVSNYKTGAAGSVFNLLSNEQLNHQAEVTAGVLAEECGEKISAFFKRRRKEKKAAKKAAKLAE

SEQ ID NO: 758 - protein, 147 aa, synthetic construct
MNDQGWMRLALEEARAARAAGEVPVGAVVVRDGEVLARAGNRVRLLCDPTAHAEIVALREAARRLGNYRLEGCTLYVTLEPCAMCAGAMIHARLDRLVYGVPNPKAGAAGSVLDVLGHPRLNHRLEVTGGVLADECGALMRDFFRAR

SEQ ID NO: 759 - protein, 147 aa, synthetic construct
MDDEGWMRLALEEARASKAAGEVPVGAVVVRDGEVIARAGNRTRELCDPTAHAEIVALREAARRLGNYRLAGCTLYVTLEPCAMCAGAMIHARLDRLVYGVPNPKAGAAGSVLDVLGHPRLNHRMEVEGGVLAEECRALLTDFFRAR

SEQ ID NO: 760 - protein, 147 aa, synthetic construct
MTDEHFMRQALAEARKAYDEGEVPVGAVVVRDGEIVATGRNQVERLKDPTAHAEMLAITAAAGHLGSKYLRGCTLYVTVEPCVMCAGACVWARVDRVVFGVRNPKAGACGSVFDIPGDPRLNHHPEVTGGVLEDECRQLLRDFFRER

SEQ ID NO: 761 - protein, 147 aa, synthetic construct
MTDEHFMRQALEEARKAYDQGEVPVGAVVVRDGEVIARGRNQVERLKDPTAHAEMLAITAAAGTLGSKYLEGCTLYVTLEPCVMCAGACIWARVERVVFGVRNPKAGACGSVFDIPGDPRLNHRPEVVGGVLEAECAALMRDFFRER

SEQ ID NO: 762 - protein, 168 aa, synthetic construct
MSEVEFSDDAGFMREALAEARAAAAAGEVPVGAVVVKDGEIIARAGNRTLRDNDPTAHAEIVALRAAARALGNHRLTGCTLYVTLEPCAMCAGAISHARIARLVYGVDNPKGGAVAHGPRFFAQPTCHHRPEVTGGVGAEEAGALLRDFFRAPRRVFNAQKKAQSSTD

SEQ ID NO: 763 - protein, 365 aa, synthetic construct
MSEVDPTDLAFMREALAEARAAAEAGEVPVGAVVVCDGRIVARAHNRPIALNDPTAHAEILALREAAKALGNYRLTGCTLYATLEPCPMCAGAILHARIARLVYGVANPKAGACGTVLDVMNHPRLNHRVEVTGGVLAEECGALLSGFFRAPRRVFNAQKKAQSSTDSGGSSGGSSGSETPGTSESATPESSGGSSGGSSEVDPTDLAFMREALAEARAAAEAGEVPVGAVVVCDGRIVARAHNRPIALNDPTAHAEILALREAAKALGNYRLTGCTLYATLEPCPMCAGAILHARIARLVYGVANPKAGACGTVLDVMNHPRLNHRVEVTGGVLAEECGALLSGFFRAPRRVFNAQKKAQSSTD

SEQ ID NO: 764 - protein, 147 aa, synthetic construct
MDDAGFMREALAEARAAAAAGEVPVGAVVVKDGEIIARAGNRTLRDNDPTAHAEIVALRAAARALGNHRLTGCTLYVTLEPCAMCAGAISHARIARLVYGVDNPKGGAVAHGPRFFAQPTCHHRPEVTGGVGAEEAGALLRDFFRAR

SEQ ID NO: 765 - protein, 147 aa, synthetic construct
MDDEGWMRLALEEARKSRAAGEVPVGAVVVRDGEVLATAGNRTRELCDPTAHAEIVALREAARRLGNYRLPGCTLYVTLEPCAMCAGAMIHARLDRLVYGVANPKAGAAGSVLDVLGHPRLNHRMEVEGGVLEAECAALLRDFFRAR

SEQ ID NO: 766 - protein, 147 aa, synthetic construct
MDDEGWMQLALEEARQSRAAGEVPVGAVVVRDGEVIARAGNRTREKCDPTAHAEIVALREAARKLGNYRLPGCTLYVTLEPCAMCAGAMVHARLDRLVYGVANPKAGAAGSVLDVLHHPRLNHRMEVTGGVLAEECGALLRDFFRAR

SEQ ID NO: 767 - protein, 147 aa, synthetic construct
MDDAGWMRLALEEARKSRARGEVPVGAVVVRDGQVLARAGNRTRELCDPTAHAEIVALRAAARKLGNYRLPGCTLYVTLEPCAMCAGAMVHARLDRLVYGVRNPKAGAAGSVLDVLGHPRLNHQMEVTGGVLADECAALLRDFFRAR

SEQ ID NO: 768 - protein, 365 aa, synthetic construct
MSEVEFDDAGFMRLALEEARAAAAAGEVPVGAVVVRDGEVLARAGNRTVRDNDPTAHAEIVALREAARRLGSYRLAGCTLYVTLEPCPMCAGAMIHARLDRLVYGVANPKAGAAGTVLDVLGHPRLNHRMEVEGGVLAAECGALLRDFFRAPRRVFNAQKKAQSSTDSGGSSGGSSGSETPGTSESATPESSGGSSGGSSEVEFDDAGFMRLALEEARAAAAAGEVPVGAVVVRDGEVLARAGNRTVRDNDPTAHAEIVALREAARRLGSYRLAGCTLYVTLEPCPMCAGAMIHARLDRLVYGVANPKAGAAGTVLDVLGHPRLNHRMEVEGGVLAAECGALLRDFFRAPRRVFNAQKKAQSSTD

SEQ ID NO: 769 - protein, 147 aa, synthetic construct
MDDAGFMELALDEARAARAAGEVPVGAVVVRDGEVLARAGNRTRELCDPTAHAEIVALREAARKLGNYRLEGCTLYVTLEPCAMCAGAMLHARLDRLVYGVANPKAGAAGSVLDVLGHPRLNHRMEVTGGVLEDECAALLRDFFRAR

SEQ ID NO: 770 - protein, 147 aa, synthetic construct
MDDAGWMQLALEEARAARAAGEVPVGAVVVRDGEVLARAGNRTRERCDPTAHAEIVALRAAARHLGNYRLEGCTLYVTLEPCAMCAGAMLHARLDRLVYGVDNPKAGAAGSVLDVLGHPRLNHRLEVTGGVLADECGQLLRDFFRAR

SEQ ID NO: 771 - protein, 147 aa, synthetic construct
MNDKGWMRLALEEARAARAAGEVPVGAVVVRDGEVIAAAGNRRRELCDPTAHAEIVALREAARRLGNYRLPGCTLYVTLEPCAMCAGAMIHARLDRLVFGVANPKAGAAGSVLDVLGHPRLNHRLEVTGGVLEEECGQLLRDFFRAR

SEQ ID NO: 772 - protein, 147 aa, synthetic construct
MSDEHFMQQALAEARKAYDLGEVPVGAVVVRDGRIIARGHNQVEKLKDPTAHAEMLAITAAAGHLGSKYLEGCTLYVTLEPCIMCAGACVWARVERVVYGVRNPKAGACGSVFDIPGDPRLNHHPEVTGGVLEAECARLMRDFFRER

SEQ ID NO: 773 - protein, 147 aa, synthetic construct
MSDEFFMKQALEEARLAYDEGEVPVGAVVVRDGEVIARGRNQIEELKDPTAHAEMLAITAAAGHLGSKYLLGCTLYVTLEPCVMCAGACVWARVDRVVYGVRNPKAGAAGSVFDIPGDPRLNHHPEVVGGVLQAECAALMRDFFRER

SEQ ID NO: 774 - protein, 365 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSDFFRMRRQEIKAQKKAQSSTDSGGSSGGSSGSETPGTSESATPESSGGSSGGSSEVEFDDAGFMRLALEEARAAAAAGEVPVGAVVVRDGEVLARAGNRTVRDCDPTAHAEIVALREAARRLGNYRLAGCTLYVTLEPCAMCAGAMIHARLDRLVYGVANPKAGAAGSVLDVLGHPRLNHRMEVEGGVLAAECGALLRDFFRAPRRVFNAQKKAQSSTD

SEQ ID NO: 775 - protein, 365 aa, synthetic construct
MSEVEFTDEYFMRQALREARRAYDEGEVPVGAVVVRDGRVIARGRNQVERLKDPTAHAEILALRAACRHLGSKYLTGCTLYVTVEPCAMCAGALVWARVERVVFGVRNPKAGACGSVLDIPGDRRLNHRPEVVGGVLEAECAALMRDFFREPRRVFNAQKKAQSSTDSGGSSGGSSGSETPGTSESATPESSGGSSGGSSEVEFTDEYFMRQALREARRAYDEGEVPVGAVVVRDGRVIARGRNQVERLKDPTAHAEILALRAACRHLGSKYLTGCTLYVTVEPCAMCAGALVWARVERVVFGVRNPKAGACGSVLDIPGDRRLNHRPEVVGGVLEAECAALMRDFFREPRRVFNAQKKAQSSTD

SEQ ID NO: 776 - protein, 149 aa, synthetic construct
MDPTDLAFMREALAEARAAAEAGEVPVGAVVVCDGRIVARAHNRPIALSDPTAHAEILALREAARALGNYRLTGCTLYATLEPCAMCAGAILHARIARLVYGVANPGTGACGSVLDVMNHPRLNHRVEVTGGVLAEECGALLSGFFRAR

SEQ ID NO: 777 - RNA, 20 nt, synthetic construct
  source 1..20: mol_type = other RNA
gaacacaaagcatagactgc

SEQ ID NO: 778 - RNA, 20 nt, synthetic construct
  source 1..20: mol_type = other RNA
ggcccagactgagcacgtga

SEQ ID NO: 779 - RNA, 20 nt, synthetic construct
  source 1..20: mol_type = other RNA
acatacttctgtaatacact

SEQ ID NO: 780 - RNA, 20 nt, synthetic construct
  source 1..20: mol_type = other RNA
ggacagcttttcctagacag

SEQ ID NO: 781 - RNA, 20 nt, synthetic construct
  source 1..20: mol_type = other RNA
gagcagatcgttgattgtag

SEQ ID NO: 782 - DNA, 23 bp, synthetic construct
  source 1..23: mol_type = other DNA
ctggtggtacttgaatcaagcac

SEQ ID NO: 783 - DNA, 26 bp, synthetic construct
  source 1..26: mol_type = other DNA
gaaggagacttgtgcacattctatag

SEQ ID NO: 784 - DNA, 20 bp, synthetic construct
  source 1..20: mol_type = other DNA
atgtgggctgcctagaaagg

SEQ ID NO: 785 - DNA, 20 bp, synthetic construct
  source 1..20: mol_type = other DNA
cccagccaaacttgtcaacc

SEQ ID NO: 786 - DNA, 53 bp, synthetic construct
  source 1..53: mol_type = other DNA
acactctttccctacacgacgctcttccgatctcaagacctggctgagctaac

SEQ ID NO: 787 - DNA, 52 bp, synthetic construct
  source 1..52: mol_type = other DNA
gactggagttcagacgtgtgctcttccgatctaaattgtccagccccatctg

SEQ ID NO: 788 - DNA, 54 bp, synthetic construct
  source 1..54: mol_type = other DNA
acactctttccctacacgacgctcttccgatcttgatcggcttcctaactgaag

SEQ ID NO: 789 - DNA, 52 bp, synthetic construct
  source 1..52: mol_type = other DNA
gactggagttcagacgtgtgctcttccgatctaagctcaccagaacagcaaa

SEQ ID NO: 790 - DNA, 53 bp, synthetic construct
  source 1..53: mol_type = other DNA
acactctttccctacacgacgctcttccgatctctggcctgggtcaatccttg

SEQ ID NO: 791 - DNA, 52 bp, synthetic construct
  source 1..52: mol_type = other DNA
gactggagttcagacgtgtgctcttccgatcttccctaggtgctggcttcca

SEQ ID NO: 792 - protein, 4 aa, synthetic construct
KRKR

SEQ ID NO: 793 - protein, 4 aa, synthetic construct
KRRR

SEQ ID NO: 794 - protein, 4 aa, synthetic construct
KKRK

SEQ ID NO: 795 - protein, 4 aa, synthetic construct
KRRK

SEQ ID NO: 796 - protein, 7 aa, synthetic construct
  VARIANT 7: note = X is any amino acid except D or E
RXXKRKX

SEQ ID NO: 797 - protein, 7 aa, synthetic construct
  VARIANT 7: note = X is any amino acid except D or E
PXXKRKX

SEQ ID NO: 798 - protein, 7 aa, synthetic construct
  VARIANT 7: note = X is any amino acid except D or E
RXXKRRX

SEQ ID NO: 799 - protein, 7 aa, synthetic construct
  VARIANT 7: note = X is any amino acid except D or E
PXXKRRX

SEQ ID NO: 800 - protein, 7 aa, synthetic construct
  VARIANT 6: note = X is any amino acid except D or E
RXXKRXK

SEQ ID NO: 801 - protein, 7 aa, synthetic construct
  VARIANT 6: note = X is any amino acid except D or E
PXXKRXK

SEQ ID NO: 802 - protein, 7 aa, synthetic construct
  VARIANT 6: note = X is any amino acid except D or E
RXXKRXR

SEQ ID NO: 803 - protein, 7 aa, synthetic construct
  VARIANT 6: note = X is any amino acid except D or E
PXXKRXR

SEQ ID NO: 804 - protein, 8 aa, synthetic construct
KRXWXXAF

SEQ ID NO: 805 - protein, 8 aa, synthetic construct
KRXFXXAF

SEQ ID NO: 806 - protein, 8 aa, synthetic construct
KRXYXXAF

SEQ ID NO: 807 - protein, 6 aa, synthetic construct
  VARIANT 6: note = X is W, F, or Y
LGKRRX

SEQ ID NO: 808 - protein, 6 aa, synthetic construct
  VARIANT 6: note = X is W, F, or Y
LGKRKX